SEQUENCE LISTING (236 sequences)

SEQ ID NO: 1 - protein, 661 aa, synthetic construct
  source 1..661: note = Engineered reverse transcriptase (MMLV RT Variant)
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 2 - protein, 248 aa, synthetic construct
  source 1..248: note = Dat1 Amino acid NCBI Reference Sequence: NP_013594.1
MAKTLAQGRKPGSGRKPGKGKTLREGRKPGSGRRRRQDTGGKETDGSQQDQESRLISSRDMEAVDALRELTHSPSSHSAHNSSAAPPPHAAAASTSLPPSLDYTHQSFMDQQQQQQQQQQQQLLQQQRVDVVPPKPFITHKILLSSTGNSGGHVNSNYNADHSINHNSNHNLNSNVNVNMNFTINGSNQDPSSSFLMGPYNYLQRPFIVKPYLDLSTSTAASNQPRTQPSPAAHITKNSDSTEKNATI

SEQ ID NO: 3 - protein, 90 aa, synthetic construct
  source 1..90: note = Dat1 D-90
MAKTLAQGRKPGSGRKPGKGKTLREGRKPGSGRRRRQDTGGKETDGSQQDQESRLISSRDMEAVDALRELTHSPSSHSAHNSSAAPPPHA

SEQ ID NO: 4 - DNA, 10 bp, synthetic construct
  source 1..10: mol_type = other DNA; note = a poly-dT sequence
tttttttttt

SEQ ID NO: 5 - protein, 60 aa, synthetic construct
  source 1..60: note = Dat1 D-60
MAKTLAQGRKPGSGRKPGKGKTLREGRKPGSGRRRRQDTGGKETDGSQQDQESRLISSRD

SEQ ID NO: 6 - protein, 48 aa, synthetic construct
  source 1..48: note = Dat1 D-48
MAKTLAQGRKPGSGRKPGKGKTLREGRKPGSGRRRRQDTGGKETDGSQ

SEQ ID NO: 7 - protein, 671 aa, synthetic construct
  source 1..671: note = Wild-type MMLV GenBank Seq ID NP_955591.1 p80 RT
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLL

SEQ ID NO: 8 - protein, 36 aa, synthetic construct
  source 1..36: note = Dat1 D-36
MAKTLAQGRKPGSGRKPGKGKTLREGRKPGSGRRRR

SEQ ID NO: 9 - protein, 35 aa, synthetic construct
  source 1..35: note = Dat1 D-35
AKTLAQGRKPGSGRKPGKGKTLREGRKPGSGRRRR

SEQ ID NO: 10 - protein, 33 aa, synthetic construct
  source 1..33: note = TSO sequence
AAGCAGTGGTATCAACGCAGAGTACATRGRGRG

SEQ ID NO: 11 - protein, 5 aa, synthetic construct
  source 1..5: note = Dat1 AT-rich interaction domain- minimal
GRKPG

SEQ ID NO: 12 - protein, 8 aa, synthetic construct
  source 1..8: note = Dat1 AT-rich interaction domain
GRKPGSGR

SEQ ID NO: 13 - protein, 35 aa, synthetic construct
  source 1..35: note = Dat1 AT-rich interaction domain- consensus
XXTLAQGRKPGXGRKPXKGKTLXEGRKPGSGRRRR

SEQ ID NO: 14 - protein, 35 aa, synthetic construct
  source 1..35: note = Dat1 AT-rich interaction domain- consensus
XXTLAQGRXXGXGRXXXKGKTLXEGRXXGSGRRRR

SEQ ID NO: 15 - protein, 9 aa, Homo sapiens
  source 1..9: note = mammalian high mobility group I protein (HMG-I, or a-protein) AT-rich interaction domain
KRPRGRPKK

SEQ ID NO: 16 - protein, 8 aa, Drosophila melanogaster
  source 1..8: note = Drosophila melanogaster Dl AT-rich interaction domain-consensus
  VARIANT 1: note = N can be K or R
NKRGRPKK

SEQ ID NO: 17 - protein, 8 aa, Drosophila melanogaster
  source 1..8: note = Drosophila melanogaster Dl AT-rich interaction domain
RKRGRPKK

SEQ ID NO: 18 - protein, 8 aa, Drosophila melanogaster
  source 1..8: note = Drosophila melanogaster Dl AT-rich interaction domain
KKRGRPKK

SEQ ID NO: 19 - protein, 5 aa, synthetic construct
  source 1..5: note = AT-rich interaction domain
RKRGR

SEQ ID NO: 20 - protein, 4 aa, synthetic construct
  source 1..4: note = AT-rich interaction domain
PKRP

SEQ ID NO: 21 - protein, 4 aa, synthetic construct
  source 1..4: note = phage 434 repressor AT-rich interaction domain
KRPR

SEQ ID NO: 22 - protein, 5 aa, Salmonella sp.
  source 1..5: note = Hin recombinase of Salmonella typhimurium AT-rich interaction domain
  VARIANT 3: note = N can be R or P
GRNPK

SEQ ID NO: 23 - protein, 5 aa, Salmonella sp.
  source 1..5: note = Hin recombinase of Salmonella typhimurium AT-rich interaction domain
GRRPK

SEQ ID NO: 24 - protein, 5 aa, Salmonella sp.
  source 1..5: note = Hin recombinase of Salmonella typhimurium AT-rich interaction domain
GRPPK

SEQ ID NO: 25 - DNA, 746 bp, Saccharomyces cerevisiae
  source 1..746: mol_type = other DNA; note = Dat1 Nucleic acid sequence NC_001145.3:44045-44791 Saccharomyces cerevisiae S288C chromosome XIII
atggcgaaaactttggcacaaggaaggaaacctggaagcggcagaaagcccggaaaagggaagacgttgagagagggaagaaagcctggcagtggtaggaggaggaggcaagatactgggggtaaagagaccgacgggtctcagcaagatcaggagtcgcgtcttattagttccagggacatggaagctgtggacgcactgagagagttgacgcacagcccgtcgtctcatcagctcataattcatcagcagcaccaccgccgcatgcagcagcggcgtcaacttcattgcctccatctctggattatacccatcagtcattcatggatcaacaacaacaacagcagcagcagcagcagcaacaactactacagcaacagagagtggacgtcgttccgcccaagccgttcatcacccacaagatcctgttatcttcaacgggaaactctggcggccacgtgaattcaaattacaacgccgaccacagcattaatcataattcaaaccacaaccttaacagcaacgtgaacgtgaacatgaactttactataaatgggagcaatcaagacccgtcatcttctttcctgatggggccctacaactacttacagcggcctttcattgtgaaaccatatcttgatctgtccacaagcacagcagcatcaaatcagccacggacgcaaccatcgccggcagcacatatcactaagaattccgattctacagagaaaaacgcaaccatatga

SEQ ID NO: 26 - protein, 5 aa, synthetic construct
GGGGS

SEQ ID NO: 27 - protein, 661 aa, synthetic construct
MTWLSDFPQAWAETGGMGLAVRQAPLIIPLKATSTPVSIKQYPLSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSNARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 28 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 29 - protein, 649 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 30 - protein, 649 aa, synthetic construct
MTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 31 - protein, 649 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 32 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 33 - protein, 649 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 34 - protein, 671 aa, synthetic construct
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLP

SEQ ID NO: 35 - protein, 671 aa, synthetic construct
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNHMAHQAARRAAITETPDTSTLP

SEQ ID NO: 36 - protein, 671 aa, synthetic construct
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLP

SEQ ID NO: 37 - protein, 671 aa, synthetic construct
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNRMAHQAARRAAITETPDTSTLP

SEQ ID NO: 38 - protein, 671 aa, synthetic construct
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLP

SEQ ID NO: 39 - protein, 671 aa, synthetic construct
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLP

SEQ ID NO: 40 - protein, 671 aa, synthetic construct
TLNIEDEHRLHETSKEPDVSLGSTWLSDFPQAWAETGGVGLAVRQALLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLP

SEQ ID NO: 41 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 42 - protein, 649 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 43 - protein, 649 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 44 - protein, 649 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 45 - protein, 649 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 46 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPADRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 47 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 48 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 49 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 50 - protein, 649 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 51 - protein, 649 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 52 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGGQKGHSAEARGNRMADQAARKAAITETPDTSTLL

SEQ ID NO: 53 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGGQKGHSAEARGNRMADQAARKAAITETPDTSTLL

SEQ ID NO: 54 - protein, 649 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 55 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 56 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 57 - protein, 679 aa, synthetic construct
MGSSHHHHHHSSGLVPRGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 58 - protein, 679 aa, synthetic construct; identical to SEQ ID NO: 57

SEQ ID NO: 59 - protein, 661 aa, synthetic construct; identical to SEQ ID NO: 1

SEQ ID NO: 60 - protein, 662 aa, synthetic construct
GSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 61 - protein, 660 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 62 - protein, 5 aa, synthetic construct
HHHHH

SEQ ID NO: 63 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLPIENSSPNSRLIN

SEQ ID NO: 64 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgaagaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcacggcgagatctatcgccgtcgcggctggctgacgtccggcggcaaagagatcaagaataaagacgaaattctggcgctgctgaaagcgctgttcctgagcaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 65 - protein, 660 aa, synthetic construct
TWLSDFPQAWAETGGLGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 66 - DNA, 1980 bp, synthetic construct
  source 1..1980: mol_type = other DNA
acttggctgtctgatttccctcaggcgtgggccgaaacgggtggcctgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 67 - protein, 660 aa, synthetic construct
TWLSDFPQAWAETGGLGLAVRQAPLIIPLKATSTPVSIKQYPLSQKARLGIKPHIQRLLDQGILVPCQSPWNTPLLPVKKPGTSDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFVEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 68 - DNA, 1980 bp, synthetic construct
  source 1..1980: mol_type = other DNA
acttggctgtctgatttccctcaggcgtgggccgaaacgggtggcctgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgagcgattaccgtccagtccaagacttgcgcgaagttaacaagcgcgttgaagatattcacccgaccgtcccgaacccgtacaatctgctgagcggtctgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgtggaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 69 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTSDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 70 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgagcgattaccgtccagtccaagacttgcgcgaagttaacaagcgcgttgaagatattcacccgaccgtcccgaacccgtacaatctgctgagcggtctgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 71 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTSDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFVEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 72 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgagcgattaccgtccagtccaagacttgcgcgaagttaacaagcgcgttgaagatattcacccgaccgtcccgaacccgtacaatctgctgagcggtctgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgtggaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 73 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTSDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFVEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 74 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgagcgattaccgtccagtccaagacttgcgcgaagttaacaagcgcgttgaagatattcacccgaccgtcccgaacccgtacaatctgctgagcggtctgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgtggaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 75 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGRAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 76 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaaattcttgggccgcgcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 77 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 78 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtctgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 79 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTSDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCRQGTRALLQTLGNLGYRASAKKAQICQKQVKYLGYLLKEGQRWLTEARKETVMGQPTPKTPRQLRKFLGRAGSCRLHIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 80 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgagcgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccgccagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaaattcttgggccgcgcgggtagctgccgcctgcatatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 81 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 82 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccggcggcaaagagatcaagaataaagacgaaattctggcgctgctgaaagcgctgttcctgccgaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 83 - protein, 660 aa, synthetic construct
TWLSDFPQAWAETWGVGLAVRQALLIIPLKATSTPVSIKQYPMSQKARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLPIENSSPNSRLIN

SEQ ID NO: 84 - DNA, 1980 bp, synthetic construct
  source 1..1980: mol_type = other DNA
acttggctgtctgatttccctcaggcgtgggccgaaacgtggggcgtgggtctggcagtgcgtcaggcactgctgattattccgctgaaagcgacgtcgaccccggtgagcatcaagcaatatccgatgtcccaaaaggcgcgcttaggtattaagccgcacattcagcgtctgctggatcaaggtattctggttccgtgtcgcagcccgtggaataccccgcttctcccggtgaagaaaccgggcacgagcgattaccgtccagtccaagacttgcgcgaagttaacaagcgcgttgaagatattcgcccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcacggcgagatctatcgccgtcgcggctggctgacgtccggcggcaaagagatcaagaataaagacgaaattctggcgctgctgaaagcgctgttcctgagcaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 85 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLPIENSSPNPRLIN

SEQ ID NO: 86 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgaagaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacggatggtagcagcctgttgcaagagggtcagcgtaaggccggtgccgcggtcaccaccgagactggcgtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcagggcgagatctatcgccgtcgcggctttctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgccgatcgagaatagcagcccgaacccgcgtctgatcaat

SEQ ID NO: 87 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLPIENSSPNSRLIN

SEQ ID NO: 88 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcacggcgagatctatcgccgtcgcggctggctgacgtccggcggcaaagagatcaagaataaagacgaaattctggcgctgctgaaagcgctgttcctgagcaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 89 - protein, 660 aa, synthetic construct
TWLSDFPQAWAETWGVGLAVRQALLIIPLKATSTPVSIKQYPMSQKARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 90 - DNA, 1980 bp, synthetic construct
  source 1..1980: mol_type = other DNA
acttggctgtctgatttccctcaggcgtgggccgaaacgtggggcgtgggtctggcagtgcgtcaggcactgctgattattccgctgaaagcgacgtcgaccccggtgagcatcaagcaatatccgatgtcccaaaaggcgcgcttaggtattaagccgcacattcagcgtctgctggatcaaggtattctggttccgtgtcgcagcccgtggaataccccgcttctcccggtgaagaaaccgggcacgagcgattaccgtccagtccaagacttgcgcgaagttaacaagcgcgttgaagatattcgcccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccggcggcaaagagatcaagaataaagacgaaattctggcgctgctgaaagcgctgttcctgccgaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 91 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 92 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgaagaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccggcggcaaagagatcaagaataaagacgaaattctggcgctgctgaaagcgctgttcctgccgaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 93 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLPIENSSPNPRLIN

SEQ ID NO: 94 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacggatggtagcagcctgttgcaagagggtcagcgtaaggccggtgccgcggtcaccaccgagactggcgtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcagggcgagatctatcgccgtcgcggctttctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgccgatcgagaatagcagcccgaacccgcgtctgatcaat

SEQ ID NO: 95 - protein, 660 aa, synthetic construct
TWLSDFPQAWAETWGVGLAVRQALLIIPLKATSTPVSIKQYPMSQKARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLPIENSSPNPRLIN

SEQ ID NO: 96 - DNA, 1980 bp, synthetic construct
  source 1..1980: mol_type = other DNA
acttggctgtctgatttccctcaggcgtgggccgaaacgtggggcgtgggtctggcagtgcgtcaggcactgctgattattccgctgaaagcgacgtcgaccccggtgagcatcaagcaatatccgatgtcccaaaaggcgcgcttaggtattaagccgcacattcagcgtctgctggatcaaggtattctggttccgtgtcgcagcccgtggaataccccgcttctcccggtgaagaaaccgggcacgagcgattaccgtccagtccaagacttgcgcgaagttaacaagcgcgttgaagatattcgcccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacggatggtagcagcctgttgcaagagggtcagcgtaaggccggtgccgcggtcaccaccgagactggtgtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcagggcgagatctatcgccgtcgcggctttctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgccgatcgagaatagcagcccgaacccgcgtctgatcaat

SEQ ID NO: 97 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLPIENSSPNSRLIN

SEQ ID NO: 98 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcacggcgagatctatcgccgtcgcggctggctgacgtccggcggcaaagagatcaagaataaagacgaaattctggcgctgctgaaagcgctgttcctgagcaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 99 - protein, 660 aa, synthetic construct
TWLSDFPQAWAETGGVGLAVRQAPLIIPLKATSTPVSIKQYPMSQKARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLPIENSSPNSRLIN

SEQ ID NO: 100 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcgcagcccgtggaataccccgcttctcccggtgaagaaaccgggcacgagcgattaccgtccagtccaagacttgcgcgaagttaacaagcgcgttgaagatattcgcccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcacggcgagatctatcgccgtcgcggctggctgacgtccggcggcaaagagatcaagaataaagacgaaattctggcgctgctgaaagcgctgttcctgagcaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 101 - protein, 660 aa, synthetic construct
TWLSDFPQAWAETWGVGLAVRQALLIIPLKATSTPVSIKQYPMSQKARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 102 - DNA, 1980 bp, synthetic construct
  source 1..1980: mol_type = other DNA
acttggctgtctgatttccctcaggcgtgggccgaaacgtggggcgtgggtctggcagtgcgtcaggcactgctgattattccgctgaaagcgacgtcgaccccggtgagcatcaagcaatatccgatgtcccaaaaggcgcgcttaggtattaagccgcacattcagcgtctgctggatcaaggtattctggttccgtgtcgcagcccgtggaataccccgcttctcccggtgaagaaaccgggcacgagcgattaccgtccagtccaagacttgcgcgaagttaacaagcgcgttgaagatattcgcccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccggcggcaaagagatcaagaataaagacgaaattctggcgctgctgaaagcgctgttcctgccgaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 103 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLPIENSSPNSRLIN

SEQ ID NO: 104 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgaagaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcacggcgagatctatcgccgtcgcggctggctgacgtccggcggcaaagagatcaagaataaagacgaaattctggcgctgctgaaagcgctgttcctgagcaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 105 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGAAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLPIENSSPNPRLIN

SEQ ID NO: 106 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgcaaatttctgggtgcagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccgatggtagcagcctgctgcaggaaggtcagcgcaaagcaggtgcagcagtgaccaccgaaaccggtgtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcagggtgaaatttatcgccgccgcggttttctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgccgattgaaaacagcagcccgaacccgcgcctgattaac

SEQ ID NO: 107 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGYSAEARGNRMAVQAARKAAITETPDTSTLLIENSSPNPRLIN

SEQ ID NO: 108 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggttatagcgcagaagcacgcggtaaccgcatggcagtgcaggcagcacgcaaagcagcaattaccgaaaccccggataccagcaccctgctgattgaaaacagcagcccgaacccgcgcctgattaac

SEQ ID NO: 109 - protein, 660 aa, synthetic construct
TWLSDFPQAWAETGGVGLAVRQAPLIIPLKATSTPVSIKQYPMSQRARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 110 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagcgcgcacgcctgggtattaaaccgcatattcagcgcctgctggatcagggtattctggtgccgtgccgcagcccgtggaacaccccgctgctgccggtgaaaaaaccgggtaccagcgattatcgcccggtgcaggatctgcgcgaagtgaacaaacgcgtggaagatattcgcccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttgaagaagcactgcgccgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 111 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 112 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccgatgaaaccgaagtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcatggtgaaatttatcgccgccgcggttggctgaccagcggtggtaaagaaattaaaaacaaagatgaaattctggcactgctgaaagcactgtttctgccgaaacgcctgagcattattcattgcccgggtcatcagaaaggtcatagcgtggaagcacgcggtaaccgcatggcacatcaggcagcacgccgcgcagcaattaccgaaaccccggataccagcaccctgccgattgaaaacagcagcccgaacagccgcctgattaac

SEQ ID NO: 113 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLPIENSSPNPRLIN

SEQ ID NO: 114 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccgatggtagcagcctgctgcaggaaggtcagcgcaaagcaggtgcagcagtgaccaccgaaaccggtgtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcagggtgaaatttatcgccgccgcggttttctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgccgattgaaaacagcagcccgaacccgcgcctgattaac

SEQ ID NO: 115 - protein, 660 aa, synthetic construct
TWLSDFPQAWAETGGVGLAVRQAPLIIPLKATSTPVSIKQYPMSQRARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 116 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagcgcgcacgcctgggtattaaaccgcatattcagcgcctgctggatcagggtattctggtgccgtgccgcagcccgtggaacaccccgctgctgccggtgaaaaaaccgggtaccagcgattatcgcccggtgcaggatctgcgcgaagtgaacaaacgcgtggaagatattcgcccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 117 - protein, 660 aa, synthetic construct
TWLSDFPQAWAETGGVGLAVRQAPLIIPLKATSTPVSIKQYPMSQRARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 118 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagcgcgcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 119 - protein, 660 aa, synthetic construct
TWLSDFPQAWAETGGVGLAVRQAPLIIPLKATSTPVSIKQYPMSQRARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 120 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagcgcgcacgcctgggtattaaaccgcatattcagcgcctgctggatcagggtattctggtgccgtgccgcagcccgtggaacaccccgctgctgccggtgaaaaaaccgggtaccagcgattatcgcccggtgcaggatctgcgcgaagtgaacaaacgcgtggaagatattcgcccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttgaagaagcactgcgccgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgagccagccgctggtgattaaagcaccgcatgcagtggaagcactggtgaaacagccggcagaacgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccgatgaaaccgaagtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcatggtgaaatttatcgccgccgcggttggctgaccagcggtggtaaagaaattaaaaacaaagatgaaattctggcactgctgaaagcactgtttctgccgaaacgcctgagcattattcattgcccgggtcatcagaaaggtcatagcgtggaagcacgcggtaaccgcatggcacatcaggcagcacgccgcgcagcaattaccgaaaccccggataccagcaccctgccgattgaaaacagcagcccgaacagccgcctgattaac

SEQ ID NO: 121 - protein, 660 aa, synthetic construct
TWLSDFPQAWAETGGVGLAVRQAPLIIPLKATSTPVSIKQYPMSQRARLGIKPHIQRLLDQGILVPCRSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 122 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagcgcgcacgcctgggtattaaaccgcatattcagcgcctgctggatcagggtattctggtgccgtgccgcagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 123 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSVEARGNRMAHQAARRAAITETPDTSTLPIENSSPNSRLIN

SEQ ID NO: 124 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgtggaagcacgcggtaaccgcatggcacatcaggcagcacgccgcgcagcaattaccgaaaccccggataccagcaccctgccgattgaaaacagcagcccgaacagccgcctgattaac

SEQ ID NO: 125 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTNSRYAFATAHIQGEIYRRRGFLTSKGKEIKNKDEILALLKALFLPKRLSIIHCPGHQKGYSAEARVNRMADQAARKAAITETPDTSTLPIENSSPNSRLIN

SEQ ID NO: 126 - DNA, 1980 bp, synthetic construct
  source 1..1980: mol_type = other DNA
acctggctgagcgattttccgcaagcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttgaagaagcactgcgccgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgttaaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtactctgtttaactggggtccggatcagcagaaagcatatcaggaaattaaacaggcactgctgaccgcaccggcactgggtctgccggatctgaccaaaccttttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggttgcagcaattgcagtgctgaccaaagatgcaggtaaactgaccatgggtcagcctctggtgattggtgcaccgcatgcagtggaagcactggtgaaacagccggcaggtcgctggctgagcaaagcacgcatgacccattatcaggcactgttactggataccgatcgcgtgcagtttggtccggtggtggcactgaacccggcaaccctgctgccgctgccggaagaaggtctgcagcataactgcctggatattctggcagaagcacatggtacccgcccggatctgactgatcagccgctgcctgatgcagatcatacctggtataccaacggtagcagcctgttacaggaaggtcagcgtaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcagggtgaaatctatcgccgccgcggtttcctgaccagcaaaggtaaagaaattaaaaacaaagatgaaattctggcactgctgaaagcactgtttctgccgaaacgcctgagcattattcattgcccgggtcatcagaaaggttatagcgcagaagcacgcgtgaaccgcatggcagatcaggcagcacgcaaagcagcaattaccgaaaccccggataccagcaccctgccgattgaaaacagcagcccgaacagccgcctgattaac

SEQ ID NO: 127 - protein, 660 aa, synthetic construct
TWLSDFPQAWAETGGFGLAVRQAPLIIPLKATSTPVSIKQYPISQKARLGIKPHIQRLLDQGILVPCRSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTNSRYAFATAHIHGEIYRRRGWLTSGGKEIKNKDEILALLKALFLPKRLSIIHCPGHQKGHSVEARDNRMAHQAARRAAITETPDTSTLPIENSSPNSRLIN

SEQ ID NO: 128 - DNA, 1980 bp, synthetic construct
  source 1..1980: mol_type = other DNA
acctggctgagcgattttccgcaggcatgggcagaaaccggtggttttggtctggcagtgcgccaggcaccgctgattattccgctgaaagcaaccagcaccccggtgagcattaaacagtatccgattagccagaaagcacgcctgggtattaaaccgcatattcagcgcctgctggatcagggtattctggtgccgtgccgcagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcatggtgaaatttatcgccgccgcggttggctgaccagcggtggtaaagaaattaaaaacaaagatgaaattctggcactgctgaaagcactgtttctgccgaaacgcctgagcattattcattgcccgggtcatcagaaaggtcatagcgtggaagcacgcgataaccgcatggcacatcaggcagcacgccgcgcagcaattaccgaaaccccggataccagcaccctgccgattgaaaacagcagcccgaacagccgcctgattaac

SEQ ID NO: 129 - protein, 660 aa, synthetic construct
TWLSDFPQAWAETGGVGLAVRQAPLIIPLKATSTPVSIKQYPISQKARLGIKPHIQRLLDQGILVPCRSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEVKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIQGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 130 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgattagccagaaagcacgcctgggtattaaaccgcatattcagcgcctgctggatcagggtattctggtgccgtgccgcagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaagtgaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcagggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 131 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 132 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgagccagccgctggtgattaaagcaccgcatgcagtggaagcactggtgaaacagccggcagaacgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccgatgaaaccgaagtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcatggtgaaatttatcgccgccgcggttggctgaccagcggtggtaaagaaattaaaaacaaagatgaaattctggcactgctgaaagcactgtttctgccgaaacgcctgagcattattcattgcccgggtcatcagaaaggtcatagcgtggaagcacgcggtaaccgcatggcacatcaggcagcacgccgcgcagcaattaccgaaaccccggataccagcaccctgccgattgaaaacagcagcccgaacagccgcctgattaac

SEQ ID NO: 133 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 134 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgagccagccgctggtgattaaagcaccgcatgcagtggaagcactggtgaaacagccggcagaacgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 135 - protein, 631 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYGGGGSVTVKFKYKGEELEVDISKIKKVWRVGKMISFTYDDNGKTGRGAVSEKDAPKELLQMLEKSGKK

SEQ ID NO: 136 - DNA, 1893 bp, synthetic construct
  source 1..1893: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagagccgagttaattgcgttaacacaggcgctgaagatggctgaagggaaaaaactgaacgtctataccgacagccgctatggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 137 - protein, 543 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDGTGGGGVTVKFKYKGEELEVDISKIKKVWRVGKMISFTYDDNGKTGRGAVSEKDAPKELLQMLEKSGKK

SEQ ID NO: 138 - DNA, 1629 bp, synthetic construct
  source 1..1629: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatgggacgggaggaggcggagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 139 - protein, 560 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPGGGGSVTVKFKYKGEELEVDISKIKKVWRVGKMISFTYDDNGKTGRGAVSEKDAPKELLQMLEKSGKK

SEQ ID NO: 140 - DNA, 1680 bp, synthetic construct
  source 1..1680: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 141 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTSDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFVEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 142 - DNA, 1980 bp, synthetic construct
  source 1..1980: mol_type = other DNA
acctggctgagcgattttccgcaagcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccagcgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttgtggaagcactgcgccgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgttaaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtactctgtttaactggggtccggatcagcagaaagcatatcaggaaattaaacaggcactgctgaccgcaccggcactgggtctgccggatctgaccaaaccttttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggttgcagcaattgcagtgctgaccaaagatgcaggtaaactgaccatgggtcagcctctggtgattggtgcaccgcatgcagtggaagcattagtgaaacagccggcaggtcgctggctgagcaaagcacgcatgacccattatcaggcactgttactggataccgatcgcgtgcagtttggtccggtggtggcactgaacccggcaaccctgctgccgctgccggaagaaggtctgcagcataactgcctggatattctggcagaagcacatggtacccgcccggatctgactgatcagccgctgcctgatgcagatcatacctggtataccaacggtagcagcctgttacaggaaggtcagcgtaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatctatcgccgccgcggttggctgaccagcaaaggtaaagaaattaaaaacaaagatgagatcctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 143 - protein, 660 aa, synthetic construct
TWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 144 - DNA, 1980 bp, synthetic construct
  source 1..1980: mol_type = other DNA
acttggctgtctgatttccctcaggcgtgggccgaaacgggtggcatgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 145 - protein, 660 aa, synthetic construct; identical to SEQ ID NO: 61

SEQ ID NO: 146 - DNA, 1980 bp, synthetic construct
  source 1..1980: mol_type = other DNA
acttggctgtctgatttccctcaggcgtgggccgaaacgggtggcatgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 147 - protein, 660 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 148 - DNA, 1980 bp, synthetic construct
  source 1..1980: mol_type = other DNA
acttggctgtctgatttccctcaggcgtgggccgaaacgggtggcatgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggtgcaccgcatgcagtcgaggcccttgttaagcaaccgccggatagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcggtgggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatctattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 149 - protein, 728 aa, synthetic construct
TWLSDFPQAWAETGGVGLAVRQAPLIIPLKATSTPVSIKQYPISQKARLGIKPHIQRLLDQGILVPCRSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEVKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIQGEIYRRRGWLTSKGKEIKNKDEILALLKALFLPKRLSIIHCPGHQKGHSAEARGNRMADQAARKAAITETPDTSTLLIENSSPNSRLINGGGGSVTVKFKYKGEELEVDISKIKKVWRVGKMISFTYDDNGKTGRGAVSEKDAPKELLQMLEKSGKK

SEQ ID NO: 150 - DNA, 2184 bp, synthetic construct
  source 1..2184: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgattagccagaaagcacgcctgggtattaaaccgcatattcagcgcctgctggatcagggtattctggtgccgtgccgcagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaagtgaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcagggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaacggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 151 - protein, 728 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLPKRLSIIHCPGHQKGHSVEARGNRMAHQAARRAAITETPDTSTLPIENSSPNSRLINGGGGSVTVKFKYKGEELEVDISKIKKVWRVGKMISFTYDDNGKTGRGAVSEKDAPKELLQMLEKSGKK

SEQ ID NO: 152 - DNA, 2184 bp, synthetic construct
  source 1..2184: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccgatgaaaccgaagtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcatggtgaaatttatcgccgccgcggttggctgaccagcggtggtaaagaaattaaaaacaaagatgaaattctggcactgctgaaagcactgtttctgccgaaacgcctgagcattattcattgcccgggtcatcagaaaggtcatagcgtggaagcacgcggtaaccgcatggcacatcaggcagcacgccgcgcagcaattaccgaaaccccggataccagcaccctgccgattgaaaacagcagcccgaacagccgcctgattaacggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 153 - protein, 728 aa, synthetic construct
TWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGGGGSVTVKFKYKGEELEVDISKIKKVWRVGKMISFTYDDNGKTGRGAVSEKDAPKELLQMLEKSGKK

SEQ ID NO: 154 - DNA, 2184 bp, synthetic construct
  source 1..2184: mol_type = other DNA
acctggctgtctgactttccgcaggcttgggcagagaccggggggatgggtttagcggtacggcaggcaccactgattataccgctgaaagcaacatcgactcctgtctcgataaagcaataccctatgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagagccgagttaattgcgttaacacaggcgctgaagatggctgaagggaaaaaactgaacgtctataccgacagccgctatgcatttgctacagcccatattcatggagaaatttatcgccggcgaggttggctcacgtcgaaaggtaaagaaataaaaaataaggatgaaattctggcacttctgaaagcgttatttctgcctaaacgcttaagtatcatacactgtcctggtcaccaaaaaggtcatagtgctgaagcgcgcgggaaccgcatggccgatcaggccgcccgtaaagctgcaattaccgaaacccccgatacatctacgctgctgattgaaaactctagcccaaatagcagactgattaatggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 155 - protein, 728 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGGGGSVTVKFKYKGEELEVDISKIKKVWRVGKMISFTYDDNGKTGRGAVSEKDAPKELLQMLEKSGKK

SEQ ID NO: 156 - DNA, 2184 bp, synthetic construct
  source 1..2184: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagagccgagttaattgcgttaacacaggcgctgaagatggctgaagggaaaaaactgaacgtctataccgacagccgctatgcatttgctacagcccatattcatggagaaatttatcgccggcgaggttggctcacgtcgaaaggtaaagaaataaaaaataaggatgaaattctggcacttctgaaagcgttatttctgcctaaacgcttaagtatcatacactgtcctggtcaccaaaaaggtcatagtgctgaagcgcgcgggaaccgcatggccgatcaggccgcccgtaaagctgcaattaccgaaacccccgatacatctacgctgctgattgaaaactctagcccaaatagcagactgattaatggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 157 - protein, 728 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGGGGSVTVKFKYKGEELEVDISKIKKVWRVGKMISFTYDDNGKTGRGAVSEKDAPKELLQMLEKSGKK

SEQ ID NO: 158 - DNA, 2184 bp, synthetic construct
  source 1..2184: mol_type = other DNA
acctggctgtctgactttccgcaggcttgggcagagaccgggggggtgggtttagcggtacggcaggcaccactgattataccgctgaaagcaacatcgactcctgtctcgataaagcaataccctatgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagagccgagttaattgcgttaacacaggcgctgaagatggctgaagggaaaaaactgaacgtctataccgacagccgctatgcatttgctacagcccatattcatggagaaatttatcgccggcgaggttggctcacgtcgaaaggtaaagaaataaaaaataaggatgaaattctggcacttctgaaagcgttatttctgcctaaacgcttaagtatcatacactgtcctggtcaccaaaaaggtcatagtgctgaagcgcgcgggaaccgcatggccgatcaggccgcccgtaaagctgcaattaccgaaacccccgatacatctacgctgctgattgaaaactctagcccaaatagcagactgattaatggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 159 - protein, 574 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLGGGGSVTVKFKYKGEELEVDISKIKKVWRVGKMISFTYDDNGKTGRGAVSEKDAPKELLQMLEKSGKK

SEQ ID NO: 160 - DNA, 1722 bp, synthetic construct
  source 1..1722: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctaggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 161 - protein, 579 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRGGGGSVTVKFKYKGEELEVDISKIKKVWRVGKMISFTYDDNGKTGRGAVSEKDAPKELLQMLEKSGKK

SEQ ID NO: 162 - DNA, 1737 bp, synthetic construct
  source 1..1737: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 163 - protein, 605 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRGGGGSVTVKFKYKGEELEVDISKIKKVWRVGKMISFTYDDNGKTGRGAVSEKDAPKELLQMLEKSGKK

SEQ ID NO: 164 - DNA, 1815 bp, synthetic construct
  source 1..1815: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagaggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 165 - skipped

SEQ ID NO: 166 - protein, 658 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNGGGGSVTVKFKYKGEELEVDISKIKKVWRVGKMISFTYDDNGKTGRGAVSEKDAPKELLQMLEKSGKK

SEQ ID NO: 167 - DNA, 1974 bp, synthetic construct
  source 1..1974: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagagccgagttaattgcgttaacacaggcgctgaagatggctgaagggaaaaaactgaacgtctataccgacagccgctatgcatttgctacagcccatattcatggagaaatttatcgccggcgaggttggctcacgtcgaaaggtaaagaaataaaaaatggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 168 - protein, 654 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKGGGGSVTVKFKYKGEELEVDISKIKKVWRVGKMISFTYDDNGKTGRGAVSEKDAPKELLQMLEKSGKK

SEQ ID NO: 169 - DNA, 1962 bp, synthetic construct
  source 1..1962: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagagccgagttaattgcgttaacacaggcgctgaagatggctgaagggaaaaaactgaacgtctataccgacagccgctatgcatttgctacagcccatattcatggagaaatttatcgccggcgaggttggctcacgtcgaaaggtaaaggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 170 - protein, 728 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLINGGGGSVTVKFKYKGEELEVDISKIKKVWRVGKMISFTYDDNGKTGRGAVSEKDAPKELLQMLEKSGKK

SEQ ID NO: 171 - DNA, 2184 bp, synthetic construct
  source 1..2184: mol_type = other DNA
acttggctgtctgatttccctcaggcgtgggccgaaacgggtggcatgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggtgcaccgcatgcagtcgaggcccttgttaagcaaccgccggatagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggctgtgggaacccgcccagaccttactgaccaaccacttcccgatgctgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatctattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaatggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 172 - protein, 671 aa, synthetic construct; identical to SEQ ID NO: 7

SEQ ID NO: 173 - protein, 660 aa, synthetic construct
  source 1..660: note = RTx N-tag: His SOLD 33 VDG SOLD 33 VDG
TWLSDFPQAWAETGGVGLAVRQAPLIIPLKATSTPVSIKQYPISQKARLGIKPHIQRLLDQGILVPCRSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 174 - protein, 755 aa, synthetic construct
  source 1..755: note = RTx N-tag: His C-Tag: Dat90 C-DAT 42B L42BL_Dat1-90
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGSGGSGAKTLAQGRKPGSGRKPGKGKTLREGRKPGSGRRRRQDTGGKETDGSQQDQESRLISSRDMEAVDALRELTHSPSSHSAHNSSAAPPPHA

SEQ ID NO: 175 - protein, 755 aa, synthetic construct
  source 1..755: note = RTx N-tag: His Dat90 N-DAT 42B L Dat1 90_42BL
AKTLAQGRKPGSGRKPGKGKTLREGRKPGSGRRRRQDTGGKETDGSQQDQESRLISSRDMEAVDALRELTHSPSSHSAHNSSAAPPPHAGSGGSGTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 176 - protein, 755 aa, synthetic construct
  source 1..755: note = RTx N-tag: His-Dat90 N-DAT-90 SOLD 01 N-DAT-90 SOLD 01
AKTLAQGRKPGSGRKPGKGKTLREGRKPGSGRRRRQDTGGKETDGSQQDQESRLISSRDMEAVDALRELTHSPSSHSAHNSSAAPPPHAGSGGSGTWLSDFPQAWAETGGLGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 177 - protein, 755 aa, synthetic construct
  source 1..755: note = RTx N-tag: His C-Tag: Dat90 C-DAT-90 SOLD 01 C-DAT-90 SOLD 01
TWLSDFPQAWAETGGLGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGSGGSGAKTLAQGRKPGSGRKPGKGKTLREGRKPGSGRRRRQDTGGKETDGSQQDQESRLISSRDMEAVDALRELTHSPSSHSAHNSSAAPPPHA

SEQ ID NO: 178 - protein, 755 aa, synthetic construct
  source 1..755: note = RTx N-tag: His-Dat90 N-DAT 42B N-DAT-90 42B
AKTLAQGRKPGSGRKPGKGKTLREGRKPGSGRRRRQDTGGKETDGSQQDQESRLISSRDMEAVDALRELTHSPSSHSAHNSSAAPPPHAGSGGSGTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 179 - protein, 755 aa, synthetic construct
  source 1..755: note = RTx N-tag: His Dat90 N-DAT 50A+ G N-DAT-90 50A+ G
AKTLAQGRKPGSGRKPGKGKTLREGRKPGSGRRRRQDTGGKETDGSQQDQESRLISSRDMEAVDALRELTHSPSSHSAHNSSAAPPPHAGSGGSGTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 180 - protein, 755 aa, synthetic construct
AKTLAQGRKPGSGRKPGKGKTLREGRKPGSGRRRRQDTGGKETDGSQQDQESRLISSRDMEAVDALRELTHSPSSHSAHNSSAAPPPHAGSGGSGTWLSDFPQAWAETGGVGLAVRQAPLIIPLKATSTPVSIKQYPISQKARLGIKPHIQRLLDQGILVPCRSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 181 - protein, 701 aa, synthetic construct
AKTLAQGRKPGSGRKPGKGKTLREGRKPGSGRRRRGSGGSGTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 182 - protein, 701 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGSGGSGAKTLAQGRKPGSGRKPGKGKTLREGRKPGSGRRRR

SEQ ID NO: 183 - protein, 913 aa, synthetic construct
AKTLAQGRKPGSGRKPGKGKTLREGRKPGSGRRRRQDTGGKETDGSQQDQESRLISSRDMEAVDALRELTHSPSSHSAHNSSAAPPPHAAAASTSLPPSLDYTHQSFMDQQQQQQQQQQQQLLQQQRVDVVPPKPFITHKILLSSTGNSGGHVNSNYNADHSINHNSNHNLNSNVNVNMNFTINGSNQDPSSSFLMGPYNYLQRPFIVKPYLDLSTSTAASNQPRTQPSPAAHITKNSDSTEKNATIGSGGSGTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 184 - protein, 913 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGSGGSGAKTLAQGRKPGSGRKPGKGKTLREGRKPGSGRRRRQDTGGKETDGSQQDQESRLISSRDMEAVDALRELTHSPSSHSAHNSSAAPPPHAAAASTSLPPSLDYTHQSFMDQQQQQQQQQQQQLLQQQRVDVVPPKPFITHKILLSSTGNSGGHVNSNYNADHSINHNSNHNLNSNVNVNMNFTINGSNQDPSSSFLMGPYNYLQRPFIVKPYLDLSTSTAASNQPRTQPSPAAHITKNSDSTEKNATI

SEQ ID NO: 185 - protein, 755 aa, synthetic construct
AKTLAQGRKPGSGRKPGKGKTLREGRKPGSGRRRRQDIGGRDADASQQDQESRPISSRDMEAVDALRELTHSPSSHPAHNPTTAPPHVAGSGGSGTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 186 - protein, 736 aa, synthetic construct
AKTLAQGRKPGSGRKPGKGKTLREGRKPGSGRRRRLDEPDRAVRSKSQDAISSRDLEAVDALRELNNSPSGSGGSGTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 187 - protein, 755 aa, synthetic construct
AKTLAQGRKPGSGRKPGKGKTLREGRKPGSGRRRRVDEEATKIQSSELHGIEISNECILGSLQRNINNELSTRDMEAVDALRGLNTSPSGSGGSGTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 188 - protein, 770 aa, synthetic construct
  source 1..770: note = RTx N-Dat1-TL-XP683 42BL N-Dat1-TL-XP683 42BL
AKTLAQGRKPGSGRKPGKGKTLREGRKPGSGRKRRDGNISNENHLIHSNDTVTSTVNSSRNGSVSNAFTEQDLEALQALNSLNKSSTSPRSTSANTSATNILPSGSGGSGTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 189 - DNA, 2268 bp, synthetic construct
  source 1..2268: mol_type = other DNA; note = RTx N-tag: His C-Tag: Dat90 C-DAT 42B L42BL_Dat1-90
acttggctgtctgatttccctcaggcgtgggccgaaacgggtggcatgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaatggttcagggggttccggtgcaaagactttggcccaggggcgcaaaccgggtagcggtcgcaaacccggaaagggcaaaactttacgtgaaggccgcaagcctggatctggtcgtcgtcgccgccaagacacagggggtaaggagactgatggatcacaacaagaccaggagtcccgtctgatctcatcgcgcgacatggaggcagtagacgctttgcgcgaattaacccatagtccgagttcacactctgcccacaattcctctgctgcaccaccaccgcacgcctga

SEQ ID NO: 190 - DNA, 2268 bp, synthetic construct
  source 1..2268: mol_type = other DNA; note = RTx N-tag: His Dat90 N-DAT 42B L Dat1 90_42BL
gcaaagactttggcccaggggcgcaaaccgggtagcggtcgcaaacccggaaagggcaaaactttacgtgaaggccgcaagcctggatctggtcgtcgtcgccgccaagacacagggggtaaggagactgatggatcacaacaagaccaggagtcccgtctgatctcatcgcgcgacatggaggcagtagacgctttgcgcgaattaacccatagtccgagttcacactctgcccacaattcctctgctgcaccaccaccgcacgccggttcagggggttccggtacttggctgtctgatttccctcaggcgtgggccgaaacgggtggcatgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaattga

SEQ ID NO: 191 - DNA, 2742 bp, synthetic construct
  source 1..2742: mol_type = other DNA
gcaaaaactttggctcaagggcgcaaacctggatcaggccgcaagccggggaagggtaaaactttgcgcgaggggcgtaagcctggatcggggcgtcgtcgtcgtcaggatactggagggaaagaaactgacggctcccaacaggatcaagaaagccgcttgatttcctcccgcgacatggaagccgtcgatgcacttcgtgagcttacacatagcccgtcaagccatagcgcccataatagctcagccgcacccccaccacatgccgcagcggctagcacttcccttccaccatcgttagattacacgcatcaatcgttcatggatcagcaacaacaacaacagcaacagcaacagcagcagcttttacagcaacagcgtgtcgacgttgtccctccgaagccctttatcacccataagatcctgttgagctctactggcaactcaggcgggcacgttaacagcaactacaacgcagatcattcgatcaaccataattcaaatcacaaccttaattccaacgttaacgtgaacatgaactttacgattaacggtagcaatcaagacccatcatccagtttcttgatgggcccatacaactatttacaacgccctttcatcgtgaagccctatttggatttatcgacttctacggcagccagtaaccaaccgcgtacacagccttccccagcggcacatatcacaaagaatagcgatagtacggagaagaacgccacaattggttcagggggttccggtacttggctgtctgatttccctcaggcgtgggccgaaacgggtggcatgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaattga

SEQ ID NO: 192 - DNA, 2742 bp, synthetic construct
  source 1..2742: mol_type = other DNA
acttggctgtctgatttccctcaggcgtgggccgaaacgggtggcatgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaatggttcagggggttccggtgcaaaaactttggctcaagggcgcaaacctggatcaggccgcaagccggggaagggtaaaactttgcgcgaggggcgtaagcctggatcggggcgtcgtcgtcgtcaggatactggagggaaagaaactgacggctcccaacaggatcaagaaagccgcttgatttcctcccgcgacatggaagccgtcgatgcacttcgtgagcttacacatagcccgtcaagccatagcgcccataatagctcagccgcacccccaccacatgccgcagcggctagcacttcccttccaccatcgttagattacacgcatcaatcgttcatggatcagcaacaacaacaacagcaacagcaacagcagcagcttttacagcaacagcgtgtcgacgttgtccctccgaagccctttatcacccataagatcctgttgagctctactggcaactcaggcgggcacgttaacagcaactacaacgcagatcattcgatcaaccataattcaaatcacaaccttaattccaacgttaacgtgaacatgaactttacgattaacggtagcaatcaagacccatcatccagtttcttgatgggcccatacaactatttacaacgccctttcatcgtgaagccctatttggatttatcgacttctacggcagccagtaaccaaccgcgtacacagccttccccagcggcacatatcacaaagaatagcgatagtacggagaagaacgccacaatttga

SEQ ID NO: 193 - protein, 11 aa, synthetic construct
SEEDEEKEEDG

SEQ ID NO: 194 - protein, 6 aa, synthetic construct
  VARIANT 6: note = N can be Q or G
ENLYFN

SEQ ID NO: 195 - protein, 5 aa, synthetic construct
DDDDK

SEQ ID NO: 196 - protein, 4 aa, synthetic construct
IEGR

SEQ ID NO: 197 - protein, 5 aa, synthetic construct
  VARIANT 4: note = N can be R or G
LVPNS

SEQ ID NO: 198 - protein, 7 aa, synthetic construct
  VARIANT 6: note = N can be Q or G
LEVLFNP

SEQ ID NO: 199 - protein, 6 aa, synthetic construct
GSGGSG

SEQ ID NO: 200 - protein, 18 aa, synthetic construct
GGTTCAGGGGGTTCCGGT

SEQ ID NO: 201 - RNA, 1315 nt, synthetic construct
  source 1..1315: mol_type = other RNA; note = GAPDH (RNA) Template
agagggaaagcagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 202 - DNA, 29 bp, synthetic construct
  source 1..29: mol_type = other DNA
tggttgagcacagggtactttattgatgg

SEQ ID NO: 203 - DNA, 33 bp, synthetic construct
  source 1..33: mol_type = other DNA
aagcagtggtatcaacgcagagtacatrgrgrg

SEQ ID NO: 204 - DNA, 29 bp, synthetic construct
  source 1..29: mol_type = other DNA
tggttgagcacagggtactttattgatgg

SEQ ID NO: 205 - protein, 5 aa, synthetic construct
HHHHH

SEQ ID NO: 206 - protein, 671 aa, unidentified; identical to SEQ ID NO: 7

SEQ ID NO: 207 - protein, 661 aa, synthetic construct; identical to SEQ ID NO: 1
  source 1..661: note = MMLV RT Variant (42B) Identical to SEQ ID NO: 1

SEQ ID NO: 208 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 209 - protein, 661 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 210 - protein, 661 aa, synthetic construct
MTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 211 - protein, 661 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 212 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 213 - protein, 661 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 214 - protein, 671 aa, synthetic construct; identical to SEQ ID NO: 34

SEQ ID NO: 215 - protein, 671 aa, synthetic construct; identical to SEQ ID NO: 35

SEQ ID NO: 216 - protein, 671 aa, synthetic construct; identical to SEQ ID NO: 36

SEQ ID NO: 217 - protein, 671 aa, synthetic construct; identical to SEQ ID NO: 37

SEQ ID NO: 218 - protein, 671 aa, synthetic construct; identical to SEQ ID NO: 38

SEQ ID NO: 219 - protein, 671 aa, synthetic construct; identical to SEQ ID NO: 39

SEQ ID NO: 220 - protein, 671 aa, synthetic construct; identical to SEQ ID NO: 40

SEQ ID NO: 221 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 222 - protein, 661 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 223 - protein, 661 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 224 - protein, 661 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 225 - protein, 661 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 226 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPADRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 227 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 228 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 229 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 230 - protein, 661 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 231 - protein, 661 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 232 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGGQKGHSAEARGNRMADQAARKAAITETPDTSTLLIENSSPNSRLIN

SEQ ID NO: 233 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGGQKGHSAEARGNRMADQAARKAAITETPDTSTLLIENSSPNSRLIN

SEQ ID NO: 234 - protein, 661 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 235 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 236 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

(19) United States
(12) Patent Application Publication
(10) Pub. No.: US 2026/0317736 A1
(43) Pub. Date: Oct. 15, 2026

(54) SEQUENCE SPECIFIC DNA BINDING PROTEINS

(71) Applicant: 10X GENOMICS, INC., Pleasanton, CA (US)

(72) Inventors: Ruijie ZHANG, Pleasanton, CA (US); Derek Hunter VALLEJO, Pleasanton, CA (US); Katherine NAKAMA, Pleasanton, CA (US); Lorita BOGHOSPOR, Pleasanton, CA (US); Lauren GUTGESELL, Pleasanton, CA (US)

(21) Appl. No.: 19/481,831

(22) PCT Filed: Jun. 12, 2024
(86) PCT No.: PCT/US2024/033519

§ 371 Date: Nov. 5, 2025

Related U.S. Application Data
(60) Provisional application No. 63/472,726, filed on Jun. 13, 2023.
(60) Provisional application No. 63/622,402, filed on Jan. 18, 2024.

(51) Int. Cl. C12N 9/12; C12P 19/34

(52) U.S. Cl. CPC C12N 9/1276; C12P 19/34; C12Y 207/07049

(57) ABSTRACT

The disclosure provides recombinant reverse transcriptases (e.g, engineered reverse transcriptase polypeptides) comprising one or more DNA binding domains conjugated to a wild-type or a mutant reversed transcriptases that exhibit one or more altered reverse transcriptase related activities, such as but not limited to, altered template switching efficiency, altered transcription efficiency or both. The disclosure further provides compositions and kits comprising the recombinant reverse transcriptases (e.g., engineered reverse transcriptase polypeptides) and methods of producing, amplifying, or sequencing nucleic acid molecules using the recombinant reverse transcriptases

### CROSS REFERENCE

This application claims priority from U.S. Provisional Patent Application No. 63/472,726, filed Jun. 13, 2023, and U.S. Provisional Patent Application No. 63/622,402, filed Jan. 18, 2024. The entire contents of which are hereby incorporated by reference for all purposes.

### FIELD OF THE INVENTION

The present invention relates to the field of protein engineering, particularly development of recombinant reverse transcriptase variants that exhibit one or more improved properties of interest.

### BACKGROUND

A variety of single cell processing and analytical methods and systems are known in the art, including analysis of specific individual cells, analysis of different cell types within populations of differing cell types, analysis and characterization of large populations of cells for environmental, human health, or epidemiological forensic. However, these methods and systems remain inefficient for a variety of reasons, including spatial heterogeneity and insufficient transcript capture.

Specifically, cells within a tissue can have different morphology and/or function due to varied analyte levels (e.g., gene and/or protein expression). The specific position of a cell within a tissue (e.g., the cell's position relative to neighboring cells or the cell's position relative to the tissue microenvironment) can also affect the cell's morphology, differentiation, fate, viability, proliferation, behavior, signaling, and cross-talk with other cells in the tissue.

This spatial heterogeneity has been previously studied using techniques that only provide data for a small handful of analytes in the context of an intact tissue or a portion of a tissue. Specifically, these techniques can provide substantial analyte data for dissociated tissues (i.e., single cells). However, they fail to provide information regarding the position of a single cell in a biological sample (e.g., tissue sample) caused in part by inefficient transcript capture. Accordingly, there is a need for improved single cell processing and analytical methods with enhanced transcript capture. Specifically, there is a need for improved reverse transcriptases with improved sensitivity, efficiency, processivity, and transcript capture for single cell and/or spatial analysis applications. The present disclosure addresses this need.

### SUMMARY

One aspect of the present disclosure provides an engineered reverse transcriptase (RT) polypeptide comprising: (a) an RT polypeptide sequence; (b) a DNA binding domain, where the DNA binding domain is from a molecule capable of binding a minor groove of a nucleic acid; and (c) a linker connecting the RT polypeptide sequence and the DNA binding domain.

In some embodiments of the engineered RT polypeptide described herein, the DNA binding domain is located at the N-terminus of the RT polypeptide sequence. In some embodiments, the DNA binding domain is located at the C-terminus of the RT polypeptide sequence. In some embodiments, the linker is G(n)S(m)G(p), and (a) n=0, 1, 2, 3, 4, 5, 6, 7, 8, 9, 10 11, 12, 13, 14, 15, 16, 17, 18, 19 or 20; (b) m=0, 1, 2, 3, 4, 5, 6, 7, 8, 9, 10 11, 12, 13, 14, 15, 16, 17, 18, 19 or 20; (c) p=0, 1, 2, 3, 4, 5, 6, 7, 8, 9, 10 11, 12, 13, 14, 15, 16, 17, 18, 19 or 20; and (d)ₙ, m, and p are selected independently. In some embodiments, the linker is a glycine-serine (GS) linker selected from the group consisting of (GS)n, (GSGGS)n, (SGGSG)n, (GGGS)n, (GGSG)n, (GGSGG)n, (GSGSG)n, (GSGGG)n, GGGSG)n, and (GSSSG)n, where n represents an integer of at least 1. In some embodiments, the linker is GGGS. In some embodiments, the linker is SGGSG.

In some embodiments of the engineered RT polypeptide described herein, the DNA binding domain specifically recognizes adenine-thymine-rich region on a nucleic acid molecule. In some embodiments, the DNA binding domain specifically recognizes oligo(dA) or oligo(dT) tracts on a nucleic acid molecule. In some embodiments, the DNA binding domain comprises at least one AT-rich interaction domain. In some embodiments, the DNA binding domain comprises at least 2, at least 3, at least 4, at least 5, at least 6, at least 7, at least 8, at least 9, or at least 10 AT-rich interaction domains.

In some embodiments, the AT-rich interaction domain comprises a core sequence, wherein the core sequence is a two base core sequence, a three base core sequence, a four base core sequence, or a five base core sequence.

In some embodiments, at least one of the bases of the core sequence comprises an arginine; a glycine and an arginine; a proline and an arginine; a lysine and an arginine; or any combination thereof. In some embodiments, the AT-rich interaction domain comprises a GRKPG (Gly-Arg-Lys-Pro-Gly) repeat, a RKRGRPKK repeat, a KKRGRPKK repeat, a RKRGR repeat, a GR*R/PPK repeat, a GR*RPK repeat, a GR*PPK repeat, a KRPR* repeat, or a K/RKRGRPKK repeat. In some embodiments, the AT-rich interaction domain comprises a core sequence comprising an amino acid selected from the group consisting of SEQ ID NO: 11-24.

In some embodiments of the engineered RT polypeptide described herein, the DNA binding domain is a DNA binding domain of any one of Saccharomyces cerevisiae datin (DAT1), high mobility group AT hook 1 (HMGA1), lysine-specific methyltransferase 2a (KMT2A), Myocyte Enhancer Factor 2C (MEF2C), Heterogeneous Nuclear Ribonucleoprotein D (HNRNPD), Structural Maintenance of Chromosomes 1A (SMC1), Structural Maintenance Of Chromosomes 2 (SMC2), Caenorhabditis elegans tbp-1, Drosophila melanogaster D1 protein, Salmonella typhimurium Hin recombinase, S. typhimurium Gin recombinase, S. typhimurium Pin recombinase, or S. typhimurium Cin recombinase, or a combination thereof.

In some embodiments, the DNA binding domain is from a S. cerevisiae DAT1. In some embodiments, the amino acid sequence of the DNA binding domain comprises a DNA binding domain consensus motif set forth in SEQ ID NO: 13, 14, 16, or 22.

In some embodiments, the DNA binding domain comprises: (a) a full-length DAT1 sequence or SEQ ID NO: 2; (b) an N-terminal truncated variant of DAT1 (D90) comprising the first 90 amino acids of the full length DAT1, or SEQ ID NO: 3; (c) a truncated variant of DAT1 (D60) comprising the first 60 amino acids of the full length DAT1 or SEQ ID NO: 5; (d) a truncated variant of DAT1 (D48) comprising the first 48 amino acids of the full length DAT1 or SEQ ID NO: 6; (e) a truncated variant of DAT1 (D36) comprising the first 36 amino acids of the full length DAT1 or SEQ ID NO: 8; (f) a truncated variant of DAT1 (D35) comprising the first 35 amino acids of full length DAT1 or SEQ ID NO: 9; (g) an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 2, 3, 5, 6, 8, or 9; or (h) an amino acid sequence having 90%, 91%, 92%, 93%, 94%, 95%, 96%, 97%, 98%, or 99% sequence identity to any one of SEQ ID NO: 2, 3, 5, 6, 8, or 9.

In some embodiments, the DNA binding domain comprises the amino acid sequence of SEQ ID NO: 11 (GRKPG). In some embodiments, the DNA binding domain optionally comprise at least 2 domains or at least 3 domains comprising SEQ ID NO: 11.

In some embodiments, the DNA binding domain comprises a mutation in any of one of SEQ ID NO: 2, 3, 5, 6, 8, 9, or 11. In some embodiments, the mutation is selected from a substitution, an insertion, a deletion, or any combination thereof.

In some embodiments of the engineered RT polypeptide described herein, the DNA binding domain comprises SEQ ID NO: 2. In some embodiments of the engineered RT polypeptide described herein, the DNA binding domain comprises the amino acid sequence of SEQ ID NO: 3, 8, or 9.

In some embodiments of the engineered RT polypeptide described herein, the RT polypeptide sequence comprises the amino acid sequence of SEQ ID NO: 7, and further comprises a combination of mutations selected from the group consisting of: (i) E69K, L139P, E302R, T306K, W313F, T330P, and N454K; and additionally one or more of M39V, P47L, Q91R, M66L, F155Y, D200N, D200E, H204R, G429S, L435G, L435K, P448A, D449G, H503V, D524N, T542D, E545G, D583N, H594Q, L603W, L603F, E607K, E607G, P627S, H634Y, H638G, A644V, D653H, K658R and L671P; and (ii) E69K, L139P, D200N, E302R, T306K, W313F, T330P, L435G, P448A, D449G, N454K, D524N, L603W, and E607K; and additionally one or more of M39V, P47L, M66L, Q91R, F155Y, H204R, G429S, H503V, T542D, E545G, D583N, H594Q, P627S, H634Y, H638G, A644V, D653H, K658R and L671P.

In some embodiments of the engineered RT polypeptide described herein, the amino acid sequence of the RT polypeptide sequence is: (a) at least 90% identical to SEQ ID NO: 1 or 143; (b) about 90% to about 99.99% identical to SEQ ID NO: 1 or 143, about 92% to about 99.99% identical to SEQ ID NO: 1 or 143, about 93% to about 99.99% identical to SEQ ID NO: 1 or 143, about 94% to about 99.99% identical to SEQ ID NO: 1 or 143, about 95% to about 99.99% identical to SEQ ID NO: 1 or 143, about 96% to about 99.99% identical to SEQ ID NO: 1 or 143, about 97% to about 99.99% identical to SEQ ID NO: 1 or 143, or about 98% to about 99.99% identical to SEQ ID NO: 1 or 143; or (c) about 90%, about 91%, about 92%, about 93%, about 94%, about 95%, about 96%, about 97%, about 98%, about 99% or about 99.5% identical to SEQ ID NO: 1 or 143.

In some embodiments, the RT polypeptide sequence comprises: (a) an amino acid sequence that is at least 95% identical to SEQ ID NO:1, 7, or 179, and; (b) a combination of mutations indexed to SEQ ID NO:7 or 178 selected from the group consisting of: (i) a combination of variants consisting of a T542D mutation, a D583N mutation, an E607G mutation, an A644V mutation, a D653H mutation, and a K658R mutation; and (ii) a combination of variants consisting of an E545G mutation, a D583N mutation, an H594Q mutation, an L603F mutation, and a S679P mutation.

In some embodiments, the amino acid sequence of the RT polypeptide sequence comprises E69K, L139P, D200N, E302R, T306K, W313F, T330P, N454K, H503V, D524N, L603W, E607K, and H634Y. In some embodiments, the amino acid variations are at any one position or combination thereof as identified in an alignment of SEQ ID NO: 1 or 143 to any one of the RT polypeptide sequences in Table 1 or Table 2.

In some embodiments, the RT polypeptide sequence comprises: (a) M39V, M66I, Q91R, I347V, and H594Q substitution in SEQ ID NO: 143; or (b) SEQ ID NO: 129 (SOLD 034). In some embodiments, the RT polypeptide sequence comprises M39V, T542D, D583N, E607G, A644V, D653H, K658R, and L671P in SEQ ID NO: 143. In some embodiments, the RT polypeptide sequence comprises: (a) M39V, T542D, D583N, E607G, A644V, D653H, K658R, L671P in SEQ ID NO: 143; or (b) SEQ ID NO: 111 (SOLD 025). In some embodiments, the RT polypeptide sequence comprises T542D, D583N, E607G, A644V, D653H, K658R, E545G, D583N, H594Q, and a L603F in SEQ ID NO: 143.

One aspect of the present disclosure provides an engineered RT polypeptide comprising: (a) an amino acid sequence that is at least 90%, at least 92%, at least 95%, at least 97%, at least 98%, or at least 99% identical to: (i) an amino acid sequence of an RT disclosed in Table 1, or Table 2; or (ii) SEQ ID NOs: 27-61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, 172, or 173; and (b) a DNA binding domain comprising an amino acid selected from the group consisting of SEQ ID NO: 2, 3, 5, 6, 8, and 9.

Another aspect of the present disclosure provides an engineered RT polypeptide comprising: (a) an amino acid sequence of an RT disclosed in Table 1 or Table 2; and (b) an amino acid sequence of DNA binding domain disclosed in Table 1.

In some embodiments, the engineered RT polypeptide comprises: (a) the amino acid sequence of any one of SEQ ID NO: 174-188; (b) an amino acid sequence having at least 90%, at least 91%, at least 92%, at least 93%, at least 94%, at least 95%, at least 96%, at least 97%, at least 98%, or at least 99% sequence identity to any one of SEQ ID NO: SEQ ID NO: 174-188; or (c) an amino acid sequence having 90%, 91%, 92%, 93%, 94%, 95%, 96%, 97%, 98%, or 99% sequence identity to any one of SEQ ID NO: SEQ ID NO: 174-188.

In some embodiments, the engineered RT comprises an amino acid sequence that is at least about 90% identical to an amino acid sequence selected from the group consisting of SEQ ID NO: 27-61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, 172, and 173.

In some embodiments, the RT polypeptide is 42B L (SEQ ID NO: 145), 50A+G (SEQ ID NO: 147), SOLD 022 (SEQ ID NO: 105), SOLD 023 (SEQ ID NO: 107), SOLD 025 (SEQ ID NO: 111), SOLD 031 (SEQ ID NO: 123), SOLD 033 (SEQ ID NO: 127), SOLD 034 (SEQ ID NO: 129), SOLD 035 (SEQ ID NO: 131), SOLD 001 (SEQ ID NO: 65), and SOLD 33 VDG (SEQ ID NO: 173), or an RT polypeptide set forth in SEQ ID NO: 143, or SEQ ID NO: 172.

In some embodiments of the engineered RT polypeptide described herein, the engineered RT comprises at least two DNA binding domains. In some embodiments, at least one DNA binding domain is located at the N-terminus of the engineered RT and at least one DNA binding domain is located at the C-terminus of the engineered RT. In some embodiments, the at least two DNA binding domains are both located at the C-terminus or N-terminus of the engineered RT.

One aspect of the present disclosure provides a recombinant reverse transcriptase (RT) protein comprising a RT polypeptide, fused to a DNA binding domain, where (a) the RT polypeptide and the DNA binding domain are separated by an amino acid linker, and (b) the DNA binding domain is fused to the C-terminus of the RT polypeptide.

One aspect of the present disclosure provides a recombinant reverse transcriptase (RT) protein comprising a RT polypeptide, fused to a DNA binding domain, where (a) the RT polypeptide and the DNA binding domain are separated by an amino acid linker, and (b) the DNA binding domain is fused to the N-terminus of the RT polypeptide.

In some embodiments, the RT polypeptide is any one of the RT polypeptides listed in Table 1 or Table 2.

In some embodiments of the recombinant reverse transcriptase (RT) protein described herein, the DNA binding domain is a DNA binding protein selected from the group consisting of S. cerevisiae datin (DAT1); high mobility group AT hook 1 (HMGA1), lysine-specific methyltransferase 2a (KMT2A), Myocyte Enhancer Factor 2C (MEF2C), Heterogeneous Nuclear Ribonucleoprotein D (HNRNPD), Structural Maintenance of Chromosomes 1A (SMC1), Structural Maintenance Of Chromosomes 2 (SMC2), C. elegans tbp-1, D. melanogaster D1 protein, Salmonella typhimurium Hin recombinase, S. typhimurium Gin recombinase, S. typhimurium Pin recombinase, or S. typhimurium Cin recombinase.

In some embodiments, the linker is a G(n)S(m)G(p) linker, where: (a) n=0, 1, 2, 3, 4, 5, 6, 7, 8, 9, 10 11, 12, 13, 14, 15, 16, 17, 18, 19 or 20; (b) m=0, 1, 2, 3, 4, 5, 6, 7, 8, 9, 10 11, 12, 13, 14, 15, 16, 17, 18, 19 or 20; (c) p=0, 1, 2, 3, 4, 5, 6, 7, 8, 9, 10 11, 12, 13, 14, 15, 16, 17, 18, 19 or 20; and (d) n, m, and p are selected independently. In some embodiments, the linker is a glycine-serine (GS) linker selected from the group consisting of (GS)n, (GSGGS)n, (SGGSG)n, (GGGS)n, (GGSG)n, (GGSGG)n, (GSGSG)n, (GSGGG)n, GGGSG)n, and (GSSSG)n, where n represents an integer of at least 1. In some embodiments, the linker is GGGS. In some embodiments, the linker is SGGSG.

In some embodiments, the DNA binding domain is a S. cerevisiae datin (DAT1) DNA binding domain or fragment thereof. In some embodiments, the DNA binding domain comprises: (a) an amino acid sequence selected from the group consisting of SEQ ID NO: 2, 3, 5, 6, 8, 9, and 11-24; or (b) a nucleic acid sequence of SEQ ID NO: 25.

In some embodiments, the RT polypeptide is selected from the group consisting of 42B L (SEQ ID NO: 145), 50A+G (SEQ ID NO: 147), SOLD 022 (SEQ ID NO: 105), SOLD 023 (SEQ ID NO: 107), SOLD 025 (SEQ ID NO: 111), SOLD 031 (SEQ ID NO: 123), SOLD 033 (SEQ ID NO: 127), SOLD 034 (SEQ ID NO: 129), SOLD 035 (SEQ ID NO: 131), SOLD 001 (SEQ ID NO: 65), SOLD 33 VDG (SEQ ID NO: 173), and an RT polypeptide set forth in SEQ ID NO: 143, SEQ ID NO: 172.

Another aspect of the present disclosure provides a recombinant RT protein as described herein comprising, consisting essentially of, or consisting of SEQ ID NO: 174-188.

In some embodiments of the engineered RT polypeptide described herein, or the recombinant RT protein described herein, the engineered RT polypeptide or the recombinant RT protein further comprises a tag protein selected from the group consisting of an affinity tag, a fluorescent tag, or an expression and/or solubility enhancement tag.

In some embodiments of the engineered RT polypeptide or the recombinant RT protein described herein, the tag is selected from hexahistidine tag (his-tag), small ubiquitin-like modifier tag (SUMO), a VariFlex C-Terminal solubility enhancement tag, a short peptide C-terminal tag, Thioredoxin (Trx) tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In some embodiments of the engineered RT polypeptide or the recombinant RT protein described herein, the tag is an affinity tag selected from hexahistidine tag (his-tag), Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In some embodiments of the engineered RT polypeptide or the recombinant RT protein described herein, the engineered RT polypeptide or the recombinant RT protein comprises: (a) an hexahistidine tag (his-tag); or (b) an amino acid sequence of SEQ ID NO: 62; or an amino acid sequence having at least 90% sequence identity to the amino acid sequence of SEQ ID NO: 62.

In some embodiments of the engineered RT polypeptide or the recombinant RT protein described herein, the engineered RT polypeptide or the recombinant RT protein comprises a solubility enhancer tag selected from the group consisting of a SUMO tag, a GST tag, a Trx tag, a VariFlex C-Terminal solubility enhancement tag, a short peptide C-terminal tag, an Fh8 tag, MBP tag, SET tag, GB1 tag, ZZ tag, HaloTag, SNUT tag, Skp tag, T7PK tag, EspA tag, Mocr tag, Ecotin tag, CaBO tag, ArsC tag, IF2-domain I tag, Expressivity tag, RpoA, tag, SlyD, tag, Tsf tag, RpoS tag, PotD tag, Crr tag, msyB tag, yigD tag, and rpoD tag.

In some embodiments of the engineered RT polypeptide or the recombinant RT protein described herein, the engineered RT polypeptide or the recombinant RT protein comprises: (a) a short peptide C-terminal tag; (b) an amino acid sequence of SEQ ID NO: 193; or (c) an amino acid sequence having at least 90% sequence identity to the amino acid sequence of SEQ ID NO: 193.

In some embodiments of the engineered RT polypeptide or the recombinant RT protein described herein, the tag further comprises: (a) an endoprotein cleavage sequence; (b) a cleavage sequence recognized by an endoprotein selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase (EnTK), gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin (Thr), tissue kallikrein, tobacco etch virus (TEV), togavirin, tryptophanyl aminopeptidase, U-plasminogen activator, V8, venombin A, venombin AB, factor Xa (Xa), and Xaa-pro aminopeptidase; or (c) an endoprotein cleavage sequence comprising the amino acid sequence of SEQ ID NO: 194, SEQ ID NO: 195, SEQ ID NO: 196, SEQ ID NO: 197, or SEQ ID NO: 198.

In some embodiments of the engineered RT polypeptide described herein, or the recombinant RT protein described herein, the engineered RT polypeptide or the recombinant RT protein exhibits increased template switching (TS) efficiency, increased processivity efficiency, increased binding affinity, increased transcription efficiency, increased chemical tolerance, improved ability to yield mitochondrial unique molecular identity (UMI) counts, improved ability to yield ribosomal unique molecular identity (UMI) counts, longer shelf life, higher strand displacement, higher end-to-end template jumping, or any combination thereof, when compared to an RT polypeptide or a recombinant RT protein lacking a conjugated DNA binding domain.

In some embodiments of the engineered RT polypeptide described herein, or the recombinant RT protein described herein, the engineered RT polypeptide or the recombinant RT protein comprises at least two or more of increased template switching (TS) efficiency, increased processivity efficiency, increased binding affinity, increased transcription efficiency, increased chemical tolerance, improved ability to yield mitochondrial unique molecular identity (UMI) counts, longer shelf life, higher strand displacement, higher end-to-end template jumping, or improved ability to yield ribosomal unique molecular identity (UMI) counts, when compared to an RT polypeptide or a recombinant RT protein lacking a conjugated DNA binding domain.

In some embodiments of the engineered RT polypeptide described herein or the recombinant RT protein described herein, the recombinant RT protein or the engineered RT exhibits increased transcript capture during amplification, when compared to an RT polypeptide or a recombinant RT protein lacking a conjugated DNA binding domain.

In some embodiments of the engineered RT polypeptide or the recombinant RT protein described herein, the DNA binding domain enhances the hybridization of a transcript and a primer during a nucleic acid amplification process, when compared to an RT polypeptide or a recombinant RT protein lacking a conjugated DNA binding domain.

In some embodiments of the engineered RT polypeptide or the recombinant RT protein described herein, the primer comprises a poly-dT or a poly(dT)VN sequence and a non-poly(dT) sequence; and the transcript comprises a poly-dA sequence.

In some embodiments of the engineered RT polypeptide or the recombinant RT protein described herein, the DNA binding domain stabilizes the oligo(A)-oligo(T) based transcript-primer complex during a nucleic acid amplification process.

In some embodiments of the engineered RT polypeptide or the recombinant RT protein described herein, the primer is a barcoded molecule.

In some embodiments of the engineered RT polypeptide or the recombinant RT protein described herein, the transcript is a nucleic acid molecule selected from an RNA, a mRNA, or a DNA.

One aspect of the present disclosure provides an isolated nucleic acid molecule encoding: (a) an engineered RT polypeptide described herein; or (b) a recombinant RT protein described herein.

In some embodiments, the nucleic acid molecule comprises a sequence selected from SEQ ID NO: 25, SEQ ID NO: 136, SEQ ID NO: 138, SEQ ID NO: 140, SEQ ID NO: 142, SEQ ID NO: 144, SEQ ID NO:146, SEQ ID NO: 148, SEQ ID NO: 150, SEQ ID NO: 152, SEQ ID NO: 154, SEQ ID NO:156, SEQ ID NO: 158, SEQ ID NO: 160, SEQ ID NO: 162, SEQ ID NO: 164, SEQ ID NO:167, SEQ ID NO:169, or SEQ ID NO: 171; or a nucleic acid sequence of Table 2.

One aspect of the present disclosure provides an expression vector comprising any isolated nucleic acid described herein.

One aspect of the present disclosure provides a host cell transfected with any expression vector described herein or any isolated nucleic acid described herein.

One aspect of the present disclosure provides a composition comprising: (a) any recombinant RT protein described herein; or (b) any engineered RT polypeptide described herein; or (d) any expression vector described herein; or (e) any host cell described herein; and (f) a buffer.

One aspect of the present disclosure provides a method for performing a reverse transcription reaction for generating a nucleic acid product from an RNA template comprising contacting under suitable conditions a biological sample or extract thereof with an engineered RT polypeptide described herein, or a recombinant RT protein describe herein.

In some embodiments, the biological sample or extract thereof comprises a cell, optionally the cell is permeabilized and/or optionally the cell is fixed. In some embodiments, the biological sample or extract thereof comprises a cell bead, optionally the cell bead is fixed. In some embodiments, the biological sample or extract thereof comprises a nucleus, optionally the nucleus is permeabilized and optionally the nucleus is fixed. In some embodiments, the biological sample or extract thereof comprises (a) a suitable cellular preparation selected from cell populations and/or single cells, or (b) a tissue.

In some embodiments, the biological sample or extract thereof comprises: (a) a cell, a cell bead, a permeabilized cell, a nucleus, where the nucleus is optionally permeabilized, and/or optionally the cell, the cell bead, the permeabilized cell and/or nucleus are fixed; (b) a suitable cellular preparation selected from cell populations and/or single cells; or (c) a tissue.

In some embodiments, the biological sample or extract thereof comprises cells in suspension, fresh cells, fixed cells, or cells and tissues immobilized on various solid surfaces.

In some embodiments, when the biological sample is a cell, a cell bead, or a nucleus, the reverse transcription reaction is part of a single cell RNA sequencing assay. In one embodiment, the single cell RNA sequencing assay further comprises, prior to the reverse transcription, partitioning the cell, the cell bead, or the nucleus into a partition. In one embodiment, the single cell RNA sequencing assay further comprises, after the reverse transcription reaction, hybridizing the nucleic acid product to an oligonucleotide molecule comprising a partition-specific barcode.

In some embodiments, when the biological sample is a cell or tissue sample immobilized on a surface, the reverse transcription reaction is part of a spatial RNA sequencing assay.

In some embodiments of the method described herein, the engineered RT polypeptide or the recombinant RT protein enhances template switching (TS) efficiency, processivity efficiency, binding affinity, transcription efficiency, chemical tolerance, ability to yield mitochondrial unique molecular identity (UMI) counts, ability to yield ribosomal unique molecular identity (UMI) counts, strand displacement, end-to-end template jumping, or any combination thereof, when compared to an RT polypeptide or a recombinant RT protein lacking a conjugated DNA binding domain.

In some embodiments, the engineered RT polypeptide or the recombinant RT protein enhances at least two or more of template switching (TS) efficiency, processivity efficiency, binding affinity, transcription efficiency, chemical tolerance, ability to yield mitochondrial unique molecular identity (UMI) counts, strand displacement, end-to-end template jumping, or ability to yield ribosomal unique molecular identity (UMI) counts, when compared to an RT polypeptide or a recombinant RT protein lacking a conjugated DNA binding domain.

In some embodiments, the recombinant RT protein or the engineered RT enhances transcript capture during amplification, when compared to an RT polypeptide or a recombinant RT protein lacking a conjugated DNA binding domain.

In some embodiments, the DNA binding domain of the recombinant RT protein or the engineered RT enhances the hybridization of a transcript and a primer during the amplification process, when compared to an RT polypeptide or a recombinant RT protein lacking a conjugated DNA binding domain.

In some embodiments, the engineered RT polypeptide or the recombinant RT protein comprises: (a) a DNA binding domain comprising an amino acid sequence selected from SEQ ID NO: 2, 3, 5, 6, 8, 9, or 11-24; and (b) an amino acid sequence selected from SEQ ID NOs: 27-61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, 172, or 173.

In some embodiments of the method described herein, the amino acid sequence of the engineered RT polypeptide or the recombinant RT protein comprises an amino acid sequence having at least about 90%, at least about 92%, at least about 95%, at least about 97%, at least about 98%, or at least about 99% sequence identity to SEQ ID NO: 174-188.

In some embodiments of the method described herein, the engineered RT polypeptide or the recombinant RT protein comprises: (a) M39V, M66I, Q91R, I347V, and H594Q substitution in SEQ ID NO: 143; (b) SEQ ID NO: 129 (SOLD 034); (c) SOLD 001 (SEQ ID NO: 65); or (d) SOLD 33 VDG (SEQ ID NO: 173).

In some embodiments, the engineered RT polypeptide or the recombinant RT protein comprises M39V, T542D, D583N, E607G, A644V, D653H, K658R, and L671P in SEQ ID NO: 1 or 143. In some embodiments, the engineered RT polypeptide or the recombinant RT protein comprises: (a) M39V, T542D, D583N, E607G, A644V, D653H, K658R, L671P in SEQ ID NO: 143; or (b) SEQ ID NO: 111 (SOLD 025).

In some embodiments, the engineered RT or the recombinant RT protein comprises a M39V, M66I, Q91R, I347V, H594Q in SEQ ID NO: 143. In some embodiments, the engineered RT polypeptide or the recombinant RT protein comprises an amino acid sequence that is at least about 90%, at least about 92%, at least about 95%, at least about 97%, at least about 98%, or at least about 99% identical to an amino acid sequence disclosed in Table 1 or Table 2.

One aspect of the present disclosure provides a method of using an engineered RT polypeptide described herein, or a recombinant RT protein described herein, the method comprising contacting the engineered RT polypeptide or the recombinant RT protein with a nucleic acid template under suitable conditions to produce a polymerized nucleic acid product, where the nucleic acid template comprises an RNA, a DNA, or a nucleic acid comprising an unnatural nucleotide. In some embodiments, the nucleic acid template comprises an RNA.

One aspect of the present disclosure provides a nucleic acid extension method comprising: (a) contacting a target nucleic acid molecule with an engineered reverse transcriptase polypeptide or a recombinant RT protein and a plurality of nucleic acid barcoded molecules comprising a barcode sequence, and (b) incubating the target nucleic acid, the engineered RT polypeptide or the recombinant RT protein and barcoded molecules under suitable conditions in which the barcoded molecules are extended by the engineered RT polypeptide or the recombinant RT protein. In some embodiments, the engineered RT polypeptide comprises the amino acid sequence of an engineered RT polypeptide described herein, or a recombinant RT protein described herein.

In some embodiments, the recombinant RT protein or the engineered RT polypeptide exhibits increased transcript capture during amplification. In some embodiments, the DNA binding domain enhances the hybridization of a transcript and a primer during a nucleic acid amplification process. In some embodiments, the primer comprises a poly-dT or a poly(dT)VN sequence and a non-poly(dT) sequence; and the transcript comprises a poly-dA sequence. In some embodiments, the DNA binding domain stabilizes the oligo(A)-oligo(T) based transcript-primer complex during a nucleic acid amplification process. In some embodiments, the primer is a barcoded molecule.

In some embodiments, the recombinant RT protein or the engineered RT polypeptide described herein performs the first strand complementary DNA (cDNA) reaction. In that embodiment, the first strand cDNA is amplified using a DNA polymerase to generate a second strand cDNA.

One aspect of the present disclosure provides a method of producing an engineered RT polypeptide or recombinant RT protein of the present disclosure, the method comprising providing a composition comprising a cell lysate and/or cellular fraction comprising the engineered RT polypeptide or the recombinant RT protein and subjecting the composition to protein purification steps so as to produce a substantially purified engineered RT enzyme.

One aspect of the present disclosure provides a kit comprising: (a) a recombinant RT protein described herein; or (b) an engineered reverse transcriptase polypeptide described herein; or (c) the isolated nucleic acid described herein; or (d) an expression vector described herein; or (e) a host cell described herein; or (f) a composition described herein; and (g) instructions.

Both the foregoing summary and the following description of the drawings and detailed description are exemplary and explanatory. They are intended to provide further details of the invention but are not to be construed as limiting. Other objects, advantages, and novel features will be readily apparent to those skilled in the art from the following detailed description of the invention.

Section headings, numerical and/or alphabetical listings, e.g., (a), (b), (i) etc., are presented merely for ease of reading the disclosure, including the specification and claims. The use of headings in the disclosure, including the specification or claims does not require the steps or elements be performed in alphabetical or numerical order or the order in which they are presented.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows and exemplary workflow for performing a templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIG. 12 shows a schematic diagram of a non-limiting embodiment of a generalized capture probe used in spatial transcriptomics and/or single cell transcriptomic analyses, exemplary applications in addition to general reverse transcription reactions where the engineered reverse transcriptase of the disclosure could be used to extend a capture probe using a captured target nucleic acid as a template, thereby generating a cDNA product.

FIG. 13 provides a schematic of an exemplary capillary electrophoresis (CE) validation assay process used to test the activity of candidate enzymes. In step 1, 5′-end labeled DNA primers were hybridized to RNA templates at room temperature (approx. 25° C.); and poly rG-labeled template switching oligonucleotides (rG-TSO) were added to the reaction mixture. In step 2, the temperature was raised to about 53° C. and first strand cDNA was synthesized with the addition of a poly-C tail (tailing). In step 3, template switching and TSO extension were performed. In step 4, the amplification product was transferred to a Genetic Analyzer for analysis.

FIGS. 14A-B provide schematics of an exemplary single cell and spatial assay for transcript capture. FIG. 14A illustrates a schematic process of the 5′ single cell assay and FIG. 14B illustrates a schematic process of the 3′ single cell assay and step 1 of Visium. The first step of both assays is the hybridization of an oligo(A) from an mRNA to an oligo(T)) of a primer and binding of reverse transcriptase to the annealed primer-template (which contains an oligo(A)-oligo(T) tract).

FIGS. 15A-B provide schematics of exemplary Visium/spatial 3′/5′ workflows. FIG. 15A illustrates a schematic of a Visium 3′ workflow demonstrating a polyA capture (box). FIG. 15B illustrates a schematic of a Visium ATP 5′ workflow demonstrating a polyA capture (box).

FIGS. 16A-B provide a non-limiting embodiment of a nucleic acid sequence (FIG. 16A) and an amino acid sequence (FIG. 16B) of an exemplary engineered reverse transcriptase (RT) polypeptide described in the present disclosure (N-Dat_42BL) comprising a DNA binding domain (e.g., DAT; bold and underline) operably linked to a reverse transcriptase (42BL) via a linker (bold).

FIGS. 17A-B provide a non-limiting embodiment of a nucleic acid sequence (FIG. 17A) and an amino acid sequence (FIG. 17B) of an exemplary engineered reverse transcriptase (RT) polypeptide described in the present disclosure (C-Dat_42BL) comprising a reverse transcriptase (42BL) operably linked to a DNA binding domain (e.g., DAT; bold and underline) via a linker (bold).

FIGS. 18A-B provide a non-limiting embodiment of a nucleic acid sequence (FIG. 18A) and an amino acid sequence (FIG. 18B) of an exemplary engineered reverse transcriptase (RT) polypeptide described in the present disclosure (N-DAT1full_42BL) comprising a DNA binding domain (e.g., full length DAT1 protein; bold and underline) operably linked to a reverse transcriptase (42BL) via a linker (bold).

FIGS. 19A-B provide a non-limiting embodiment of a nucleic acid sequence (FIG. 19A) and an amino acid sequence (FIG. 19B) of an exemplary engineered reverse transcriptase (RT) polypeptide described in the present disclosure (C-DAT1full_42BL) comprising a reverse transcriptase (42BL) operably linked to a DNA binding domain (e.g., full length DAT1 protein; bold and underline) via a linker (bold).

FIG. 20 shows the performance of two engineered RTs described herein (N-DAT 42BL; SEQ ID NO: 175) and C-DAT 42BL (SEQ ID NO: 174)) in a Single Cell 5′ (SC-5′) gene expression assay when compared to two control MMLV variants (SOLD 001 (SEQ ID NO: 65) and SOLD 33 VDG (SEQ ID NO: 175)); and illustrates the superiority of the DAT engineered RTs single cell assays over control non-DAT engineered RTs based on the median genes and UMIs per cell at 20 k and 50 k raw-reads per cell (rrpc).

FIG. 21 shows the performance of two engineered RTs described herein (N-DAT 42BL; SEQ ID NO: 175) and C-DAT 42BL (SEQ ID NO: 174)) in a Single Cell 5′ (SC-5′) gene expression assay when compared to two control MMLV variants (SOLD 001 (SEQ ID NO: 65) and SOLD 33 VDG (SEQ ID NO: 175)); and illustrates the superiority of the DAT engineered RTs for single cell assays over control non-DAT engineered RTs based on spatial transcriptomics and single cell transcriptomic analyses.

FIGS. 22A-B provide a bar graph (FIG. 22A) and a quantification (FIG. 22B) of the relative differences in performance between three reverse transcriptases when compared to a control RT and illustrating that a clear performance gains can be seen for a DAT fusion on either the N-terminal or C-terminal domain. In particular, FIGS. 22A-B show median genes and UMIs/cell at 50 k raw-reads per cell of three reverse transcriptases with and without a DAT fusion domain.

FIGS. 23A-D provide graphs illustrating the performance of various engineered RTs disclosed herein at maximum normalization depth; and showing median genes (FIG. 23A) and UMIs/cell (FIG. 23B) at maximum normalized read depth comparing library complexity of three reverse transcriptases with and without the DAT fusion domain. FIGS. 23C-D show saturation curves of the median genes (FIG. 23C) and counts/cell (FIG. 23C) as a function of read depth. At maximum normalized read depth, the benefit of the DAT fusion on each RT backbone can clearly be seen.

FIGS. 24A-F provide graphs illustrating the differential gene expression of some engineered RTs comprising DAT1 at the N-terminus. FIGS. 24A, C, and E feature scatter plots showing gene expression correlation of three reverse transcriptases with and without the DAT fusion domain. FIGS. 24B, D, and F feature volcano plots showing the number of differentially expressed genes between three reverse transcriptases with and without the DAT fusion domain.

FIGS. 25A-F provide graphs illustrating the differential gene expression of some engineered RTs comprising DAT1 at the C-terminus. FIGS. 25A, C, and E feature scatter plots showing gene expression correlation comparison of engineered RTs comprising N-Terminal DAT Domain and C-Terminal DAT Domain. FIGS. 25B (42BL versus 42BL NDAT), D, and F feature volcano plots comparing the number of differentially expressed genes between various RTs. Gains in performance were still present but were not as significant as a C-terminal DAT fusion. For example, the C-terminal DAT fusion did not perform as well as the N-terminal DAT fusion, but both N-terminal DAT fusion and C-terminal DAT fusion showed performance gains when compared to a control RT variant. The control RT variant was a non-DAT fusion RT comprising the same RT backbone (e.g., RT backbone alone; SEQ ID NO: 145).

FIGS. 26A-D provide performance comparison of the impact of the DAT domain across three reverse transcriptase backbones. FIGS. 26A-B summarize median genes (FIG. 26A) and UMIs/cell (FIG. 26B) at maximum normalization depth comparing the performance among three MMLV RT variants 42B, 42BL, and 50A+G backbones with and without a N-terminal DAT fusion domain; and illustrate a clear performance benefit from the DAT domain. FIG. 26C shows gene expression correlation. FIG. 26D shows differential gene expression. MMLV RT variants without a DAT fusion domain were used as controls.

FIG. 27 provides a schematic of exemplary Visium/spatial 3′ workflows highlighting the improvements associated with the engineered RT variant comprising DAT1 at the N-terminus (NDAT1) disclosed herein. Six areas of target optimization (stars) are shown when using the NDAT1 RT variant disclosed herein. During the permeabilization step, the NDAT1 RT variant reduced transcript mislocalization and increased target capture. Cleaving oligos after annealing and reducing steric hindrance also enhanced optimization. During reverse transcription, there was an improved template switching and increased RT efficiency. During the second strand synthesis, there was an improved synthesis efficiency and increased product. During the cDNA amplification, amplification was increased without additional noise, and enough sample for long reads was provided. During the repair, A-tailing, and ligation steps, repair and efficiency were improved and no data was lost.

FIGS. 28A-F provide UMI heat maps showing globally detected gene expression in two replicates of spatial assay performed using a first slide configuration, on a human tonsil tissue, using an NDAT1 MMLV RT variant (FIGS. 28C-F) or a control MMLV RT variant (FIGS. 28A-B) and three different buffer formulations-a commercially available RT reagent (FIG. 28A and FIG. 28C), Buffer X.4 (FIG. 28D), and Buffer X.5 (FIG. 28B, FIG. 28E, and FIG. 28F). In particular, FIGS. 28A-F show that NDAT1 RT variant improved the sensitivity of the assay while maintaining good spatial resolution when compared to the control RT variant, with all three buffer conditions.

FIGS. 29A-E provide graphs quantifying the quality, sensitivity, and detection of gene expression under the same six conditions shown in FIGS. 28A-F. In particular, the following metrics were evaluated: fraction reads in spots under tissue (FIG. 29A), reads mapped confidently to transcriptome (FIG. 29B), total genes detected (FIG. 29C), GRch38 median UMI counts per spot (30 k mapped spot-reads per spot) (FIG. 29D), and GRch38 median genes per spot (30 k mapped spot-reads per spot) (FIG. 29E). The quantification confirmed that NDAT1 RT variant improved the sensitivity of the assay while maintaining good spatial resolution when compared to the control RT variant (FIGS. 29D-E). A MMLV RT variant without a DAT fusion domain was used as a control.

FIGS. 30A-F provide UMI heat maps showing gene expression of RGS3 in a human tonsil tissue analyzed using NDAT1 RT variant (FIGS. 30C-F) or a control RT variant (FIGS. 30A-B) and three different buffer formulations-RT reagent (FIG. 30A and FIG. 30C), Buffer X.4 (FIG. 30D), and Buffer X.5 (FIG. 30B, FIG. 30E, and FIG. 30F). A MMLV RT variant without a DAT fusion domain was used as a control. Under sandwich configuration conditions, some evidence of undesirable flow of transcripts and/or target molecules or analytes (black circle on upper right quadrant indicates images with a tail) was observed. UMI heat maps are shown as a log 10 (UMI) ranging from cold to hot (0, 0.5, 1.0, 1.5, and 2.0).

FIGS. 31A-C provide UMI heat maps showing gene expression of KRT5 in two replicates of a spatial assay performed using a first slide configuration on human tonsil tissues analyzed using NDAT1 RT variant (FIGS. 31C-F) or a control RT variant (FIGS. 31A-B; SEQ ID NO: 1, 142, 143, or 172) and three different buffer formulations-RT reagent (FIG. 31A and FIG. 31C), Buffer X.4 (FIG. 31D), and Buffer X.5 (FIG. 31B, FIG. 31E, and FIG. 31F). A MMLV RT variant without a DAT fusion domain was used as a control. Under sandwich configuration conditions, some evidence of undesirable flow of transcripts and/or target molecules or analytes (black circle on upper right quadrant) was observed. UMI heat maps are shown as a log 10 (UMI) ranging from cold to hot (0, 0.5, 1.0, 1.5, 2.0, and 2.5).

FIGS. 32A-E provide UMI heat maps showing globally detected gene expression (FIGS. 32A-B) or gene expression of Crgm1 (FIG. 32C), KRT5 (FIG. 32D), and Rho (FIG. 32E) in a spatial assay performed using a first slide configuration on Zebrafish (non-human or mouse tissue) (e.g., Visium standard definition (SD) spatial gene expression) analyzed using NDAT1 RT variant (42BL-NDAT1) and RT reagent buffer under sandwich configuration conditions. UMI heat maps are shown as a count log 10 ranging from cold to hot (0.5, 1, 1.5, 2, 2.5, 3, 4, 4.5, and 5), or log normalized per experiment for CRYGM1 (0.00-5.0), KRT5 (0.0-7.0) or RHO (0.0-8.0). The quality, and sensitivity metrics were: fraction reads in spots under tissue (0.9), fraction reads usable (0.4), fraction reads mapped to genome (0.56), and number of reads (250M).

FIGS. 33A-D provide UMI heat maps showing globally detected gene expression (FIG. 33A) or gene expression of CRGM1 (FIG. 33B), KRT5 (FIG. 33C), and RHO (FIG. 33D) in a spatial assay performed using a second slide configuration (e.g., Visium high definition (HD) spatial gene expression or next generation spatial gene expression) on Zebrafish (non-human or mouse tissue) analyzed using NDAT1 RT variant (42BL-NDAT1) and RT reagent buffer under sandwich configuration conditions. UMI heat maps are shown as a log normalized per experiment for CRYGM1 (0.00-4.0), KRT5 (0.0-9.0) or RHO (0.0-7.0). The quality, and sensitivity metrics were fraction reads mapped to genome (0.72).

### DETAILED DESCRIPTION

### I. Overview

### A. Enhancement of Transcript Capture

One goal of next generation single-cell and spatial platforms is to improve the sensitivity (or UMI and transcript capture) of single cell assays. An early, and pivotal step, of transcript capture in single cell 5′, single cell 3′, and spatial assays (i.e., Visium) is onboarding of reverse transcriptase enzyme to hybridized oligo(A)-oligo(T) primer-template tract, of which the poly-dT arises from the primer and the poly-A from the mRNA transcript.

In current assays and in published protocols, methods to optimize transcript capture using sequence specific DNA binding proteins have not been described. In order to improve the transcript capture (and therefore sensitivity) of existing assays, the present disclosure leverages functional characteristics of DNA binding proteins to engineer RT variants that increase the sensitivity of the RT in single cell applications. In particular, the present disclosure provides engineered RT variants (e.g., engineered reverse transcriptase (RT) polypeptide or recombinant RT proteins) to increase transcript capture during single application using an accessory protein that is sequence specific for oligo(A)-oligo(T) tract.

The accessory protein contemplated by the present disclosure is a DNA binding protein or amino acid motif which is sequence specific to oligo(A)-oligo(T) tract. The accessory protein can function as a “fusion” partner with the reverse transcriptase (e.g., MMLV RT or variant thereof) in single cell 5′, single cell 3′, and/or spatial assays (e.g., 10× Genomics single cell 5′, single cell 3′, and/or spatial assays). This strategy enabled improvement of transcript capture, and therefore sensitivity of the assays.

### B. DAT1

Dating (DAT1) or a truncation thereof was identified as a candidate sequence specific DNA binding motif. DAT1 is a yeast protein (e.g., Saccharomyces cerevisiae) that specifically recognizes the minor groove of non-alternating oligo(A)-oligo(T) tracts (e.g., >10 bp oligo(A)-oligo(T) tract). See e.g., Reardon et al., PNAS 90, 11327 (1993); Reardon et al. Nucleic Acids Research, 23, 4900 (1995); and Winter & Varshavsky, EMBO J., 8:1867 (1989). The sequence specific recognition may be determined by three repeated pentads of G-R-K-P-G (SEQ ID NO: 11). The N-terminal 90 amino acids (D90) and/or the N-terminal 36 amino acids (D36) can bind in a sequence specific manner to oligo(A)-oligo(T) tract. DAT1 (D-90) can specifically bind to A-T tracts with K_{d} of about 3×10⁻¹⁰ M (or 3×10⁻⁹ M); and DAT1 (D-36) protein can bind to A-T tracts with K_{d} of 4×10⁻¹⁰ M. DAT1 (D-90) can also be more resistant to degradation by bacterial proteases than longer and shorter DAT1 derivatives. The DNA binding activity of DAT1 (D-90) can also be resistant to heat (boiling in water bath for 10 min) and chemical treatment (6 M guanidine HCl). DAT1 (D-90) can also be highly soluble in physiologic salt and pH conditions.

While DNA binding proteins have been used in combination with a reverse transcriptase as fusion proteins to improve processivity of the reverse transcriptase, sequence specific DNA binding proteins have not been explored. See e.g., Oscorbin et al., FEBS Lett., 594, 4338 (2020). Furthermore, sequence specific DNA binding proteins have not been applied to sequencing applications of any type. Fusion proteins with DAT1 have also not been constructed.

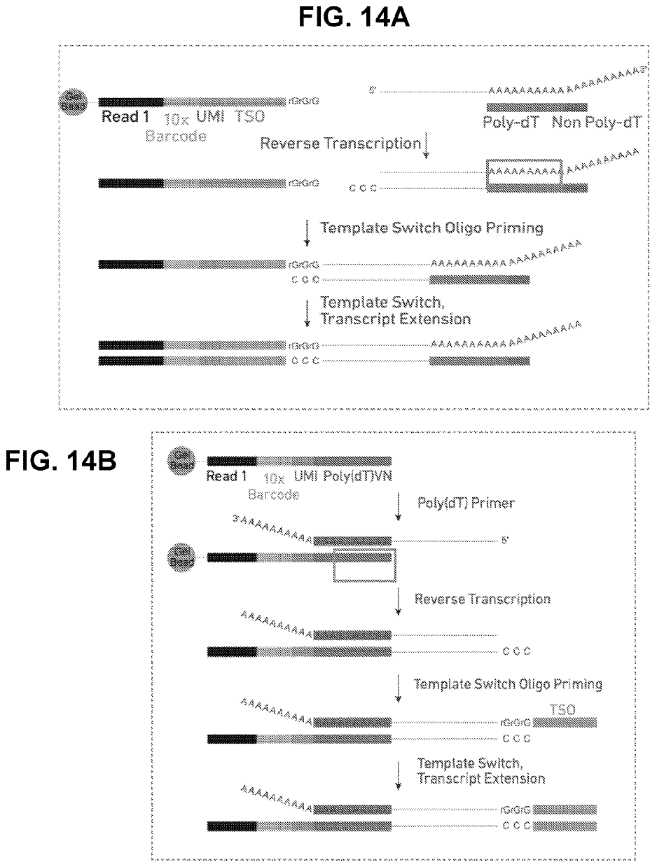

As such, engineered RT molecules comprising DAT1 or fragments thereof were investigated as methods to improve the sensitivity of single cell assays. The binding properties of DAT1 was found to be highly tunable. DAT1 was combined with reverse transcriptase (42B or other MMLV variants). Both N-terminal and C-terminal fusions were explored. In addition, different truncations of DAT1 were tested (i.e., DAT1 (90), first 90 N-terminal amino acids; or DAT1 (36), a 36 residue minimal DAT1 binding domain). The present disclosure shows that DAT1 binding domain can assist a reverse transcriptase in binding to primed transcripts (FIGS. 14A-B and 15A-B; box), thereby increasing the assay sensitivity. The DAT1 constructs were optimized for: (1) truncation of DAT1 binding domain; (2) tuning the binding strength of DAT1 to oligo(A)-oligo(T) tract through sequence modification (e.g., removal or alteration of G-R-K-P-G binding pentad); and (3) identification and testing of other sequence-specific DNA binding proteins.

### C. Experimental Results

Accordingly, the present disclosure provides engineered reverse transcriptase (RT) polypeptides comprising an RT polypeptide sequence; a DNA binding domain; and a linker connecting the RT polypeptide sequence and the DNA binding domain. The DNA binding domain is from a molecule capable of binding a minor groove of a nucleic acid (e.g., DAT 1). In some aspect, the present disclosure provides recombinant reverse transcriptase (RT) proteins comprising a RT polypeptide, fused to a DNA binding domain, where the RT polypeptide and the DNA binding domain are separated by an amino acid linker, and the DNA binding domain is fused to the N-terminus of the RT polypeptide. In another aspect, the present disclosure provides recombinant reverse transcriptase (RT) proteins comprising a RT polypeptide, fused to a DNA binding domain, where the RT polypeptide and the DNA binding domain are separated by an amino acid linker, and the DNA binding domain is fused to the C-terminus of the RT polypeptide. Also provided are compositions comprising the engineered RT or the recombinant RT protein and methods of using the engineered RT or the recombinant RT protein for performing reverse transcription reactions in a variety of applications. An exemplary DNA binding protein is DAT1. A full-length DNA binding protein, truncations or fragments thereof, and/or peptide motifs with sequence specific binding can be used to engineer the RT contemplated by the present disclosure.

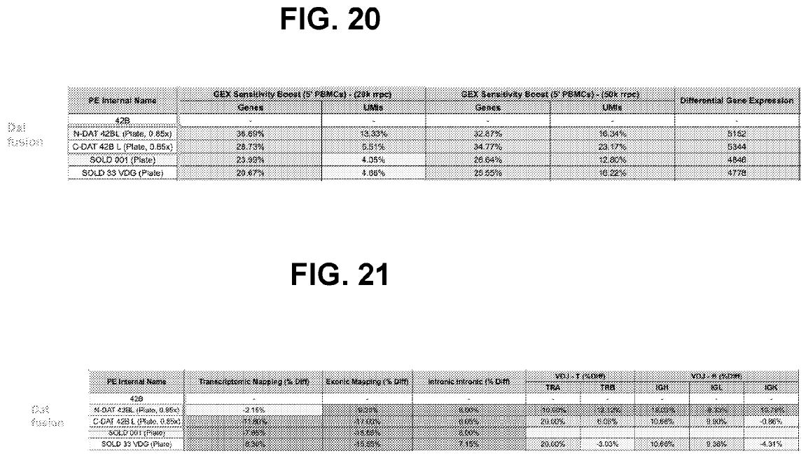

The engineered RT polypeptides and/or the recombinant RT proteins disclosed herein were tested in 5′ single cell assay, 3′ single cell assay, and/or spatial platforms and were shown to significantly enhance the sensitivity of various single cell assays. (FIGS. 20-33).

FIG. 20 and FIG. 21 show that DAT1 in combination with reverse transcriptase MMLV, or MMLV variants thereof (e.g., SOLD 001 or SOLD 033 VDG), either fused at the N-terminus or the C-terminus of the RT improved the assay (GEX) sensitivity even at low sequencing depth. Improvement was observed with both gene expression, which was increased by up to ~37% and UMI, which was increased by up to 13% as captured at 20 k rrpc. Gains were even more significant at higher read depth. In addition, the engineered RT molecules disclosed herein exhibited large change in differential gene expression in single cell assays. The engineered RT molecules comprising DAT1 or variants thereof disclosed herein picked-up up to about 5000 additional genes when compared to a non-DAT1 RT control (e.g., MMLV variant alone). FIGS. 20-26. The engineered RT molecules disclosed herein also exhibited increase in median UMI counts per spot and median gene counts per spot in spatial assay. Moreover, the engineered RT molecules disclosed herein gave decrease in fraction of reads mapped to exons with gain in fraction mapped to introns. See e.g., FIG. 21. A performance difference between FPLC and plate purified proteins was also demonstrated.

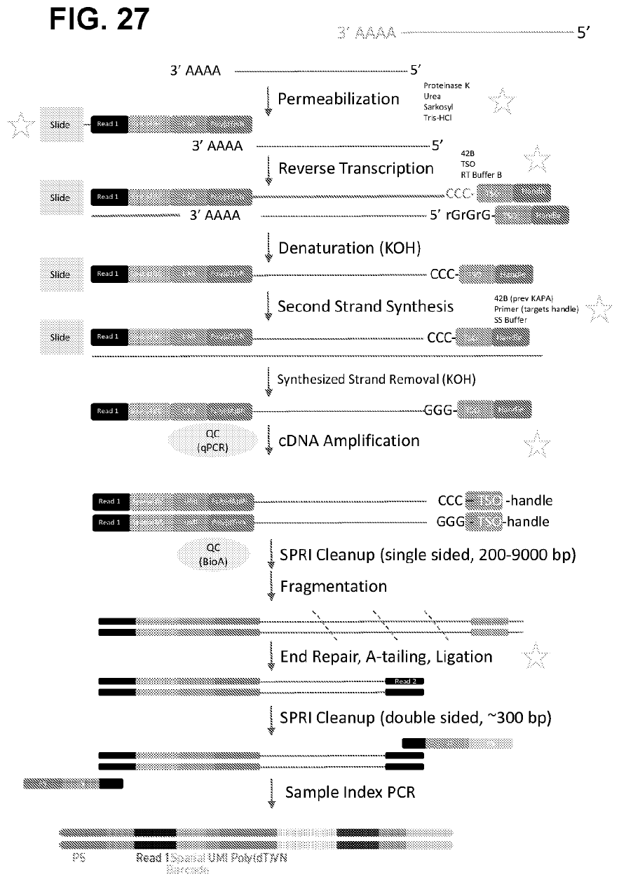

The engineered RT polypeptides and/or the recombinant RT proteins disclosed herein were tested in spatial platforms using the 3′ workflow shown in FIG. 27 under sandwich configuration conditions and using three different buffer formulations described herein. The engineered N-DAT1 RT variants disclosed herein enhanced the quality and the sensitivity of the spatial assay metrics while maintaining good spatial resolution when compared to a control RT lacking the DAT1 domain (also referred to as “control RT”). As used herein, a control RT, in the context of DAT1-RT fusion, refers to a non-DAT1 fusion RT comprising the same RT backbone as the N-DAT1 RT or the C-DAT1 RT. In some embodiments, the control RT is a MMLV variant (SEQ ID NOs: 1, 143, or 172) or MMLV variant L (SEQ ID NO: 145).

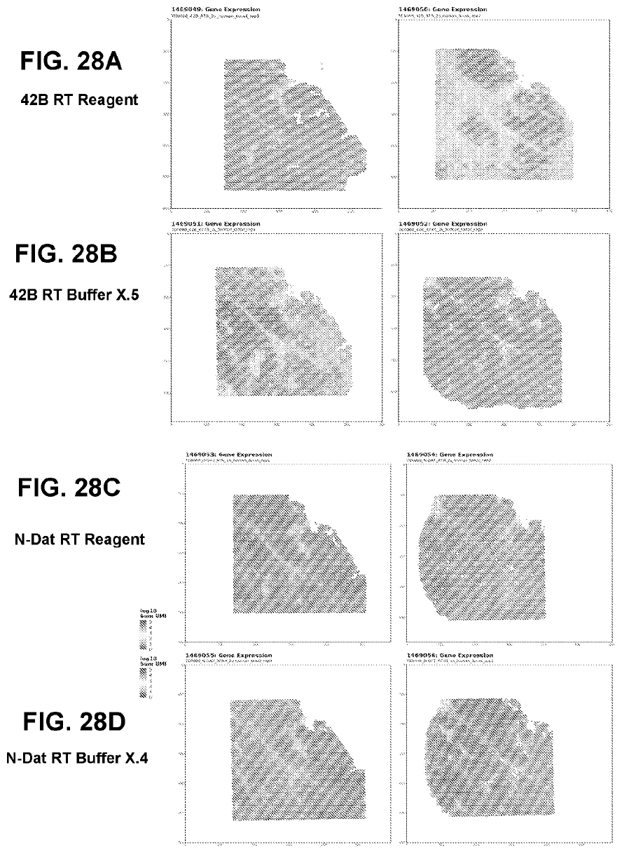

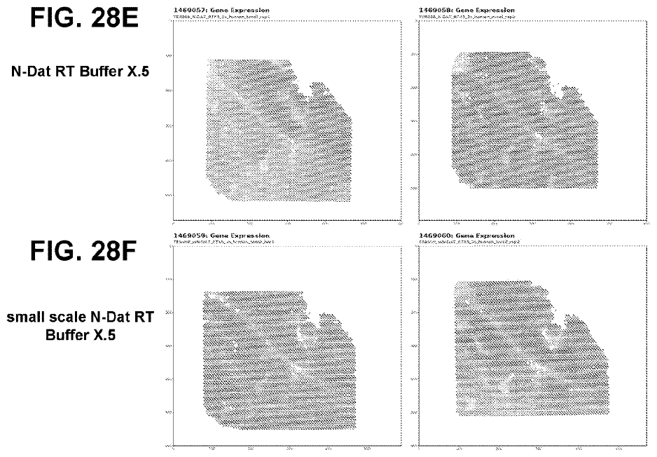

As shown in FIGS. 28A-F, the engineered RT polypeptides and/or the recombinant RT proteins disclosed herein maintained a good spatial resolution (e.g., an engineered RT variant comprising DAT-1 at the N-terminus (N-DAT)). FIGS. 28A-F show UMI heat maps showing globally detected gene expression in a spatial assay performed using a first slide configuration, on human tonsil tissue analyzed using NDAT1 RT variant (FIGS. 28C-F) compared to a control RT variant (FIGS. 28A-B) using three different buffer formulations.

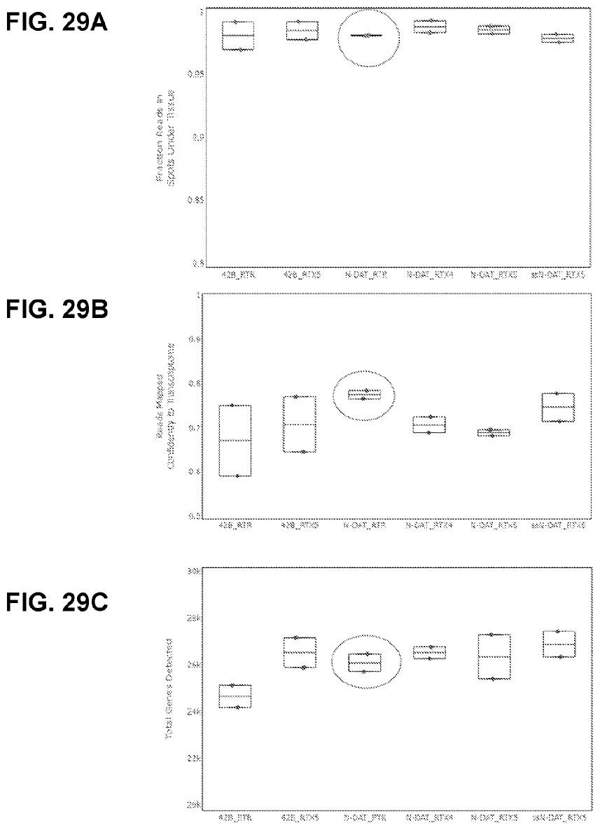

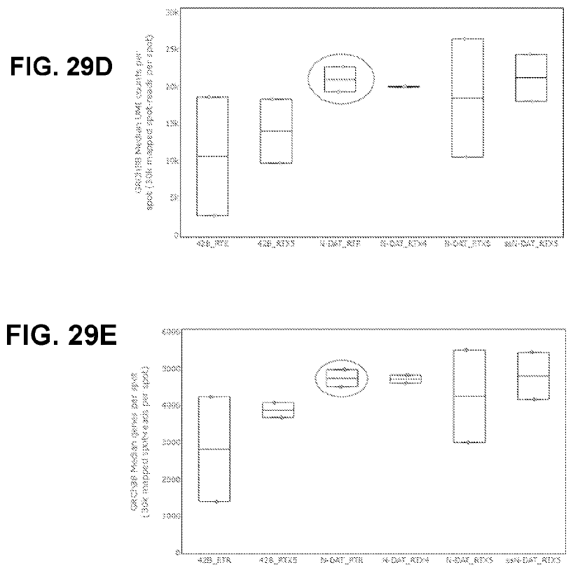

FIGS. 29A-F show that the engineered RT polypeptides and/or the recombinant RT proteins disclosed herein (e.g., an engineered RT variant comprising DAT-1 at the N-terminus (N-DAT)) also significantly enhanced the quality and the sensitivity metrics of the spatial assays. FIGS. 29A-F quantify the quality, sensitivity, and detection of gene expression metrics under the same six conditions shown in FIGS. 28A-F. In particular, fraction reads in spots under tissue (FIG. 29A) was substantially the same among the 6 conditions tested. However, the standard deviation of the sample comprising the N-DAT RT and RT reagent buffer (N-DAT_RTR) was smaller. The “fraction reads under tissue” refers to the ratio between reads in the area of direct interaction between the array and the tissue over total reads. The fraction reads under tissue showed the diffusion or transcript mislocalization that may have occurred during transcript release from the tissue or transcript capture onto the array.

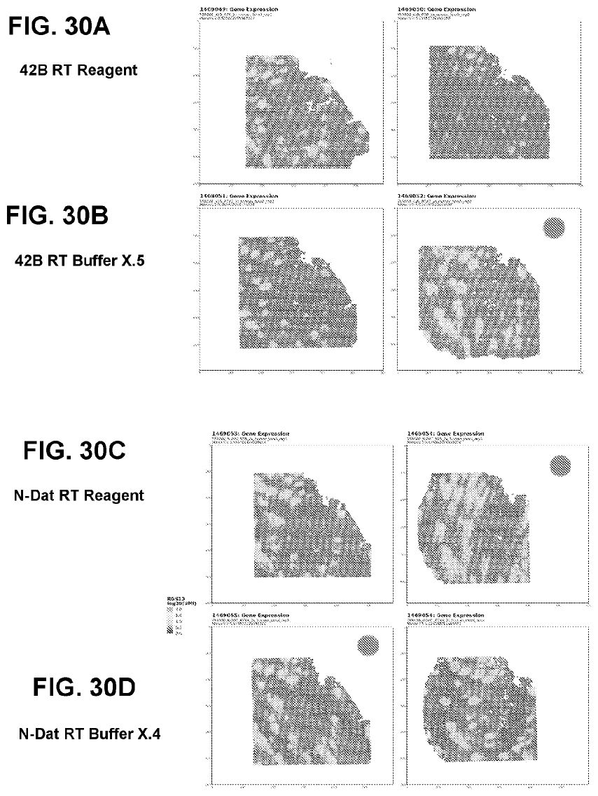

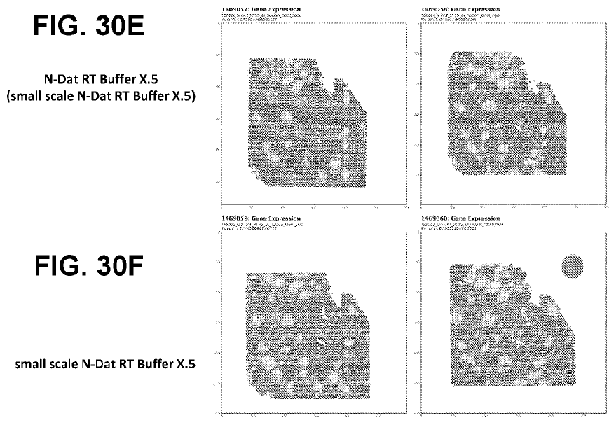

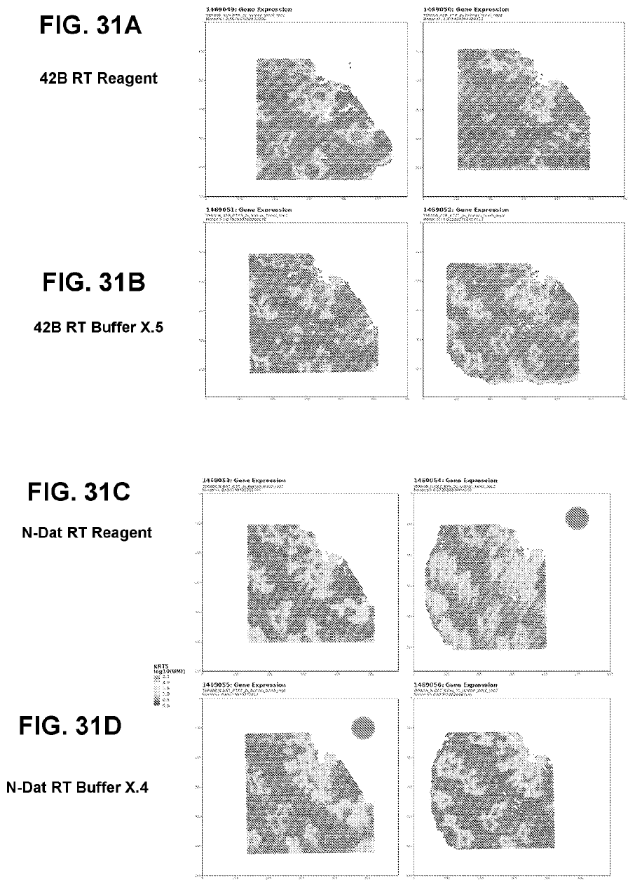

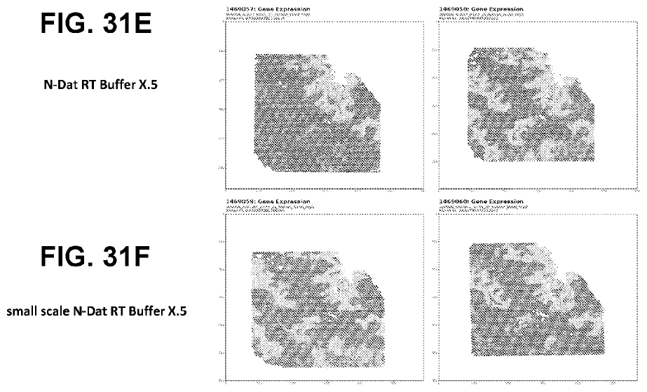

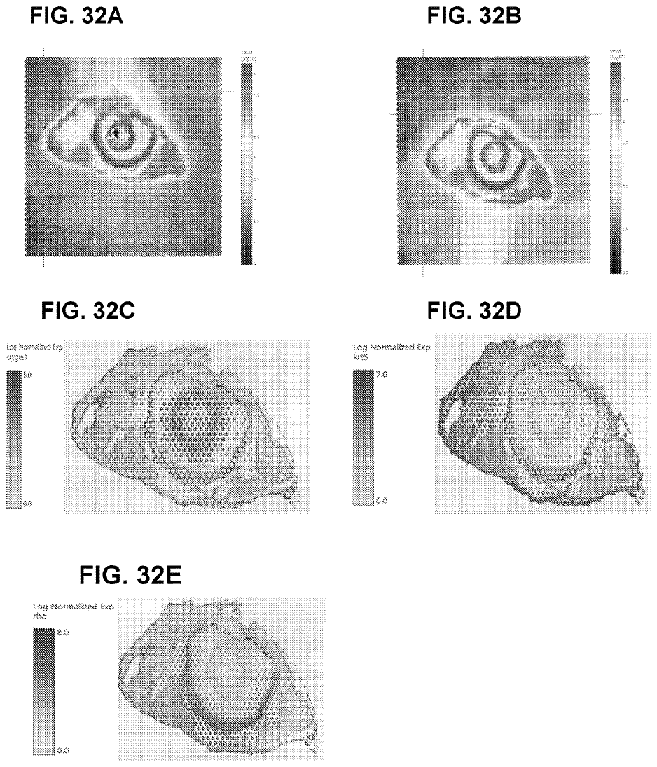

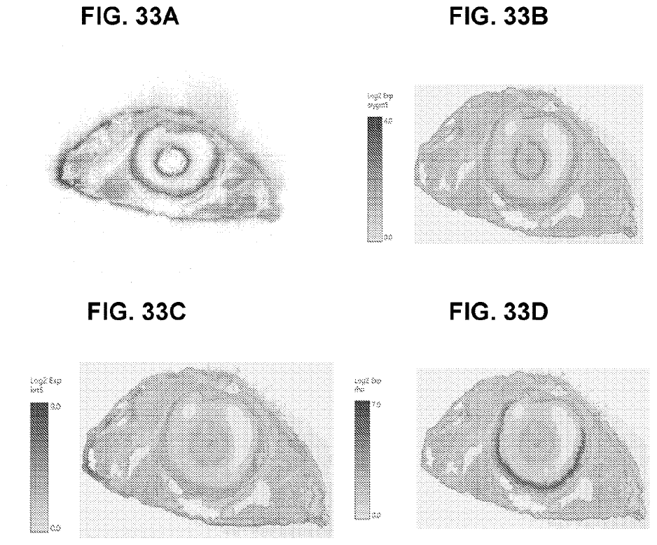

Reads mapped confidently to transcriptome (FIG. 29B) were significantly increased in the samples containing the N-DAT_RT when compared to the five other conditions. The total genes detected (FIG. 29C) appeared the same in all conditions tested, except in samples including the control RT variant and using RT reagent buffer (42B_RTR), which showed significantly reduced total genes detected. Analysis of GRch38 median UMI counts per spot (30 k mapped spot-reads per spot) (FIG. 29D) showed that median UMI counts per spot were significantly enhanced in all samples comprising an N-DAT RT variant when compared to the control RT samples regardless of the buffer used. Unexpectedly, samples comprising the N-DAT RT variant and Buffer X.4 showed less variability. In addition, the GRch38 median genes per spot (30 k mapped spot-reads per spot) (FIG. 29E) analysis showed that median genes per spot were significantly enhanced in all samples comprising an N-DAT1 RT variant when compared to the control RT samples regardless of the buffer used. Assessment of individual gene expression also showed substantially similar results as globally detected gene expression. FIGS. 30A-F show UMI heat maps illustrating the gene expression of RGS3 in a human tonsil tissue. FIGS. 31A-C show UMI heat maps illustrating the gene expression of KRT5 in a human tonsil tissue. Additionally, spatial assays conducted with an N-DAT RT variant were successful using two different slide configurations: the first slide configuration (e.g., Visium standard definition (SD) spatial gene expression assay) or the second slide configuration (e.g., Visium high definition (HD) or next generation spatial gene expression assay), with a sample from Zebrafish (FIGS. 32A-E and FIGS. 33A-D).

As such, the engineered RT polypeptides and/or the recombinant RT proteins disclosed herein improved the sensitivity of the spatial assay while maintaining good spatial resolution when compared to the control RT variant (FIGS. 29D-E). Furthermore, in some metrics, such as reads mapping confidently to the genome, use of the N-DAT RT variant with RT Reagent was particularly superior.

Thus, the present disclosure demonstrates for the first time that incorporation of DAT1 or any molecule having substantially similar molecular function, in single and spatially assay increased transcript capture and single cell assay sensitivity. The DNA binding domain enhances the enzymatic activity of the engineered reverse transcriptase. For example, the addition of the DNA binding domain can enhance the template switching (TS) efficiency, higher end-to-end template jumping/switching, processivity efficiency, binding affinity, transcription efficiency, chemical tolerance, ability to yield mitochondrial unique molecular identifier (UMI) counts, ability to yield ribosomal unique molecular identifier (UMI) counts, shelf life, higher strand displacement, increased thermostability, improved thermoreactivity, and any combination thereof, for the engineered (i.e., recombinant) reverse transcriptase when compared to a control RT (SEQ ID NO: 1, 143, 145, or 172), WT MMLV, or known MMLV variants.

TS efficiency: Small RNAs (<200 nucleotides) are for the most part non-coding regulatory elements and play a key role in gene expression. Small RNAs regulate gene expression in plants, animals, and many fungi—including several roles in development, proliferation, differentiation, immune reaction, apoptosis, tumorigenesis and adaptation to stress. Given their importance in regulation, miRNAs are candidates as biomarkers for several human diseases. Thus, developing accurate and reproducible ways to study these and other small RNAs is necessary to further decipher their biological consequences.

The main sources of bias in a typical library preparation workflow are the enzymatic ligations that introduce 5′ and 3′ sequencing adaptors to single-stranded templates. Template switching permits ligation-free incorporation of the 5′ adapter during reverse transcription. Template switching-based methods depend upon the natural tendency of MMLV-type reverse transcriptases to add nontemplated nucleotides at the 3′ end of the emerging cDNA strand. These nontemplated additions serve as an anchoring unit for annealing complementary nucleotides in a provided template switching oligonucleotide (TSO); upon reaching the cDNA-TSO cross-junction, the reverse transcriptase effectively switches templates, continuing cDNA synthesis out of the TSO sequence. By incorporating the 5′ adapter sequence into the TSO, and using polyadenylation to prime reverse transcription, ligation steps can be avoided altogether. For applications where the total RNA input is limited, such as single-cell RNA sequencing, template switching offers a critical advantage as it reduces the number of steps and sample loss during library preparation. Thus, the engineered reverse transcriptase described herein, exhibiting improved TS efficiency, is highly desirable.

Higher end-to-end template jumping or switching: End-to-end template jumping or switching refers to the ability of a reverse transcriptase to template-switch from the 5′ end of one template to the 3′ end of another. Improved end-to-end template jumping or switching can result in an improved process efficiency. Thus, the engineered reverse transcriptase described herein, exhibiting improved or higher end-to-end template jumping or switching, is highly desirable.

DNA binding affinity: To initiate reverse transcription, reverse transcriptases require a short DNA oligonucleotide called a primer to bind to its complementary sequences on the RNA template and serve as a starting point for synthesis of a new strand. Improved binding affinity results in a more efficient process, particularly when limited amounts of RNA are available. Thus, the engineered reverse transcriptase described herein, exhibiting improved DNA binding affinity, is highly desirable.

Transcription efficiency: The RNA-to-cDNA conversion step in transcriptomics experiments is widely recognized as inefficient and variable. This issue is particularly significant for transcriptomics at the single cell level, which is preferable due to greater recognition of sample heterogeneity. Transcriptomics measurements almost invariably include a reverse transcription (RT) step, where RNA transcripts are used as templates to generate cDNA transcripts for quantification. This significantly complicates data interpretation as techniques are not directly measuring RNA transcript number, and results are therefore dependent on the efficiency of the RNA to cDNA conversion. Thus, the engineered reverse transcriptase described herein, exhibiting improved transcription efficiency, is highly desirable.

Chemical tolerance: Reverse transcriptases function in an environment that may include processing chemicals, such as cell fixation chemicals or processing reagents, which can negatively impact the function and activity of the enzyme. Thus, the engineered reverse transcriptase described herein, exhibiting improved chemical tolerance, is highly desirable.

Ability to yield mitochondrial and/or ribosomal unique molecular identifier (UMI) counts: Unique molecular identifier (UMI) counting is a gene expression quantification scheme used in single-cell RNA-sequencing (scRNA-seq) analysis. Single-cell RNA-sequencing (scRNA-seq) technology provides transcriptome profiles of individual cells, enabling the dissection of the heterogeneity of different cell populations and tissues. The paucity of starting material for reverse transcription remains an inherent limitation of scRNA-seq protocols and contributes to the relatively low rate at which messenger RNA (mRNA) molecules in individual cells are converted to cDNA molecules that can be captured and sequenced. The miniscule quantity of transcripts captured from a single cell requires cDNA amplification for library construction; this inevitably results in large amplification bias. To mitigate this bias, some scRNA-seq protocols employ an additional step in which individual transcripts are barcoded with unique molecular identifiers (UMIs) before amplification, resulting in a more accurate quantification of the transcript count. UMIs incorporate a unique barcode onto each molecule within a given sample library. By incorporating individual barcodes on each original DNA fragment, variant alleles present in the original sample (true variants) can be distinguished from errors introduced during library preparation, target enrichment, or sequencing. Thus, the engineered reverse transcriptase described herein, exhibiting an improved ability to yield mitochondrial and/or ribosomal UMI counts, is highly desirable.

Shelf life and/or stability: In another aspect of the disclosure, the engineered reverse transcriptase described herein, exhibit improved stability and/or shelf life. A longer period of stability, and/or shelf life, is desirable as it can result in more efficient processes.

Higher strand displacement: Strand displacement is the process through which two strands with partial or full complementarity hybridize to each other, displacing one or more pre-hybridized strands in the process. Reverse transcriptase first transcribes a complementary strand of DNA to make an RNA: DNA hybrid. Next, reverse transcriptase or RNase H degrades the RNA strand of the hybrid. The single-stranded DNA is then used as a template for synthesizing double-stranded DNA (cDNA). Thus, reverse transcriptase (RT) catalyzes the conversion of RNA into an integration-competent double-stranded DNA, with a variety of enzymatic activities that include the ability to displace a non-template strand concomitantly with polymerization. RT are capable of efficiently unwinding duplexes in the template during polymerization. This strand displacement synthesis activity by RT is required for the polymerization on the highly structured RNA and the removal of RNA fragments which cannot be cleaved by the enzymes' RNase H activity. In addition, strand displacement synthesis on a DNA duplex is particularly important to complete the plus- and minus-strands by polymerizing on the long terminal repeats. As such, an RT with a higher strand displacement property is more efficient. Accordingly, the engineered reverse transcriptase described herein, exhibiting an improved strand displacement property, is highly desirable.

Any of the engineered RT enzymes of the present disclosure, including without limitation any of the enzymes comprising the amino acid sequence and/or nucleic acid sequences shown in Table 1 or Table 2 could be analyzed in any suitable assay, including without limitation the assays described herein. Assays include without limitation 5′ gene expression analyses, with or without VDJ analysis, 3′ gene expression analysis, epigenetic analysis, or multiomic analyses. In non-limiting embodiments, experiments are carried out as found in the manufacturer's instructions for the Chromium Single Cell 5′ Gene Expression Assay kit (10× Genomics); Chromium Single Cell 3′ Gene Expression Assay kit (10× Genomics), including any of multiomic extensions or applications.

### II. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363 (6434): 1463-1467, 2019; Lee et al., Nat. Protoc. 10 (3): 442-458, 2015; Trejo et al., PLOS ONE 14 (2): e0212031, 2019; Chen et al., Science 348 (6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples-which can be from different tissues or organisms-assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

The biological sample as used herein can be any suitable biological sample described herein or known in the art. In some embodiments, the biological sample is a tissue. In some embodiments, the tissue sample is a solid tissue sample. In some embodiments, the biological sample is a tissue section. In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed using cryosectioning. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plants, an insect, an arachnid, a nematode (e.g., Caenorhabditis elegans), a fungi, an amphibian, or a fish (e.g., zebrafish)). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning. In some instances, the biological sample is not fixed with paraformaldehyde (PFA). In some instances, when the biological sample is fixed with a fixative including an alcohol (e.g., methanol or acetone-methanol mixture), it is not decrosslinked afterward. In some preferred embodiments, the biological sample is fixed with a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample e.g., using acetone and/or alcohol (e.g., methanol, acetone-methanol) is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing e.g., by formalin or PFA, the biological sample is dehydrated via sucrose gradient. In some instances, the fixed biological sample is treated with a sucrose gradient and then embedded in a matrix e.g., OCT compound. In some instances, the fixed biological sample is not treated with a sucrose gradient, but rather is embedded in a matrix e.g., OCT compound after fixation. In some embodiments when a fixed frozen tissue sample is treated with a sucrose gradient, it can be rehydrated with an ethanol gradient. In some embodiments, the PFA or formalin fixed biological sample, which can be optionally dehydrated via sucrose gradient and/or embedded in OCT compound, is then frozen e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated in an ethanol gradient.

In some instances, the biological sample (e.g., a fixed frozen tissue sample) is treated with a citrate buffer. Citrate buffer can be used for antigen retrieval to decrosslink antigens and fixation medium in the biological sample. Thus, any suitable decrosslinking agent can be used in addition to or alternatively to citrate buffer. In some embodiments, for example, the biological sample (e.g., a fixed frozen tissue sample) is decrosslinked with TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, it is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, it can be rehydrated with an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene®. For example, the biological sample can be fixed using PAXgene® in addition, or alternatively to, a fixative disclosed herein or known in the art (e.g., alcohol, acetone, acetone-alcohol, formalin, paraformaldehyde). PAXgene® is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1; 9 (10): 5188-96; Kap M. et al., PLOS One.; 6 (11): e27704 (2011); and Mathieson W. et al., Am J Clin Pathol.; 146 (1): 25-40 (2016), each of which are hereby incorporated by reference in their entirety, for a description and evaluation of PAXgene® for tissue fixation. Thus, in some embodiments, when the biological sample, e.g., the tissue sample, is fixed in a fixative including alcohol, the fixative is PAXgene®. In some embodiments, a fresh frozen tissue sample is fixed with PAXgene®. In some embodiments, a fixed frozen tissue sample is fixed with PAXgene®

In some embodiments, the biological sample, e.g., the tissue sample is fixed, for example in methanol, acetone, acetone-methanol, PFA, PAXgene® or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probes that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Non-limiting examples of stains include histological stains (e.g., hematoxylin and/or eosin) and immunological stains (e.g., fluorescent stains). The biological sample can be stained using Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. In some instances, PAS staining is performed after formalin or acetone fixation. In some embodiments, a biological sample (e.g., a fixed and/or stained biological sample) can be imaged. Biological samples are also described in Section (I)(d) of PCT Publication No. WO2020/176788 and/or U.S. Patent Application Publication No. 2020/0277663.

The following embodiments can be used with any of the methods described herein. In some embodiments, the biological sample is imaged. In some embodiments, the biological sample is visualized or imaged using bright field microscopy. In some embodiments, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding the primer to the biological sample.

In some embodiments, the method includes staining the biological sample. In some embodiments, the staining includes the use of hematoxylin and eosin. In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label selected from the group consisting of a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d) (ii) (13) or the Exemplary Embodiments Section of PCT Publication No. WO2020/176788 and/or U.S. Patent Application Publication No. 2020/0277663. Briefly, in any of the methods described herein, the method includes a step of permeabilizing the biological sample. For example, the biological sample can be permeabilized to facilitate transfer of the extension products to the capture probes on the array. In some embodiments, the permeabilizing includes the use of an organic solvent (e.g., acetone, ethanol, and methanol), a detergent (e.g., saponin, Triton X-100™, Tween-20™, or sodium dodecyl sulfate (SDS)), an enzyme (an endopeptidase, an exopeptidase, a protease), or combinations thereof. In some embodiments, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™, Tween-20™, or combinations thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, the entire contents of which are incorporated herein by reference.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663. Generation of capture probes can be achieved by any appropriate method, including those described in Section (II)(d)(ii) of PCT Publication No. WO2020/176788 and/or U.S. Patent Application Publication No. 2020/0277663.

In some embodiments, the biological sample is mounted on a first substrate and the substrate comprising the array of capture probes is a second substrate. During this process, one or more analytes or analyte derivatives (e.g., intermediate agents; e.g., ligation products) are released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described e.g., in U.S. Patent Application Pub. No. 2021/0189475 and PCT Pub. Nos. WO 2021/252747 A1, WO 2022/061152 A2, and WO 2022/140028 A1.

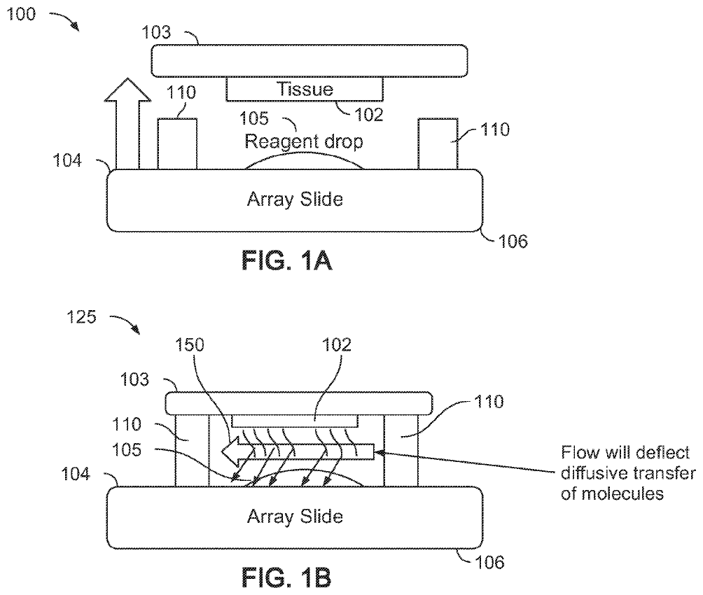

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788, and US. Patent Application Pub. No. 2021/0189475, each of which is hereby incorporated by reference.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104).

In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate, and the second substrate may reduce or prevent flow from undesirable convective movement of transcripts and/or molecules over the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., US. Patent Application Pub. No. 2021/0189475, and PCT Publ. No. WO 2022/061152 A2, each of which are incorporated by reference in their entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

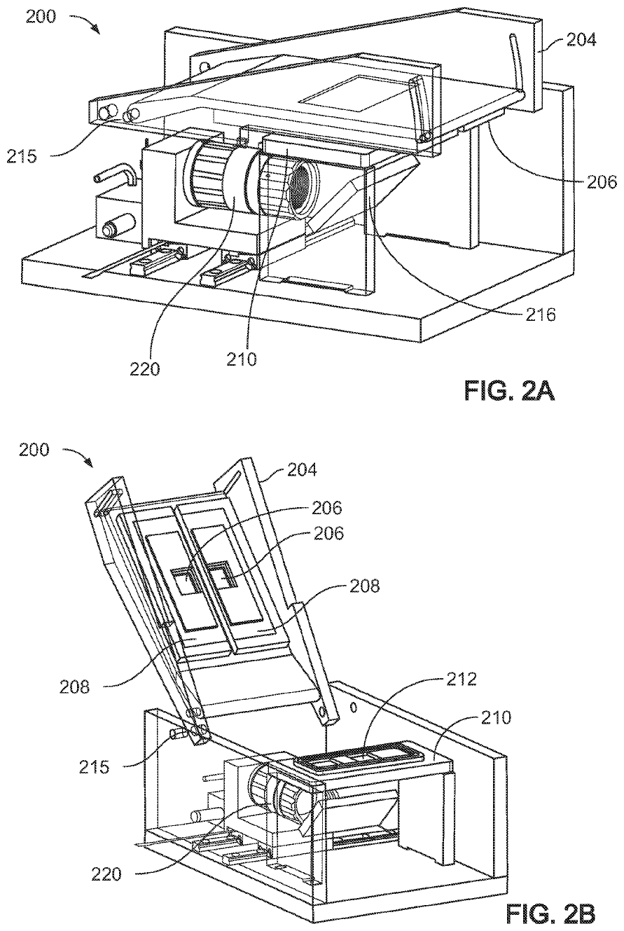

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

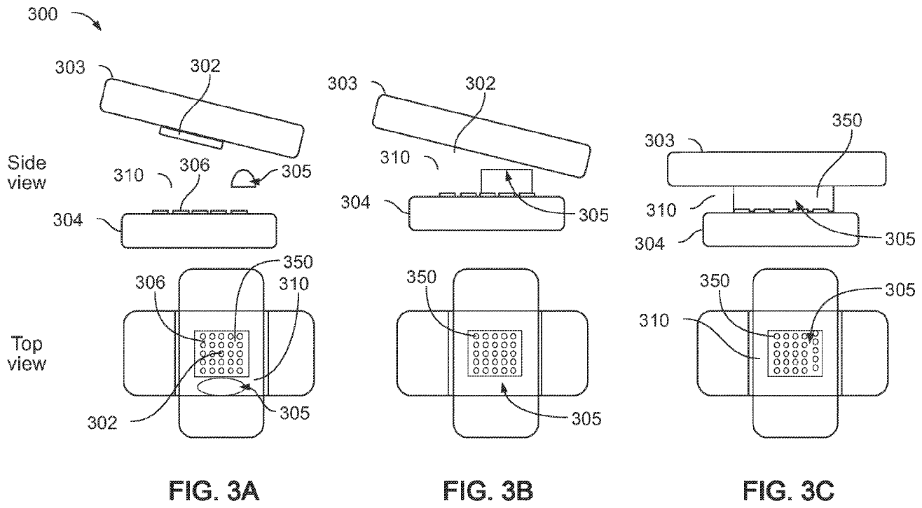

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., the slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right hand side of side view, it should be understood that such depiction is not meant to be limiting as to the location of the reagent medium on the spacer.

FIG. 3B shows that as the first substrate lowers, and/or as the second substrate rises, the dropped side of the first substrate (e.g., a side of the slide 303 angled toward the second substrate) may contact the reagent medium 305. The dropped side of the first substrate may urge the reagent medium 305 toward the opposite direction (e.g., towards an opposite side of the spacer 310, towards an opposite side of the first substrate relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

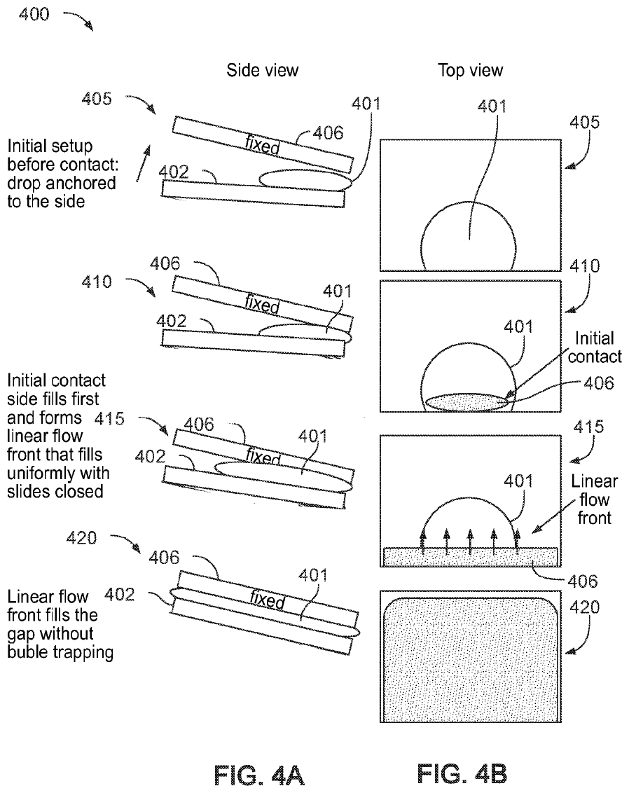

FIG. 4A is a side view of the angled closure workflow 400 in accordance with some exemplary implementations. FIG. 4B is a top view of the angled closure workflow 400 in accordance with some exemplary implementations. As shown at 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills uniformly with the slides closed.

At step 415, the substrate 406 is further lowered toward the substrate 402 (or the substrate 402 is raised up toward the substrate 406) and the dropped side of the substrate 406 may contact and may urge the reagent medium toward the side opposite the dropped side and creating a linear or low curvature flow front that may prevent or reduce bubble trapping between the substrates.

At step 420, the reagent medium 401 fills the gap between the substrate 406 and the substrate 402. The linear flow front of the liquid reagent may form by squeezing the 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening sample holder). Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, pepsin, elastase, and proteinase K. In some embodiments, the reagent medium comprises a nuclease. In some embodiments, the nuclease comprises an RNase. In some embodiments, the Rnase is selected from Rnase A, Rnase C, Rnase H, and Rnase I. In some embodiments, the reagent medium comprises one or more of sodium dodecyl sulfate (SDS) or a sodium salt thereof, proteinase K, pepsin, N-lauroylsarcosine, and RNAse.

In some embodiments, the reagent medium comprises polyethylene glycol (PEG). In some embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

In certain embodiments a dried permeabilization reagent is applied or formed as a layer on the first substrate or the second substrate or both prior to contacting the biological sample and the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for the template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended by a reverse transcriptase. In some embodiments, the capture probe is extended using one or more DNA polymerases. In some embodiments, the extended capture probes include the sequence of the capture domain and the sequence of the spatial barcode of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II) (a) of PCT Publication No. WO2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II) (g) of PCT Publication No. WO2020/176788 and/or U.S. Patent Application Publication No. 2020/0277663. Some quality control measures are described in Section (II) (h) of PCT Publication No. WO2020/176788 and/or U.S. Patent Application Publication No. 2020/0277663.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up- and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II) (c) of PCT Publication No. WO2020/176788 and/or U.S. Patent Application Publication No. 2020/0277663. Exemplary features and geometric attributes of an array can be found in Sections (II) (d) (i), (II) (d) (iii), and (II) (d) (iv) of PCT Publication No. WO2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II) (e) of PCT Publication No. WO2020/176788 and/or U.S. Patent Application Publication No. 2020/0277663.

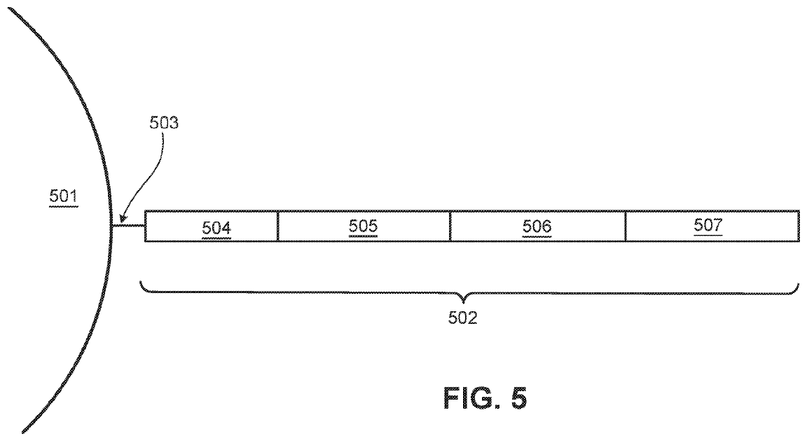

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that are useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

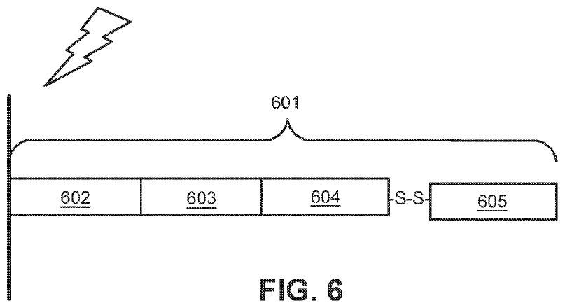

FIG. 6 is a schematic illustrating a cleavable capture probe, wherein the cleaved capture probe can enter into a non-permeabilized cell and bind to analytes within the sample. The capture probe 601 contains a cleavage domain 602, a cell penetrating peptide 603, a reporter molecule 604, and a disulfide bond (—S—S—). 605 represents all other parts of a capture probe, for example a spatial barcode and a capture domain.

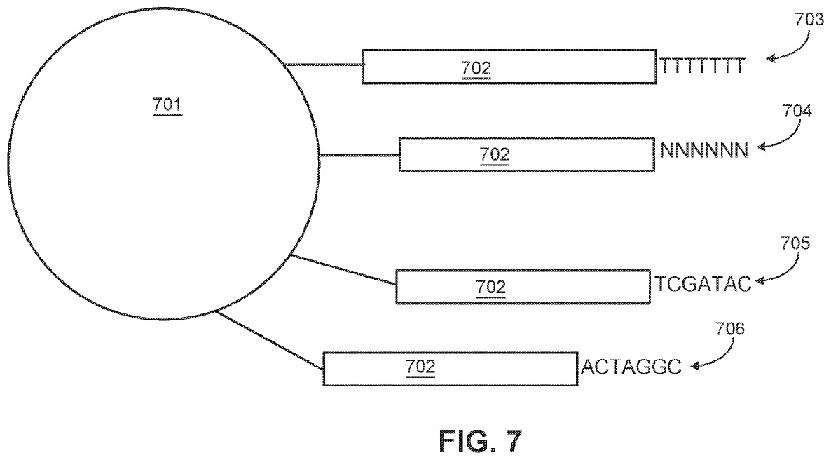

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 702. One type of capture probe associated with the feature includes the spatial barcode 702 in combination with a poly(T) capture domain 703, designed to capture mRNA target analytes. A second type of capture probe associated with the feature includes the spatial barcode 702 in combination with a random N-mer capture domain 704 for gDNA analysis. A third type of capture probe associated with the feature includes the spatial barcode 702 in combination with a capture domain complementary to the analyte capture agent of interest 705. A fourth type of capture probe associated with the feature includes the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents.

The functional sequences can generally be selected for compatibility with any of a variety of different sequencing systems, e.g., Ion Torrent Proton or PGM, Illumina® sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina® sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequences 504 is common to all of the probes attached to a given feature. In some embodiments, the UMI sequence 506 of a capture probe attached to a given feature is different from the UMI sequence of a different capture probe attached to the given feature.

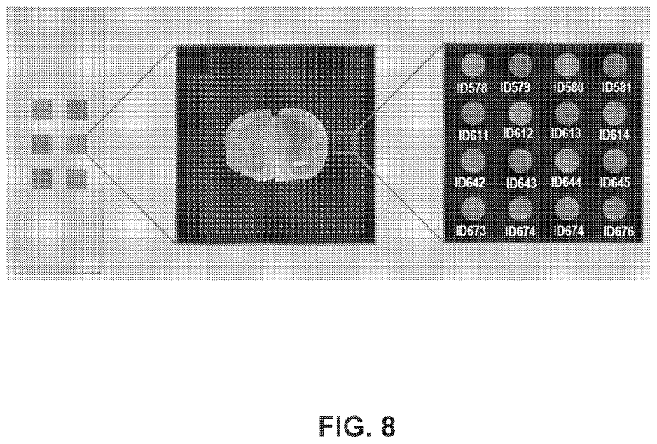

FIG. 8 depicts an exemplary arrangement of barcoded features within an array. From left to right, FIG. 8 shows (L) a slide including six spatially-barcoded arrays, (C) an enlarged schematic of one of the six spatially-barcoded arrays, showing a grid of barcoded features in relation to a biological sample, and (R) an enlarged schematic of one section of an array, showing the specific identification of multiple features within the array (labelled as ID578, ID579, ID560, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45 (14): e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture binding capture domain (e.g., a poly(A) sequence, a non-homopolymeric sequence). After hybridization to the analyte, a ligase (e.g., a T4 RNA ligase (Rnl2), a PBCV-1 DNA Ligase or Chorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location and optionally the abundance of the analyte in the biological sample.

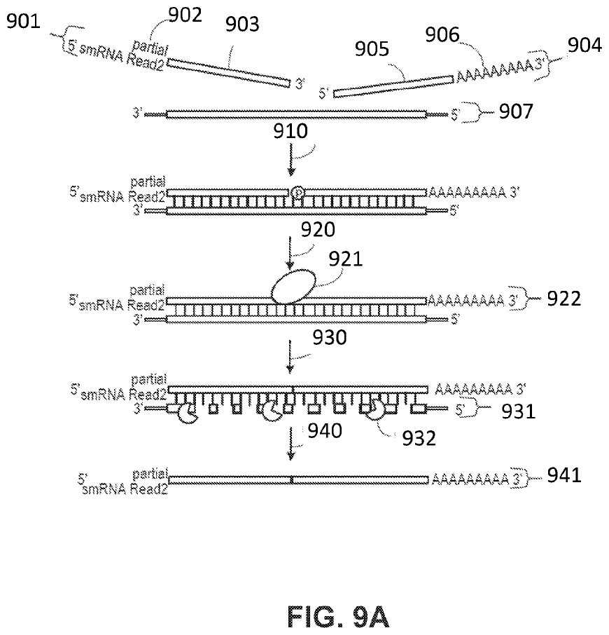

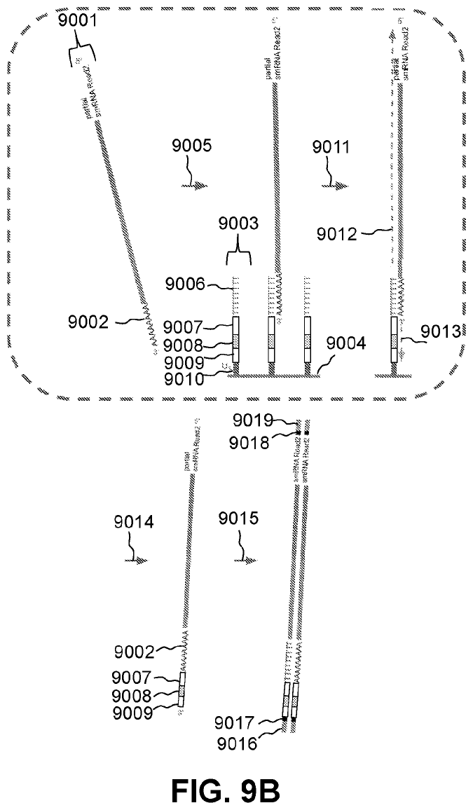

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly-A sequence) 906, the first probe 901 and a second probe 904 hybridize 910 to an analyte 907. A ligase 921 ligates 920 the first probe to the second probe thereby generating a ligation product 922. The ligation product is released 930 from the analyte 931 by digesting the analyte using an endoribonuclease 932. The sample is permeabilized 940 and the ligation product 941 is able to hybridize to a capture probe on the substrate. Methods and composition for spatial detection using templated ligation have been described in PCT Publ. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily bind to the captured ligated probe (i.e., compared to no permeabilization). In some embodiments, reverse transcription (RT) reagents can be added to permeabilized biological samples. Incubation with the RT reagents can extend the capture probes 9011 to produce spatially-barcoded full-length cDNA 9012 and 9013 from the captured analytes (e.g., polyadenylated mRNA). Second strand reagents (e.g., second strand primers, enzymes) can be added to the biological sample on the slide to initiate second strand synthesis.

In some embodiments, cDNA can be denatured 9014 from the capture probe template and transferred (e.g., to a clean tube) for amplification, and/or library construction. The spatially-barcoded, full-length cDNA can be amplified 9015 via PCR prior to library construction. The cDNA can then be enzymatically fragmented and size-selected in order to optimize the cDNA amplicon size. P5 9016, i5 9017, i7 9018, and P7 9019, and can be used as sample indexes, and TruSeq™ Read 2 can be added via End Repair, A-tailing, Adaptor Ligation, and PCR. The cDNA fragments can then be sequenced using paired-end sequencing using TruSeq™ Read 1 and TruSeq™ Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663.

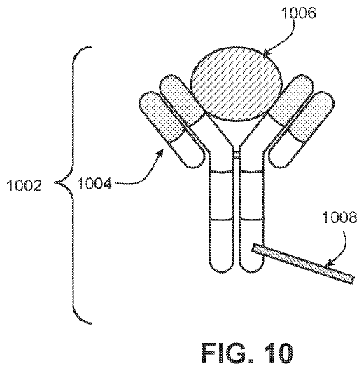

FIG. 10 is a schematic diagram of an exemplary analyte capture agent 1002 comprised of an analyte-binding moiety 1004 and an analyte-binding moiety barcode domain 1008. The exemplary analyte-binding moiety 1004 is a molecule capable of binding to an analyte 1006 and the analyte capture agent is capable of interacting with a spatially-barcoded capture probe. The analyte-binding moiety can bind to the analyte 1006 with high affinity and/or with high specificity. The analyte capture agent can include an analyte-binding moiety barcode domain 1008, a nucleotide sequence (e.g., an oligonucleotide), which can hybridize to at least a portion or an entirety of a capture domain of a capture probe. The analyte-binding moiety barcode domain 1008 can comprise an analyte binding moiety barcode and a capture handle sequence described herein. The analyte-binding moiety 1004 can include a polypeptide and/or an aptamer. The analyte-binding moiety 1004 can include an antibody or antibody fragment (e.g., an antigen-binding fragment).

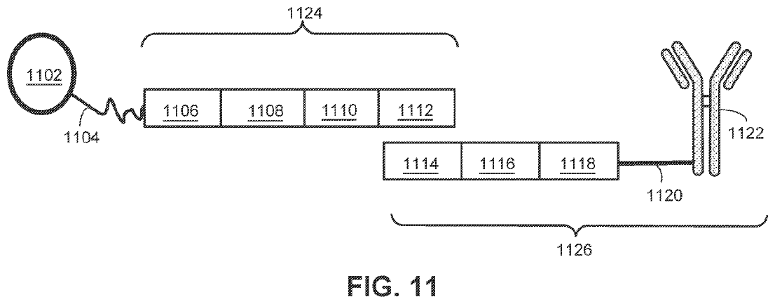

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequences 1106 and UMI 1110, as described elsewhere herein. The capture probe can be affixed 1104 to a feature (e.g., bead) or array 1102. The capture probe can also include a capture domain 1112 that is capable of binding to an analyte capture agent 1126. The analyte capture agent 1126 can include a functional sequence 1118, analyte binding moiety barcode 1116, and a capture handle sequence 1114 that is capable of binding to the capture domain 1112 of the capture probe 1124. The analyte capture agent can also include a linker 1120 that allows the capture agent barcode domain 1116 to couple to the analyte binding moiety 1122.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663. See also, e.g., the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022).

In some embodiments, spatial analysis can be performed using dedicated hardware and/or software, such as any of the systems described in Sections (II) (e) (ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### III. Engineered Reverse Transcriptases

Reverse transcriptases or reverse transcription (RT) enzymes are RNA-dependent DNA polymerases, typically used to create a copy of an RNA sequence thereby generating a cDNA molecule. Reverse transcription is initiated by hybridization of a priming sequence to an RNA molecule which is extended by a reverse transcription enzyme in a template directed fashion. A reverse transcription enzyme adds a plurality of non-template nucleotides to a nucleotide strand, thereby producing complementary deoxyribonucleic acid (cDNA) molecules. The resultant cDNA can then be dehybridized from the template RNA molecule in any number of ways as known in the art. Engineered and/or recombinant are used interchangeably with respect to reverse transcriptase (RT) variant and/or fusion RT.

One aspect of the present disclosure provides an engineered reverse transcriptase (RT) polypeptide comprising an RT polypeptide sequence; a DNA binding domain, and a linker connecting the RT polypeptide sequence and the DNA binding domain. The DNA binding domain can be from a molecule capable of binding a minor groove of a nucleic acid (e.g., RNA or DNA).

Another aspect of the present disclosure provides a recombinant reverse transcriptase (RT) protein comprising a RT polypeptide fused to a DNA binding domain. In some embodiments, the RT polypeptide and the DNA binding domain are separated by an amino acid linker, and the DNA binding domain is fused to the C-terminus of the RT polypeptide. In some embodiments, the RT polypeptide and the DNA binding domain are separated by an amino acid linker, and the DNA binding domain is fused to the N-terminus of the RT polypeptide.

A. DNA Binding domains

A DNA binding domain is a protein, or a defined region of a protein, that binds to a nucleic acid in a sequence-independent matter. For example, binding of the protein to DNA does not exhibit any preference for a particular sequence. The DNA binding domain may be single or double stranded. The nucleic acid binding domain can comprise a single stranded DNA binding protein; a double stranded DNA binding protein; a single stranded RNA binding protein; a double stranded RNA binding protein; a continuous RNA-DNA hybrid binding protein; or a discontinuous RNA-DNA hybrid binding protein.

The nucleic acid binding domain can help stabilize the interaction between the RNA template and the DNA primer during reverse transcription. For example, the nucleic acid binding domain can enhance the efficiency and/or processivity of the engineered RT polypeptide during reverse transcription. Suitable DNA binding domains of the present disclosure can be identical to or substantially identical to a known DNA binding protein over a comparison window of about 25 amino acids, about 50 to about 100 amino acids, any value in-between these two parameters of 25 and 100 amino acids (e.g., about 55 to about 75 amino acids), or over the length of the entire protein. The sequence can be compared and aligned for maximum correspondence over a comparison window, or designated region as measured using one of the described comparison algorithms or by manual alignment and visual inspection. For purposes of this disclosure, percent amino acid identity is determined by the default parameters of BLAST and or CLUSTAL W.

DNA binding domain (DBD) proteins or polypeptides are capable of binding DNA. DNA binding domains may include, but are not limited to, one or more DNA binding domains from an archaeal DNA binding protein, single-stranded DNA binding domains and/or 7 kDa DNA binding domains. The DNA binding domain can be a DNA binding domain of any one of Saccharomyces cerevisiae datin (DAT1), high mobility group AT hook 1 (HMGA1), lysine-specific methyltransferase 2a (KMT2A), Myocyte Enhancer Factor 2C (MEF2C), Heterogeneous Nuclear Ribonucleoprotein D (HNRNPD), Structural Maintenance of Chromosomes 1A (SMC1), Structural Maintenance Of Chromosomes 2 (SMC2), Caenorhabditis elegans tbp-1, Drosophila melanogaster D1 protein, Salmonella typhimurium Hin recombinase, S. typhimurium Gin recombinase, S. typhimurium Pin recombinase, or S. typhimurium Cin recombinase, or a combination thereof. The sequence specific DNA binding protein of any one of these molecules can be altered to produce an engineered RT polypeptide or a recombinant RT protein disclosed herein. Any proteins having substantially the same function as DAT1 and comprising any peptide motifs with sequence specific DNA binding function can be used to engineer the recombinant RT protein or engineered RT polypeptide described herein.

Specifically, the DNA binding domain can be from a S. cerevisiae DAT1. DAT1 is a yeast protein (e.g., Saccharomyces cerevisiae) that specifically recognizes the minor groove of non-alternating oligo(A)-oligo(T) tracts (e.g., >10 bp oligo(A)-oligo(T) tract). See e.g., Reardon et al., PNAS 90, 11327 (1993); Reardon et al. Nucleic Acids Research, 23, 4900 (1995). In some embodiments of the present disclosure, the DNA binding domain can comprise SEQ ID NO: 2. In some embodiments, the DNA binding domain is encoded by SEQ ID NO: 25. In some embodiments, the amino acid sequence of the DNA binding domain comprises a DNA binding domain consensus motif set forth in SEQ ID NO: 13, 14, 16, or 22.

The sequence specific recognition may be determined by to three repeated pentads of G-R-K-P-G (SEQ ID NO: 11). Accordingly, in some embodiments of the present disclosure, the DNA binding domain comprises the amino acid sequence of SEQ ID NO: 11 (GRKPG). Alternatively, the DNA binding domain can comprise at least 2 domains, at least 3 domains, at least 4 domains, at least five domains, at least six domains, at least seven domain, at least eight domains, at least nine domain, or at least ten domain comprising SEQ ID NO: 11.

In some embodiments, the DNA binding domain can specifically recognize adenine-thymine-rich region on a nucleic acid molecule. The DNA binding domain can specifically recognize oligo(dA) or oligo(dT) tracts on a nucleic acid molecule. The DNA binding domain can comprise or consist of at least one AT-rich interaction domain. For example, the DNA binding domain can comprise; consist of, or consist essentially of at least 2, at least 3, at least 4, at least 5, at least 6, at least 7, at least 8, at least 9, or at least 10 AT-rich interaction domains. The AT-rich interaction domain can comprise a core sequence. This core sequence can be a two-base core sequence, a three-base core sequence, a four-base core sequence, or a five-base core sequence. In some embodiments, the at least one of the bases of the core sequence can comprise an arginine.

In some embodiments, the at least one of the bases of the core sequence can comprise a glycine and an arginine. In some embodiments, the at least one of the bases of the core sequence can comprise a proline and an arginine. In some embodiments, the at least one of the bases of the core sequence can comprise a lysine and an arginine, or any combination thereof. In some embodiments, the at least one of the bases of the core sequence comprises an arginine; a glycine and an arginine; a proline and an arginine; a lysine and an arginine; or any combination thereof.

The AT-rich interaction domain contemplated by the present disclosure comprises a GRKPG (Gly-Arg-Lys-Pro-Gly) repeat, a RKRGRPKK repeat, a KKRGRPKK repeat, a RKRGR repeat, a GR*R/PPK repeat, a GR*RPK repeat, a GR*PPK repeat, a KRPR* repeat, or a K/RKRGRPKK repeat. In some embodiments, the AT-rich interaction domain can be a GRKPG (Gly-Arg-Lys-Pro-Gly) repeat or SEQ ID NO: 11. In some embodiments, the AT-rich interaction domain can be a RKRGRPKK repeat or SEQ ID NO: 16 or 17. In some embodiments, the AT-rich interaction domain can be KKRGRPKK repeat or SEQ ID NO: 18. In some embodiments, the AT-rich interaction domain can be a RKRGR repeat or SEQ ID NO; 19. In some embodiments, the AT-rich interaction domain can be a GR*R/PPK repeat or SEQ ID NO: 22. In some embodiments, the AT-rich interaction domain can be a GR*RPK repeat, or SEQ ID NO: 23. In some embodiments, the AT-rich interaction domain can be a GR*PPK repeat, or SEQ ID NO: 24. In some embodiments, the AT-rich interaction domain can be a KRPR* repeat, or SEQ ID NO: 21. In some embodiments, the AT-rich interaction domain can be a K/RKRGRPKK repeat, or SEQ ID NO: 16.

In some embodiments, the AT-rich interaction domain can be a mammalian high mobility group I protein (HMG-I, or a-protein) AT-rich interaction domain or SEQ ID NO: 15. In some embodiments, the AT-rich interaction domain can comprise a Drosophila melanogaster D1 protein AT-rich interaction domain-consensus domain or SEQ ID NO: 16. In some embodiments, the AT-rich interaction domain can be a Drosophila melanogaster D1 protein AT-rich interaction domain or SEQ ID NO: 17 or SEQ ID NO: 18. In some embodiments, the AT-rich interaction domain can comprise a phage 434 repressor AT-rich interaction domain or SEQ ID NO: 21. In some embodiments, the AT-rich interaction domain can comprise a Hin recombinase of Salmonella typhimurium AT-rich interaction domain or SEQ ID NO: 22, SEQ ID NO: 23, or SEQ ID NO: 24. In some embodiments, the AT-rich interaction domain comprises a core sequence comprising an amino acid selected from the group consisting of SEQ ID NO: 11-24.

In some embodiments, the DNA binding domain is a S. cerevisiae datin (DAT1) DNA binding domain or fragment thereof. Several fragments were tested and shown to maintain DNA binding activity. For example, the N-terminal 90 amino acids (D90) and/or the N-terminal 36 amino acids (D36) bind in a sequence specific manner to oligo(A)-oligo(T) tract. DAT1 (D-90) can specifically bind to A-T tracts with K_{d} of about 3×10⁻¹⁰ M (or 3×10⁻⁹ M); and DAT1 (D-36) protein can bind to A-T tracts with K_{d} of 4×10⁻¹⁰ M. DAT1 (D-90) can also be more resistant to degradation by bacterial proteases than longer and shorter DAT1 derivatives. The DNA binding activity of DAT1 (D-90) was also resistant to heat (boiling in water bath for 10 min) and chemical treatment (6 M guanidine HCl). DAT1 (D-90) was also shown to be highly soluble in physiologic salt and pH conditions.

Accordingly, in some embodiments of the engineered RT polypeptide disclosed herein, the DNA binding domain can comprise a full-length DAT1 sequence. In some embodiments, the DNA binding domain comprises SEQ ID NO: 2. The DNA binding domain can also comprise an N-terminal truncated variant of DAT1 (D90) comprising the first 90 amino acids of the full length DAT1. In that embodiment, the DNA binding domain can comprise SEQ ID NO: 3.

The DNA binding domain can also comprise a truncated variant of DAT1 (D60) comprising the first 60 amino acids of the full length DAT1. In that embodiment, the DNA binding domain can comprise SEQ ID NO: 5. The DNA binding domain can also comprise a truncated variant of DAT1 (D48) comprising the first 48 amino acids of the full length DAT1. In that embodiment, the DNA binding domain can comprise SEQ ID NO: 6. The DNA binding domain can also comprise a truncated variant of DAT1 (D36) comprising the first 36 amino acids of the full length DAT1. In that embodiment, the DNA binding domain can comprise SEQ ID NO: 8. The DNA binding domain can also comprise a truncated variant of DAT1 (D35) comprising the first 35 amino acids of full length DAT1. In that embodiment, the DNA binding domain can comprise SEQ ID NO: 9.

In some embodiments, the DNA binding domain can comprise an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 2, 3, 5, 6, 8, or 9. In some embodiments, the DNA binding domain can also comprise an amino acid sequence having 90%, 91%, 92%, 93%, 94%, 95%, 96%, 97%, 98%, or 99% sequence identity to any one of SEQ ID NO: 2, 3, 5, 6, 8, or 9. In some embodiments of the engineered RT polypeptide or recombinant RT protein described herein, the DNA binding domain can comprise the amino acid sequence of SEQ ID NO: 3, 8, or 9.

In some embodiments of the engineered RT polypeptide or recombinant RT protein described herein, the DNA binding domain can comprise a mutation in any of one of SEQ ID NO: 2, 3, 5, 6, 8, 9, or 11. The mutation can be selected from a substitution, an insertion, a deletion, or any combination thereof. In some embodiments, the mutation can further enhance the sensitivity and/or performance of the engineered RT polypeptide as described herein.

One aspect of the present disclosure provides an engineered reverse transcriptase (RT) polypeptide comprising an RT polypeptide sequence; at least two DNA binding domains, and a linker connecting the RT polypeptide sequence and the at least two DNA binding domains. In that embodiment, each DNA binding domain can be from a molecule capable of binding a minor groove of a nucleic acid. When two DNA binding domains are present, at least one DNA binding domain can be located at the N-terminus of the engineered RT and at least one DNA binding domain can be located at the C-terminus of the engineered RT. Alternatively, the at least two DNA binding domains can both be located at the C-terminus or N-terminus of the engineered RT. In some embodiments, the at least two DNA binding domains can be derived from the same molecule. In some embodiments, the at least two DNA binding domains can be derived from different molecules. In some embodiments, the at least two DNA binding domains can be derived from the same organism. In some embodiments, the at least two DNA binding domains can be derived from different organisms.

### B. Linkers

The engineered reverse transcriptase (RT) polypeptide or the recombinant RT protein described herein comprises a linker. The linker connects the RT polypeptide sequence and the DNA binding domain. Any functional linker known in the art can be used. Any suitable linker, including without limitation any variation of G(n)S(m)G(p) linker can be inserted between the RT polypeptide and the DNA binding protein. In this embodiment, n=0, 1, 2, 3, 4, 5, 6, 7, 8, 9, 10 11, 12, 13, 14, 15, 16, 17, 18, 19 or 20, m=0, 1, 2, 3, 4, 5, 6, 7, 8, 9, 10 11, 12, 13, 14, 15, 16, 17, 18, 19 or 20, p=0, 1, 2, 3, 4, 5, 6, 7, 8, 9, 10 11, 12, 13, 14, 15, 16, 17, 18, 19 or 20, and n, m, and p are selected independently. In some embodiments, the linker can be a glycine-serine linker selected from the group consisting of (GS)n, (GSGGS)n, (SGGSG)n, (GGGS)n, (GGSG)n, (GGSGG)n, (GSGSG)n, (GSGGG)n, GGGSG)n, and (GSSSG)n, where n represents an integer of at least 1. In some embodiments, the linker can be GGGS. In some embodiments, the linker can be SGGSG. The linker can comprise GGGGS or SEQ ID NO: 26. The linker can comprise GSGGSG or SEQ ID NO: 199. In some embodiments, the linker can be encoded by SEQ ID NO: 200 or GGTTCAGGGGGTTCCGGT.

The DNA binding domain described herein can be located at the N-terminus of the RT polypeptide sequence. The DNA binding domain described herein can be located at the C-terminus of the RT polypeptide sequence.

### C. Tag Proteins

One aspect of the present disclosure provides an engineered reverse transcriptase (RT) polypeptide comprising an RT polypeptide sequence; a DNA binding domain from a molecule capable of binding a minor groove of a nucleic acid; a linker connecting the RT polypeptide sequence and the DNA binding domain; and a tag protein.

Another aspect of the present disclosure provides a recombinant reverse transcriptase (RT) protein comprising a RT polypeptide, fused to a DNA binding domain and a tag protein. In that embodiment, the RT polypeptide and the DNA binding domain are separated by an amino acid linker, and the DNA binding domain is fused to the C-terminus of the RT polypeptide. In that embodiment, the RT polypeptide and the DNA binding domain are separated by an amino acid linker, and the DNA binding domain is fused to the N-terminus of the RT polypeptide

The tag protein can be selected from the group consisting of an affinity tag, a fluorescent tag, or an expression and/or solubility enhancement tag. In some embodiments, the tag protein is selected from hexahistidine tag (his-tag), Fasciola hepatica 8-kDa antigen tag (Fh8), Glutathione-S-transferase (GST) tag, maltose-binding protein tag (MBP), Flag tag peptide (FLAG tag), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin), small ubiquitin-like modifier tag (SUMO), a strep tag, Thioredoxin (Trx) tag, a VariFlex C-Terminal solubility enhancement tag, a short peptide C-terminal tag, Solubility-enhancer peptide sequences (SET) tag, IgG domain B1 of Protein G (GB1) tag, IgG repeat domain ZZ of Protein A (ZZ) tag, Mutated dehalogenase tag (HaloTag), Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Stress-responsive proteins tag (e.g., RpoA, tag, SlyD Tsf tag, RpoS tag, PotD tag, or Crr tag), and E. coli acidic proteins tag (e.g., msyB tag, yigD tag, and rpoD tag). Additional affinity tags and solubility enhancer tags are known to those skill in the art. See Costa et al., Front. Microbiol., 63 (5): (2014); Esposito and Chatterjee Curr. Opin. Biotechnol., 17:353-358 (2006); Malhotra, A. “Tagging for protein expression,” in Guide to Protein Purification, 2nd Edn, eds. R. R. Burgess and M. P. Deutscher (San Diego, CA: Elsevier), 463:239-258 (2009).

In some embodiments, the tag is selected from hexahistidine tag (his-tag), small ubiquitin-like modifier tag (SUMO), a VariFlex C-Terminal solubility enhancement tag, a short peptide C-terminal tag, Thioredoxin (Trx) tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), or fungal avidin-like protein (Tamavidin).

Tags used in the practice of any invention disclosed herein may serve any number of purposes and a number of tags may be added to impart one or more different functions to the engineered reverse transcriptase, and/or derivatives thereof, of the disclosure. For example, tags may (1) contribute to protein-protein interactions both internally within a protein and with other protein molecules, (2) make the protein amenable to particular purification methods, (3) enable one to identify whether the protein is present in a composition; or (4) give the protein other functional characteristics.

In one embodiment, the tag is an affinity tag selected from a histidine tag such as a hexahistidine tag (his-tag or 6 His-tag), Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), or fungal avidin-like protein (Tamavidin). In one embodiment, the tag is a hexahistidine tag.

In some embodiments, the tag is selected from a small ubiquitin-like modifier tag (SUMO), a VariFlex C-Terminal solubility enhancement tag, a short peptide C-terminal tag, Thioredoxin (Trx) tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In some embodiments, the solubility enhancer tag is selected from the group consisting of a SUMO tag, a GST tag, a Trx tag, a VariFlex C-Terminal solubility enhancement tag, a short peptide C-terminal tag, an Fh8 tag, MBP tag, SET tag, GB1 tag, ZZ tag, HaloTag, SNUT tag, Skp tag, T7PK tag, EspA tag, Mocr tag, Ecotin tag, CaBO tag, ArsC tag, IF2-domain I tag, Expressivity tag, RpoA, tag, SlyD, tag, Tsf tag, RpoS tag, PotD tag, Crr tag, msyB tag, yigD tag, and rpoD tag.

In some embodiments, the tag is an affinity tag. In one embodiment, the tag is an affinity tag and comprises a histidine purification tag. In one embodiment, the tag is a hexahistidine tag (his tag). In one embodiment, the tag comprises an amino acid sequence of the sequence HHHHHH (SEQ ID NO: 62). In one embodiment, the tag is a solubility enhancer tag. In one embodiment, the solubility enhancer tag is a short peptide C-terminal tag. In one embodiment, the solubility enhancer tag comprises an amino acid sequence of SEEDEEKEEDG (SEQ ID NO: 193) or an amino acid sequence having at least 90% sequence identity to the amino acid sequence of SEQ ID NO: 193.

In some embodiments, the tag further comprises an endoprotein cleavage site selected from ENLYFQ/G (SEQ ID NO: 194), DDDDK/(SEQ ID NO: 195), IEGR/(SEQ ID NO: 196), LVPR/GS (SEQ ID NO: 197), or LEVLFQ/GP (SEQ ID NO: 198).

In some embodiments, the engineered nucleic acid processing enzyme or a derivative thereof further comprises a protease cleavage sequence. In some embodiments, the cleavage of the protease cleavage sequence by a protease results in cleavage of the affinity tag from the engineered reverse transcriptase polypeptide or recombinant RT protein or a derivative thereof. In some instances, the protease cleavage sequence/site is recognized by a protease including, but not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase (EnTK), gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin (Thr), tissue kallikrein, tobacco etch virus (TEV), togavirin, tryptophanyl aminopeptidase, U-plasminogen activator, V8, venombin A, venombin AB, factor Xa (Xa), and Xaa-pro aminopeptidase. In some embodiments, the protease cleavage sequence is a thrombin cleavage sequence.

In some embodiments, the tag is cleaved or removed from the engineered nucleic acid processing enzyme or derivatives thereof via the cleavage site. In one embodiment, the tag is cleaved or removed using an endoprotein selected from the group consisting of tobacco etch virus protease (Tev), enterokinase (EntK), factor Xa (Xa), thrombin (Thr), genetically engineered derivative of human rhinovirus 3C protease (PreScission), Catalytic core of Ulp1 (SUMO protease). In one embodiment, the tag is cleaved at ENLYFQ/G (SEQ ID NO: 194) using tobacco etch virus protease (Tev). In another embodiment, the tag is cleaved at DDDDK/(SEQ ID NO: 195) using Enterokinase (EntK). In another embodiment, the tag is cleaved at IEGR/(SEQ ID NO: 196) using Factor Xa (Xa). In another embodiment, the tag is cleaved at LVPR/GS (SEQ ID NO: 197) using thrombin (Thr). In another embodiment, the tag is cleaved at LEVLFQ/GP (SEQ ID NO: 198) using a genetically engineered derivative of human rhinovirus 3C protease. In another embodiment, the tag is cleaved with Catalytic core of Ulp1 (SUMO protease). Catalytic core of Ulp1 recognizes SUMO tertiary structure and cleaves at the C-terminal end of the conserved Gly-Gly sequence in SUMO.

In some embodiments, the engineered RT polypeptide, the recombinant RT protein, or derivatives thereof comprises an affinity tag at the N-terminus or at the C-terminus of the amino acid sequence. In some embodiments, the affinity tag include, but is not limited to, albumin binding protein (ABP), AU1 epitope, AU5 epitope, T7-tag, V5-tag, B-tag, Chloramphenicol Acetyl Transferase (CAT), Dihydrofolate reductase (DHFR), AviTag, Calmodulin-tag, polyglutamate tag, E-tag, FLAG-tag, HA-tag, Myc-tag, NE-tag, S-tag, SBP-tag, Doftag 1, Softag 3, Spot-tag, tetracysteine (TC) tag, Ty tag, VSV-tag, Xpress tag, biotin carboxyl carrier protein (BCCP), green fluorescent protein tag, HaloTag, Nus-tag, thioredoxin-tag, Fc-tag, cellulose binding domain, chitin binding protein (CBP), choline-binding domain, galactose binding domain, maltose binding protein (MBP), Horseradish Peroxidase (HRP), Strep-tag, HSV epitope, Ketosteroid isomerase (KSI), KT3 epitope, LacZ, Luciferase, PDZ domain, PDZ ligand, Polyarginine (Arg-tag), Polyaspartate (Asp-tag), Polycysteine (Cys-tag), Polyphenylalanine (Phe-tag), Profinity eXact, Protein C, S1-tag, S1-tag, Staphylococcal protein A (Protein A), Staphylococcal protein G (Protein G), Small Ubiquitin-like Modifier (SUMO), Tandem Affinity Purification (TAP), TrpE, Ubiquitin, Universal, glutathione-S-transferase (GST), and poly(His) tag. In some instances, the affinity tag is at least 5 histidine amino acids.

In some embodiments, engineered reverse transcription enzymes, engineered reverse transcriptases, engineered reverse transcriptase polypeptides, or recombinant RT proteins described herein may comprise an affinity tag at the N-terminus or at a C-terminus of the amino acid sequence. In some instances, the affinity tag may include, but is not limited to, albumin binding protein (ABP), AU1 epitope, AU5 epitope, T7-tag, V5-tag, B-tag, Chloramphenicol Acetyl Transferase (CAT), Dihydrofolate reductase (DHFR), AviTag, Calmodulin-tag, polyglutamate tag, E-tag, FLAG-tag, HA-tag, Myc-tag, NE-tag, S-tag, SBP-tag, Doftag 1, Softag 3, Spot-tag, tetracysteine (TC) tag, Ty tag, VSV-tag, Xpress tag, biotin carboxyl carrier protein (BCCP), green fluorescent protein tag, HaloTag, Nus-tag, thioredoxin-tag, Fc-tag, cellulose binding domain, chitin binding protein (CBP), choline-binding domain, galactose binding domain, maltose binding protein (MBP), Horseradish Peroxidase (HRP), Strep-tag, HSV epitope, Ketosteroid isomerase (KSI), KT3 epitope, LacZ, Luciferase, PDZ domain, PDZ ligand, Polyarginine (Arg-tag), Polyaspartate (Asp-tag), Polycysteine (Cys-tag), Polyphenylalanine (Phe-tag), Profinity eXact, Protein C, S1-tag, S1-tag, Staphylococcal protein A (Protein A), Staphylococcal protein G (Protein G), Small Ubiquitin-like Modifier (SUMO), Tandem Affinity Purification (TAP), TrpE, Ubiquitin, Universal, glutathione-S-transferase (GST), and poly(His) tag. In some instances, said affinity tag is at least 6 histidine amino acids (SEQ ID NO: 26).

In some embodiments, an engineered reverse transcriptase polypeptide and/or a recombinant reverse transcriptase protein described herein can comprise a protease cleavage sequence. In that embodiment, cleavage by a protease results in cleavage of the affinity tag from the engineered reverse transcription enzyme. In some instances, the protease cleavage sequence is recognized by a protease including, but not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase. In some instances, the protease cleavage sequence is a thrombin cleavage sequence.

### D. Reverse Transcriptase Polypeptides

Reverse transcriptases or reverse transcription enzymes are known in the art to perform a reverse transcription reaction. As used herein, “Reverse transcriptase” and “reverse transcription enzyme” are synonymous. Reverse transcription is initiated by hybridization of a priming sequence to an RNA molecule which is extended by an engineered reverse transcription enzyme in a template directed fashion. A reverse transcription enzyme adds a plurality of non-template oligonucleotides to a nucleotide strand. The reverse transcription reaction can produce single stranded complementary deoxyribonucleic acid (cDNA) molecules each having a molecular tag on a 5′ end thereof, followed by amplification of cDNA to produce a double stranded DNA having the molecular tag on the 5′ end and a 3′ end of the double stranded DNA.

As used herein, the term “wild-type” refers to a gene or gene product that has the characteristics of that gene or gene product when isolated from a naturally occurring source. For example, the amino acid sequence set forth in SEQ ID NO: 7 is a wild-type MMLV amino acid sequence.

In some embodiments of the engineered RT polypeptide or the recombinant RT protein disclosed herein, the RT polypeptide can comprise an amino acid sequence that is at least 90% identical to SEQ ID NO: 1. In some embodiments, the RT polypeptide can comprise an amino acid sequence that is 90-99.99% identical to SEQ ID NO: 1. In some embodiments, the RT polypeptide can comprise an amino acid sequence that is 92-99.99% identical to SEQ ID NO: 1. In some embodiments, the RT polypeptide can comprise an amino acid sequence that is 93-99.99% identical to SEQ ID NO: 1. In some embodiments, the RT polypeptide can comprise an amino acid sequence that is 94-99.99% identical to SEQ ID NO: 1. In some embodiments, the RT polypeptide can comprise an amino acid sequence that is 95-99.99% identical to SEQ ID NO: 1. In some embodiments, the RT polypeptide can comprise an amino acid sequence that is 96-99.99% identical to SEQ ID NO: 1. In some embodiments, the RT polypeptide can comprise an amino acid sequence that is 97-99.99% identical to SEQ ID NO: 1. In some embodiments, the RT polypeptide can comprise an amino acid sequence that is 98-99.99% identical to SEQ ID NO: 1. In some embodiments of the engineered RT polypeptide or the recombinant RT protein disclosed herein, the RT polypeptide can comprise an amino acid sequence that is 90%, 91%, 92%, 93%, 94%, 95%, 96%, 97%, 98%, 99% or 99.5% identical to SEQ ID NO: 1. The amino acid variation are at any one position or combination thereof as identified in an alignment of SEQ ID NO: 1 to any one of the RT polypeptide sequences in Table 1, or Table 2.

In some embodiments, the RT polypeptide can comprise the amino acid sequence set forth in SEQ ID NO: 7. The engineered reverse transcriptase can exhibit an altered reverse transcriptase activity as compared to a reverse transcriptase having the amino acid sequence set forth in SEQ ID NO: 1 or 7.

The RT polypeptide can be a variant MMLV reverse-transcriptase having one or more mutations. Specifically, the RT polypeptide contemplated by the present disclosure can comprise a combination of mutations in the amino acid sequence of either the wild-type MMLV (SEQ ID NO 7 or 178) or in a MMLV variant (SEQ ID NO: 1, 143 or 179).

The amino acid sequence of the RT polypeptide sequence contemplated by the present disclosure can be at least 90% identical to SEQ ID NO: 1 or 143. The amino acid sequence of the RT polypeptide sequence can be about 90% to about 99.99% identical to SEQ ID NO: 1 or 143, about 92% to about 99.99% identical to SEQ ID NO: 1 or 143, about 93% to about 99.99% identical to SEQ ID NO: 1 or 143, about 94% to about 99.99% identical to SEQ ID NO: 1 or 143, about 95% to about 99.99% identical to SEQ ID NO: 1 or 143, about 96% to about 99.99% identical to SEQ ID NO: 1 or 143, about 97% to about 99.99% identical to SEQ ID NO: 1 or 143, or about 98% to about 99.99% identical to SEQ ID NO: 1 or 143. In some embodiments, the amino acid sequence of the RT polypeptide sequence can be about 90%, about 91%, about 92%, about 93%, about 94%, about 95%, about 96%, about 97%, about 98%, about 99% or about 99.5% identical to SEQ ID NO: 1 or 143.

As used herein, a “Mutation” refers to a change introduced into a parental or wild type DNA sequence that changes the amino acid sequence encoded by the DNA, including, but not limited to, substitutions, insertions, deletions, point mutations, mutation of multiple nucleotides or amino acids, transposition, inversion, frame shift, nonsense mutations, truncations or other forms of aberration that differentiate the polynucleotide or protein sequence from that of a wild-type sequence of a gene or gene product. The consequences of a mutation include, but are not limited to, the creation of a new character, property, function, or trait not found in the protein encoded by the parental DNA, including, but not limited to, N terminal truncation, C terminal truncation or chemical modification. A “mutation”” also includes an N- or C-terminal extension. In some embodiments, the mutations disclosed herein are substitutions.

In particular, the present disclosure relates to engineered RT polypeptides or recombinant RT polypeptide comprising a wild-type RT or modified reverse transcriptases that comprise one or more (e.g., one, two, three, four, five, ten, twelve, fifteen, twenty, etc.) amino acid changes. These amino acid changes render the reverse transcriptase more efficient for nucleic acid synthesis (e.g., single cell profiling assay) requiring very small volume, as compared to an unmutated or an unmodified reverse transcriptase. As will be appreciated by those skilled in the art, one or more of the amino acids identified may be deleted and/or replaced with one or a number of amino acid residues. In a preferred aspect, any one or more of the amino acids may be substituted with any one or more amino acid residues such as Ala, Arg, Asn, Asp, Cys, Gln, Glu, Gly, His, He, Leu, Lys, Met, Phe, Pro, Ser, Thr, Trp, Tyr, and/or Val.

In some embodiments, the RT polypeptide described herein comprises the amino acid sequence of SEQ ID NO:7, and comprises a combination of mutations selected from E69K, L139P, E302R, T306K, W313F, T330P, or N454K; and one or more of M39V, P47L, M66L, F155Y, D200N, D200E, H204R, G429S, L435G, L435K, P448A, D449G, H503V, D524N, T542D, E545G, D583N, H594Q, L603W, L603F, E607K, E607G, P627S, H634Y, H638G, A644V, D653H, K658R or L671P. The engineered polypeptide can comprise the amino acid sequence of SEQ ID NO:7, and comprises a combination of mutations selected from E69K, L139P, D200N, E302R, T306K, W313F, T330P, L435G, P448A, D449G, N454K, D524N, or L603W, and E607K and one or more of M39V, P47L, M66L, F155Y, H204R, G429S, H503V, T542D, E545G, D583N, H594Q, P627S, H634Y, H638G, A644V, D653H, K658R or L671P.

The RT polypeptide sequence can comprise an amino acid sequence that is at least 95% identical to SEQ ID NO:1, 7, or 179, and a combination of mutations indexed to SEQ ID NO: 7 or 178 selected from a combination of variants consisting of a T542D mutation, a D583N mutation, an E607G mutation, an A644V mutation, a D653H mutation, and a K658R mutation. The RT polypeptide sequence can comprise an amino acid sequence that is at least 95% identical to SEQ ID NO:1, 7, or 179, and a combination of mutations indexed to SEQ ID NO:7 or 178 selected from a combination of variants consisting of an E545G mutation, a D583N mutation, an H594Q mutation, an L603F mutation, and a S679P mutation.

In some embodiments, the RT polypeptide comprises an amino acid sequence that is at least 90% identical to an amino acid sequence selected from: SEQ ID NO: 14, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, or SEQ ID NO: 55.

In some embodiments, the RT polypeptide can comprise SEQ ID NO: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, 172, or 173. The RT polypeptide can comprise an amino acid sequence listed in Table 1 or 2.

The amino acid sequence of the RT polypeptide can also comprise E69K, L139P, D200N, E302R, T306K, W313F, T330P, N454K, H503V, D524N, L603W, E607K, and H634Y. In some embodiments, the amino acid sequence of the RT polypeptide comprises a combination of mutations selected from: M66L and L435G; M39V, M66L, and L435K; M39V and L435K; M66L, L435G, P448A and D449G; M39V, M66L, L435G, P448A and D449G; or M66L.

In some embodiments, the amino acid sequence of the RT polypeptide comprises E69K, L139P, D200N, E302R, T306K, W313F, T330P, L435G, P448A, D449G, N454K, D524N, L603W, and E607K; and further comprises a combination of mutations selected from M66L; M66L and H503V; M66L and H634Y; and M66L, H503V, or H634Y.

In some embodiments, the amino acid sequence of the RT polypeptide comprises M39V, E69K, L139P, a D200 mutation, E302R, T306K, W313F, T330P, G429S, P448A, a D449 mutation, L435K, N454K, a L603 mutation, a E607 mutation, and L671P and further comprises a second combination of mutations selected from D524N, T542D, P627S, A644V, D653H, or K658R mutation. In that embodiment, the D200 mutation is a D200N mutation, the D449 mutation is a D449G, the L603 mutation is an L603W, or the E607 mutation is an E607G mutation.

In some embodiments, the amino acid sequence of the RT polypeptide comprises M39V, E69K, L139P, a D200 mutation, E302R, T306K, W313F, T330P, G429S, P448A, a D449 mutation, L435K, N454K, a L603 mutation, a E607 mutation, and L671P and further comprises D524N, T542D, A644V, D653H, an R650H and K658R. In that embodiment, the D200 mutation is a D200N mutation, the D449 mutation is a D449E mutation, the L603 mutation is an L603W mutation, and the E607 mutation is an E607G mutation.

In some embodiments, the amino acid sequence of the RT polypeptide comprises M39V, E69K, L139P, a D200 mutation, E302R, T306K, W313F, T330P, G429S, P448A, a D449 mutation, L435K, N454K, a L603 mutation, a E607 mutation, and L671P and further comprises E545G, D583N, and H594Q. In that embodiment, the D200 mutation is a D200N mutation, the D449 mutation is a D449G mutation, the L603 mutation is an L603F mutation, and the E607 mutation is an E607K mutation.

In some embodiments, the amino acid sequence of the RT polypeptide comprises M39V, E69K, L139P, a D200 mutation, E302R, T306K, W313F, T330P, G429S, P448A, a D449 mutation, L435K, N454K, a L603 mutation, a E607 mutation, and L671P and further comprises D524N, T542D, A644V, D653H, and K658R. In that embodiment, the D200 mutation is a D200N mutation, the D449 mutation is a D449E mutation, the L603 mutation is an L603W mutation, and the E607 mutation is an E607G mutation.

In some embodiments, the amino acid sequence of the RT polypeptide comprises M39V, E69K, L139P, a D200 mutation, E302R, T306K, W313F, T330P, G429S, P448A, a D449 mutation, L435K, N454K, a L603 mutation, a E607 mutation, and L671P and further comprises H204R, D524N, T542D, P627S, D583N, A644V, D653H and K658R. In that embodiment, the D200 mutation is a D200E mutation, the D449 mutation is a D449G mutation, the L603 mutation is an L603W mutation, and the E607 mutation is an E607G mutation.

In some embodiments, the amino acid sequence of the RT polypeptide comprises M39V, E69K, L139P, a D200 mutation, E302R, T306K, W313F, T330P, G429S, P448A, a D449 mutation, L435K, N454K, a L603 mutation, a E607 mutation, and L671P and further comprises H204R, E545G, D583N, and H594Q. In that embodiment, the D200 mutation is a D200E mutation, the D449 mutation is a D449G mutation, the L603 mutation is an L603F mutation, and the E607 mutation is an E607K mutation.

In some embodiments, the amino acid sequence of the RT polypeptide comprises M39V, E69K, L139P, a D200 mutation, E302R, T306K, W313F, T330P, G429S, P448A, a D449 mutation, L435K, N454K, a L603 mutation, a E607 mutation, and L671P and further comprises P47L, D524N, T542D, D583N, P627S, A644V, D653H, and K658R. In that embodiment, the D200 mutation is a D200N mutation, the D449 mutation is a D449G mutation, the L603 mutation is an L603W mutation, and the E607 mutation is an E607G mutation.

In some embodiments, the RT polypeptide comprises an amino acid sequence that is at least 95% identical to SEQ ID NO: 1 and the amino acid sequence of the engineered reverse transcriptase comprises at least one mutation indexed to SEQ ID NO:7 selected from a M17 mutation; an A32 mutation, a M44 mutation, a M39 mutation, a K47 mutation, a P51 mutation, an M66 mutation, an S67 mutation, an E69 mutation, a L72 mutation, a W94 mutation, a K103 mutation, an R110 mutation, a P117 mutation, an L139 mutation, an F155 mutation, an N178 mutation, an E179 mutation, a T197 mutation, a D200 mutation, an E201 mutation, an H204 mutation, a Q221 mutation, a V223 mutation, a V238 mutation, a G248 mutation, a T265 mutation, an E268 mutation, an R279 mutation, an R280 mutation, a K284 mutation, a T287 mutation, a F291 mutation, an E302 mutation, an E302K mutation, an E302R mutation, a T306 mutation, a T306R mutation, a T306K mutation a P308 mutation, an F309 mutation, a W313 mutation, a T330 mutation, a Y344 mutation, an 1347 mutation, a C387 mutation, a W388 mutation, an R389 mutation, a C409 mutation, an R411 mutation, a G413 mutation, an A426 mutation, a G427 mutation, an L435 mutation, an L435G mutation, an L435K mutation, a P448 mutation, a D449 mutation, an R450 mutation, a n N454 mutation, an A480 mutation, an H481 mutation, a N502 mutation, an A502 mutation, an H503 mutation, a D524 mutation, an H572 mutation, a W581 mutation, a D583 mutation, a K585 mutation, an H594 mutation, an L603 mutation, an E607 mutation, an H612 mutation, a P614 mutation, a G615 mutation, an H634 mutation, a P636 mutation, a G637 mutation, an H638 mutation, a D653 mutation, an L671 mutation, or a combination thereof.

In some embodiments, the RT polypeptide comprises an amino acid sequence that is at least 95% identical to SEQ ID NO: 1 and the amino acid sequence of the engineered reverse transcriptase comprises an M39 mutation, a K47 mutation, an L435 mutation, a D449 mutation, a D524 mutation, an E607 mutation, a D653 mutation, and an L671 mutation as indexed to SEQ ID NO: 7 and comprising at least one mutation indexed to SEQ ID NO:7 selected from a M17 mutation; an A32 mutation, a M44 mutation, a M39V mutation, a P51 mutation, an M66 mutation, an S67 mutation, an E69 mutation, a L72 mutation, a W94 mutation, a K103 mutation, an R110 mutation, a P117 mutation, an L139 mutation, an F155 mutation, an N178 mutation, an E179 mutation, a T197 mutation, a D200 mutation, an E201 mutation, an H204 mutation, a Q221 mutation, a V223 mutation, a V238 mutation, a G248 mutation, a T265 mutation, an E268 mutation, an R279 mutation, an R280 mutation, a K284 mutation, a T287 mutation, a F291 mutation, an E302 mutation, an E302K mutation, an E302R mutation, a T306 mutation, a T306R mutation, a T306K mutation a P308 mutation, an F309 mutation, a W313 mutation, a T330 mutation, a Y344 mutation, an 1347 mutation, a C387 mutation, a W388 mutation, an R389 mutation, a C409 mutation, an R411 mutation, a G413 mutation, an A426 mutation, a G427 mutation, an L435G mutation, an L435K mutation, a P448 mutation, a D449G mutation, an R450 mutation, a n N454 mutation, an A480 mutation, an H481 mutation, a N502 mutation, an A502 mutation, an H503 mutation, a D524N mutation, an H572 mutation, a W581 mutation, a D583 mutation, a K585 mutation, an H594 mutation, an L603 mutation, an H612 mutation, a P614 mutation, a G615 mutation, an H634 mutation, a P636 mutation, a G637 mutation, an H638 mutation, or a combination thereof.

In other embodiments, the engineered RT polypeptide exhibits an altered reverse transcriptase related activity when compared to a reverse transcriptase having the amino acid sequence set forth in SEQ ID NO: 1.

In some embodiments, an RT polypeptide comprises an amino acid sequence that is at least 95% identical to SEQ ID NO: 1. In other embodiments, the engineered reverse transcriptase exhibits an altered reverse transcriptase related activity as compared to a reverse transcriptase having the amino acid sequence set forth in SEQ ID NO: 1. In additional embodiments, the RT polypeptide comprises a combination of mutations indexed to SEQ ID NO:7 selected from: (i) an E69K mutation, an E302R mutation, a T306K mutation, a W313F mutation, a L435G mutation, or an N454K mutation, and comprising at least one mutation selected from an M39V mutation, an M66L mutation, an L139P mutation, an F155Y mutation, a D200N mutation, an E201Q mutation, a T287A mutation, a T330P mutation, an R411F mutation, a P448A mutation, a D449G mutation, an H503V mutation, an H594K mutation, L603W mutation, an E607K mutation, an H634Y mutation, a G637R mutation and an H638G mutation; (ii) an L139P mutation, a D200N mutation, a T330P mutation, an L603W mutation, or an E607K mutation, and comprising at least one mutation selected from: an M39V mutation, an M66L mutation an E69K mutation, an F155Y mutation, an E201Q mutation, a T287A mutation, an E302R mutation, a T306K mutation, a W313F mutation, an R411F mutation, an L435G mutation, a P448A mutation, a D449G mutation, an N454K mutation, an H503V mutation, an H594K mutation, an H634Y mutation, a G637R mutation or an H638G mutation; (iii) an A32V mutation, an L72R mutation, a D200C mutation, a G248C mutation, an E286R mutation, an E302R mutation, a W388R mutation, and an L435G mutation; or (iv) a Y344L mutation and an I347L mutation.

In some embodiments, the RT polypeptide comprises an amino acid sequence that is at least 95% identical to a reverse transcriptase having the amino acid sequence set forth in SEQ ID NO: 1. In some embodiments, the engineered reverse transcription enzyme comprises an amino acid sequence that is at least 95% identical to SEQ ID NO: 1 and has at least one mutation selected from the group consisting of an M39V mutation, a P47L mutation, M66L mutation, an E69K mutation, an L139P mutation, a D200N mutation, an H204R mutation, an E302R mutation, a T306K mutation, a W313F mutation, a T330P mutation, an L435G mutation, a G429S mutation, an L435K mutation, a P448A mutation, a D449G mutation, a N454K mutation, an H503V mutation, a D524N mutation, a T542 mutation, an E545G mutation, a D583N mutation, an H594Q mutation, an L603W mutation, an E607K mutation, a P627S mutation, an H634Y mutation, an A644V mutation, an R650H mutation, a D653H mutation, a K658R mutation, an L671P mutation, or an S679P mutation; and the engineered RT polypeptide or recombinant RT protein described herein exhibits an altered reverse transcriptase related activity.

In some embodiments, the disclosure provides an engineered RT polypeptide comprising an amino acid sequence that is at least 95% identical to SEQ ID NO:1 and the amino acid sequence of the engineered reverse transcriptase comprises a combination of mutations indexed to SEQ ID NO:7 or 178 selected from the group consisting of (a) an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation; (b) an M66L mutation, an E69K mutation, an L139P mutation, a D200N mutation, an E302R mutation, a T306K mutation, a W313F mutation, a T330P mutation, a N454K mutation, a D524N mutation, an H503V mutation, an L603W mutation, an E607K mutation, and an H634Y mutation, and at least one mutation selected from the group consisting of an L435G mutation, an L435K mutation, an M39V mutation, a P448A mutation and a D449G mutation; (c) an M39V mutation, an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation; and (d) an M39V mutation, an E69K mutation, an L139P mutation, a D200 mutation, an E302R mutation, a T306K mutation, a W313F mutation, a T330P mutation, an L435K mutation, a G429S mutation, a P448A mutation, a D449 mutation, an N454K mutation, an L603 mutation, an E607 mutation and an L671P mutation. In that embodiment, the D200 mutation is selected from the group consisting of D200N and D200E. In that embodiment, the D449 mutation is selected from the group consisting of D449G an D449E. In that embodiment, the L603 mutation is selected from the group consisting of L603W and L603F. In that embodiment, the E607 mutation is selected from the group consisting of E607G and E607K. In another embodiment, the engineered RT polypeptide further comprises at least one mutation selected from the group consisting of P47L, H204R, D524N, T542D, E545G, D583N, H594Q, P627S, A644V, R650H, D653H, K658R, L671P, and S679P.

In some embodiments, an engineered reverse transcriptase of the present application has an amino acid sequence that is at least 95% identical to SEQ ID NO: 1 and the amino acid sequence of said engineered reverse transcriptase comprises a combination of mutations indexed to SEQ ID NO:7 or 178; and the amino acid sequence of said engineered reverse transcriptase comprises a combination of mutations selected from the group consisting of: an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation and further comprising a second combination of mutations selected from the group consisting of: (a) an M66L mutation and an L534G mutation, (b) an M39V mutation, an M66L mutation and an L435K mutation, (c) an M39V mutation and an L435K mutation, (d) an M66L mutation, an L435G mutation, a P448 mutation, and D449G mutation, and (e) an M39V mutation, an M66L mutation, an L435G mutation, a P448 mutation and a D449G mutation.

In some embodiments, an engineered reverse transcriptase of the present application has an amino acid sequence that is at least 95% identical to SEQ ID NO: 1 and the amino acid sequence of the engineered reverse transcriptase comprises a combination of mutations selected from the group consisting of: an M39V mutation, an E69K mutation, an L139P mutation, a D200 mutation, an E302R mutation, a T306K mutation, a W313F mutation, a T330P mutation, a G429S mutation a P448A mutation, a D449 mutation, an L435K mutation, a N454K mutation, an L603 mutation, an E607 mutation, and an L671P mutation and further comprising a second combination of mutations selected from the group consisting of: (a) a D524N mutation, a T542D mutation, an A644V mutation, a D653H mutation, a K658R mutation, a S679P mutation, and wherein said D200 mutation is a D200N mutation, said D449 mutation is a D449G, said L603 mutation is an L603W, said E607 mutation is an E607G mutation, and a P627S mutation; (b) a D524N mutation, a T542D mutation, an A644V mutation, a D653H mutation, an R650 mutation and a K658R mutation, and wherein said D200 mutation is a D200N mutation, said D449 mutation is a D449E mutation, said L603 mutation is an L603W mutation, and said E607 mutation is an E607G mutation; (c) an E545G mutation, a D583N mutation, an H594Q mutation, and an S679P mutation, and wherein said D200 mutation is a D200N mutation, said D449 mutation is a D449G mutation, said L603 mutation is an L603F mutation, said E607 mutation is an E607K mutation; (d) a D524N mutation, a T542D mutation, an A644V mutation, a D653H mutation, and a K658R mutation, and wherein said D200 mutation is a D200N mutation, said D449 mutation is a D449E mutation, said L603 mutation is an L603W mutation, said E607 mutation is an E607G mutation; (e) an H204R mutation, a D524N mutation, a T542D mutation, a D583N mutation, an A644V mutation, a D653H mutation, and a K658R mutation, wherein said D200 mutation is a D200E mutation, said D449 mutation is a D449G mutation, said L603 mutation is an L603W mutation, said E607 mutation is an E607G mutation, and a P627S mutation, (f) an H204R mutation, an E454G mutation, a D583N mutation, an H594Q mutation, and an S679P mutation, wherein said D200 mutation is a D200E mutation, said D449 mutation is a D449G mutation, said L603 mutation is an L603F mutation, said E607 mutation is an E607K mutation; and (g) a P47 mutation, a D524N mutation, a T542D mutation, a D583N mutation, an A644V mutation, a D653H mutation, a K658R mutation and an S679P mutation. In that embodiment, the P47 mutation is a P47L mutation, the D200 mutation is a D200N mutation, the D449 mutation is a D449G mutation, the L603 mutation is an L603W mutation, the E607 mutation is an E607G mutation, and the P627 mutation is a P627S mutation.

A variant may comprise a first combination of mutations or alterations and may comprise an additional or second combination of mutations.

A first combination of mutations or alterations may include, but is not limited to, a combination of: (1) a M39 mutation, a K47 mutation, an L435 mutation, a D449 mutation, a D524 mutation, an E607 mutation, a D653 mutation and an L671 mutation; (2) an M39V mutation, a K47 mutation, an L435K mutation, a D449G mutation, a D524N mutation, an E607 mutation, a D653 mutation and an L671 mutation; (3) an M39 mutation, an M66 mutation, an E302 mutation, a T306 mutation, an L435 mutation, a D449 mutation, a D524 mutation, an E607 mutation, a D653 mutation and an L671 mutation; (4) an M39 mutation, an M66 mutation, an E302 (K or R) mutation, a T306 (R or K) mutation, an L435 (K or G), a D449 mutation, a D524 mutation, an E607 (G or K) mutation, a D653 mutation, and an L671 mutation; and (5) an M39V mutation, an M66 mutation, an E302 (K or R) mutation, a T306 (R or K) mutation, an L435 (K or G), a D449G mutation, a D524N mutation, an E607 (G or K) mutation, a D653 mutation, and an L671 mutation.

The second combination of mutations in a first engineered reverse transcriptase may comprise either a different set of mutations or a partially different second set of mutations as in a second engineered reverse transcriptase. A second combination of mutations or alterations may include but is not limited to: (a) one or more mutations selected from an M17 mutation; an A32 mutation, a M44 mutation, a P51 mutation, an M66 mutation, an S67 mutation, an E69 mutation, a L72 mutation, a W94 mutation, a K103 mutation, an R110 mutation, a P117 mutation, an L139 mutation, an F155 mutation, an N178 mutation, an E179 mutation, a T197 mutation, a D200 mutation, an E201 mutation, an H204 mutation, a Q221 mutation, a V223 mutation, a V238 mutation, a G248 mutation, a T265 mutation, an E268 mutation, an R279 mutation, an R280 mutation, a K284 mutation, a T287 mutation, a F291 mutation, an E302 mutation, an E302K mutation, an E302R mutation, a T306 mutation, a T306R mutation, a T306K mutation, a P308 mutation, an F309 mutation, a W313 mutation, a T330 mutation, a Y344 mutation, an I347 mutation, a C387 mutation, a W388 mutation, an R389 mutation, a C409 mutation, an R411 mutation, a G413 mutation, an A426 mutation, a G427 mutation, an L435G mutation, an L435K mutation, a P448 mutation, a D449G mutation, an R450 mutation, an N454 mutation, an A480 mutation, an H481 mutation, a N502 mutation, an A502 mutation, an H503 mutation, a D524N mutation, an H572 mutation, a W581 mutation, a D583 mutation, a K585 mutation, an H594 mutation, an L603 mutation, an H612 mutation, a P614 mutation, a G615 mutation, an H634 mutation, a P636 mutation, a G637 mutation, or an H638 mutation; (b) an E69K mutation, an E302R mutation, a T306K mutation, a W313F mutation, an L435G mutation, and an N454K mutation, and comprising at least one mutation selected from the group consisting of an M39V mutation, an M66L mutation, an L139P mutation, an F155Y mutation, a D200N mutation, an E201Q mutation, a T287A mutation, a T330P mutation, an R411F mutation, a P448A mutation, a D449G mutation, an H503V mutation, an H594K mutation, L603W mutation, an E607K mutation, an H634Y mutation, a G637R mutation and an H638G mutation; (c) an L139P mutation, a D200N mutation, a T330P mutation, an L603W mutation, and an E607K mutation, and comprising at least one mutation selected from the group consisting of: an M39V mutation, an M66L mutation, an E69K mutation, an F155Y mutation, an E201Q mutation, a T287A mutation, an E302R mutation, a T306K mutation, a W313F mutation, an R411F mutation, an L435G mutation, a P448A mutation, a D449G mutation, an N454K mutation, an H503V mutation, an H594K mutation, an H634Y mutation, a G637R mutation and an H638G mutation; (d) an A32V mutation, an L72R mutation, a D200C mutation, a G248C mutation, an E286R mutation, an E302R mutation, a W388R mutation, or an L435G mutation; and (e) a Y344L mutation and an I347L mutation. It is recognized that the second combination of mutations may comprise a group of mutations as described herein and one or more additional mutations.

In non-limiting embodiments, the engineered RT variants of the present disclosure comprise a M39V, M66I, Q91R, I347V, H594Q, or a combination thereof in the RT backbone of SEQ ID NO: 143 or SEQ ID NO: 7. In non-limiting embodiments, the engineered RT polypeptide comprises: M39V, M66I, Q91R, I347V, and H594Q (SEQ ID NO: 129, SOLD 034). In non-limiting embodiments, the engineered RT variants of the present disclosure comprise M39V, T542D, D583N, E607G, A644V, D653H, K658R, L671P, or a combination thereof in the RT backbone of SEQ ID NO: 143 or SEQ ID NO: 7. In non-limiting embodiments, the engineered RT polypeptide comprises: M39V, T542D, D583N, E607G, A644V, D653H, K658R, and L671P (SEQ ID NO: 111, SOLD 025).

In some embodiments, the engineered RT polypeptide comprises: M39V, M66I, Q91R, I347V, H594Q, or a combination thereof, and optionally M39V, M66I, Q91R, I347V, H594Q, or the combination thereof (substituted) in the RT sequence of SEQ ID NO: 143 (SEQ ID NO: 129, SOLD 034).

In some embodiments, the engineered RT polypeptide comprises: M39V, T542D, D583N, E607G, A644V, D653H, K658R, L671P, or a combination thereof, and optionally M39V, T542D, D583N, E607G, A644V, D653H, K658R, L671P, or the combination thereof substituted in the RT sequence of SEQ ID NO: 143 (or SEQ ID NO: 7) (SEQ ID NO: 111, SOLD 025). In some embodiments, the engineered RT polypeptide comprises SOLD 33 VDG or SEQ ID NO: 173.

In some embodiments, the engineered RT polypeptide described herein comprises an amino acid sequence that is at least about 90% identical to an amino acid sequence selected from the group consisting of SEQ ID NO: 27-61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, 172, and 173.

In some embodiments of the engineered RT polypeptide described herein, the RT polypeptide is 42B L (SEQ ID NO: 145), 50A+G (SEQ ID NO: 147), SOLD 022 (SEQ ID NO: 105), SOLD 023 (SEQ ID NO: 107), SOLD 025 (SEQ ID NO: 111), SOLD 031 (SEQ ID NO: 123), SOLD 033 (SEQ ID NO: 127), SOLD 034 (SEQ ID NO: 129), SOLD 035 (SEQ ID NO: 131), SOLD 001 (SEQ ID NO: 65), SOLD 33 VDG (SEQ ID NO: 173), or an RT polypeptide set forth in SEQ ID NO: 143, or SEQ ID NO: 172.

### E. Engineered DAT1 Reverse Transcriptases

One aspect of the present disclosure provides an engineered RT polypeptide comprising an amino acid sequence that is at least 90%, at least 92%, at least 95%, at least 97%, at least 98%, or at least 99% identical to an amino acid sequence of an RT disclosed in Table 1, or Table 2, and a DNA binding domain comprising an amino acid sequence selected from the group consisting of SEQ ID NO: 2, 3, 5, 6, 8, and 9.

Another aspect of the present disclosure provides an engineered RT polypeptide comprising an amino acid sequence that is at least 90%, at least 92%, at least 95%, at least 97%, at least 98%, or at least 99% identical to an amino acid sequence of SEQ ID NOs: 27-61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, 172, or 173; and a DNA binding domain comprising an amino acid sequence selected from the group consisting of SEQ ID NO: 2, 3, 5, 6, 8, and 9.

Another aspect of the present disclosure provides an engineered RT polypeptide comprising an amino acid sequence that is at least 90%, at least 92%, at least 95%, at least 97%, at least 98%, or at least 99% identical to an amino acid sequence of SEQ ID NOs: 27-61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, 172, or 173; and a DNA binding domain comprising an amino acid sequence selected from the group consisting of SEQ ID NO: 11-24.

Another aspect of the present disclosure provides an engineered RT polypeptide comprising an amino acid sequence of an RT disclosed in Table 1 or Table 2; and an amino acid sequence of DNA binding domain disclosed in Table 1.

In some embodiments, the engineered RT polypeptide described herein comprises the amino acid sequence of any one of SEQ ID NO: 174-188.

The engineered RT polypeptide described herein can comprise 42B L RT (SEQ ID NO: 145) operably linked to a full length DAT1 at the N-terminus. In that embodiment, the engineered RT can comprise the amino acid sequence of SEQ ID NO: 183. The engineered RT polypeptide described herein can comprise 42B L RT operably linked to a full length DAT1 at the C-terminus. In that embodiment, the engineered RT can comprise the amino acid sequence of SEQ ID NO: 184.

DAT1 (90) can be fused to any reverse transcriptase enzymes described herein, such as variants of MMLV reverse transcriptases disclosed in Table 1 or Table 2. While the majority of engineered RT polypeptide embodiments disclosed herein are N-terminus fusion proteins, the present disclosure also contemplates engineered RT polypeptides where the DNA binding domain, e.g., DAT1 or variant thereof is operably linked to the C-terminus of any RT polypeptide described herein. Accordingly, the present disclosure provides any combination of DNA binding domain and RT polypeptide described in Table 1 or Table 2. Non-exhaustive list of possible engineered RT polypeptides or recombinant RT proteins can comprise reverse transcriptases fused to full length DAT protein, DAT1 truncated to 36 amino acids, or DAT1 (90) homologs.

For example, the present disclosure provides an engineered RT comprising the amino acid sequence of SEQ ID NO: 178 (e.g., N-DAT-90 42B). The engineered RT can comprise the amino acid sequence of SEQ ID NO: 179 (e.g., N-DAT-90 50A+G). The engineered RT can comprise the amino acid sequence of SEQ ID NO: 180 (e.g., N-DAT-90 SOLD 33 VDG). The engineered RT can comprise the amino acid sequence of SEQ ID NO: 176 (e.g., N-DAT-90 SOLD 01). The engineered RT can comprise the amino acid sequence of SEQ ID NO: 177 (e.g., C-DAT-90 SOLD 01). As disclosed in FIGS. 20-33, an engineered RT polypeptide comprising N-DAT-90 42B (SEQ ID NO: 178) and/or N-DAT-90 50A+G (SEQ ID NO: 178) were shown to enhance the sensitivity of the 5′ single cell assay as shown by enhanced transcript capture.

In some embodiments, the engineered RT polypeptide described herein comprises 42B L RT (SEQ ID NO: 145) operably linked to an N-terminal truncated variant of DAT1 (D90) comprising the first 90 amino acids of the full length DAT1 at the C-terminus. In that embodiment, the engineered RT can comprise the amino acid sequence of SEQ ID NO: 174. The engineered RT polypeptide described herein can comprise 42B L RT operably linked to an N-terminal truncated variant of DAT1 (D90) comprising the first 90 amino acids of the full length DAT1 at the N-terminus. In that embodiment, the engineered RT can comprise the amino acid sequence of SEQ ID NO: 175.

The engineered RT polypeptide described herein can comprise 42B RT operably linked to an N-terminal truncated variant of DAT1 (D90) comprising the first 90 amino acids of the full length DAT1 at the N-terminus. In that embodiment, the engineered RT can comprise the amino acid sequence of SEQ ID NO: 178.

The engineered RT polypeptide described herein can comprise SOLD 01 RT operably linked to an N-terminal truncated variant of DAT1 (D90) comprising the first 90 amino acids of the full length DAT1 at the N-terminus. In that embodiment, the engineered RT can comprise the amino acid sequence of SEQ ID NO: 176. The engineered RT polypeptide described herein can comprise SOLD 01 operably linked to an N-terminal truncated variant of DAT1 (D90) comprising the first 90 amino acids of the full length DAT1 at the C-terminus. In that embodiment, the engineered RT can comprise the amino acid sequence of SEQ ID NO: 177.

The engineered RT polypeptide described herein can comprise 50A+G RT operably linked to an N-terminal truncated variant of DAT1 (D90) comprising the first 90 amino acids of the full length DAT1 at the N-terminus. In that embodiment, the engineered RT can comprise the amino acid sequence of SEQ ID NO: 179.

The engineered RT polypeptide described herein can comprise SOLD 33 VDG RT operably linked to an N-terminal truncated variant of DAT1 (D90) comprising the first 90 amino acids of the full length DAT1 at the N-terminus. In that embodiment, the engineered RT can comprise the amino acid sequence of SEQ ID NO: 180.

The DAT1 protein can be truncated to a minimal binding domain of 36 amino acids. In such embodiments, the engineered RT polypeptide described herein can comprise 42B L RT (SEQ ID NO: 145) operably linked to an N-terminal truncated variant of DAT1 (D36) comprising the first 36 amino acids of the full length DAT1 at the N-terminus (e.g., N-Dat36_42BL). In that embodiment, the engineered RT can comprise the amino acid sequence of SEQ ID NO: 181.

The engineered RT polypeptide described herein can comprise 42B L RT (SEQ ID NO: 145) operably linked to an N-terminal truncated variant of DAT1 (D36) comprising the first 36 amino acids of the full length DAT1 at the C-terminus (e.g., C-DAT36_42BL). In that embodiment, the engineered RT can comprise the amino acid sequence of SEQ ID NO: 182.

Homologs of DAT1 from non-yeast organisms can also be used to engineer the engineered the RT polypeptide or recombinant RT protein disclosed herein. Exemplary embodiments of such engineered RT polypeptides include, but not limited to an engineered RT polypeptide comprising the amino acid sequence of SEQ ID NO: 185 (e.g., N-DAT1-TL-QID040 42BL). The engineered RT polypeptide can comprise the amino acid sequence of SEQ ID NO: 186 (e.g., N-DAT1-TL-XP361 42BL). The engineered RT polypeptide can comprise the amino acid sequence of SEQ ID NO: 187 (e.g., N-DAT1-TL-XP558 42BL). The engineered RT polypeptide can comprise the amino acid sequence of SEQ ID NO: 188 (e.g., N-DAT1-TL-XP683 42BL). In some embodiments, SEQ ID NO: 185-188 can comprise a DAT1 protein from different budding yeasts

While embodiments disclosed herein only provide N-terminal fusion protein. The present disclosure contemplates engineered RT proteins comprising DAT1 from other organisms where the DAT1 RT proteins are C-terminal fusions. In some embodiments of the engineered RT polypeptide described herein, the DNA binding domain (e.g., DAT1 protein or variant thereof) can contain at least three repeated pentads of G-R-K-P-G.

In some embodiments, the engineered RT polypeptide described herein comprises an amino acid sequence having at least 90%, at least 91%, at least 92%, at least 93%, at least 94%, at least 95%, at least 96%, at least 97%, at least 98%, or at least 99% sequence identity to any one of SEQ ID NO: SEQ ID NO: 174-188. In some embodiments, the engineered RT polypeptide described herein comprises an amino acid sequence having 90%, 91%, 92%, 93%, 94%, 95%, 96%, 97%, 98%, or 99% sequence identity to any one of SEQ ID NO: SEQ ID NO: 174-188.

In some embodiments, the engineered RT disclosed herein further comprises a Tag protein as disclosed herein. The tag protein can be selected from the group consisting of an affinity tag, a fluorescent tag, or an expression and/or solubility enhancement tag, the tag is selected from hexahistidine tag (his-tag), small ubiquitin-like modifier tag (SUMO), a VariFlex C-Terminal solubility enhancement tag, a short peptide C-terminal tag, Thioredoxin (Trx) tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), or fungal avidin-like protein (Tamavidin)

The engineered RT polypeptide or the recombinant RT protein can comprise an hexahistidine tag (his-tag). Alternatively, the engineered RT polypeptide or the recombinant RT protein can comprise an amino acid sequence of SEQ ID NO: 62; or an amino acid sequence having at least 90% sequence identity to the amino acid sequence of SEQ ID NO: 62.

### F. Enhanced Reverse Transcriptase Activity

The engineered reverse transcriptase of the present disclosure can be a variant MMLV reverse-transcriptase with increased or enhanced reverse transcriptase activity. The term “increased” reverse transcriptase activity refers to the level of reverse transcriptase activity of a variant (e.g., mutant reverse transcriptase enzyme (e.g., MMLV variants disclosed herein)) as compared to its wild-type form (e.g., WT MMLV or MMLV having the amino acid of SEQ ID NO: 7) or a known variant (e.g., MMLV having the amino acid of SEQ ID NO: 1). A mutant enzyme is said to have an “increased” reverse transcriptase activity if the level of its reverse transcriptase activity (as measured by methods described herein or known in the art) is at least 10% or more than its wild-type or a known variant. For example, the variant can have at least 10%, 15%, 20%, 25%, 30%, 40%, 50%, 60%, 70%, 80%, 90%, 100% more or at least 2-fold, 3-fold, 4-fold, 5-fold, or 10-fold or more activity than the wild-type or known variant.

An engineered reverse transcriptase may exhibit one or more reverse transcriptase related activities including but not limited to, an RNA-dependent DNA polymerase activity, an RNAse H activity, a DNA-dependent DNA polymerase activity, an RNA binding activity, a DNA binding activity, a polymerase activity, a primer extension activity, a strand-displacement activity, a helicase activity, a strand transfer activity, a template binding activity, transcription template switching, transcription efficiencies, template switching efficiencies, processivity efficiencies, incorporation efficiencies, fidelity efficiencies, polymerization efficiencies, altered specificity, altered non-templated base addition, altered thermostability, altered tailing, altered adapter binding, binding efficiencies, ability to yield unique molecular identifiers (UMI), ability to yield median UMI, transcription efficiency, template switching efficiency, processivity, incorporation efficiency, Kd, distribution, fidelity, polymerization efficiency, Km, specificity, non-templated base addition, thermostability, tailing, adapter binding, binding efficiency, binding affinity (Km/Kcat), Vmax and ability to yield median UMI/cell and altered binding affinities.

A change in any activity may increase, decrease or have no effect on a different reverse-transcriptase related activity. In addition, a change in one activity may alter multiple properties of a reverse transcriptase. When multiple properties are affected, the properties may be altered similarly or differently. Methods of evaluating reverse transcriptase related activities are known in the art. A change in a reverse transcriptase related activity may alter one or more of the following results including but not limited to the yield of unique molecular identifiers (UMI), the median UMI obtained, the yield of mitochondrial UMI counts, and/or the yield of ribosomal UMI counts. A change or alteration in the yield of UMI the median UMI obtained, the yield of mitochondrial UMI counts, and/or the yield of ribosomal UMI counts may indicate one or more altered reverse transcriptase related activities.

The engineered reverse transcription enzyme variants of the present disclosure unexpectedly provide an altered or improved reverse transcriptase activity, such as but not limited to, improved template switching (TS) efficiency, higher end-to-end template jumping/switching, improved processivity efficiency, improved binding affinity, improved transcription efficiency, improved chemical tolerance, improved ability to yield mitochondrial unique molecular identifier (UMI) counts, improved ability to yield ribosomal unique molecular identifier (UMI) counts, improved shelf life, higher strand displacement, increased thermostability, improved thermoreactivity, and any combination thereof. An engineered reverse transcription enzyme of the current application may exhibit an altered base-biased template switching activity such as an increased base-biased template switching activity, decreased base-biased template switching activity or an altered base-bias to the template switching activity.

In some embodiments of the engineered RT polypeptide described herein, or the recombinant RT protein described herein, the engineered RT polypeptide or the recombinant RT protein exhibits increased template switching (TS) efficiency, increased processivity efficiency, increased binding affinity, increased transcription efficiency, increased chemical tolerance, improved ability to yield mitochondrial unique molecular identity (UMI) counts, improved ability to yield ribosomal unique molecular identity (UMI) counts, longer shelf life, higher strand displacement, higher end-to-end template jumping, or any combination thereof, when compared to an RT polypeptide or a recombinant RT protein lacking a conjugated DNA binding domain.

In some embodiments of the engineered RT polypeptide described herein, or the recombinant RT protein described herein, the engineered RT polypeptide or the recombinant RT protein comprises at least two or more of increased template switching (TS) efficiency, increased processivity efficiency, increased binding affinity, increased transcription efficiency, increased chemical tolerance, improved ability to yield mitochondrial unique molecular identity (UMI) counts, longer shelf life, higher strand displacement, higher end-to-end template jumping, or improved ability to yield ribosomal unique molecular identity (UMI) counts, when compared to an RT polypeptide or a recombinant RT protein lacking a conjugated DNA binding domain.

In some embodiments of the engineered RT polypeptide described herein or the recombinant RT protein described herein, the recombinant RT protein or the engineered RT exhibits increased transcript capture during amplification, when compared to an RT polypeptide or a recombinant RT protein lacking a conjugated DNA binding domain.

In some embodiments of the engineered RT polypeptide or the recombinant RT protein described herein, the DNA binding domain enhances the hybridization of a transcript and a primer during a nucleic acid amplification process, when compared to an RT polypeptide or a recombinant RT protein lacking a conjugated DNA binding domain.

The engineered reverse transcription enzyme variants of the present disclosure unexpectedly provided an altered reverse transcriptase activity, such as but not limited to, improved thermal stability, processive reverse transcription, non-templated base addition, binding affinity, and template switching ability. An engineered reverse transcription enzyme of the current application may exhibit an altered base-biased template switching activity such as an increased base-biased template switching activity, decreased base-biased template switching activity or an altered base-bias to the template switching activity. An engineered reverse transcriptase variant may exhibit enhanced template switching with a 5′-G cap on the substrate. Furthermore, an engineered reverse transcription enzyme variants described herein may also exhibit unexpectedly higher resistance to cell lysate (i.e., are less inhibited by cell lysate) than that exhibited by an enzyme having the amino acid sequence set forth in SEQ ID NO:1. Lastly, an engineered reverse transcription enzyme variants of the present disclosure may have an unexpectedly greater ability to capture full-length transcripts (e.g., in T-cell receptor paired transcriptional profiling), as compared to that exhibited by an enzyme having the amino acid sequence set forth in SEQ ID NO:1.

It is recognized that mutation of one or more residues may alter a first reverse transcriptase activity differently than a second reverse transcriptase activity. Further it is recognized that a different combination of mutations, such as different sites or residue changes may alter a reverse transcriptase activity similarly or differently. The variants that can template switch in the 5′ assay share the following alterations: E69K, E302R, T306K, W313F, L/K435G, and N454K. These variants may further comprise additional alterations that may affect one or more reverse transcriptase related activities. M39V and M66L may improve template switching. Without being limited by mechanism, variants comprising a M39V or a M66L mutation that do not exhibit altered performance in the 5′ GEM assay may exhibit an altered processivity, an altered ka or both. L435K mutants may improve thermostability in the presence of primer template. In the absence of primer template L435K variants may exhibit a thermal denaturation profile similar to that of the wild-type protein. L435K, P448 and D449 are residues in the connection domain; altering these residues may result in increased conformational flexibility. Additionally, the connection domain is thought to impact the conformational flexibility of the RNAse H domain. H503 and H634 occur within the RNAse H domain. The H503V and H634Y variants may impact primer-template contacting, processivity or both primer-template contacting and processivity.

Some variants share the following alterations: (a) the combination of variants consisting of a T542D mutation, a D583N mutation, an E607G mutation, an A644V mutation, a D653H mutation, and a K658R mutation. Some variants share the following alterations: (b) the combination of variants consisting of an E545G mutation, a D583N mutation, an H594Q mutation, an L603F mutation, and a S679P mutation. These variants may further comprise additional alterations that may affect one or more reverse transcriptase related activities. The combination of variants consisting of a T542D mutation, a D583N mutation, an E607G mutation, an A644V mutation, a D653H mutation, and a K658R mutation and the combination of variants consisting of an E545G mutation, a D583N mutation, an H594Q mutation, an L603F mutation and a S679P mutation may exhibit an altered RNAse H activity.

### 1. RNase H Activity

In some embodiments, the engineered reverse transcriptase polypeptide or recombinant RT protein is engineered to have reduced and/or abolished RNase activity. RNase H activity refers to endoribonuclease degradation of the RNA of a DNA-RNA hybrid to produce 5′ phosphate terminated oligonucleotides that are 2-9 bases in length. RNase H activity does not include degradation of single-stranded nucleic acids, duplex DNA, or double-stranded RNA. Removal of the RNase H activity of reverse transcriptase can eliminate the problem of RNA degradation of the RNA template and improve the efficiency of reverse transcription.

In some embodiments, the engineered reverse transcriptases or the recombinant RT proteins of the present disclosure can have a reduced or substantially reduced RNase H activity. The reduction or substantial reduction or complete removal of the RNase H activity of a reverse transcriptase (e.g., MMLV) can prevent the degradation of an RNA template before the initiation of the RT reaction, thereby improving the efficiency of reverse transcription.

In some embodiments, the engineered reverse transcriptases or the recombinant RT proteins of the present disclosure substantially lacks RNase H activity. In that embodiment, the engineered reverse transcriptases or the recombinant RT proteins of the present disclosure can have less than 10%, 5%, 1%, 0.5%, or 0.1% of the RNAse H activity of a wild type enzyme or a variant having the amino acid of SEQ ID NO: 1. In some embodiments, the engineered reverse transcriptases or the recombinant RT proteins of the present disclosure lack RNase H activity. In that embodiment, the engineered reverse transcriptases or the recombinant RT proteins of the present disclosure have undetectable RNase H activity or have an RNase H activity that is less than about 1%, 0.5%, or 0.1% of the RNase H activity of a wild-type enzyme or a variant comprising the amino acid of SEQ ID NO: 1.

As used herein, the term “reduced RNase H activity” means that the enzyme has less than 50%, e.g., less than 40%, less than 30%, less than 25%, or less than 20%, more preferably less than 15%, less than 10%, or less than 7.5%, and most preferably less than 5% or less than 2% of the RNase H activity of the corresponding wild type enzyme or a variant comprising the amino acid of SEQ ID NO: 1. The RNase H activity of an enzyme may be determined by assays known in the art. In some embodiments, the engineered reverse transcriptases or the recombinant RT proteins that have reduced and/or abolished RNase H activity comprise a D524 mutation in SEQ ID NO: 1 or 7.

### 3. Transcription Efficiency

In some embodiments, the engineered reverse transcriptase polypeptide or recombinant RT protein disclosed herein exhibits enhanced transcription efficiency when compared to the transcription efficiency of a reverse transcriptase having the amino acid sequence set forth in SEQ ID NO:1 or SEQ ID NO: 7 or any RT lacking a DNA binding domain. As noted herein, the conversion of mRNA into cDNA by reverse transcriptase-mediated reverse transcription is an essential step in single cell profiling and gene expression analyses. However, the use of an unmodified reverse transcriptase to catalyze reverse transcription is inefficient for all the reasons disclosed herein. The engineered reverse transcriptases or recombinant RT proteins of the disclosure are preferably modified or mutated such that the transcription efficiency of the engineered RT polypeptide or recombinant RT protein is increased or enhanced.

Further, engineered reverse transcription polypeptide or recombinant RT proteins of the present disclosure may have an unexpectedly greater ability to associate or bind to full-length transcripts (e.g., in T-cell receptor paired transcriptional profiling), as compared to that exhibited by an enzyme having the amino acid sequence set forth in SEQ ID NO:1 or non-DAT1 engineered RT.

It is recognized that salt concentration, the concentration of a cell fixation chemical and/or the concentration of a process reagent in a reverse transcriptase reaction may impact function of a reverse transcriptase. For example, “chemical tolerance” is intended that an the engineered reverse transcriptase or the recombinant RT protein of the current application may exhibit a reverse transcriptase related activity in either an expanded salt concentration range or in the presence of an increased concentration of a cell fixation chemical or process reagent, or in both an expanded salt concentration range and in the presence of an increased concentration of a cell fixation chemical or process reagent, as compared to the reverse transcriptase related activity of an enzyme having the amino acid sequence set forth in SEQ ID NO: 1, 143, 145, or 172 or non-DAT1 engineered RT.

An altered transcription efficiency may be an increased transcription efficiency or a decreased transcription efficiency as compared to the transcription efficiency of a reverse transcriptase having the amino acid sequence set forth in SEQ ID NO: 1. Altered transcription efficiency may be at least 0.1×, 0.2×, 0.3×, 0.4×, 0.5×, 0.6×, 0.7×, 0.8×, 0.9×, 1×, 1.5×, 2×, 2.5×, 3×, 3.5×, 4×, 4.5×, 5×, 5.5×, 6×, 6.5×, 7×, 7.5×, 8×, 8.5×, 9×, 10×, 15×, 20×, 25× or at least 30× greater than the transcription efficiency of a reverse transcriptase having the amino acid sequence set forth in SEQ ID NO: 1, 143, 145, or 172 or non-DAT1 engineered RT.

Transcription efficiency may be calculated as the sum of the area under the curve for the elongation, elongation plus tail, incomplete template switching (TSO) and complete template switching (TSO) regions over the total area under the curve for all products. Transcription efficiency reflects all those products for which transcription was successfully completed. Template switching oligonucleotide efficiency may be calculated as the area under the curve for the complete template switching region over the total area under the curve for all full-length products. An engineered reverse transcriptase may have an increased transcription efficiency, an increased TSO efficiency or both an increased transcription efficiency and an increased TSO efficiency.

### 4. Processivity

In some embodiments, the engineered reverse transcriptase polypeptide or recombinant RT protein described herein possesses one or more of the following characteristics when compared to a wild-type polymerase and/or reverse transcriptase: increased thermostability; increased thermoreactivity; increased resistance to reverse transcriptase inhibitors; increased ability to reverse transcribe difficult templates; increased speed; increased processivity; increased specificity; enhanced polymerization activity; increased sensitivity, or any combination thereof.

Processivity is defined as the ability of a polymerase or reverse transcriptase to carry out continuous nucleic acid synthesis on a template nucleic acid without frequent dissociation. It can be measured by the average number of nucleotides incorporated by a polymerase on a single association/disassociation event. DNA polymerase or reverse transcriptase alone produces short DNA product strand per binding event. Most DNA polymerases or reverse transcriptases are intrinsically low-processivity enzymes. The low processivity of DNA polymerase or reverse transcriptase alone is insufficient for the timely replication of a large genome.

In some embodiments, the polymerization activity of the engineered reverse transcriptase polypeptide or recombinant RT protein as described herein is enhanced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type reverse transcriptase.

In some embodiments, the engineered reverse transcriptase enzyme or engineered reverse transcriptase polypeptide or recombinant RT protein reverse transcribes a RNA molecule having at least about 100, at least about 200, at least about 300, at least about 400, at least about 500, at least about 600, at least about 700, at least about 800, at least about 900, or at least about 1000 nucleotides. In another embodiment, the engineered reverse transcriptase polypeptide or recombinant RT protein reverse transcribes a RNA molecule that is at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb, at least about 13 kb, at least about 14 kb, or at least about 15 kb. In another embodiment, the engineered reverse transcriptase polypeptide or recombinant RT protein reverse transcribes a RNA molecule that is at least about 7 kb or at least about 8 kb.

In some embodiments, the increase in thermoreactivity, resistance to reverse transcriptase inhibitors, ability to reverse transcribe difficult templates, speed, processivity, specificity, or sensitivity of the engineered reverse transcriptase polypeptide or recombinant RT protein as described herein has is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase.

In some embodiments, the enhanced reverse transcriptase activity is an increased binding affinity and template switching efficiency as compared to a reverse transcriptase having the amino acid sequence set forth in SEQ ID NO:1, 143, 145, or 172 or non-DAT1 engineered RT. In some embodiments, the enhanced reverse transcriptase activity is an enhanced processivity as compared to the processivity of a reverse transcriptase having the amino acid sequence set forth in SEQ ID NO: 1, 143, 145, or 172 or non-DAT1 engineered RT.

Processivity relates to a reverse transcriptase's ability to remain associated with the template while incorporating nucleotides. Measurements of processivity may include but are not limited to the number of nucleotides incorporated in a single binding event of a reverse transcriptase molecule. Processivity also relates to the affinity of the enzyme for the substrate; thus, an enzyme with increased processivity may be more resistant to the presence of an inhibitor.

### IV. Nucleic Acids and Expression Vectors

### A. Nucleic Acids

One aspect of the present disclosure provides an isolated nucleic acid molecule encoding the engineered reverse transcriptase, the recombinant RT protein or derivatives thereof as described herein. One aspect of the present disclosure provides an isolated nucleic acid molecule encoding any of the engineered RT polypeptides or the recombinant RT protein described herein.

In some embodiments, the engineered reverse transcriptase polypeptide or the recombinant RT protein disclosed herein can been coded by a nucleic acid set forth herein or readily derived in light of polypeptide information provided herein and known in the art. In some embodiments, the isolated nucleic acid molecule encoding the RT polypeptide can comprise a sequence selected from SEQ ID NO; 25; SEQ ID NO: 136, SEQ ID NO:138, SEQ ID NO:140, SEQ ID NO:142, SEQ ID NO: 144, SEQ ID NO: 146, SEQ ID NO: 148, SEQ ID NO: 150, SEQ ID NO: 152, SEQ ID NO:154, SEQ ID NO: 156, SEQ ID NO: 158, SEQ ID NO: 160, SEQ ID NO: 162, SEQ ID NO: 164, SEQ ID NO: 167, SEQ ID NO: 169, or SEQ ID NO: 171; or a non-limiting embodiment of a nucleic acid sequence of Table 1 or Table 2.

The reverse transcriptase polypeptides, or the DNA binding domains need not be encoded by any specific nucleic acid exemplified herein. For example, redundancy in the genetic code allows for variations in nucleotide codon sequences that nevertheless encode the same amino acid. Accordingly, engineered polymerases of the present disclosure can be produced from nucleic acid sequences that are different from those set forth herein, for example, being codon optimized for a particular expression system. Codon optimization can be carried out, for example, as set forth in Athey et al., BMC Bioinformatics, 18:391-401 (2017).

Wild type nucleic acids (e.g., RT or DNA binding domain) may be isolated from naturally occurring sources to be used as starting material to generate novel polymerases. Generally, the nomenclature and the laboratory procedures in recombinant DNA technology described below are those well-known and commonly employed in the art. Standard techniques for cloning, DNA and RNA isolation, amplification and purification are known. Generally enzymatic reactions involving DNA ligase, DNA polymerase, restriction endonucleases are the like are performed according to the manufacturer's specifications. These techniques and various other techniques are generally performed according to Sambrook & Russell, Molecular Cloning-A Laboratory Manual, Cold Spring Harbor Laboratory, Cold Spring Harbor, N.Y., (1989) or Ausubel et al., Current Protocols in Molecular Biology, Vol. 1-3, John Wiley & Sons, Inc. (1994-1998).

The isolation of nucleic acids (e.g., RT or DNA binding domain) may be accomplished by a variety of techniques. The nucleic acids of the present disclosure can be generated from the wild type sequences. The wild type sequences are altered to create modified sequences. Wild type molecules (e.g., RT or DNA binding domain) can be modified to create the engineered RT described in the present application using methods that are well known in the art. Exemplary modification methods are site-directed mutagenesis, point mismatch repair, or oligonucleotide-directed mutagenesis.

### B. Vectors

Another aspect of the present disclosure provides an expression vector comprising the isolated nucleic acid encoding the engineered reverse transcriptase polypeptides or derivatives thereof as described herein. A “vector” refers to a polynucleotide, which when independent of the host chromosome, is capable replication in a host organism. Preferred vectors include plasmids and typically have an origin of replication. Vectors can comprise, e.g., transcription and translation terminators, transcription and translation initiation sequences, and promoters useful for regulation of the expression of the particular nucleic acid. The polymerases of the present disclosure can be expressed in a variety of host cells, including E. coli, other bacterial hosts, yeasts, filamentous fungi, and various higher eukaryotic cells such as the COS, CHO and HeLa cells lines and myeloma cell lines. Techniques for gene expression in microorganisms are described in, for example, Smith, Gene Expression in Recombinant Microorganisms (Bioprocess Technology, Vol. 22), Marcel Dekker, 1994. Examples of bacteria that are useful for expression include, but are not limited to, Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus. Filamentous fungi that are useful as expression hosts include, for example, the following genera: Aspergillus, Trichoderma, Neurospora, Penicillium, Cephalosporium, Achlya, Podospora, Mucor, Cochliobolus, and Pyricularia. Synthesis of heterologous proteins in yeast is well known and described in the literature. There are many expression systems for producing the polymerase polypeptides of the present invention that are well known to those of ordinary skill in the art.

### C. Host Cells

Another aspect of the present disclosure provides a host cell transfected with the expression vector comprising the isolated nucleic acid encoding the engineered reverse transcriptase as described herein. Eukaryotic expression systems for mammalian cells, yeast, and insect cells are well known in the art and are also commercially available. In yeast, vectors include Yeast Integrating plasmids (e.g., YIp5) and Yeast Replicating plasmids (the YRp series plasmids) and pGPD-2. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Once expressed, the engineered reverse transcriptase or a derivative thereof can be purified according to standard procedures of the art, including ammonium sulfate precipitation, affinity purification columns, column chromatography, gel electrophoresis and the like. Substantially pure compositions of at least about 90 to about 95% homogeneity are preferred, and about 98 to about 99% or more homogeneity are most preferred. Once purified, partially or to homogeneity as desired, the polypeptides may then be used (e.g., as immunogens for antibody production).

To facilitate purification of the engineered reverse transcriptase or a derivative thereof, the nucleic acids that encode the engineered reverse transcriptase or derivatives thereof can also include a coding sequence for an epitope or “tag” for which an affinity binding reagent is available. Examples of suitable epitopes include the myc and V-5 reporter genes; expression vectors useful for recombinant production of fusion polypeptides having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the fusion proteins of the disclosure, and corresponding detection systems are known to those of skill in the art as described herein, and several are commercially available (e.g., FLAG″ (Kodak, Rochester N.Y.). Another example of a suitable tag is a polyhistidine sequence, which is capable of binding to metal chelate affinity ligands. Typically, six adjacent histidines are used (6His-tag, his-tag), although one can use more or less than six. Suitable metal chelate affinity ligands that can serve as the binding moiety for a polyhistidine tag include nitrilo-tri-acetic acid (NTA).

One of skill in the art would recognize that after biological expression or purification, the engineered reverse transcriptase or derivatives thereof may possess a conformation substantially different than the native conformations of the constituent polypeptides. In this case, it may be necessary or desirable to denature and reduce the engineered reverse transcriptase or a derivative thereof and cause the engineered reverse transcriptase or a derivative thereof to re-fold into the preferred conformation. Methods of reducing and denaturing proteins and inducing re-folding are well known to those of skill in the art.

### V. Compositions and Reaction Mixtures

The present disclosure further provides compositions comprising a variety of components in various combinations needed for nucleic acid amplification using the engineered RT polypeptides or recombinant proteins disclosed herein.

One aspect of the present disclosure provides a composition comprising any of the recombinant RT proteins described herein. One aspect of the present disclosure provides a composition comprising any of the engineered RT polypeptides described herein. One aspect of the present disclosure provides a composition comprising any of the engineered RT polypeptides described herein. One aspect of the present disclosure provides a composition comprising any of the expression vectors described herein. One aspect of the present disclosure provides a composition comprising the host cells described herein. In some embodiments, any one of the compositions described herein further comprise a buffer.

In some embodiments of the present disclosure, the compositions are formulated by admixing one or more engineered reverse transcriptase polypeptides or recombinant RT proteins, or derivatives thereof of the present disclosure in a buffered salt solution. One or more DNA polymerases and/or one or more nucleotides, and/or one or more primers may optionally be added to create the compositions of the invention. These compositions can be used in the methods disclosed herein to produce, analyze, quantitate and otherwise manipulate nucleic acid molecules (e.g., using reverse transcription or one-step RT-PCR procedures).

In some embodiments, the engineered reverse transcriptases or the recombinant RT proteins disclosed herein are provided at working concentrations (e.g., 1×) in stable buffered salt solutions.

The terms “stable” and “stability” as used herein generally mean the retention by a composition, such as an enzyme (e.g., engineered reverse transcriptase or the recombinant RT protein) composition, of at least 70%, preferably at least 80%, and most preferably at least 90%, of the original enzymatic activity (in units) after the enzyme (e.g., engineered reverse transcriptase or the recombinant RT protein) or composition containing the enzyme has been stored for about one week at a temperature of about 4° C., about two to six months at a temperature of about −20° C., and about six months or longer at a temperature of about −80° C.

As used herein, the term “working concentration” means the concentration of an enzyme (e.g., engineered reverse transcriptase or the recombinant RT protein) that is at or near the optimal concentration used in a solution to perform a particular function such as reverse transcription of nucleic acids.

Such compositions can also be formulated as concentrated stock solutions (e.g., 2×, 3×, 4×, 5×, 6×, 10×, etc.). In some embodiments, having the composition as a concentrated (e.g., 5×) stock solution allows a greater amount of nucleic acid sample to be added (such as, for example, when the compositions are used for nucleic acid synthesis). The water used in forming the compositions of the present invention is preferably distilled, deionized and sterile filtered (through a 0.1-0.2 micrometer filter) and is free of contamination by DNase and RNase enzymes. Such water is available commercially, for example from Life Technologies (Carlsbad, Calif.) or may be made as needed according to methods well known to those skilled in the art.

### VI. Methods for Using Engineered Reverse Transcriptases

The engineered reverse transcriptases of the present disclosure may be used in any application in which a reverse transcriptase with the indicated altered activity is desired. Methods of using reverse transcriptases are known in the art and one skilled in the art may select any of the engineered reverse transcriptases disclosed herein. In some embodiments, a reverse transcription reaction introduces a barcode. In some embodiments, the barcoding reaction is an enzymatic reaction. In some embodiments, the barcoding reaction is a reverse transcription amplification reaction that generates complementary deoxyribonucleic acid (cDNA) molecules upon reverse transcription of ribonucleic acid (RNA) molecules of the cell. In some embodiments, the RNA molecules are released from the cell. In some embodiments, the RNA molecules are released from the cell by lysing the cell. In some embodiments, the RNA molecules are messenger RNA (mRNA).

### A. Amplification Methods

One aspect of the present disclosure provides a method for performing a reverse transcription reaction for generating a nucleic acid product from an RNA template using an engineered reverse transcriptase or recombinant RT protein described herein. The engineered reverse transcriptases or recombinant RT protein of the present application may be used in any application in which a reverse transcriptase with the indicated altered activity is desired. Methods of using reverse transcriptases are known in the art. One skilled in the art may select any of the engineered reverse transcriptases disclosed herein.

One aspect of the present disclosure provides a method for performing a reverse transcription reaction for generating a nucleic acid product from an RNA template comprising contacting under suitable conditions a biological sample or extract thereof with an engineered RT polypeptide, or a recombinant RT protein described herein. The cell can be fixed. The cell can be permeabilized. Alternatively, the cell can be permeabilized and fixed. In some embodiments, the cell is a cell bead. In some embodiments, the cell bead is fixed. In some embodiments, the nucleus is permeabilized or fixed. In some embodiments, the nucleus is permeabilized and fixed. In some embodiments, the biological sample comprises a suitable cellular preparation selected from cell populations and/or single cells. In some embodiments, the biological sample comprises a tissue.

In some embodiments, when the biological sample is a cell, a cell bead, or a nucleus, the reverse transcription reaction is part of a single cell RNA sequencing assay. In one embodiment, the single cell RNA sequencing assay further comprises, prior to the reverse transcription, partitioning the cell, the cell bead, or the nucleus into a partition. In one embodiment, the single cell RNA sequencing assay further comprises, after the reverse transcription reaction, hybridizing the nucleic acid product to an oligonucleotide molecule comprising a partition-specific barcode.

In some embodiments, when the biological sample is a cell or tissue sample immobilized on a surface, the reverse transcription reaction is part of a spatial RNA sequencing assay.

In some embodiments of the method described herein, the engineered RT polypeptide or the recombinant RT protein enhances template switching (TS) efficiency, processivity efficiency, binding affinity, transcription efficiency, chemical tolerance, ability to yield mitochondrial unique molecular identity (UMI) counts, ability to yield ribosomal unique molecular identity (UMI) counts, strand displacement, end-to-end template jumping, or any combination thereof, when compared to an RT polypeptide or a recombinant RT protein lacking a conjugated DNA binding domain.

In some embodiments, the engineered RT polypeptide or the recombinant RT protein enhances at least two or more of template switching (TS) efficiency, processivity efficiency, binding affinity, transcription efficiency, chemical tolerance, ability to yield mitochondrial unique molecular identity (UMI) counts, strand displacement, end-to-end template jumping, or ability to yield ribosomal unique molecular identity (UMI) counts, when compared to an RT polypeptide or a recombinant RT protein lacking a conjugated DNA binding domain.

In some embodiments, the recombinant RT protein or the engineered RT enhances transcript capture during amplification, when compared to an RT polypeptide or a recombinant RT protein lacking a conjugated DNA binding domain.

In some embodiments, the DNA binding domain of the recombinant RT protein or the engineered RT enhances the hybridization of a transcript and a primer during the amplification process, when compared to an RT polypeptide or a recombinant RT protein lacking a conjugated DNA binding domain.

The engineered RT polypeptide or the recombinant RT protein can comprise a DNA binding domain comprising an amino acid sequence selected from SEQ ID NO:2, 3, 5, 6, 8, 9, or 11-24; and an amino acid sequence selected from SEQ ID NOs: 27-61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, 172, or 173.

In some embodiments, the amino acid sequence of the engineered RT polypeptide or the recombinant RT protein comprises an amino acid sequence having at least about 90%, at least about 92%, at least about 95%, at least about 97%, at least about 98%, or at least about 99% sequence identity to SEQ ID NO: 174-188.

The engineered RT polypeptide or the recombinant RT protein can comprise M39V, M66I, Q91R, I347V, and H594Q substitution in SEQ ID NO: 143. The engineered RT polypeptide or the recombinant RT protein can comprise SEQ ID NO: 129 (SOLD 034). The engineered RT polypeptide or the recombinant RT protein can comprise SOLD 001 (SEQ ID NO: 65). The engineered RT polypeptide or the recombinant RT protein can comprise SOLD 33 VDG (SEQ ID NO: 173). In some embodiments, the engineered RT polypeptide or the recombinant RT protein can comprise M39V, T542D, D583N, E607G, A644V, D653H, K658R, and L671P in SEQ ID NO: 1 or 143.

The engineered RT polypeptide or the recombinant RT protein can comprise M39V, T542D, D583N, E607G, A644V, D653H, K658R, and/or L671P in SEQ ID NO: 143. The engineered RT polypeptide or the recombinant RT protein can comprise SEQ ID NO: 111 (SOLD 025). The engineered RT or the recombinant RT protein can comprise a M39V, M66I, Q91R, I347V, and/or H594Q in SEQ ID NO: 143.

In some embodiments, the engineered RT polypeptide or the recombinant RT protein can comprise an amino acid sequence that is at least about 90%, at least about 92%, at least about 95%, at least about 97%, at least about 98%, or at least about 99% identical to an amino acid sequence disclosed in Table 1 or Table 2.

One aspect of the present disclosure provides a method of using the engineered RT polypeptide, or the recombinant RT protein described herein, the method comprising contacting the engineered RT polypeptide or the recombinant RT protein with a nucleic acid template under suitable conditions to produce a polymerized nucleic acid product. In some embodiments, the nucleic acid template comprises an RNA, a DNA, or a nucleic acid comprising an unnatural nucleotide. In some embodiments, the nucleic acid template comprises an RNA.

In some embodiments, the engineered reverse transcriptase comprises an M39 mutation, a K47 mutation, an L435 mutation, a D449 mutation, a D524 mutation, an E607 mutation, a D653 mutation and an L671 mutation in SEQ ID NO:7. In some embodiments, the engineered reverse transcriptase comprises a mutation selected from a K13 mutation, a K13L mutation, a D36 mutation, an N37 mutation, a V2 mutation, a D36L mutation, an insertion, and a combination thereof.

In some embodiments, the engineered reverse transcriptases or the recombinant RT proteins, or derivatives thereof of the present disclosure are used in reverse transcription reactions, such as RT-PCR, or other known reactions in the art where nucleic acids, for example RNA molecules, are reverse transcribed using a reverse transcriptase.

The engineered reverse transcriptase, the recombinant RT protein or a derivative thereof as described herein may be used to make nucleic acid molecules from one or more templates. Such methods can comprise mixing one or more nucleic acid templates (e.g., RNA, such as non-coding RNA (ncRNA), messenger RNA (mRNA), micro RNA (miRNA), and small interfering RNA (siRNA) molecules) with one or more of the engineered reverse transcriptases of the disclosure and incubating the mixture under conditions sufficient to generate one or more nucleic acid molecules complementary to all or a portion of the one or more nucleic acid templates. Other methods of cDNA synthesis which may advantageously use the engineered reverse transcriptase or the recombinant RT protein of the present disclosure will be readily apparent to one of ordinary skill in the art.

In some embodiments, the method of using the engineered reverse transcriptase, or the recombinant RT protein or a derivative thereof as described herein comprises the amplification of one or more nucleic acid molecules comprising mixing one or more nucleic acid templates with one of the engineered reverse transcriptase polypeptide or recombinant RT proteins or a derivative thereof of the disclosure, and incubating the mixture under conditions sufficient to amplify one or more nucleic acid molecules complementary to all or a portion of the one or more nucleic acid templates. In one embodiment, the method may comprise the use of one or more DNA polymerases and may be employed as in standard reverse transcription-polymerase chain reaction (RT-PCR) reactions.

In some embodiments, the method of using the engineered reverse transcriptase, recombinant RT protein or a derivative thereof as described herein may be one-step (e.g., one-step RT-PCR) or two-step (e.g., two-step RT-PCR) reactions. In one embodiment, the one-step RT-PCR type reactions may be accomplished in one tube thereby lowering the possibility of contamination. Such one-step reactions can comprise (a) mixing a nucleic acid template (e.g., mRNA) with one or more engineered reverse transcriptase polypeptides or recombinant RT proteins or derivatives thereof of the present disclosure and one or more polymerases and (b) incubating the mixture under conditions sufficient to amplify a nucleic acid molecule complementary to all or a portion of the template.

In another embodiment, two-step RT-PCR reactions may be accomplished in two separate steps. Such methods can comprise (a) mixing a nucleic acid template (e.g., mRNA) with an engineered reverse transcriptase polypeptide or a recombinant RT protein or a derivative thereof of the present disclosure, (b) incubating the mixture under conditions sufficient to make a nucleic acid molecule (e.g., a DNA molecule) complementary to all or a portion of the template, (c) mixing the nucleic acid molecule with one or more DNA polymerases and (d) incubating the mixture of step (c) under conditions sufficient to amplify the nucleic acid molecule. For amplification of long nucleic acid molecules (i.e., greater than about 3-5 kb in length), a combination of DNA polymerases and the engineered reverse transcriptase polypeptide or recombinant RT protein or a derivative thereof of the present disclosure may be used.

Amplification methods which may be used in accordance with the present invention (e.g., using one or more engineered reverse transcriptase polypeptides or recombinant RT proteins or derivatives thereof of the present disclosure) include PCR, Isothermal Amplification, Strand Displacement Amplification (SDA), and Nucleic Acid Sequence-Based Amplification (NASBA); as well as more complex PCR-based nucleic acid fingerprinting techniques such as Random Amplified Polymorphic DNA (RAPD) analysis, Arbitrarily Primed PCR (AP-PCR) DNA Amplification Fingerprinting (DAF); microsatellite PCR; Directed Amplification of Minisatellite-region DNA (DAVID); digital droplet PCT (ddPCR) and Amplification Fragment Length Polymorphism (AFLP) analysis. In some embodiments, the engineered reverse transcriptase disclosed herein may be used in methods of amplifying or sequencing a nucleic acid molecule comprising one or more polymerase chain reactions (PCRs), such as any of the PCR-based methods described above.

Methods of producing an engineered reverse transcriptase, an engineered reverse transcriptase or a derivative thereof of the present disclosure are known to those of skill in the art of molecular biology or molecular genetics. For example, nucleic acids encoding the wild-type polymerase or nucleic acid binding domains can be generated using routine techniques in the field of recombinant genetics.

### B. Nucleic Acid Sample Processing

Another aspect of the present disclosure provides a nucleic acid extension method comprising contacting a target nucleic acid molecule with an engineered reverse transcriptase or a recombinant RT protein and a plurality of nucleic acid barcoded molecules comprising a barcode sequence, and incubating the target nucleic acid, the engineered reverse transcriptase or the recombinant RT protein and barcoded molecules under conditions in which the barcoded molecules are extended by the engineered reverse transcriptase or the recombinant RT protein. In some embodiments, the engineered reverse transcriptase or the recombinant RT protein comprises the amino acid sequence of an engineered RT or an recombinant RT protein described herein or a derivatives thereof. The target nucleic acid hybridizes to one of the plurality of barcoded molecules and the hybridized barcoded molecule is extended by the engineered reverse transcriptase or the recombinant RT protein described herein.

The novel engineered reverse transcriptase polypeptide or the recombinant RT protein described herein can be used to generate a Single Cell 3′ (SC-3′) and/or 5′ (SC-5′) gene expression libraries. The SC-3′ and SC-5′ assays are similar but capture different ends of the polyadenylated transcript in the final library. Both solutions use poly-dT primer for reverse transcription). In the SC-3′ assay (FIG. 14B), the poly-dT sequence is located on the gel bead oligo. In the SC-5′ assay (FIG. 14A), the poly-dT is supplied as an RT primer. A template switching oligo(TSO) is used in both assays to reverse transcribe the full-length transcript.

After amplifying the cDNA, transcripts are randomly fragmented under conditions that favor 300-400 bp length fragments. Downstream of fragmentation, only transcripts containing both (1) a 10× Barcode and (2) an Illumina® Read 2 adaptor, which is ligated on to the cDNA after fragmentation, can be amplified during the Sample Index PCR. This results in final 10× libraries that either represent the 3′ end of the transcript (as the 10× Barcode is adjacent to the poly A tail on the 3′ end of the transcript) or the 5′ end of the transcript (as the 10× Barcode is adjacent to the TSO and the 5′ end of the transcript). See e.g., kb. 10×genomics.com/hc/en-us/articles/360000939852-What-is-the-difference-between-Single-Cell-3-and-5-Gene-Expression-libraries-.

### 1. RNA Template

In some embodiments, the nucleic acid is a ribonucleic acid (RNA) molecule; and the engineered reverse transcriptase polypeptide or recombinant RT protein reverse transcribes the RNA molecule thereby generating a first strand cDNA.

A first strand cDNA reaction can be optionally performed using template switching oligonucleotides. For example, a template switching oligonucleotide can hybridize to a poly(C) tail added to a 3′ end of the cDNA by the engineered reverse transcriptase polypeptide or recombinant RT protein described herein. The original mRNA template and template switching oligonucleotide can then be denatured from the cDNA and a barcoded capture probe can then hybridize with the cDNA and a complement of the cDNA can be generated. The first strand cDNA can then be purified and collected for downstream amplification steps. The first strand cDNA can be amplified using PCR, where the forward and reverse primers flank the spatial barcode and target analyte regions of interest, generating a library associated with a particular spatial barcode. In some embodiments, the cDNA comprises a sequencing by synthesis (SBS) primer sequence. The library amplicons are sequenced and analyzed to decode spatial information.

Exemplary steps for sample preparation, permeabilization, DNA generation (e.g., first strand cDNA generation and second strand generation), DNA amplification (e.g., cDNA amplification) and quality control, and spatial gene expression library construction are disclosed for example in WO 2020/047002, WO 2020/047004, WO 2020/047005, WO 2020/047007, and WO 2020/047010, all of which are incorporated herein by reference in their entireties.

In some embodiments, a reverse transcription reaction introduces a barcode. In some embodiments, the barcoding reaction is an enzymatic reaction. In some embodiments, the barcoding reaction is a reverse transcription amplification reaction that generates complementary deoxyribonucleic acid (cDNA) molecules upon reverse transcription of ribonucleic acid (RNA) molecules of the cell. In some embodiments, the RNA molecules are released from the cell. In some embodiments, the RNA molecules are released from the cell by lysing the cell. In some embodiments, the RNA molecules are released from the cell by permeabilizing the cell, or a tissue which comprises a plurality of the same and/or different cell types. In some embodiments, the RNA molecules are messenger RNA (mRNA).

In some embodiments, a reverse transcription reaction using the engineered reverse transcriptase, the recombinant RT protein or derivative thereof of the present disclosure is initiated at the point of hybridization of the capture sequences to the RNA molecules, with the capture probe being extended by the engineered reverse transcriptase polypeptide or recombinant RT protein of the present disclosure in a template directed fashion using the hybridized mRNA as a template. The recombinant RT protein or the engineered RT polypeptide can exhibit increased transcript capture during amplification. In that embodiment, the DNA binding domain of the engineered Rt polypeptide or recombinant RT protein can enhance the hybridization of a transcript and a primer during a nucleic acid amplification process. In some embodiments, the primer can comprise a poly-dT or a poly(dT)VN sequence and a non-poly(dT) sequence; and the transcript can comprise a poly-dA sequence. The DNA binding domain (e.g., DAT1 or variant thereof) can stabilize the oligo(A)-olgo(T) based transcript-primer complex during a nucleic acid amplification process. In some embodiments, the primer is a barcoded molecule.

In some embodiments, the reverse transcription reaction produces single stranded cDNA molecules each having a molecular tag and barcode associated with the cDNA, followed by amplification of cDNA to produce a double stranded cDNA that includes the sequences of the barcoded molecules.

In some embodiments, the plurality of nucleic acid barcoded molecules comprise an oligo(dT) sequence. In that embodiment, the engineered reverse transcriptase polypeptide or recombinant RT protein reverse transcribes the mRNA molecule into a complementary DNA molecule using the mRNA hybridized to the oligo(dT) sequence of the nucleic acid barcoded molecules as a template, and the nucleic acid binding domain binds and stabilizes the mRNA-oligo(dT) hybrid during the reverse transcription. Following reverse transcription, the engineered reverse transcriptase polypeptide or recombinant RT protein as described herein further amplifies the complementary DNA molecule comprising the barcode sequence, thereby generating an amplified DNA product comprising the barcode sequence, molecular tag sequence, or complements thereof.

In some embodiments of the nucleic acid extension method described herein, the method can comprise a second nucleic acid molecule comprising an oligo(dT) sequence. In that embodiment, the plurality of nucleic acid barcoded molecules comprise an oligo(dT) sequence; and the nucleic acid binding domain of the engineered reverse transcriptase polypeptide or recombinant RT protein binds and stabilizes the mRNA-Oligo (dT) hybrid, while the polymerase domain of the engineered reverse transcriptase polypeptide or recombinant RT protein reverse transcribes the mRNA molecule using the second nucleic acid molecule comprising the oligo(dT) sequence, thereby generating a complementary DNA molecule. In this embodiment, the engineered reverse transcriptase polypeptide or recombinant RT protein further amplifies the complementary DNA molecule, thereby generating an amplified DNA product comprising a barcode sequence.

In some embodiments, the nucleic acid extension method comprises a cell, a population of cells, or a tissue and the template nucleic acid molecule is from the cell, population of cells or the tissue.

In some embodiments, the molecular tags are coupled to priming sequences and the barcoding reaction is initiated by hybridization of the priming sequences to the RNA molecules. In some embodiments, each priming sequence comprises a random N-mer sequence. In some embodiments, the random N-mer sequence is complementary to a 3′ sequence of a ribonucleic acid molecule of the cell. In some embodiments, the random N-mer sequence comprises a poly-dT sequence having a length of at least 5 bases. In some embodiments, the random N-mer sequence comprises a poly-dT sequence having a length of at least 10 bases.

In some embodiments, the barcoding reaction is performed by extending the priming sequences in a template directed fashion using reagents for reverse transcription. In some embodiments, the reagents for reverse transcription comprise a reverse transcription enzyme (e.g., engineered RT polypeptide or recombinant RT protein), a buffer and a mixture of nucleotides. In some embodiments, the reverse transcription enzyme adds a plurality of non-template oligonucleotides upon reverse transcription of a ribonucleic acid molecule. In some embodiments, the reverse transcription enzyme is an engineered RT polypeptide or recombinant RT protein as disclosed herein.

In some embodiments, the barcoding reaction produces single stranded complementary deoxyribonucleic acid (cDNA) molecules each having a molecular tag from said molecular tags on a 5′ end thereof, followed by amplification of cDNA to produce a double stranded cDNA having the molecular tag on the 5′ end and a 3′ end of the double stranded CDNA.

In some embodiments, a molecular tag which comprises a barcode plus additional functional sequences, or only additional functional sequences, is further included into a cDNA molecule generated during a reverse transcription reaction. In some embodiments, the reagents for reverse transcription comprise a reverse transcription enzyme (e.g., the engineered reverse transcriptase or the recombinant RT protein described herein), a buffer, and a mixture of nucleotides. In some embodiments, the reverse transcription enzyme adds a plurality of non-template oligonucleotides upon reverse transcription of a ribonucleic acid molecule from the nucleic acid molecules. In some embodiments, the reverse transcription enzyme is an engineered reverse transcriptase or a recombinant RT protein as disclosed herein.

In one aspect, the present disclosure provides methods that utilize the engineered reverse transcriptase polypeptides or the recombinant RT protein described herein for nucleic acid sample processing. In one embodiment, the method comprises contacting a template ribonucleic acid (RNA) molecule with an engineered reverse transcriptase to reverse transcribe the RNA molecule to a complementary DNA (cDNA) molecule. The contacting step may be in the presence of a plurality of nucleic acid barcode molecules, wherein each nucleic acid barcode molecule comprises a barcode sequence. The nucleic acid barcode molecule may comprise a sequence configured to couple to a template RNA molecule. Suitable sequences include, without limitation, an oligo(dT) sequence, a random N-mer primer, or a target-specific primer. The nucleic acid barcode molecule may comprise a template switching sequence.

In other embodiments, the RNA molecule is a messenger RNA (mRNA) molecule. In one embodiment, the contacting step provides conditions suitable to allow the engineered reverse transcriptase to: (i) transcribe the mRNA molecule into the cDNA molecule with the oligo(dT) sequence and/or (ii) perform a template switching reaction, thereby generating the cDNA molecule which comprises the barcode sequence, or a derivative thereof.

In another embodiment, the contacting step may occur in (i) a partition having a reaction volume (e.g., as further described herein and see e.g., U.S. Pat. Nos. 10,400,280 and 10,323,278, each of which is incorporated herein by reference in its entirety); (ii) in a bulk reaction where the reaction components (e.g., template RNA and engineered reverse transcriptase) are in solution; or (iii) on a nucleic acid array (see e.g., U.S. Pat. Nos. 10,480,022 and 10,030,261 as well as WO/2020/047005 and WO/2020/047010, each of which is incorporated herein by reference in its entirety). Further, the reverse transcription reaction may occur in a tissue (e.g., in situ reverse transcription), on a template that is associated with a sequence on a substrate, such as practiced in spatial transcriptomics, or further in a RT-PCR or other reverse transcription reaction in vitro on a purified target, partially purified target or unpurified target as found for example in a cellular lysate.

Examples of assays involving nucleic acid sample processing may include, but are not limited to, single-cell transcription profiling, single-cell sequence analysis, immune profiling of individual T and B cells, single-cell chromatin accessibility analysis (e.g., ATAC seq analysis), single cell processing and analysis, paired single cell TCR sequencing, paired TCRα and TCRβ. These exemplary assays may be carried out using commercially available systems for encapsulating biological samples, gel beads, barcodes, and/or other compounds/materials in droplets, such as The Chromium System (10× Genomics, Pleasanton CA USA). Engineered RT polypeptide or recombinant RT protein may be used in methods of profiling a T-Cell receptor (TCR).

In various embodiments, the poly-dT sequence may be extended in a reverse transcription reaction using the mRNA as a template to produce a cDNA transcript complementary to the mRNA and also includes sequence of a barcode oligonucleotide. Terminal transferase activity of the reverse transcriptase can add additional bases to the cDNA transcript (e.g., polyC). The switch oligo may then hybridize with the additional bases added to the cDNA transcript and facilitate template switching. A sequence complementary to the switch oligo sequence can then be incorporated into the cDNA transcript via extension of the cDNA transcript using the switch oligo as a template. Within any given partition, all the cDNA transcripts of the individual mRNA molecules include a common barcode sequence. However, by including the unique random N-mer sequence, the transcripts made from different mRNA molecules within a given partition will vary at this unique sequence. As described elsewhere herein, this provides a quantification feature that can be identifiable even following any subsequent amplification of the contents of a given partition, e.g., the number of unique segments associated with a common barcode can be indicative of the quantity of mRNA originating from a single partition, and thus, a single cell. The cDNA transcript may then be amplified with PCR primers. The amplified product may then be purified (e.g., via solid phase reversible immobilization (SPRI)). The amplified product can be ligated to additional functional sequences, and further amplified (e.g., via PCR). The functional sequences may include a sequencer specific flow cell attachment sequence such as but not limited to., a P7 sequence for Illumina® sequencing systems, as well as functional sequence, which may include a sequencing primer binding site, e.g., for a R2 primer for Illumina® sequencing systems, as well as functional sequence, which may include a sample index, e.g., an i7 sample index sequence for Illumina® sequencing systems.

Although described in terms of specific sequence references used for certain sequencing systems, e.g., Illumina® systems, it will be understood that the reference to these sequences is for illustration purposes only, and the methods described herein may be configured for use with other sequencing systems incorporating specific priming, attachment, index, or other operational sequences used in those systems, e.g., systems available from Ion Torrent, Oxford Nanopore, Genia, Pacific Biosciences, Complete Genomics, and the like.

### 2. Volume

As described herein, wild-type and variants MMLV RT are not optimal for reverse transcription of mRNA when using high throughput amplification reaction assays (e.g., spatial array and single cell transcriptomics assay) and the like. This is because high throughput amplification reaction assays require reaction volumes that are usually less than about 1 nanoliter. Accordingly, the present disclosure provides novel engineered reverse transcriptase polypeptide or recombinant RT proteins that function efficiently in high throughput amplification reaction assays that require reaction volumes of less than about 1 nanoliter.

In some embodiments, the method comprises providing a reaction volume which comprises an engineered reverse transcriptase and a template ribonucleic acid (RNA) molecule. In one other embodiment, the contacting occurs in a reaction volume, which may be less than 1 nanoliter, less than 750 picoliters, or less than 500 picoliters. In other embodiments, the reaction volume is present in a partition, such as a droplet or well (including a microwell or a nanowell).

In some embodiments, the engineered reverse transcriptases, the recombinant RT protein, or derivatives thereof as described herein are used in a reaction volume less than about 1 nanoliter (nL). In some embodiments, the engineered reverse transcriptases, the recombinant RT proteins, or derivatives thereof, as described herein are used in a reaction volume that is less than about 500 picoliter (pL). In some embodiments, the reaction volume is contained within a partition. In some embodiments, the reaction volume is contained within a droplet. In some embodiments, the reaction volume is contained within a droplet in an emulsion. In some embodiments, the reaction volume is contained within a droplet emulsion having a reaction volume of less than about 1 nL. In some embodiments, the reaction volume is contained within a droplet emulsion having a reaction volume of less than about 500 μL.

In some embodiments, the reaction volume is contained within a well. In some embodiments, the reaction volume is contained within a well having a reaction volume less than about 1 nL. In some embodiments, the reaction volume is contained within a well. In some embodiments, the reaction volume is contained within a well having a reaction volume less than about 500 μL. In some embodiments, the reaction volume is contained within a well in an array of wells having an extracted nucleic acid molecule, and the template nucleic acid molecule is the extracted nucleic acid molecule. In some embodiments, the reaction volume is contained within a well in an array of wells having a cell comprising a template nucleic acid molecule, and where the template nucleic acid molecule is released from the cell.

In another embodiment, a method comprises providing a reaction volume, which comprises an engineered reverse transcriptase and a template ribonucleic acid (RNA) molecule and is considered a “low volume reaction”. The reaction volume may comprise a plurality of nucleic acid barcode molecules, and each nucleic acid barcode molecule comprises a barcode sequence. In an embodiment, the contacting occurs in a reaction volume, a low volume reaction, which may be less than 1 nanoliter, less than 750 picoliters, or less than 500 picoliters. In other embodiments, the reaction volume is present in a partition, such as a droplet or well (including a microwell or a nanowell).

### 3. Unique Molecular Identifier (UMI)

In some embodiments, the barcoding reaction produces single stranded complementary deoxyribonucleic acid (cDNA) molecules each having a molecular tag on a 5′ end thereof, followed by amplification of the cDNA to produce a double stranded DNA having the molecular tag on the 5′ end and a 3′ end of the double stranded DNA.

In some embodiments, the molecular tags (e.g., barcode oligonucleotides) include unique molecular identifiers (UMIs). In some embodiments, the UMIs are oligonucleotides. In some embodiments, the molecular tags are coupled to priming sequences. In some embodiments, each of the priming sequences comprises a random N-mer sequence. In some embodiments, the random N-mer sequence is complementary to a 3′ sequence of the RNA molecules. In some embodiments, the priming sequence comprises a poly-dT sequence having a length of at least 5 bases. In some embodiments, the priming sequence comprises a poly-dT sequence having a length of at least 10 bases (SEQ ID NO: 4). In some embodiments, the priming sequence comprises a poly-dT sequence having a length of at least 5 bases, at least 6 bases, at least 7 bases, at least 8 bases, at least 9 bases, at least 10 bases.

Unique molecular identifiers (UMIs), e.g., in the form of nucleic acid sequences are assigned or associated with individual cells or populations of cells, in order to tag or label the cell's components (and as a result, its characteristics). These unique molecular identifiers may be used to attribute the cell's components and characteristics to an individual cell or group of cells, additionally to be used as a method for counting the individual cells or groups of cells by their incorporation.

In some aspects, the unique molecular identifiers are provided in the form of nucleic acid molecules (e.g., oligonucleotides) that comprise nucleic acid barcode sequences that may be attached to or otherwise associated with the nucleic acid contents of individual cell, or to other components of the cell, and particularly to fragments of those nucleic acids. The nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

Moreover, when a population of barcodes is partitioned, the resulting population of partitions can also include a diverse barcode library that may include at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some embodiments, the enhanced reverse transcriptase activity of the engineered reverse transcriptase disclosed herein is an enhanced ability to yield mitochondrial UMI counts as compared to a reverse transcriptase having the amino acid sequence set forth in SEQ ID NO:1 or 15. In some embodiments, the enhanced reverse transcriptase activity is an enhanced ability to yield increased ribosomal UMI counts as compared to a reverse transcriptase having the amino acid sequence set forth in SEQ ID NO:1 or 15. Read counting and UMI counting are the principal gene expression quantification schemes used in single-cell RNA-sequencing (scRNA-seq) analysis, as such with increased ribosomal UMI counts sensitivity and accuracy increases for a scRNA-seq assay in determining transcriptome profiles for any given cell, group of cells or tissues. Numerous metrics can be used for quality control of single-cell RNA-sequencing, including percent of reads mapping to ribosomal genes, percent of reads mapping to mitochondrial genes, total number of UMIs detected, or number of features to which 50% of the reads map.

Beneficially, even following any subsequent amplification of the contents of a given partition, the number of different UMIs can be indicative of the quantity of mRNA originating from a given partition, and thus from the cell. As noted above, the transcripts can be amplified, purified and sequenced to identify the sequence of the cDNA transcript of the mRNA, as well as to sequence the barcode segment and the UMI segment. While a poly-dT primer sequence is described, other targeted or random primer sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents.

It is recognized that certain reverse transcriptase enzymes may increase UMI reads from genes of a desired length or length of interest. The desired length of genes may be selected from lengths comprising less than 500 nucleotides, between 500 and 1000 nucleotides, between 1000 and 1500 nucleotides and greater than 1500 nucleotides. It is recognized that a reverse transcriptase may preferentially increase UMI reads from genes of one length range. It is recognized that an engineered reverse transcriptase may perform similarly, differently or comparably in a 3′-reverse transcription assay or a 5′-reverse transcription assay. It is similarly recognized that an engineered reverse transcriptase may preferentially increase UMI reads from a length of genes in a 3′-reverse transcription assay than in a 5′-reverse transcription assay.

### 4. Gel Bead

The engineered reverse transcriptases or the recombinant RT protein of the present disclosure may be suitable for use in methods in which a cell can be co-partitioned along with a nucleic acid barcode molecule bearing bead. The nucleic acid barcode molecules can be released from the bead in the partition. By way of example, in the context of analyzing sample RNA, the poly-dT (poly-deoxythymine, also referred to as oligo(dT)) segment of one of the released nucleic acid molecules can hybridize to the poly-A tail of a mRNA molecule. Reverse transcription results in a cDNA transcript of the mRNA, but that transcript includes each of the sequence segments of the nucleic acid molecule. Without being limited by mechanism, because the nucleic acid molecule comprises an anchoring sequence, it may be more likely hybridize to and prime reverse transcription at the sequence end of the poly-A tail of the mRNA. Within any given partition, all of the cDNA transcripts of the individual mRNA molecules may include a common barcode sequence segment. However, the transcripts made from the different mRNA molecules within a given partition may vary at the unique UMI segment.

Beneficially, even following any subsequent amplification of the contents of a given partition, the number of different UMIs can be indicative of the quantity of mRNA originating from a given partition, and thus from the cell. As noted above, the transcripts can be amplified, cleaned up and sequenced to identify the sequence of the cDNA transcript of the mRNA, as well as to sequence the barcode segment and the UMI segment. While a poly-dT primer sequence is described, other targeted or random priming sequences may also be used in priming the reverse transcription reaction.

In some embodiments of the nucleic acid extension method described herein, the plurality of nucleic acid barcoded molecules are attached to a support (e.g., a particle, a slide, a chip, a bead, etc.). In one embodiment, the support is selected from an array, a bead, a gel bead, a microparticle, and a polymer. In some embodiments, the nucleic acid barcoded molecules attached to a support comprise molecular tags (UMIs), primer sequences, capture sequences, cleavage sequences, or additional functional sequences. In some embodiments, the support is a gel bead. In that embodiment, the nucleic acid barcoded molecules are releasably attached to the gel bead. In some embodiments, the gel bead comprises a polyacrylamide polymer.

In some embodiments, a cross-section of the gel bead is less than about 100 μm. In some embodiments, a cross-section of a gel bead is less than about 60 μm. In some embodiments, a cross-section of a gel bead is less than about 50 μm. In some embodiments, a cross-section of a gel bead is less than about 40 μm. In some embodiments, a cross-section of a gel bead is less than about 100 μm, less than about 99 μm, less than about 98 μm, less than about 97 μm, less than about 96 μm, less than about 95 μm, less than about 94 μm, less than about 93 μm, less than about 92 μm, less than about 91 μm, less than about 90 μm, less than about 89 μm, less than about 88 μm, less than about 87 μm, less than about 86 μm, less than about 85 μm, less than about 84 μm, less than about 83 μm, less than about 82 μm, less than about 81 μm, less than about 80 μm, less than about 79 μm, less than about 78 μm, less than about 77 μm, less than about 76 μm, less than about 75 μm, less than about 74 μm, less than about 73 μm, less than about 72 μm, less than about 71 μm, less than about 70 μm, less than about 69 μm, less than about 68 μm, less than about 67 μm, less than about 66 μm, less than about 65 μm, less than about 64 μm, less than about 63 μm, less than about 62 μm, less than about 61 μm, or less than about 60 μm.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina® sequencing. In some cases, the nucleic acid molecule can comprise a barcode sequence. In some cases, the primer can comprise a unique molecular identifier (UMI). In some cases, the primer can comprise an R1 sequence for use in Illumina® sequencing workflows. In some cases, the primer can comprise an R2 sequence for use in Illumina® sequencing workflows. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference. However, the present invention is not limited as to a composition of any nucleic acid molecule or derivative thereof, or any particular sequencing platform and these characterizations serve as examples only which may be useful in a reverse transcription workflow.

In operation, a cell can be co-partitioned along with a barcode bearing bead. The barcoded nucleic acid molecules affixed to a bead can be released from the bead in the partition. By way of example, in the context of analyzing sample RNA, the poly-dT (poly-deoxythymine, also referred to as oligo(dT)) segment of one of the released nucleic acid molecules can hybridize to (e.g., capture)_the poly-A tail of a mRNA molecule. Reverse transcription may result in a cDNA transcript of the mRNA which cDNA transcript also includes each of the sequence segments of the nucleic acid molecule. Because the nucleic acid molecule comprises additional functional sequences (e.g., capture domains, primer domains, UMIs, barcodes, etc.), it can hybridize to and prime reverse transcription of the mRNA using the hybridized mRNA as a template. Within any given partition, all of the cDNA transcripts of the individual mRNA molecules may include a common barcode sequence. However, the transcripts made from the different mRNA molecules within a given partition may vary with respect to unique molecular identifying sequences (e.g., UMIs). Beneficially, following any subsequent amplification of the contents of a given partition, the number of different UMIs can be indicative of the quantity of mRNA originating from a given partition, and thus from the cell. As noted above, the transcripts can be amplified and sequenced to identify the sequence of the original mRNA captured template, as well as the sequence of the associated barcode and UMI. While a poly-dT capture sequence is described, other targeted or random capture sequences may also be used in capture or hybridize to a template for initiating the reverse transcription reaction.

In various embodiments, the poly-dT segment may be extended in a reverse transcription reaction using the mRNA as a template to produce a cDNA transcript complementary to the mRNA and also includes sequence segments of a barcode oligonucleotide. Terminal transferase activity of the reverse transcriptase can add additional bases to the cDNA transcript (e.g., polyC). The switch oligo may then hybridize with the additional bases added to the cDNA transcript and facilitate template switching. A sequence complementary to the switch oligo sequence can then be incorporated into the cDNA transcript via extension of the cDNA transcript using the switch oligo as a template. Within any given partition, all the cDNA transcripts of the individual mRNA molecules include a common barcode sequence segment. However, by including the unique random N-mer sequence, the transcripts made from different mRNA molecules within a given partition will vary at this unique sequence. As described elsewhere herein, this provides a quantification feature that can be identifiable even following any subsequent amplification of the contents of a given partition, e.g., the number of unique segments associated with a common barcode can be indicative of the quantity of mRNA originating from a single partition, and thus, a single cell. The cDNA transcript may then be amplified with PCR primers. The amplified product may then be purified (e.g., via solid phase reversible immobilization (SPRI)). The amplified product may be sheared, ligated to additional functional sequences, and further amplified (e.g., via PCR).

Any of the engineered RT enzymes of the present disclosure, including without limitation any of the enzymes comprising the amino acid sequence and/or non-limiting embodiment of nucleic acid sequences shown in Table 1, or Table 2, could be analyzed in any suitable assay, including without limitation the assays described herein. Assays include without limitation 5′ gene expression analyses, with or without VDJ analysis, 3′ gene expression analysis, epigenetic analysis, or multiomic analyses. In non-limiting embodiments, experiments are carried out as found in the manufacturer's instructions for the Chromium Single Cell 5′ Gene Expression Assay kit (10× Genomics); Chromium Single Cell 3′ Gene Expression Assay kit (10× Genomics), including any of multiomic extensions or applications.

### C. Biological Sample

Methods disclosed herein can be performed on any type of sample. The sample can comprise a cell, a cell bead, a permeabilized cell, or a nucleus. The cell can be fixed. The cell can be permeabilized. Alternatively, the cell can be permeabilized and fixed. In some embodiments, the cell bead is fixed. In some embodiments, the nucleus is permeabilized or fixed. In some embodiments, the nucleus is permeabilized and fixed. In some embodiments, the biological sample comprises a suitable cellular preparation selected from cell populations and/or single cells. In some embodiments, the biological sample comprises a tissue.

The sample can also be a suitable cellular preparation selected from cell populations and/or single cells. The sample can be a tissue. In some embodiments, the sample comprises cells in suspension, fresh cells, or fixed cells. The sample can also comprise cells and tissues immobilized on various solid surfaces.

In some embodiments, when the biological sample is a cell, a cell bead, or a nucleus, the reverse transcription reaction described herein is part of a single cell RNA sequencing assay. In one embodiment, the single cell RNA sequencing assay further comprises, prior to the reverse transcription, partitioning the cell, the cell bead, or the nucleus into a partition. In one embodiment, the single cell RNA sequencing assay further comprises, after the reverse transcription reaction, hybridizing the nucleic acid product to an oligonucleotide molecule comprising a partition-specific barcode.

In some embodiments, when the biological sample is a cell or a tissue sample immobilized on a surface, the reverse transcription reaction described herein is part of a spatial RNA sequencing assay.

In some embodiments, the sample is a fresh tissue. In some embodiments, the sample is a frozen sample. In some embodiments, the sample was previously frozen. In some embodiments, the sample is a formalin-fixed, or paraffin embedded (FFPE) sample. FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some embodiments, a biological sample (e.g., tissue section) can be fixed with methanol, stained with hematoxylin and eosin, and imaged. In some embodiments, fixing, staining, and imaging occurs before one or more oligonucleotide probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the methods of targeted RNA capture as disclosed herein include hybridization of multiple probe oligonucleotides. In some embodiments, the methods include 2, 3, 4, or more probe oligonucleotides that hybridize to one or more analytes of interest. In some embodiments, the methods include two probe oligonucleotides. In some embodiments, the probe oligonucleotide includes sequences complementary that are complementary or substantially complementary to an analyte. For example, in some embodiments, the probe oligonucleotide includes a sequence that is complementary or substantially complementary to an analyte (e.g., an mRNA of interest (e.g., to a portion of the sequence of an mRNA of interest)). Methods provided herein may be applied to a single nucleic acid molecule or a plurality of nucleic acid molecules. A method of analyzing a sample comprising a nucleic acid molecule may comprise providing a plurality of nucleic acid molecules (e.g., RNA molecules), where each nucleic acid molecule comprises a first target region (e.g., a sequence that is 3′ of a target sequence or a sequence that is 5′ of a target sequence) and a second target region (e.g., a sequence that is 5′ of a target sequence or a sequence that is 3′ of a target sequence), a plurality of first probe oligonucleotides, and a plurality of second probe oligonucleotides.

In some embodiments, the templated ligation methods that allow for targeted RNA capture as provided herein include a first probe oligonucleotide and a second probe oligonucleotide. The first and second probe oligonucleotides each include sequences that are substantially complementary to the sequence of an analyte of interest. By substantially complementary, it is meant that the first and/or second probe oligonucleotide is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a sequence in an analyte. In some instances, the first probe oligonucleotide and the second probe oligonucleotide hybridize to adjacent sequences on an analyte.

In some embodiments, the first and/or second probe as disclosed herein includes one of at least two ribonucleic acid bases at the 3′ end; a functional sequence; a phosphorylated nucleotide at the 5′ end; and/or a capture probe binding domain. In some embodiments, the functional sequence is a primer sequence. The capture probe binding domain is a sequence that is complementary to a particular capture domain present in a capture probe. In some embodiments, the capture probe binding domain includes a poly(A) sequence. In some embodiments, the capture probe binding domain includes a poly-uridine sequence, a poly-thymidine sequence, or both. In some embodiments, the capture probe binding domain includes a random sequence (e.g., a random hexamer or octamer). In some embodiments, the capture probe binding domain is complementary to a capture domain in a capture probe that detects a particular target(s) of interest.

In some embodiments, a capture probe binding domain blocking moiety that interacts with the capture probe binding domain is provided. In some instances, the capture probe binding domain blocking moiety includes a nucleic acid sequence. In some instances, the capture probe binding domain blocking moiety is a DNA oligonucleotide. In some instances, the capture probe binding domain blocking moiety is an RNA oligonucleotide. In some embodiments, a capture probe binding domain blocking moiety includes a sequence that is complementary or substantially complementary to a capture probe binding domain. In some embodiments, a capture probe binding domain blocking moiety prevents the capture probe binding domain from binding the capture probe when present. In some embodiments, a capture probe binding domain blocking moiety is removed prior to binding the capture probe binding domain (e.g., present in a ligated probe) to a capture probe. In some embodiments, a capture probe binding domain blocking moiety comprises a poly-uridine sequence, a poly-thymidine sequence, or both.

In some embodiments, the first probe oligonucleotide hybridizes to an analyte. In some embodiments, the second probe oligonucleotide hybridizes to an analyte. In some embodiments, both the first probe oligonucleotide and the second probe oligonucleotide hybridize to an analyte. Hybridization can occur at a target having a sequence that is 100% complementary to the probe oligonucleotide(s). In some embodiments, hybridization can occur at a target having a sequence that is at least (e.g., at least about) 80%, at least (e.g., at least about) 85%, at least (e.g., at least about) 90%, at least (e.g., at least about) 95%, at least (e.g., at least about) 96%, at least (e.g., at least about) 97%, at least (e.g., at least about) 98%, or at least (e.g., at least about) 99% complementary to the probe oligonucleotide(s).

After hybridization of the first and second probe oligonucleotides, in some embodiments, the first probe oligonucleotide is extended. After hybridization, in some embodiments, the second probe oligonucleotide is extended. Extending probes can be accomplished using any method disclosed herein. In some instances, a polymerase (e.g., a DNA polymerase) extends the first and/or second oligonucleotide.

In some embodiments, methods disclosed herein include a wash step. In some instances, the wash step occurs after hybridizing the first and the second probe oligonucleotides. The wash step removes any unbound oligonucleotides and can be performed using any technique or solution disclosed herein or known in the art. In some embodiments, multiple wash steps are performed to remove unbound oligonucleotides.

In some embodiments, after hybridization of probe oligonucleotides (e.g., first and the second probe oligonucleotides) to the analyte, the probe oligonucleotides (e.g., the first probe oligonucleotide and the second probe oligonucleotide) are ligated together, creating a single ligated probe that is complementary to the analyte. Ligation can be performed enzymatically or chemically, as described herein.

In some embodiments of the method described herein, the engineered RT polypeptide or the recombinant RT protein enhances template switching (TS) efficiency, processivity efficiency, binding affinity, transcription efficiency, chemical tolerance, ability to yield mitochondrial unique molecular identity (UMI) counts, ability to yield ribosomal unique molecular identity (UMI) counts, strand displacement, end-to-end template jumping, or any combination thereof, when compared to an RT polypeptide or a recombinant RT protein lacking a conjugated DNA binding domain.

In some embodiments, the engineered RT polypeptide or the recombinant RT protein enhances at least two or more of template switching (TS) efficiency, processivity efficiency, binding affinity, transcription efficiency, chemical tolerance, ability to yield mitochondrial unique molecular identity (UMI) counts, strand displacement, end-to-end template jumping, or ability to yield ribosomal unique molecular identity (UMI) counts, when compared to an RT polypeptide or a recombinant RT protein lacking a conjugated DNA binding domain.

In some embodiments, the recombinant RT protein or the engineered RT enhances transcript capture during amplification, when compared to an RT polypeptide or a recombinant RT protein lacking a conjugated DNA binding domain.

In some embodiments, the DNA binding domain of the recombinant RT protein or the engineered RT enhances the hybridization of a transcript and a primer during the amplification process, when compared to an RT polypeptide or a recombinant RT protein lacking a conjugated DNA binding domain.

### D. Immunoprofiling

Engineered reverse transcriptase polypeptides or recombinant RT proteins described herein may be used in methods of a T-Cell receptor (TCR) and a B-cell receptor (BRC) profiling.

In some embodiments, an engineered reverse transcriptase is used in methods including but not limited to processing of a TCR from an individual T cell(s) or groups of T cell(s), determining the nucleotide sequence of the TCR(s) of T cell(s), and obtaining TCR repertoire profile. In some methods, a nucleic acid barcode sequence is appended to a nucleic acid molecule encoding for a TCR (e.g. a molecule derived from a T cell containing a nucleic acid sequence encoding for a TCR, such as a TCRα and/or a TCRβ mRNA) resulting in a barcoded nucleic acid molecule comprising a sequence corresponding to a nucleic acid sequence of the TCR (e.g. comprises a V(D)J region of a TCR gene or a reverse complement thereof) and a sequence corresponding to the barcode sequence (which in some instances is the reverse complement of the barcode sequence present in the nucleic acid barcode molecule). A barcoded nucleic acid molecule may serve as a template, such as a template polynucleotide, that can be further processed (e.g., amplified) and sequenced to obtain the target nucleic acid sequence. For example, a barcoded nucleic acid molecule may be further processed (e.g., amplified) and sequenced to obtain the nucleic acid sequence of the TCR.

TCR is a molecule found on the surface of T cells. Typically binding of the TCR by an antigenic molecule results in cell activation and response. The TCR is a heterodimer composed of two different protein chains. In many T cells, these two proteins are alpha (α) and beta (β) chains. In a smaller percentage of T cells, these two proteins are gamma (γ) and delta (δ) chains. The ratio of TCRs comprised of α/β chains versus γ/δ chains may change during a diseased state such as cancer, tumor, infectious disease, inflammatory disease or autoimmune disease. Engagement of the TCR with a peptide-MHC activates a T cell through a series of biochemical events mediated by associated enzymes, co-receptors, specialized adaptor molecules, and activated or released transcription factors.

Each of the two chains of a TCR contains multiple copies of gene segments-a variable ‘V’ gene segment, a diversity ‘D’ segment and a joining ‘J’ segment. The TCR alpha chain is generated by recombination of V and J segments, while the beta chain is generated by recombination of V, D and J segments. Similarly, generation of the TCR gamma chain involves recombination of V and J segments. Generation of the TCR delta chain occurs by recombination of V, D and J gene segments. The intersection of these specific regions (V and J for the alpha or gamma chain, or V, D, J for the beta or delta chain) corresponds to the CDR3 region involved in antigen-MHC recognition. Complementarity determining regions (e.g., CDR1, CDR2 and CDR3) or hypervariable regions are sequences in the variable domains of antigen receptors (e.g., T cell receptor and immunoglobulin) that can complement an antigen. Most of the diversity of CDRs is found in CDR3, with the diversity being generated by somatic recombination events during the development of T lymphocytes. CDR3, which is encoded by the junctional region between the V and J or D and J genes, is highly variable. CDR3 is often used as a region of interest to determine T cell clonotypes, a unique nucleotide sequence that arises during the gene rearrangement process, as it is highly unlikely that two T cells will express the same CDR3 nucleotide sequence unless they are derived from the same clonally expanded T cell.

Because an active TCR consists of paired chains within single T cells, determination of the active paired chains within single T cells, determination of the active paired chains requires the sequencing of single T cells. TCR gene sequences may include, but are not limited to, sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes) and T cell receptor delta constant genes (TRDC genes).

### VII. Kits

One aspect of the present disclosure provides a kit comprising the engineered reverse transcriptase polypeptide or recombinant RT protein, the DNA binding domains or a derivative thereof as described herein. In some embodiments, the kit comprises one or more of a vector, a nucleotide, a buffer, a composition, a salt, and/or instructions. In another embodiment, a kit may comprise an engineered reverse transcriptase polypeptide or recombinant RT protein or a derivative thereof for use in reverse transcription or amplification of a nucleic acid molecule. In yet another embodiment, a kit may be used for single cell profiling of the transcriptome. In yet another embodiment, a kit may be used for spatial transcriptomics methods and assays. In yet another embodiment, a kit may be used for in situ methods and assays.

The kit may include suitable reaction buffers, dNTPs, one or more primers, one or more control reagents, or any other reagents disclosed for performing the methods of the present disclosure. The engineered reverse transcriptase polypeptide or recombinant RT protein or a derivative thereof, reaction buffer, and dNTPs may be provided separately or may be provided together in a master mix solution. When the engineered reverse transcriptase polypeptide or recombinant RT protein or a derivative thereof, reaction buffer, and dNTPs are provided in a master mix, the master mix is present at a concentration at least two times the working concentration indicated in instructions for use in an extension reaction. In other cases, the master mix may be present at a concentration at least three times, at least four times, at least five times, at least six times, at least seven times, at least eight times, at least nine times, or at least ten times, the working concentration indicated. The primer in the kits may be a poly-dT primer, a random N-mer primer, or a target-specific primer.

The kits may further include one, two, three, four, five or more, up to all of partitioning fluids, including both aqueous buffers and non-aqueous partitioning fluids or oils, nucleic acid barcode capture probes that are releasably associated with beads, as described herein, microfluidic devices, reagents for disrupting cells, reagents for amplifying nucleic acids, as well as instructions for using any of the foregoing in the methods described herein.

The instructions for using any of the methods are generally recorded on a suitable recording medium (e.g., printed on a substrate such as paper or plastic), or available in a digital format. As such, the instructions may be present in the kits as a package insert, in the labeling of the container of the kit or components thereof (i.e., associated with the packaging or subpackaging). In some cases, the instructions may be present as an electronic storage data file present on a suitable computer readable storage medium. In other cases, the actual instructions may not be present in the kit but means for obtaining the instructions from a remote source, e.g., via the internet, may be provided. For example, a kit that includes a web address where the instructions may be viewed and/or from which the instructions may be downloaded. As with the instructions, this means for obtaining the instructions is recorded on a suitable substrate.

Kits according to this aspect of the present disclosure comprise a carrier means, such as a box, carton, tube or the like, having in close confinement therein one or more container means, such as vials, tubes, ampoules, bottles and the like, wherein a first container means contains one or more of the engineered reverse transcriptase polypeptide or recombinant RT proteins or derivatives thereof of the present disclosure having reverse transcriptase activity. When more than one polypeptide having reverse transcriptase activity is used, they may be in a single container as mixtures of two or more engineered reverse transcriptase polypeptide or recombinant RT proteins or derivatives thereof, or in separate containers. The kits of the disclosure can also comprise (in the same or separate containers) one or more DNA polymerases, a suitable buffer, one or more nucleotides and/or one or more primers.

The kits of the disclosure can also comprise one or more hosts or cells including those that are competent to take up nucleic acids (e.g., DNA molecules including vectors). Preferred hosts may include chemically competent or electrocompetent bacteria such as E. coli (including DH5, DH5α, DH10B, HB101, Top 10, and other K-12 strains as well as E. coli B and E. coli W strains)

In a specific aspect of the present disclosure, the kits of the disclosure (e.g., reverse transcription and amplification kits) can include one or more components (in mixtures or separately) including one or more engineered reverse transcriptase polypeptides or recombinant RT proteins or derivatives thereof having reverse transcriptase activity of the disclosure, one or more nucleotides (one or more of which may be labeled, e.g., fluorescently labeled) used for synthesis of a nucleic acid molecule, and/or one or more primers (e.g., oligo(dT) for reverse transcription, randomers for extension reactions, etc.). Such kits can comprise one or more DNA polymerases.

### VIII. Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. “A and/or B” is used herein to include all of the following alternatives: “A”, “B”, “A or B”, and “A and B”. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “About” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

In some embodiments, the term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Numeric ranges are inclusive of the numbers defining the range. The term about is used herein to mean plus or minus ten percent (10%) of a value. For example, “about 100” refers to any number between 90 and 110.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

As used herein, the term “Analyte” is intended a biological molecule. Analytes include but are not limited to a DNA analyte, an RNA analyte, an oligonucleotide, a reporter molecule, a reporter molecule configured to directly couple to a protein, a reporter molecule configured to indirectly couple to a protein, a reporter molecule configured to directly couple to a metabolite, and a reporter molecule configured to indirectly couple to a metabolite.

The terms “Adaptor(s),” “Adapter(s)” and “Tag(s)” may be used synonymously. An adaptor or tag can be coupled to a polynucleotide sequence to be “tagged” by any approach, including ligation, hybridization, or other approaches.

As used herein, the term “Barcoded nucleic acid molecule” generally refers to a nucleic acid molecule that results from, for example, the processing of a nucleic acid barcoded molecule with a nucleic acid sequence (e.g., nucleic acid sequence complementary to a nucleic acid primer sequence encompassed by the nucleic acid barcoded molecule). The nucleic acid sequence may be a targeted sequence or a non-targeted sequence. The nucleic acid barcoded molecule may be coupled to or attached to the nucleic acid molecule comprising the nucleic acid sequence. For example, a nucleic acid barcoded molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcoded molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcoded molecule to generate the barcoded nucleic acid molecule, or the nucleic acid molecule comprising the nucleic acid sequence may be attached to the nucleic acid barcoded molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

A nucleic acid barcoded molecule of a plurality of nucleic acid molecules may be used to generate a “barcoded nucleic acid molecule.” In some cases, a barcoded molecule comprises a different reporter barcode sequence that identifies a second analyte. A different reporter barcode sequence or an analyte-specific barcode sequence may identify a protein, a lipid, a metabolite or other second analyte.

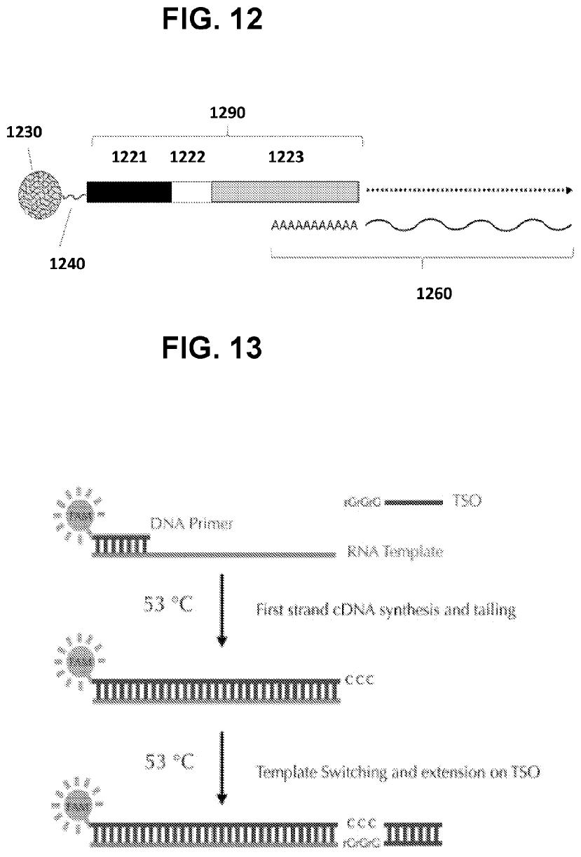

Barcoded nucleic acids may be generated (e.g., via a nucleic acid reaction, such as nucleic acid extension or ligation) from the constructs described in FIG. 12. For example, capture handle sequence may then be hybridized to complementary sequence, such as capture sequence 1223 to generate (e.g., via a nucleic acid reaction, such as nucleic acid extension or ligation) a barcoded nucleic acid molecule comprising cell (e.g., partition specific) barcode sequence 1222 (or a reverse complement thereof) and reporter barcode sequence 1222 (or a reverse complement thereof). In some embodiments capture handle sequence 4323 comprises a sequence complementary to a template switching oligonucleotide on the capture sequence 1223. In some embodiments, the nucleic acid barcoded molecule 1290 (e.g., partition-specific barcoded molecule) further includes a UMI (not shown). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. In some instances, analysis of an analyte (e.g., a nucleic acid, a polypeptide, a carbohydrate, a lipid, a glycan, a glycan motif, a metabolite, a protein, etc.) comprises a workflow as generally depicted in FIG. 12. A nucleic acid barcoded molecule 1290 (e.g., partition specific barcoded molecule) may be co-partitioned with the one or more analytes. In some instances, nucleic acid barcoded molecule 1290 is attached to a support 1230 (e.g., a bead, such as a gel bead), such as those described elsewhere herein. For example, nucleic acid barcoded molecule 1290 may be attached to support 1230 via a releasable linkage 1240 (e.g., comprising a labile bond), such as those described elsewhere herein. Nucleic acid barcoded molecule 1290 may comprise a functional sequence 1221 and optionally comprise other additional sequences, for example, a barcode sequence 1222 (e.g., common barcode, partition-specific barcode, or other functional sequences described elsewhere herein), and/or a UMI sequence (not shown). The nucleic acid barcoded molecule 1290 may comprise a capture sequence 1223 that may be complementary to another nucleic acid sequence, such that it may hybridize to a particular sequence, e.g., capture handle sequence 1223.

For example, capture sequence 1223 may comprise a poly-T sequence and may be used to hybridize to mRNA. Referring to FIG. 12, in some embodiments, nucleic acid barcoded molecule 1290 comprises capture sequence 1223 complementary to a sequence of RNA molecule 1260 from a cell. In some instances, capture sequence 1223 comprises a sequence specific for an RNA molecule. Capture sequence 1223 may comprise a known or targeted sequence or a random sequence. In some instances, a nucleic acid extension reaction may be performed, thereby generating a barcoded nucleic acid product comprising capture sequence 12323, the functional sequence 1221, barcode sequence 1222, any other functional sequence, and a sequence corresponding to the RNA molecule 1260.

In another example, capture sequence 1223 may be complementary to an overhang sequence or an adapter sequence that has been appended to an analyte. Any suitable agent may degrade beads. Suitable agents may include, but are not limited to, changes in temperature, changes in pH, reduction, oxidation and exposure to water or other aqueous solutions.

In some instances, a cell that is bound to labelling agent which is conjugated to oligonucleotide and support 1230 (e.g., a bead, such as a gel bead) comprising nucleic acid barcoded molecule 1290 is partitioned into a partition amongst a plurality of partitions (e.g., a droplet of a droplet emulsion, a well of a microwell array, a fixed cell and/or nucleus, a fixed and permeabilized cell and/or nucleus).

The term “Bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “Each,” when used in reference to a collection of items, is intended to identify an individual item in the collection but does not necessarily refer to every item in the collection, unless expressly stated otherwise, or unless the context of the usage clearly indicates otherwise.

As used herein, the term “Efficiency” in the context of a nucleic acid modifying enzyme of this invention refers to the ability of the enzyme to perform its catalytic function under specific reaction conditions. Typically, “efficiency” as defined herein is indicated by the amount of product generated under given reaction conditions.

As used herein, the term “Enhances” in the context of an enzyme refers to improving the activity of the enzyme, i.e., increasing the amount of product per unit enzyme per unit time.

As used herein, the term “Fidelity” refers to the accuracy of polymerization, or the ability of the reverse transcriptase to discriminate correct from incorrect substrates, (e.g., nucleotides) when synthesizing nucleic acid molecules which are complementary to a template. The higher the fidelity of a reverse transcriptase, the less the reverse transcriptase misincorporates nucleotides in the growing strand during nucleic acid synthesis; that is, an increase or enhancement in fidelity results in a more faithful reverse transcriptase having decreased error rate or decreased misincorporation rate.

As used herein, the term “% homology,” which is used interchangeably with the term “% identity,” refers to the level of nucleic acid or amino acid sequence identity between the nucleic acid sequence that encodes any one of the inventive polypeptides (e.g., variant reverse transcriptases) or the inventive polypeptide's amino acid sequence, when aligned using a sequence alignment program.

As used herein, the term “Identical” in the context of two nucleic acids or polypeptide sequences refers to the residues in the two sequences that are the same when aligned for maximum correspondence, as measured using a sequence comparison algorithms. Sequence comparison algorithms are known to those skill in the art. See. e.g., ebi.ac.uk/Tools/msa/clustalo/.

As used herein, the term “Inhibitor resistance” refers to the ability of a reverse transcriptase to perform reverse transcription in the presence of a compound, chemical, protein, buffer, etc. that is typically inhibitory to the reverse transcriptase (prevents or inhibits reverse transcriptase activity).

As used herein, the term “Low volume reaction” means a reaction volume less than 1 nanoliter, less than 750 picoliters, or less than 500 picoliters.

The term “Molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

As used herein, the term “mutation” or “mutant” or “variant” indicates a change or changes introduced in a wild-type DNA sequence or a wildtype amino acid sequence. Examples of mutations or variants include, but are not limited to, substitutions, insertions, deletions, and point mutations. Mutations can be made either at the nucleic acid level or at the amino acid level.

As used herein, the term “Operably linked” or “conjugated” or “fusion” means that, in relation to the engineered RT polypeptide or the recombinant RT protein sequence, there are one or more sequences at the N or C terminus that, when transcribed and translated, create additional polypeptides in association with the enzyme amino acid sequence, thereby created a conjugation or fusion of one or more polypeptides from one expression vector.

The term “Partition,” as used herein, generally, refers to a space or volume that may be suitable to contain one or more species or conduct one or more reactions. A partition may be a physical compartment, such as a droplet, well or a fixed and/or permeabilized cell and/or nucleus. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “Partitioning” as used herein is intended to encompass parting, dividing, depositing, separating, or compartmentalizing into one or more partitions. Systems and methods for partitioning of one or more particles (such as, but not limited to, biological particles, macromolecular constituents of biological particles, beads, reagents, etc.) into discrete compartments or partitions (referred to interchangeably here as partitions), wherein each partition maintains separation of its own content from the contents of other partitions are known in the art. See for example US 2020/0032335, herein incorporated by reference in its entirety. The partition can be a droplet in an emulsion. A partition may comprise one or more other partitions.

A “plurality of nucleic acid barcoded molecules” may comprise at least about 500 nucleic acid barcoded molecules, at least about 1,000 nucleic acid barcoded molecules, at least about 5,000 nucleic acid barcoded molecules, at least about 10,000 nucleic acid barcoded molecules, at least about 50,000 nucleic acid barcoded molecules, at least about 100,000 nucleic acid barcoded molecules, at least about 500,000 nucleic acid barcoded molecules, at least about 1,000,000 barcoded molecules, at least about 5,000,000 nucleic acid barcoded molecules, at least about 10,000,000 nucleic acid barcoded molecules, at least about 100,000,000 nucleic acid barcoded molecules, at least about 1,000,000,000 nucleic acid barcoded molecules. In some cases, a plurality of nucleic acid barcoded molecules comprise a partition-specific barcode sequence.

Each of the plurality of nucleic acid barcoded molecules may include an identifier sequence separate from the partition-specific barcode sequence, where the identifier sequence is different for each nucleic acid partition-specific barcoded molecule of the plurality of nucleic acid partition specific barcoded molecules. In some cases, such an identifier sequence is a unique molecular identifier (UMI) as described elsewhere herein. As described elsewhere herein, UMI sequences can uniquely identify a particular nucleic acid molecule that is barcoded, which may be identifying particular nucleic acid molecules that are analyzed, counting particular nucleic acid molecules that are analyzed, etc. Furthermore, in some cases, each of the plurality of nucleic acid barcoded molecules can comprise the partition specific barcode sequence and the bead can be from plurality of beads, such as a population of barcoded beads. Each of the partition specific barcode sequences can be different from partition specific barcode sequences of nucleic acid barcoded molecules of other beads of the plurality of beads. Where this is the case, a population of barcoded beads, with each bead comprising a different partition specific barcode sequence can be analyzed.

As used herein, the term “Processivity” refers to the ability of a reverse transcriptase to continuously extend a primer without disassociating from the nucleic acid template. The length of a template a reverse transcriptase or polymerase is capable of replicating can also be used to describe the processivity of that reverse transcriptase or polymerase. In some embodiments, “Processivity” refers to the ability of a polymerase to remain bound to the template or substrate and perform DNA synthesis. Processivity is measured by the number of catalytic events that take place per binding event.

As used herein, “Purified” means that a molecule is present in a sample at a concentration of at least 95% by weight, or at least 98% by weight of the sample in which it is contained.

As used herein, the term “Reverse transcriptase activity,” “reverse transcription activity,” or “reverse transcription” indicates the capability of an enzyme to synthesize a DNA strand (that is, complementary DNA or cDNA) using RNA as a template. Reverse transcriptase activity may be measured by incubating an enzyme in the presence of an RNA template and deoxynucleotides, in the presence of an appropriate buffer, under appropriate conditions, for example as described in the Example below. Methods for measuring RT activity are provided in the example below and also are well known in the art. Bosworth, et al., Nature 1989, 341:167-168.

As used herein, the term “recombinant RT” comprises the engineered RT fusion protein described herein or the engineered RT variant described herein.

As used herein, the term “Reverse transcriptase (RT)” is used in its broadest sense to refer to any enzyme that exhibits reverse transcription activity as measured by methods disclosed herein or known in the art. A “reverse transcriptase” of the present invention, therefore, includes reverse transcriptases from retroviruses, other viruses, as well as a DNA polymerase exhibiting reverse transcriptase activity, such as Tth DNA polymerase, Taq DNA polymerase, Tne DNA polymerase, Tma DNA polymerase, etc. RT from retroviruses include, but are not limited to, Moloney Murine Leukemia Virus (M-MLV) RT, Human Immunodeficiency Virus (HIV) RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus UR2AV RT, Avian Sarcoma Virus Y73 Helper Virus YAV RT, Rous Associated Virus (RAV) RT, and Myeloblastosis Associated Virus (MAV) RT, and as described in U.S. Patent Application 2003/0198944 (hereby incorporated by reference in its entirety). For review, see e.g., Levin, 1997, Cell, 88:5-8; Brosius et al. 5 1995, Virus Genes 11:163-79. Known reverse transcriptases from viruses require a primer to synthesize a DNA transcript from an RNA template. Reverse transcriptase has been used primarily to transcribe RNA into cDNA, which can then be cloned into a vector for further manipulation or used in various amplification methods such as polymerase chain reaction (PCR), nucleic acid sequence-based amplification (NASBA), transcription mediated amplification (TMA), or self-sustained sequence replication (3SR).

The term “Sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from blood, plasma, serum, urine, saliva, mucosal excretions, sputum, stool and tears.

As used herein, the term “Sequencing,” generally refers to methods and technologies for determining the sequence of nucleotide bases in one or more polynucleotides. Any method of sequencing known in the art may be used to evaluate the products of a reaction performed by an engineered reverse transcriptase of the current application. Sequencing can be performed by various systems currently available, such as, without limitation, a sequencing system by Illumina®, Pacific Biosciences (PacBio®), Oxford Nanopore®, or Life Technologies (Ion Torrent®). Alternatively, or in addition, sequencing may be performed using nucleic acid amplification, polymerase chain reaction (PCR) (e.g., digital PCR, quantitative PCR, or real time PCR), or isothermal amplification. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

As used herein, the term “Substantially complementary” means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “Subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

As used herein, the term “Thermoreactivity” or “Thermoreactive” refers to the ability of a reverse transcriptase to exhibit enzyme activity at elevated temperatures.

As used herein, “Thermostability” or “thermostable” refers to the ability of a reverse transcriptase to withstand exposure to elevated temperatures, but not necessarily show activity at such elevated temperatures. In some embodiments, a thermostable reverse transcriptase or polymerase refers to any enzyme that catalyzes polynucleotide synthesis by addition of nucleotide units to a nucleotide chain using DNA or RNA as a template and has an optimal activity at a temperature above 53° C.

As used herein, the terms “Unique molecular identifier”, “Unique molecular identifying sequence”, “UMI” and “UMI sequence” are used synonymously. Individual barcoded molecules may comprise a common barcode sequence such as a partition specific sequence or a spatial array where every capture probe has a unique barcode sequence.

By “Binding sequence” is intended a nucleic acid sequence capable of binding to an analyte.

As used herein, the term “Variant” means a protein which is derived from a precursor protein (such as the native protein, for example MMLV native protein as set forth in SEQ ID NO: 7) by addition of one or more amino acids to either or both the C- and N-terminal end, substitution of one or more amino acids at one or a number of different sites in the amino acid sequence, deletion of one or more amino acids at either or both ends of the protein or at one or more sites in the amino acid sequence, or addition of a fusion domain. SEQ ID NO: 1 is a variant of MMLV. The preparation of an enzyme variant is preferably achieved by modifying a DNA sequence which encodes for the wild-type protein, transformation of that DNA sequence into a suitable host, and expression of the modified DNA sequence to form the derivative enzyme. A variant reverse transcriptase of the invention includes a protein comprising altered amino acid sequences in comparison with a precursor enzyme amino acid sequence wherein the variant reverse transcriptase retains the characteristic enzymatic nature of the precursor enzyme but which may have altered properties in some specific aspect. For example, an engineered reverse transcriptase variant may have an altered pH optimum or increased temperature stability but may retain its characteristic transcriptase activity.

A “Variant” may have at least about 45%, at least about 50%, at least about 55%, at least about 60%, at least about 65%, at least about 70%, at least about 75%, at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, or at least about 99.5% sequence identity to a polypeptide sequence when optimally aligned for comparison. Percent identity may pertain to the percent identity of the DNA binding domain or the engineered reverse transcriptase portion of an engineered reverse transcriptase. As used herein, a variant residue position is described in relation to the wild-type or precursor amino acid sequence set forth in SEQ ID NO:7; the amino acid position is indexed to SEQ ID NO:7. A fusion variant comprises at least one fusion domain selected from DNA binding domains described elsewhere herein.

As used herein, a protein having a certain percent (e.g., at least 90%, at least 91%, at least 92%, at least 93%, at least 94%, at least 95%, at least 96%, at least 97%, at least 98%, or at least 99%) of sequence identity with another sequence means that, when aligned, that percentage of bases or amino acid residues are the same in comparing the two sequences. This alignment and the percent homology or identity can be determined using any suitable software program known in the art, for example those described in CURRENT PROTOCOLS IN MOLECULAR BIOLOGY, Ausubel et al., eds., 1987, Supplement 30, section 7.7.18. Representative programs include the Vector NTI Advance™ 9.0 (Invitrogen Corp. Carlsbad, CA), GCG Pileup, FASTA (Pearson et al. (1988) Proc. Natl Acad. ScL USA 85:2444-2448), and BLAST (BLAST Manual, Altschul et al., Nat'l Cent. Biotechnol. Inf., Nat'l Lib. Med. (NCIB NLM NIH), Bethesda, Md., and Altschul et al., (1997) Nucleic Acids Res. 25:3389-3402) programs. Another typical alignment program is ALIGN Plus (Scientific and Educational Software, PA), generally using default parameters. Other sequence alignment software programs that find use are the TFASTA Data Searching Program available in the Sequence Software Package Version 6.0 (Genetics Computer Group, University of Wisconsin, Madison, WI and CLC Main Workbench (Qiagen) Version 20.0. The present disclosure is not limited to the software being used to align two or more sequences.

As used herein, the term “Wild-type” or “WT” refers to a gene or gene product that has the characteristics of that gene or gene product when isolated from a naturally occurring source. The amino acid sequence set forth in SEQ ID NO:7 is a WT Murine Moloney Leukemia Virus (MMLV) sequence (Genbank NP_955591.1 p80 RT).

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

### EXAMPLES

It will be understood that the reference to the below examples is for illustration purposes only and do not limit the scope of the claims. Each aspect, embodiment, or feature of the invention may be combined with any other aspect, embodiment, or feature the invention unless clearly indicated to the contrary. Unless defined otherwise, all technical and scientific terms used herein have the meaning commonly understood by a person skilled in the art to which this invention belongs.

Exemplary engineered RTs comprising a MMLV RT variant (42BL) operably linked N-terminally to a DNA binding protein derived from a budding yeast DAT1 were generated. However, engineered RT comprising C-terminal fusions can also be constructed. Dat from different budding yeasts that contains at least three repeated pentads of G-R-K-P-G can be used. An DAT1 N-terminal fusion protein was generated; and an DAT1C-terminal fusion protein was generated. The DAT1 fusion proteins are produced with an N-terminal 6× His Tag and thrombin cleavage site. The 6× His Tag was used for purification purposes and removed by thrombin cleavage.

Exemplary engineered RTs (e.g., variant MMLV) comprising an RT operably linked N-terminally to a truncated DAT1 molecule comprising 90 amino acid (DAT (90) was also generated. While the majority of exemplary engineered RT tested below are of N-terminal fusions, C-terminal RT fusions can also be constructed. In fact, a non-exhaustive list of possible fusion proteins can be generated. These different reverse transcriptases can also be fused to full length DAT protein, DAT1 truncated to 36 amino acids, or DAT1 (90) homologs.

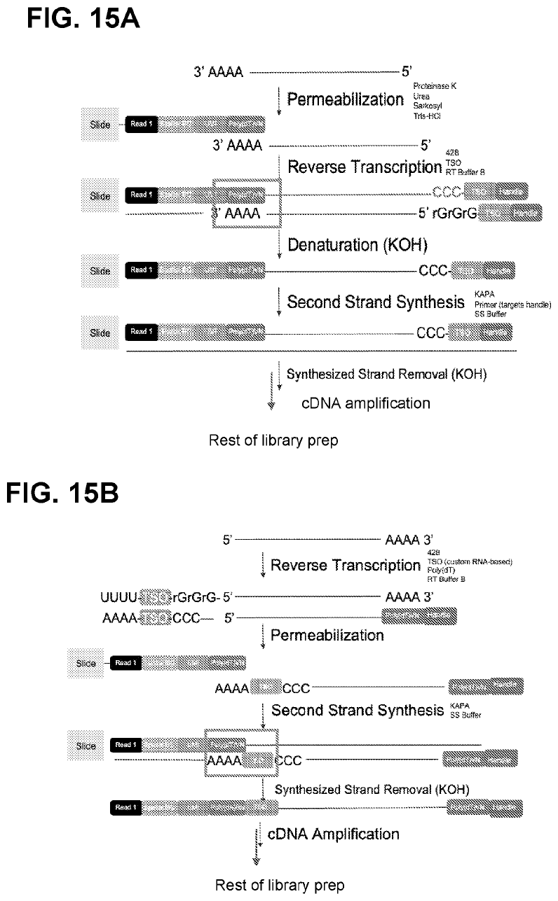

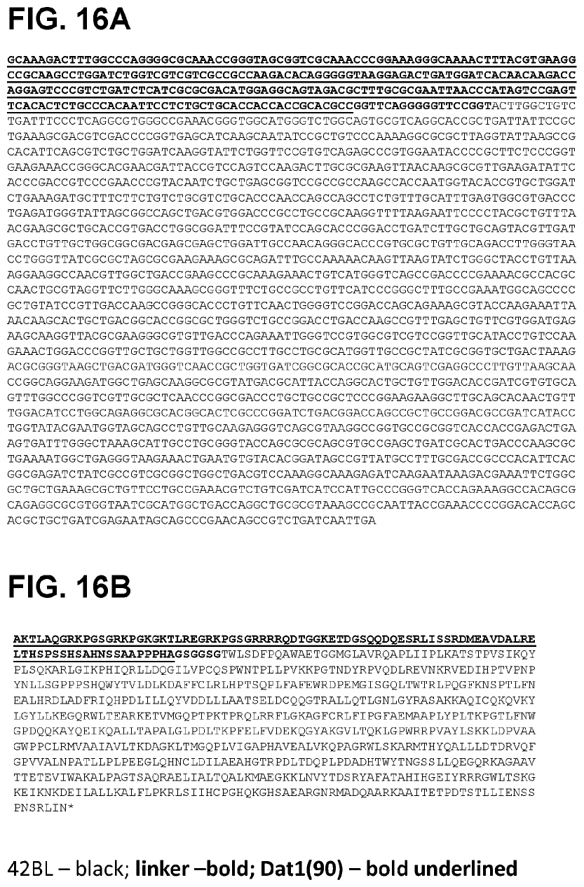

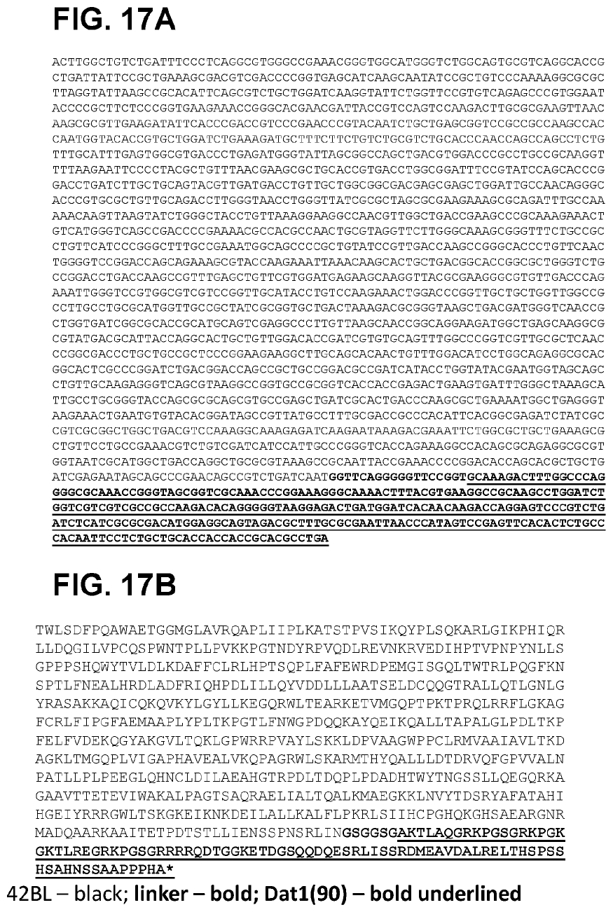

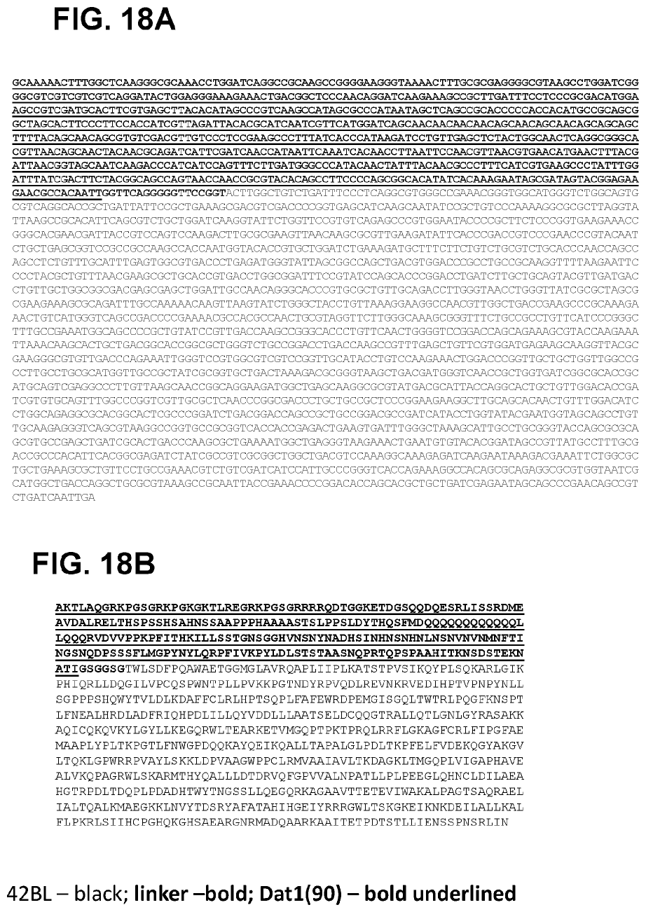

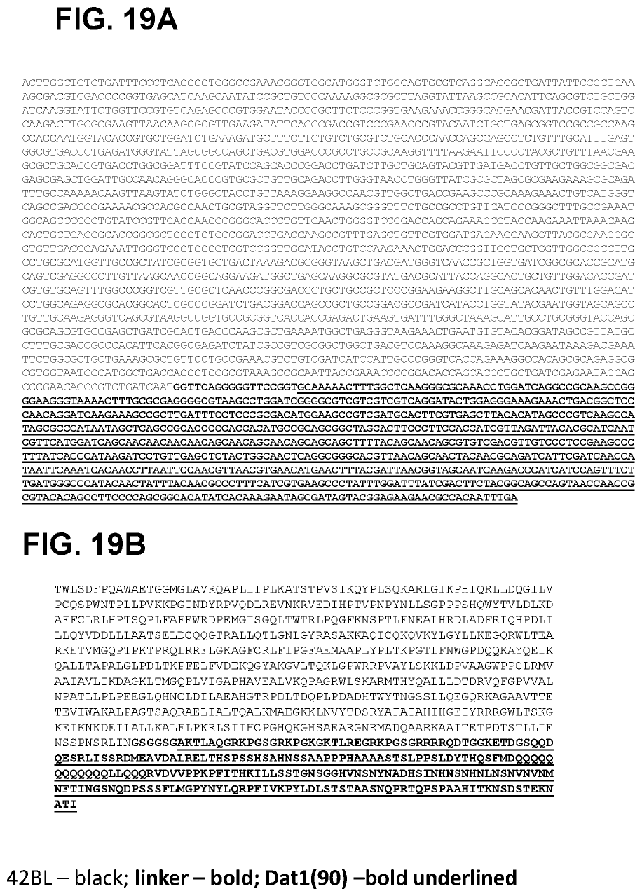

In vitro characterizations of an exemplary engineered RT (N-Dat_42BL) were conducted as shown below. These results showed the enhanced performance of engineered reverse transcriptases comprising DAT1 described herein in a capillary electrophoresis (CE) assay (FIG. 13); a Single Cell 5′ (SC-5′) or a Single Cell 3′ (SC-3′) gene expression assay (FIGS. 14A-B); or Visium ATP reverse transcriptase based assay (FIGS. 15A-B).

It was demonstrated that DAT1 can be truncated to a minimal binding domain of 36 amino acids as shown by N-Dat36_42BL and C-Dat36_42BL. In addition, homologs of DAT1 from other organisms can also be used to generate the engineered reverse transcriptase described herein as shown by N-DAT1-TL-QID040 42BL; N-DAT1-TL-XP361 42BL; N-DAT1-TL-XP558 42BL; or N-DAT1-TL-XP683 42BL.

DAT1 (90) can be fused to other reverse transcriptase enzymes, such as other variants of MMLV reverse transcriptases as shown by N-DAT-90 42B; N-DAT-90 50A+G; N-DAT-90 SOLD 33 VDG; N-DAT-90 SOLD 01; C-DAT-90 SOLD 01.

### Example 1: Capillary Electrophoresis Assay Validation

Reverse transcription and sequencing reactions were prepared. The reaction volume was 50 μl and reactions contained 5′-end labeled FAM Reverse Transcriptase primer 2, RT Reagent B (Chromium Next GEM Single Cell Reagent, 10× Genomics), RNA template (RNA Temp 2), template switching oligo 1 (TSO1), and the indicated engineered reverse transcriptase.

TABLE 3A
Capillary Electrophoresis Assay Reactants

| | | |
|---|---|---|
| Reagent | Stock | Final |

| | | |
|---|---|---|
| RT Reagent B | 2.66x | 1.00X |

| | | | | |
|---|---|---|---|---|
| FAM.RT.Primer2 | 100.00 | uM | 0.50 | uM |
| RNA.Temp2.CE | 84.4 | uM | 0.50 | uM |
| TSO1.Oligo | 91.20 | uM | 5.00 | uM |
| Enzyme | 15.40 | uM | 0.50 | uM |

| | | |
|---|---|---|
| Water | — | — |

TABLE 3B
Capillary Electrophoresis Assay Reactants-GAPDH template

| | | |
|---|---|---|
| Reagent | Stock | Final |

| | | | | |
|---|---|---|---|---|
| GEM-U Reagent | 4.00 | x | 1.00 | x |
| GAPDH or GRch38 Primer | 10.00 | uM | 25 | nM |

TABLE 3A
Capillary Electrophoresis Assay Reactants

| Reagent | Stock | Final |
|---|---|---|
| GAPDH or GRch38 | 5.6 uM | 50 nM |
| TSO1.Oligo | 93.00 uM | 12.00 uM |
| Enzyme | Variable uM | 0.8 uM |
| Water | — | — |

Experimental workflow replicated that of the Chromium Single Cell Gene Expression 5′ kit (10× Genomics, Inc), except the reverse transcriptase was altered for a particular reaction. Stock concentrations and final concentrations in the reactions are shown in Tables 3A-B. Variations of the assay stock concentrations and final concentrations in the reactions shown in Table 4 were used. The reactions included stoichiometrically equal amounts of enzyme and template for single turnover conditions.

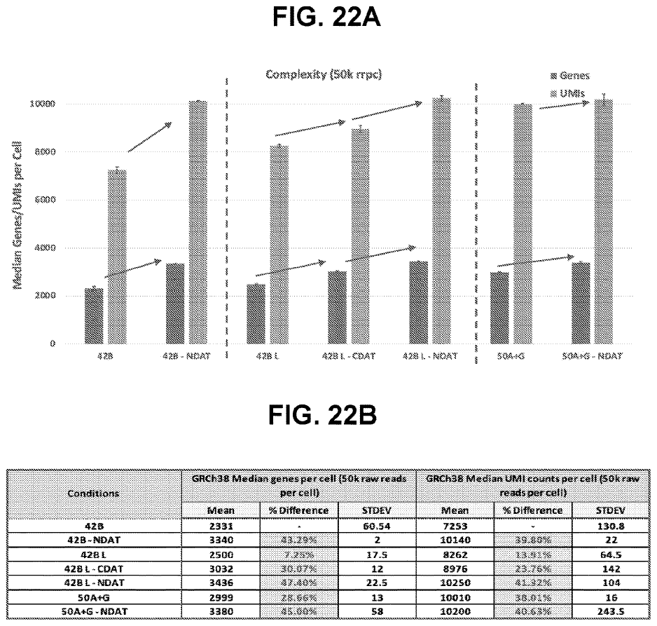

Additionally, reverse transcription and sequencing reactions were prepared using GAPDH or GRCh38 as a template. The reaction volume was 50 μl; reactions contained 5′-end labeled GAPDH or GRCh38 Primer, GEM-U reagent, RNA template (GAPDH or GRCh38 template, template switching oligo 1 (TSO1, and the indicated engineered reverse transcriptase. Stock concentrations and final concentrations in the reactions are shown in Table 3B. The reactions included stoichiometrically equal amounts of enzyme and template for single turnover conditions. Reactants were incubated at 53° C. for 45 minutes, then diluted 1:20 in HiDi formamide. The formamide mixture was heated to 95° C. for 5 mins, then chilled on ice for 2 mins. Samples were loaded on the CE, the DS-33 dye set was selected and long fragment analysis was performed using the GS1200LIZ size standard. The GEM-U reagent approximates the formulation of the actual reagent mixture in a GEM assay when the contents of the Z1 and Z2 channels are mixed. Results from one such experiment are shown in FIGS. 22-26.

Reactants were incubated at 53° C. for one hour, then diluted 1:40 in water and then 1:20 in HiDi formamide. The formamide mixture was heated to 95° C. for 5 mins, then chilled on ice for 2 mins. Samples were loaded on a Seqstudio™ (Thermo Fisher Scientific) and fragment analysis by capillary electrophoresis was carried out with the appropriate dye channels and size standards. The assay was validated with synthetically sized oligonucleotides and with a transcription positive, template switching null engineered reverse transcriptase and a transcription positive, template switching positive reverse transcriptase (Enzyme Mix C,). The GEM-U reagent approximates the formulation of the actual reagent mixture in a GEM assay when the contents of the Z1 and Z2 channels are mixed. Capillary Electrophoresis Assay Reactants are disclosed in Table 1A, Capillary Electrophoresis Assay template, Primer and TSO sequences are shown in Table 4A.

Reverse transcription and sequencing reactions were also prepared using GAPDH or GRCh38 as a template. The reaction volume was 50 μl; reactions contained 5′-end labeled GAPDH or GRCh38 primer, GEM-U reagent, RNA template (GAPDH or GRCh38 template), template switching oligo 1 (TSO1), and the indicated engineered reverse transcriptase. Stock concentrations and final concentrations in the reactions are shown in Table 4B. The reactions included stoichiometrically equal amounts of enzyme and template for single turnover conditions. Reactants were incubated at 53° C. for 45 minutes, then diluted 1:20 in HiDi formamide. The formamide mixture was heated to 95° C. for 5 mins, then chilled on ice for 2 mins. Samples were loaded on the CE, the DS-33 dye set was selected and long fragment analysis was performed using the GS1200LIZ size standard. The GEM-U reagent approximates the formulation of the actual reagent mixture in a GEM assay when the contents of the Z1 and Z2 channels are mixed.

Results from one such experiment are shown in FIGS. 22-26.

In particular, the reaction volume was 50 μl; reactions contained 5′-end labeled GAPDH primer, GEM-U reagent, RNA template (GAPDH template), template switching oligo 1 (TSO1), and the indicated engineered reverse transcriptase(s). The final concentrations in the reactions are shown in Table 4B. The reaction buffer was SOP for SC-5′ and the reaction time was 45 minutes.

Tables 4A-B show Capillary Electrophoresis (CE) Assay Reactants and Template, Primer and TSO sequences (SEQ ID NOS: 173, 175, 176, respectively in order of appearance.)

TABLE 4A
Capillary Electrophoresis Assay Template, Primer and TSO sequences

| Reagent | Stock | | Final | | Volume | | | |
|---|---|---|---|---|---|---|---|---|
| RT Reagent B (2000165) | 4.00 | x | 1.00 | x | 9.54 | ul | 76.34 | ul |
| FAM.RT.Primer2 (Variable) | 100.00 | uM | 0.5000 | uM | 0.250 | ul | 2.000 | ul |
| RNA.Temp2.CE (Variable) | 84.40 | uM | 1.00 | uM | 0.59 | ul | 4.74 | ul |
| TSO1.Oligo (Variable) | 1000.00 | uM | 64.00 | uM | 3.20 | ul | 25.60 | ul |
| DTT | 1000.00 | mM | 20.00 | mM | 1.00 | ul | 8.00 | ul |
| Gel Bead Buffer (2000018) | 1.00 | x | 0.24x | | 11.83 | ul | 94.66 | ul |
| Polyacylamide Solution | 10% | | 0.50% | | 2.50 | ul | 20.00 | ul |
| (2000052) | | | | | | | | |
| Enzyme | 15.40 | uM | 0.50 | uM | 1.62 | ul | 12.99 | ul |
| Water | | | | | 19.46 | ul | 155.68 | ul |

| | | | | | |
|---|---|---|---|---|---|
| Total | — | 50.00 | ul | 400.00 | ul |

TABLE 4B
Capillary Electrophoresis Assay GAPDH
Template, Primer and TSO sequences

| | | |
|---|---|---|
| Reagent | Stock | Final |

| | | | | |
|---|---|---|---|---|
| GEM-U Reagent | 4.00 | x | 1.00 | x |
| GAPDH Primer | 10.00 | uM | 25 | nm |
| GAPDH | 5.00 | uM | 50 | nM |
| TSO1.Oligo | 93.00 | uM | 12.00 | uM |
| Enzyme | 15.40 | uM | 1.31 | uM |

| | | |
|---|---|---|
| Water | — | — |

### Example 2. Construction, Cloning and Expression of Engineered Reverse Transcriptases

Several mutants were constructed using a Q5 mutagenesis kit (NEB) with mutagenic primers per manufacturing instructions. Linearized products were circularized by KLD treatment (kinase, ligase, DpN1) and cloned. Several mutants were synthesized as whole plasmids and furnished by Twist Biosciences, South San Francisco CA.

Briefly, a vector comprising the DAT1 sequence was obtained from Integrated DNA Technologies (IDT, Coralville, IA). Cloning was performed using a Gibson Assembly kit from New England Biolabs (NEB, Ipswitch, ME). Q5 polymerase was used to generate Gibson vectors. Amplification conditions were an initial denaturation at 95° C. for 2.5 minutes, 30 cycles of denature (95° C., 30 sec), a 45 sec gradient annealing and extension at 72° C. for 6 minutes, 35 sec, followed by a final extension at 72° C. for 2 minutes. Amplification reactions with multiple annealing gradient temperatures (65.2° C., 67° C., 68.5° C. and 69.6° C.) were performed.

Amplification products were evaluated on a 1.2% agarose E-Gel using SYBR-Safe. Products were pooled prior to clean-up. Cloning and expression were performed in the Acella cell line from EdgeBio (San Jose, CA). Cells were selected on LB-Kanamycin plates. DAT1 N-terminal and C-terminal fusions to an engineered reverse transcriptase having the amino acid sequence set forth in SEQ ID NO: 1 were obtained by screening of bacterial colonies. The sequences of the fusion proteins were confirmed using method known in the art.

### Example 3: Single Cell Sensitivity and Mapping

Various engineered reverse transcriptases were evaluated in single cell experiments with human peripheral blood monocytes (PBMCs) and mouse peripheral blood monocyte cells (C57B/L6) using 3′ and 5′ reaction conditions as described above herein. Sensitivity and mapping were evaluated. Results from engineered reverse transcriptase (N-Dat 42BL (SEQ ID NO: 175) and C-DAT 42B L; SEQ ID NO: 174) were compared to results obtained from a commercially available engineered MMLV or MMLV variants (SOLD 001 (SEQ ID NO: 65); and SOLD 33 VDG (SEQ ID NO: 173).

Results from one such series of experiments are summarized in FIG. 20. The percent change is as compared to a commercially available variant MMLV reverse transcriptase. The change in median genes and median UMI's queried at 20,000 reads per cell and the change in reads mapped to the transcriptome and reads mapped to exons are shown. A commercially available engineered reverse transcriptase was used as the control. Improvements in both the 5′ and 3′ chemistries were more pronounced in the mouse PBMC's than in the human PBMCs.

FIG. 20 and FIG. 21 show that DAT1 in combination with reverse transcriptase (MMLV, 42B, or other 42B variant thereof (e.g., SOLD 001 or SOLD 033 VDG)), either fused at the N-terminus or C-terminus of the RT improved GEX sensitivity even at low sequencing depth. Improvement was observed with both gene expression, which was increased by up to ~37% and UMI, which was increased by up to 13% as captured at 20 k rrpc. Gains were even more significant at higher read depth. In addition, the engineered RT disclosed herein exhibited large change in differential gene expression in single cell assays. The engineered RT molecules comprising DAT1 or variant thereof disclosed herein picked-up to about 5000 additional genes when compared to a non-DAT1 RT (e.g., 42B). FIGS. 20-26. These engineered RT polypeptide also exhibited increase in median UMI counts per spot and median gene counts per spot in spatial assay. Moreover, the engineered RT molecules gave decrease in fraction of reads mapped to exons with gain in fraction mapped to introns. See e.g., FIG. 21. A performance difference between FPLC and plate purified proteins was performed.

### Example 4: Analysis of Engineered RT Comprising a DAT1 DNA Binding Domain in 5′ Single Cell Assay

This example shows that an engineered reverse transcriptase described herein (e.g., 42B-L-dat fusion) significantly improved the sensitivity of a 5′ single cell assay.

To determine the effectiveness of the engineered RT and/or recombinant RT protein described herein, the RT enzymes were analyzed in 5′ gene expression assay. Analysis of 42B-NDAT, 42B L-CDAT, 42B L-NDAT and 50A+G-NDATin 5′ single cell assay was performed and compared to an assay using 42B, 42B L, or 50A+G RT. All RT enzymes were used at 1.31 uM concentration, and all purified by the same method. RT enzymes tested included 42B (SEQ ID NO: 1, SEQ ID NO: 143, or SEQ ID NO: 172), 50A+G (Table 2; SEQ ID NO: 147), 42B_L (Table 2; SEQ ID NO: 145).

FIGS. 22A-B show the relative differences in performance of the engineered RT polypeptides or control non-DAT RT compared to control RT (42B). The median genes and UMIs/cell at 50 k raw-reads per cell were used to compare the sensitivity of three reverse transcriptases with and without the DAT fusion domain. 42B-NDAT showed 43.29% (Median genes/cell) and 39.80% (median UMIs/cell) enhancement over 42B alone. 42BL-CDAT showed 30.07% (Median genes/cell) and 23.76% (median UMIs/cell) enhancement over 42B alone. 42BL-NDAT showed 47.40% (Median genes/cell) and 41.32% (median UMIs/cell) enhancement over 42B alone. 50A+G-NDAT showed 45.00% (Median genes/cell) and 40.63% (median UMIs/cell) enhancement over 42B alone. In the same assay, non-DAT RT, 42 B L only showed 7.25% (Median genes/cell) and 13.91% (median UMIs/cell) enhancement over 42B alone. The non-DAT RT, 50A+G only showed 28.66% (Median genes/cell) and 38.01% (median UMIs/cell) enhancement over 42B alone.

Furthermore, both a C-terminal fusion and an N-terminal fusion of DAT to 42BL increased median genes per cell and median UMIs per cell in the single cell assays. The C-terminal fusion increased median genes per cell by 21% compared to 42BL (3032 versus 2500) and median UMIs per cell by 9% (8976 versus 8262) as compared to 42BL. The N-terminal fusion increased median genes per cell by 37% (3436 versus 2500) and median UMIs per cell by 24%. Additionally, the 50A+G-DAT fusion showed a 13% increase in genes per cell (3380 versus 2999) and a 2% increase in UMIs per cell (10200 versus 10010). Thus, each DAT RT fusion tested demonstrated improved quality metrics in a transcriptomics assay as compared to a matched control including the same RT but lacking the DAT domain.

These results showed clear performance gains in engineered RT polypeptides comprising a DAT fusion on either the N-terminal or C-terminal domain. The results also demonstrated the enhanced sensitivity of a single cell assay using an engineered reverse transcriptase polypeptide or an engineered recombinant reverse transcriptase comprising DAT1 described herein as shown by the median genes identified per cell (Median genes/cell) or the median UMIs identified per cell (median UMIs/cell). Specifically, an engineered RT of the present disclosure significantly improved the RT sensitivity when compared to a non-DAT1 RT; and an engineered reverse transcriptase comprising a DAT1 binding domain further significantly increased the gain in sensitivity of the engineered reverse transcriptase described herein.

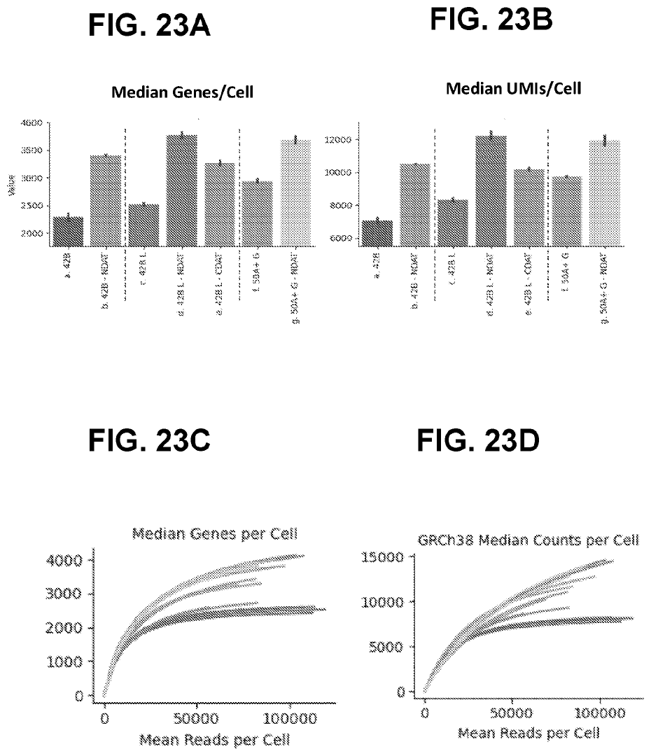

The performance of various engineered RT disclosed herein were also analyzed at maximum normalization depth using the median genes/cell (FIG. 23A) or the median UMIs/Cell (FIG. 23B) to compare the generated library complexity of three reverse transcriptases (42B, 42B L, and 50A+G) with (N-DAT or C-DAT) and without the DAT domain. FIG. 23 C-D show saturation curves of the median genes (FIG. 23C) and counts/cell (FIG. 27C) as a function of read depth, which further demonstrate that the median genes and counts/cell were higher using the engineered RT with a DAT DNA binding domain when compared to MMLV variants lacking the DAT DNA binding domain. FIG. 23 demonstrates a clear benefit of using the DAT DNA binding domain in a Single Cell 5′ (SC-5′) gene expression assay.

To further determine the properties of the engineered RTs described herein, these RT enzymes were analyzed in a 5′ gene expression assay. All RT enzymes were used at 1.31 μM concentration, and all purified by the same method.

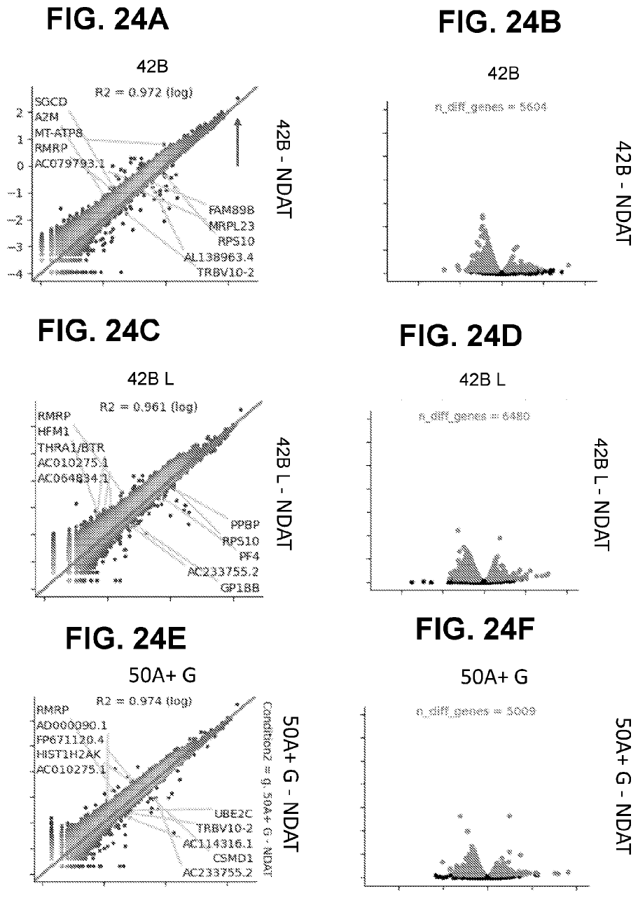

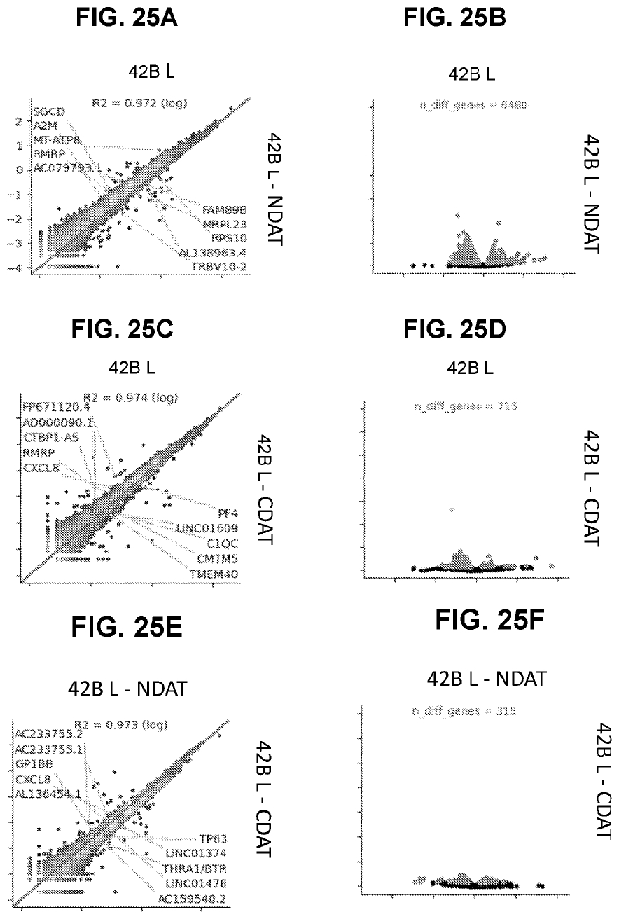

A good correlation in differential gene expression (e.g., gene calling) between engineered RT variants comprising the DAT DNA binding domain) relative to non-DAT control was observed (FIGS. 24-25). FIGS. 24A-F and FIGS. 25A-F showed significant levels of differential gene expression with 42B L-CDAT, 42B L-NDAT, 50A+G-NDAT. FIGS. 24A-F shows the differential gene expression of some engineered RT comprising DAT1 at the N-terminus. FIGS. 24A, C, and E feature scatter plots showing gene expression correlation of three reverse transcriptases with and without the DAT fusion domain. FIGS. 24B, D, and F feature volcano plots showing the number of differentially expressed genes between three reverse transcriptases with and without the DAT fusion domain.

FIGS. 25A-F illustrate the differential gene expression of some engineered RT comprising DAT1 at the C-terminus. FIGS. 25A, C, and E feature scatter plots showing gene expression correlation comparison of engineered RT comprising N-Terminal DAT Domain and C-Terminal DAT Domain. FIGS. 25B, D, and F feature volcano plots comparing the number of differentially expressed genes between various RT. Gains in performance, were still present, but not as significant with a C-terminal fusion. For example, the C-DAT terminal fusion did not perform as well as the N-terminal DAT fusion, but both N-terminal DAT fusion and C-DAT terminal fusion showed performance gains when compared to the RT backbone (42B L; SEQ ID NO: 145) alone. As the conditions become more similar in complexity (42B L-NDAT >42B L-CDAT >42B L, see FIGS. 22-23), there are lower levels of DEGs and the feature scatter plots begin to correlate better. More genes will begin to pass the differential gene expression threshold (expression >=0.3 and p-value <=0.05) as the complexity gap widens, but it is not a 1:1 correlation.

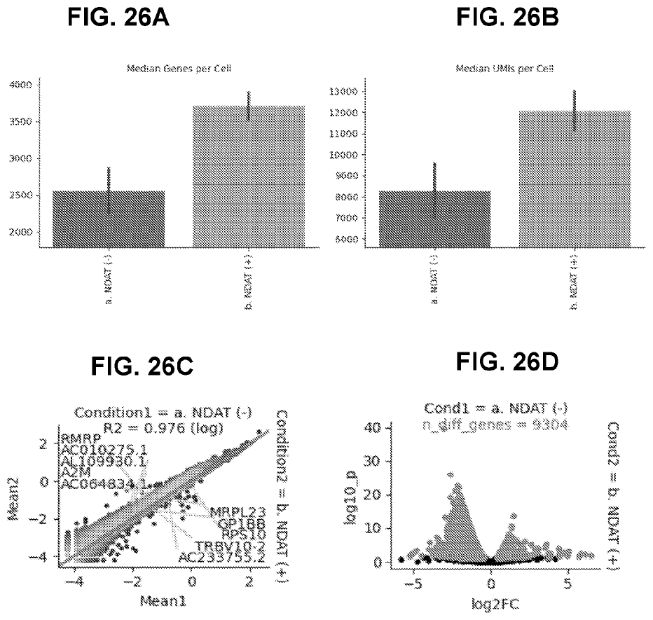

FIGS. 26A-D show graphs illustrating the performance comparison of the impact of the DAT fusion domain across three reverse transcriptase backbones based on median genes (FIG. 26A) and UMIs/cell at maximum normalization depth (FIG. 26B), gene expression correlation (FIG. 26C), and differential gene expression (FIG. 26D). The aggregated metrics comparing the performance among 42B, 42B L, and 50A+G backbones with and without the DAT fusion showed a clear performance benefit from the DAT domain, such as e.g., enhanced sensitivity. Therefore, the 42B L-DAT1 fusion RT significantly improve in single cell assay performance.

### Example 5: Assays for Analyses of Engineered RT Polypeptides

Any of the engineered RT enzymes of the invention, including without limitation any of the enzymes described in Table 1, or Table 2 could be analyzed in any suitable assay, including without limitation the assays described herein. Assays include without limitation 5′ gene expression analyses, with or without VDJ analysis, 3′ gene expression analysis, epigenetic analysis, or multiomic analyses. In non-limiting embodiments, experiments are carried out as found in the manufacturer's instructions for the Chromium Single Cell 5′ Gene Expression Assay kit (10× Genomics); Chromium Single Cell 3′ Gene Expression Assay kit (10× Genomics), including any of multiomic extensions or applications.

### Example 6: Single Cell 3′ and 5′ cDNA Yields

Various engineered reverse transcriptases can be evaluated in single cell experiments with peripheral blood monocytes (PBMCs) at a cell load of 1,000, using the 3′ and 5′ configurations. Emulsion droplets can contain gel beads with either barcoded poly-dT primer sequences (3′ configuration) or barcoded template switch oligo sequences (5′ configuration) that also include a UMI and Illumina® Read 1 sequence. When cells are lysed within the droplet, the poly-dT primer hybridizes to the poly-A tail of the cellular mRNA, which is extended by the reverse transcriptase. Once the end of the template is reached, the reverse transcriptase will exhibit terminal transferase activity to add an overhang of three non-templated deoxycytidines (CCC) to the 3′ end of the synthesized cDNA. The CCC overhang will hybridize to the 3 riboguanosines (rGrGrG) present on the 3′ end of the template switch oligo, allowing the reverse transcriptase to “switch” templates and continue synthesis to the 5′ end of the template switch oligo. Depending on the which configuration of gel bead is used (3′ or 5′) the barcode and UMI will allow either the 3′ or 5′-end of the mRNA molecule to be identified in the final sequencing library. Following reverse transcription at 48° C. or 53° C. for 45 mins, and a 5 min heat-kill at 85° C., droplets were broken and the cDNA was purified with Dynabeads. The cDNA was then amplified via PCR, purified with a 0.6× SPRI, and quantified with an Agilent Bioanalyzer using the DNA High Sensitivity Kit. The cDNA yield (ng) was then obtained.

### Example 7: Single Cell 3′ Quality Metrics

Various engineered reverse transcriptases can be evaluated in single cell experiments with peripheral blood monocytes (PBMCs) using the 3′ and 5′ reaction conditions. Either 10 μL of the amplified cDNA (3′ conditions) or 20 μL containing a maximum of 50 ng of amplified cDNA (5′ conditions) can then be fragmented and A-tailed, cleaned with a double-sided SPRI (0.6×/0.8×), ligated to functional adaptors with an Illumina® Read 2 sequence, cleaned with a 0.8× SPRI, and then can be further amplified with sample indexing primers that include the P5 and P7 priming sites and the i5 and i7 sample indexes. The amplification product can be cleaned up with a double-side (0.6×/0.8×) SPRI, and the average size can be determined with an Agilent Bioanalyzer using the DNA High Sensitivity Kit. The material can then be quantified by qPCR and pooled for next generation sequencing on an Illumina® Novaseq targeting a sequencing depth of at least 50,000 reads per cell and using the following run parameters (Read 1:28 cycles, 17 Index: 10 cycles, i5 Index: 10 cycles, Read 2:90 cycles). Data can be collected, demultiplexed, and processed. Standard quality metrics were obtained.

Generally, the single cell 5′ reactions use less enzyme and TSO oligo than the single cell 3′ reactions. The 5′ TSO oligo is also twice the length of the 3′ TSO oligo with varied sequence context due to the presence of the UMI and the barcode. The single cell 5′ reaction conditions are generally considered a more stringent test of performance than the 3′ single cell reaction conditions.

It is expected that the results from these experiments will show that the engineered reverse transcriptase polypeptide disclosed herein will show improved sensitivity at 20 or 50 kilo reads per cell (krpc).

Most of the engineered RT polypeptides will yield metrics within parity for valid UMI's, valid barcodes, ribosomal UMI's, mitochondrial UMI's, transcript coverage, and reads with any poly A sequence, reads with any switch oligo sequence and reads with primer or homopolymer sequence.

FIG. 21 summarizes results from a series of experiments using the 5′ reaction conditions. The figure summarizes metrics of the 5′ single cell experiments, including 20 k read metrics, 50K read metrics and reads mapped to the transcriptome. The engineered reverse transcriptase variants have the amino acid sequences provided set forth in SEQ ID NO: 65 (SOLD 001), SEQ ID NO: 173 (SOLD 33 VDG), SEQ ID NO: 174 (C-DAT 42BL), and SEQ ID NO: 175 (N-DAT 42BL). The percent indicates the percent change from the results obtained with an engineered reverse transcriptase having the amino acid sequence set forth in SEQ ID NO: 1 or 179 (42B). In particular, the engineered reverse transcriptase polypeptides showed a significant improvement in sensitivity.

FIG. 21 shows additional metrics related to results obtained from the indicated engineered reverse transcriptase in single cell 5′ experiments. Most of the variants yielded metrics within parity for valid UMI's, valid barcodes, ribosomal UMI's, mitochondrial UMI's, transcript coverage, reads with any poly A sequence, reads with any switch oligo sequence and reads with primer or homopolymer sequence.

### Example 8: Immunoprofiling and TCR Improvements

Immune profiling is an extension of the 5′ chemistry to profile genes specifically for T-cell and/or B-cell receptors in the mRNA pool. Methods of immune profiling are known in the art and generally include additional rounds of PCR on the cDNA with a pool of sequence specific primers to allow for targeted enrichment of T-cell and/or B-cell receptor genes. Immune profiling assays may also detect UMIs for B-cell receptor genes, namely IGH, IGK, and IGL (Immunoglobulin heavy chain (IGH), kappa (IGK), and light (IGL) chain). Immune profiling data is informative for immunology research and is an extension of standard gene expression evaluation. Methods of immune profiling include but are not limited to Chromium Next Gen Single Cell™ kits (10× Genomics, Pleasanton CA).

Amplified cDNA (2 μl) from the 5′ configuration of reverse transcription reactions can be subjected to two additional rounds of PCR enrichment with TCR immune profiling, which included a double-sided (0.5×/0.8×) SPRI clean-up between the first and second round of thermal cycling reactions. The amplified products can then be cleaned-up with a subsequent double-sided (0.5×/0.8×) SPRI, fragmented and A-tailed, ligated to functional adaptors with an Illumina® Read 2 sequence, cleaned up with a 0.8× SPRI, and then further amplified with sample indexing primers that include the P5 and P7 priming sites and the i5 and i7 sample indexes. The amplification product can be cleaned up with a 0.8× SPRI, and average size can be determined with an Agilent Bioanalyzer using the DNA High Sensitivity Kit. The material can then be quantified by qPCR and can be pooled for next generation sequencing on an Illumina® Novaseq targeting a sequencing depth of at least 5,000 reads per cell and using the following run parameters (Read 1:28 cycles, i7 Index: 10 cycles, i5 Index: 10 cycles, Read 2:90 cycles). Data can be collected, demultiplexed, and single-cell V(D)J analysis can be performed. Results that are obtained from engineered reverse transcriptases can be compared to results are obtained from a commercially available enzyme or an RT lacking the DAT 1 DNA binding domain. The percent change in median TRA UMI's and median TRB UMI's from mouse and human PBMCs for each RT tested can be shown as a percent change in median IGH, IGK and IGL from mouse PBMC's. It is expected that the median TRA UMIs and median TRB UMIs obtained with any engineered reverse transcriptase described herein h will be greater than those obtained with a commercially available reverse transcriptase or non-DAT RT in both human PBMCs and mouse PBMCs. Engineered reverse transcriptases will also exhibit IG sensitivity that is either comparable or improved IG sensitivity as compared to previous results.

### Example 9: Spatial Assays

This example shows various methods for performing spatial analysis using the engineered RT enzymes of the present disclosure. The Example provides an exemplary method for detecting an individual gene expression or globally-expressed RNA transcripts in a fresh or frozen tissue using an engineered RT of SEQ ID NO: 175, which includes N-Terminal fusion of the first 90a.a. of Dat 1 (“N-DAT”) fused to a 42B variant of SEQ ID NO: 1, 143, 145, or 172) compared to a control RT variant (having the same RT enzyme but lacking the N-DAT or SEQ ID NO: _1, 143, 145, or 172) using three different reverse transcription buffer formulations, the commercially available RT reagent, buffer X.4 and buffer X.5. The commercially available RT reagent comprises 2% Glycerol, 50 mM Tris pH 8.3, 3 mM MgCl₂, 75.04 mM KCl, 0.5% Supersonic F-108, 0.489 mg/ml BSA, and 3.16 mM dNTPs. Buffer X.4 and buffer X.5 have the same components as the commercial RT reagent with the following differences. Buffer X.4 contains 1.05% Glycerol, 158.3 mM NaCl, 0.579 mg/ml BSA, 4.685 mM dNTPs, 1.5 mM dCTP, and 0.775 mM GTP. Buffer X.5 contains 133.8 mM NaCl, 1% Supersonic F-108, 3.96 mM dNTPs, 1.31 mM dCTP, and 0.655 mM GTP.

Sample preparation. A formalin-fixed, paraffin-embedded (FFPE) human tonsil sample, a frozen Human tonsil sample, or a fresh Human tonsil sample can be used for the method described herein. Human tonsil FFPE sections on standard slides (for sandwich conditions; FIGS. 1A-B, FIGS. 2A-B, and FIGS. 3A-C) or gene expression (GEx) slides (for non-sandwich control conditions) were deparaffinized, H&E stained, and imaged. After the imaging, the human tonsil sections were hematoxylin-destained with HCL solution. The sections were then decrosslinked by incubating at 70° C. for 1 hour in decrosslinking solution. Decrosslinking solution was removed and the tissues were incubation in 1× PBS-Tween for 15 minutes.

Spatial analysis of the prepared fresh human tonsil sample or the human tonsil FFPE sections can be analyzed using the 3′ workflow shown in FIG. 27.

Sample permeabilisation. The human tonsil sample or the human tonsil FFPE sections were incubated with a permeabilization solution at 37° C. for 30 minutes. The permeabilization solution was washed out and the human tonsil sample was prepared for analyte capture by adding 0.1×SSC buffer and subjected to a pre-equilibration thermocycling protocol (e.g., lid temperature and pre-equilibrate at 53° C., reverse transcription at 53° C. for 45 minutes, and then hold at 4° C.). The SSC buffer was removed.

cDNA generation. A Master Mix comprising a commercially available RT reagent buffer, buffer X.4, or buffer X.5, nuclease-free water, a template switch oligo, a reducing agent, and a reverse transcriptase (control 42B RT, N-DAT-42B RT, or small scale-purified N-DAT-42B RT) was added to the human tonsil sample and subjected to a thermocycling protocol, which comprised performing a reverse transcription reaction at 53° C. for 45 minutes and bold at 4° C. A second strand synthesis was performed on the sample by subjecting the sample to a thermocycling protocol, which comprised e.g., pre-equilibrating at 65° C., synthesizing the second strand at 65° C. for 15 minutes, then holding at 4° C. The Master Mix reagents were removed from the sample and 0.8M KOH was added and incubated for 5 minutes at room temperature. The KOH was removed and followed by the addition of an elution buffer.

A Second Strand Mix, including a second strand reagent, a second strand primer, and a second strand-polymerizing enzyme, can be added to the sample and the sample can be sealed and incubated. At the end of the incubation, the reagents can be removed and elution buffer can be added and removed from the sample, and 0.8 M KOH can be added again to the sample and the sample can be incubated for 10 minutes at room temperature. Tris-HCl can be added and the reagents can be mixed. The sample can be transferred to a new tube, vortexed, and placed on ice.

cDNA amplification and quality control. A. qPCR Mix, including nuclease-free water, qPCR Master Mix, and cDNA primers, was prepared and pipetted into wells in a qPCR plate. A small amount of the human tonsil sample was added to the plated qPCR Mix, and thermocycled according to a predetermined thermocycling protocol (e.g., step 1: 98° C. for 3 minutes, step 2: 98° C. for 5 seconds, step 3: 63° C. for 30 seconds, step 4: record amplification signal, step 5: repeating 98° C. for 5 seconds, 63° C. for 30 seconds for a total of 25 cycles). After completing the thermocycling, a cDNA amplification mix, including amplification mix and cDNA primers was prepared and combined with the remaining sample and mixed. The sample was incubated and thermocycled (e.g., lid temperature at 105° C. for-45-60 minutes, step 1: 98° C. for 3 minutes, step 2: 98° C. for 15 seconds, step 3: 63° C. for 20 seconds, step 4: 72° C. for one minute, step 5: [the number of cycles determined by qPCR Cq Values], step 6: 72° C. for 1 minute, and step 7: hold at 4° C.). The sample can then be stored at 4° C. for up to 72 hours or at −20° C. for up to 1 week or use immediately.

SPRI Cleanup. The cDNA sample was resuspended in 0.6× SPRI select Reagent and pipetted to ensure proper mixing. The sample was incubated for 5 minutes at room temperature and cleared by placing the sample on a magnet (e.g., the magnet is in the high position). The supernatant was removed and 80% ethanol was added to the pellet and incubated for 30 seconds. The ethanol was removed and the pellet washed. The sample was centrifuged and placed on a magnet (e.g., the magnet is on the low position). Any remaining ethanol was removed and the sample was air dried for up to 2 minutes. The magnet was removed and elution buffer was added to the sample, mixed, and incubated for 2 minutes at room temperature. The sample was then placed on the magnet (e.g., on low position) until the solution cleared. The sample was transferred to a new tube strip and stored at 4° C. for up to 72 hours or at −20° C. for up to 4 weeks.

A portion of the sample was run on an Agilent Bioanalyzer High Sensitivity chip, where a region was selected and the cDNA concentration was measured to calculate the total cDNA yield. Alternatively, the quantification can be determined by Agilent Bioanalyzer or Agilent TapeStation.

Fragmentation, End-repair, and A-tailing. Following SPRI Cleanup, the sample was processed for spatial gene expression library construction. A Fragmentation Mix, including a fragmentation buffer and a fragmentation enzyme was prepared on ice. Elution buffer and fragmentation mix was added to each sample, mixed, and centrifuged. The sample mix was then placed in a thermocycler and cycled according to a predetermined protocol (e.g., lid temperature at 65° C. for ~35 minutes, pre-cool block down to 4° C. before fragmentation at 32° C. for 5 minutes, End-repair and A-tailing at 65° C. for 30 minutes and holding at 4° C.).

SPRI Cleanup. The 0.6× SPRI select Reagent was added to the sample and incubated for 5 minutes at room temperature. The sample was placed on a magnet (e.g., in the high position) until the solution cleared, and the supernatant was transferred to a new tube strip. 0.8× SPRI select Reagent was added to the sample, mixed, and incubated for 5 minutes at room temperature. The sample was placed on a magnet (e.g., in the high position) until the solution cleared. The supernatant was removed and 80% ethanol was added to the pellet, incubated for 30 seconds, and washed. The ethanol wash was repeated and the sample placed on a magnet (e.g., in the low position) until the solution cleared. The remaining ethanol was removed and elution buffer was added to the sample, mixed, and incubated for 2 minutes at room temperature. The sample was placed on a magnet (e.g., in the high position) until the solution cleared, and a portion of the sample was moved to a new tube strip.

Ligation. An Adaptor Ligation Mix, including ligation buffer, DNA ligase, and adaptor oligos, was prepared and centrifuged. The Adaptor Ligation Mix was added to the sample, pipette-mixed, and centrifuged briefly. The sample was then thermocycled using a predetermined protocol (e.g., lid temperature at 30° C. for ~15 minutes, step 1: 20° C. for 15 minutes, step 2: 4° C. hold). The sample was vortexed to resuspend SPRI select Reagent for another SPRI cleanup as described above.

Sample Index PCR. An amplification mix was prepared and added to the sample. An individual Dual Index TT Set A was added to the sample, pipette-mixed and subjected to a predetermined thermocycling protocol (e.g., lid temperature at 105° C. for-25-40 minutes, step 1: 98° C. for 45 seconds, step 2: 98° C. for 20 seconds, step 3: 54° C. for 30 seconds; step 4: 72° C. for 20 seconds, step 5: reverting to step 2 for a predetermined number of cycles, step 6: 72° C. for 1 minute, and 4° C. on hold). Vortex to resuspend the SPRI select Reagent for cleaning.

The cleaned sample was stored at 4° C. for up to 72 hours, or at −20° C. for long-term storage. The average fragment size was determined using a Bioanalyzer trace or an Agilent TapeStation. The library was sequenced using a sequencing platform, for example, MiSeq, NextSeq 500/550, HiSeq 2500, HiSeq 3000/4000, NovaSeq, and iSeq. See, Illumina®, Indexed Sequencing Overview Guides, February 2018, Document 15057455v04; and Illumina® Adapter Sequences, May 2019, Document #1000000002694vl 1, each of which is hereby incorporated by reference, for information on P5, P7, i7, 15, TruSeq™ Read 2, indexed sequencing, and other reagents described herein.

### Example 10: N-DAT RT Variant Improved the Sensitivity of the Spatial Assay while Maintaining Good Spatial Resolution in Human Samples

To determine the improvement associated with the engineered N-DAT-42B RT variant, the effect of the 42B RT control variant (SEQ ID NO: 1, 143, 145, or 172) and the N-DAT-42B RT variant were compared using the three buffer types as previously described. The human tonsil samples were permeabilized using a Kryptonite permeabilization buffer comprising 6% Sarkosyl, 1M urea, and 4 ug/ul proteinase K. The reverse transcriptase reaction was performed on human tonsil samples were prepared as disclosed above using six different conditions: (1) the 42B RT control variant was used with RT reagent; (2) the 42B RT control variant was used with Buffer X.5; (3) the engineered N-DAT-42B RT variant was used with the RT reagent; (4) the engineered N-DAT-42B RT variant was used with buffer X.4; (5) the engineered N-DAT-42B RT variant was used with buffer X.5; and (6) the small-scale purified engineered N-DAT-42B RT variant was used with buffer X.5. Most of the engineered N-DAT-42B RT variants tested were purified by FPLC. However, some N-DAT-42B RT variants underwent a small scale purification. RT was performed using a 2 step protocol: 45 min at 53° C. and 30 min at 42° C.

As shown in FIGS. 28A-F, the engineered N-DAT-42B RT variant maintained a good spatial resolution using all three buffers tested. FIGS. 28A-F show UMI heat maps showing globally detected gene expression in a human tonsil tissue analyzed using N-DAT-42B RT variant (FIGS. 28C-F) or 42B RT variant (FIGS. 28A-B) and the three different buffer formulations. The NDAT1 RT variant was either FPLC-purified or underwent a small scale purification (FIG. 28F). The global detection of gene expression is shown as a heat map. The RT Reagent (FIG. 28A and FIG. 28C) showed better mapping metrics when compared to buffer X.4 (FIG. 28D), and buffer X.5 (FIG. 28B, FIG. 28E, and FIG. 28F). The commercially available RT reagent comprised 2% Glycerol, 50 mM Tris pH 8.3, 3 mM MgCl₂, 75.04 mM KCl, 0.5% Supersonic F-108, 0.489 mg/ml BSA, and 3.16 mM dNTPs. Buffer X.4 and buffer X.5 have the same components as the commercial RT reagent with the following differences. Buffer X.4 contains 1.05% Glycerol, 158.3 mM NaCl, 0.579 mg/ml BSA, 4.685 mM dNTPs, 1.5 mM dCTP, and 0.775 mM GTP. Buffer X.5 contains 133.8 mM NaCl, 1% Supersonic F-108, 3.96 mM dNTPs, 1.31 mM dCTP, and 0.655 mM GTP. A MMLV RT variant without a DAT fusion domain was used as a control (e.g., a RT variant comprising the amino acid sequence of SEQ ID NO: 1, 143, 145, or 172). The NDAT1 RT variant was either FPLC-purified or underwent a small-scale purification (FIG. 28F). In particular, FIGS. 28A-F show that NDAT1 RT variant improved the sensitivity of the assay while maintaining good spatial resolution when compared to the control RT variant, with all three buffer conditions.

FIGS. 29A-F show that the engineered N-DAT-42B RT also increased the quality and the sensitivity metrics of the spatial assays. FIGS. 29A-F show graphs comparing the quality, sensitivity, and detection of gene expression under the same six conditions shown in FIGS. 28A-F. In particular, fraction reads in spots under tissue (FIG. 29A) was substantially the same among the 6 conditions tested. However, the smallest standard deviation across the six conditions tested was obtained using RT reagent and with the engineered N-DAT-42B RT. Reads mapped confidently to transcriptome (FIG. 29B) were significantly increased in the samples containing the N-DAT-42B using the RT reagent, as compared to using buffers X.4 and X.5, and compared to all three buffer conditions using the RT control variant. The total genes detected (FIG. 29C) appeared the same in all conditions tested, with the exception of the RT control variant using the RT reagent (RTR), which showed significantly reduced total genes detected. Analysis of median UMI counts per spot (30 k mapped spot-reads per spot, mapped to GRch38 reference genome assembly) (FIG. 29D) showed that median UMI counts per spot were significantly enhanced in all three conditions using an N-DAT RT variant when compared to the three conditions using the RT control variant. Unexpectedly, N-DAT RT in buffer X.4 (N-DAT_RTX4) showed less variability. In addition, the GRch38 median genes per spot (30 k mapped spot-reads per spot) (FIG. 29E) analysis showed that median genes per spot were significantly enhanced in all samples comprising an N-DAT RT variant when compared to the 42B RT samples regardless of the buffer used. As such, engineered NDAT1 RT variant improved the sensitivity of the spatial assay while maintaining good spatial resolution when compared to the 42B RT variant (FIGS. 29D-E). The RT Reagent appeared to show better mapping metrics when compared to Buffer X.4, and Buffer X.5. RT Reagent B with N-DAT at various concentrations and timings can also be tested and will likely show similar results as the RT Reagent.

Assessment of individual gene expression also showed substantially similar results as globally detected gene expression. FIGS. 30A-F show UMI heat maps shown as a log 10 (UMI) ranging from cold to hot (0, 0.5, 1.0, 1.5, and 2.0), which illustrate the gene expression of RGS3 in a human tonsil tissue analyzed using N-DAT RT variant (FIGS. 30C-F) or 42BRT control variant (FIGS. 30A-B) under the three different buffer formulations (RT reagent (FIG. 30A and FIG. 30C), Buffer X.4 (FIG. 30D), and Buffer X.5 (FIG. 30B, FIG. 30E, and FIG. 30F)).

FIGS. 31A-C show UMI heat maps shown as a log 10 (UMI) ranging from cold to hot (0, 0.5, 1.0, 1.5, 2.0, and 2.5) illustrating the gene expression of KRT5 in a human tonsil tissue analyzed using N-DAT RT variant (FIGS. 31C-F) or RT control variant (FIGS. 31A-B) using three different buffer formulations (a commercially available RT reagent (FIG. 31A and FIG. 31C), Buffer X.4 (FIG. 31D), and Buffer X.5 (FIG. 31B, FIG. 31E, and FIG. 31F)). Under sandwich configuration conditions used in the assay shown in FIGS. 30 and 31, some evidence of undesirable flow of transcripts and/or target molecules or analytes was observed. A black circle on upper right quadrant identifies images with blurry tail.

### Example 11: Spatial Resolution in Non-Human/Mouse Tissue

To further determine the effectiveness of the engineered N-Dat RT variant (e.g., 42BL-NDAT), a spatial assay was performed using a zebrafish sample using Visium standard definition (SD) spatial gene expression assay (e.g., with a first slide configuration) or Visium high definition (HD) spatial gene expression assay. Visium HD slides contain two 6.5×6.5 mm Capture Areas with a continuous lawn of oligonucleotides arrayed in ~11 million 2×2 μm barcoded squares without gaps, achieving single cell-scale spatial resolution. The data are output at 2 μm, as well as multiple bin sizes. The 8×8 μm bin is the recommended starting point for visualization and analysis. See e.g., 10×genomics.com/platforms/visium; 10×genomics.com/products/visium-hd-spatial-gene-expression.

The zebrafish samples were permeabilized using a Kryptonite permeabilization buffer comprising 6% Sarkosyl, 1M urea, and 4 ug/ul proteinase K. The reverse transcriptase reaction was performed on zebrafish samples using T reagent, the engineered N-DAT-42B RT (NDAT-42BL) variant, TSO, and dTT. The RT was performed using a 2 step protocol-45 min at 53° C. and 30 min at 42° C. The single strand synthesis was performed using a control RT (e.g., MMLV TR variant comprising an amino acid sequence of SEQ ID NO: 1, 143, 145, or 172), RT Reagent, and SS Primers. Under current experimental conditions, N-DAT fusion did not sufficiently process the synthesis of the second strand cDNA in the Zebrafish tissue. This was likely caused by the N-DAT fusion configuration or the RT variant configuration. For example, the configuration may have slowed the enzyme from initiating or continuing the synthesis of the second strand. However, the N-DAT fusion sufficiently process the synthesis of the first strand cDNA in the Zebrafish tissue.

FIGS. 32A-B show UMI heat maps shown as a count log 10 ranging from cold to hot (0.5, 1, 1.5, 2, 2.5, 3, 4, 4.5, and 5), illustrating globally detected gene expression in Zebrafish (non-human or mouse tissue) using a spatial assay with a first slide configuration. FIGS. 32C-D show heat maps illustrating individual gene expression. FIG. 32C shows Crgm1 gene expression as log normalized per experiment ranging from cold to hot (0.00-5.0). FIG. 32D shows KRT5 gene expression as log normalized per experiment ranging from cold to hot (0.0-7.0). FIG. 32E shows Rho gene expression as log normalized per experiment ranging from cold to hot (0.0-8.0). The quality, and sensitivity metrics were: fraction reads in spots under tissue (0.9), fraction reads usable (0.4), Reads mapped confidently to genome (0.56), and number of reads (250M). Fraction reads mapped to genome are the fraction of reads that mapped to a unique gene in the genome. In general, the read must be consistent with annotated splice junctions and are considered for UMI counting.

FIGS. 33A-D also show UMI heat maps showing globally detected gene expression (FIG. 33A) or individual gene expression (FIGS. 33B-C) in Zebrafish (non-human or mouse tissue) analyzed using NDAT1 RT variant (42BL-N-DAT1) and RT reagent buffer under sandwich configuration conditions for a spatial assay with a second slide configuration. FIG. 33B shows Crgm1 gene expression as log normalized per experiment ranging from cold to hot (0.00-4.0). FIG. 33C shows KRT5 gene expression as log normalized per experiment ranging from cold to hot (0.0-9.0). FIG. 33D shows Rho gene expression as log normalized per experiment ranging from cold to hot (0.0-7.0). The quality, and sensitivity metrics were: fraction reads mapped to genome (0.72).

The engineered N-DAT RT variants disclosed herein enhanced the quality and the sensitivity of the spatial assay metrics while maintaining good spatial resolution when compared to a control RT variant. The control RT variant lacked the N-DAT but comprised the same RT backbone as the engineered N-DAT RT variant. The control RT variant consisted of the amino acid sequence of SEQ ID NO: 1, 142, 143, or 172.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

### INCORPORATION BY REFERENCE

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Table 1 shows listing of non-limiting embodiments of RT enzymes of the present disclosure.

Tables 2 and 5 shows additional listing of amino acid and nucleic acid sequences of non-limiting embodiments of the engineered RTs of the present disclosure.

TABLE 1
Non-limiting embodiments of RT variants of the invention

| SEQ ID NO | Description | Sequences |
|---|---|---|
| 1 | Engineered | MTWLSDFPQAWAETGGMGLAVRQAPLIIPLKATSTPVS |
| | reverse | IKQYPMSQKARLGIKPHIQRLLDQGILVPCQSPWNTPLL |
| | transcriptase | PVKKPGTNDYRPVQDLREVNKRVEDIHPTVPNPYNLLS |
| | (MMLV RT | GPPPSHQWYTVLDLKDAFFCLRLHPTSQPLFAFEWRDP |
| | Variant) | EMGISGQLTWTRLPQGFKNSPTLFNEALHRDLADFRIQ |
| | | HPDLILLQYVDDLLLAATSELDCQQGTRALLQTLGNLG |
| | | YRASAKKAQICQKQVKYLGYLLKEGQRWLTEARKETV |
| | | MGQPTPKTPRQLRRFLGKAGFCRLFIPGFAEMAAPLYP |
| | | LTKPGTLFNWGPDQQKAYQEIKQALLTAPALGLPDLTK |
| | | PFELFVDEKQGYAKGVLTQKLGPWRRPVAYLSKKLDP |
| | | VAAGWPPCLRMVAAIAVLTKDAGKLTMGQPLVIGAPH |
| | | AVEALVKQPAGRWLSKARMTHYQALLLDTDRVQFGP |
| | | VVALNPATLLPLPEEGLQHNCLDILAEAHGTRPDLTDQP |
| | | LPDADHTWYTNGSSLLQEGQRKAGAAVTTETEVIWAK |
| | | ALPAGTSAQRAELIALTQALKMAEGKKLNVYTDSRYA |
| | | FATAHIHGEIYRRRGWLTSKGKEIKNKDEILALLKALFL |
| | | PKRLSIIHCPGHQKGHSAEARGNRMADQAARKAAITET |
| | | PDTSTLLIENSSPNSRLIN |
| 2 | Dat 1 | MAKTLAQGRK PGSGRKPGKG KTLREGRKPG |
| | Amino acid | SGRRRRQDTG GKETDGSQQD QESRLISSRD |
| | NCBI Reference | MEAVDALREL THSPSSHSAH NSSAAPPPHA |
| | Sequence: | AAASTSLPPS LDYTHQSFMD QQQQQQQQQQ |
| | NP_013594.1 | QQLLQQQRVD VVPPKPFITH KILLSSTGNS |
| | | GGHVNSNYNA DHSINHNSNH NLNSNVNVNM |
| | | NFTINGSNQD PSSSFLMGPY NYLQRPFIVK |
| | | PYLDLSTSTA ASNQPRTQPS PAAHITKNSD STEKNATI |
| 3 | Dat1 D-90 | MAKTLAQGRK PGSGRKPGKG KTLREGRKPG |
| | | SGRRRRQDTG GKETDGSQQD |
| | | QESRLISSRDMEAVDALREL THSPSSHSAH |
| | | NSSAAPPPHA |
| 4 | a poly-dT | TTTTTTTTTT |
| | sequence | |
| 5 | Dat1 D-60 | MAKTLAQGRK PGSGRKPGKG KTLREGRKPG |
| | | SGRRRRQDTG GKETDGSQQD QESRLISSRD |
| 6 | Dat1 D-48 | MAKTLAQGRK PGSGRKPGKG KTLREGRKPG |
| | | SGRRRRQDTG GKETDGSQ |
| 7 | Wild-type MMLV | TLNIEDEHRLHETSKEPDVSLGSTWLSDFPQAWAETGG |
| | GenBank Seq ID | MGLAVRQAPLIIPLKATSTPVSIKQYPMSQEARLGIKPHI |
| | NP_955591.1 p80 | QRLLDQGILVPCQSPWNTPLLPVKKPGTNDYRPVQDLR |
| | RT | EVNKRVEDIHPTVPNPYNLLSGLPPSHQWYTVLDLKDA |
| | | FFCLRLHPTSQPLFAFEWRDPEMGISGQLTWTRLPQGFK |
| | | NSPTLFDEALHRDLADFRIQHPDLILLQYVDDLLLAATSE |
| | | LDCQQGTRALLQTLGNLGYRASAKKAQICQKQVKYLG |
| | | YLLKEGQRWLTEARKETVMGQPTPKTPRQLREFLGTAG |
| | | FCRLWIPGFAEMAAPLYPLTKTGTLFNWGPDQQKAYQE |
| | | IKQALLTAPALGLPDLTKPFELFVDEKQGYAKGVLTQKL |
| | | GPWRRPVAYLSKKLDPVAAGWPPCLRMVAAIAVLTKD |
| | | AGKLTMGQPLVILAPHAVEALVKQPPDRWLSNARMTH |
| | | YQALLLDTDRVQFGPVVALNPATLLPLPEEGLQHNCLDI |
| | | LAEAHGTRPDLTDQPLPDADHTWYTDGSSLLQEGQRKA |
| | | GAAVTTETEVIWAKALPAGTSAQRAELIALTQALKMAE |
| | | GKKLNVYTDSRYAFATAHIHGEIYRRRGLLTSEGKEIKN |
| | | KDEILALLKALFLPKRLSIIHCPGHQKGHSAEARGNRMA |
| | | DQAARKAAITETPDTSTLL |
| 8 | Dat1 D-36 | MAKTLAQGRK PGSGRKPGKG KTLREGRKPG |
| | | SGRRRR |
| 9 | Dat1 D-35 | AKTLAQGRK PGSGRKPGKG KTLREGRKPG SGRRRR |
| 10 | TSO sequence | AAGCAGTGGT ATCAACGCAG AGTACATrGrGrG |
| 11 | Dat 1 | GRKPG |
| | AT-rich | |
| | interaction | |
| | domain-minimal | |
| 12 | Dat 1 | GRKPGSGR |
| | AT-rich | |
| | interaction domain | |
| 13 | Dat1 AT-rich | xxTLAQGRK PGxGRKPxKG KTLxEGRKPG SGRRRR |
| | interaction | |
| | domain- | |
| | consensus | |
| 14 | Dat1 AT-rich | xxTLAQGRX xGxGRxxxKG KTLxEGRxxG SGRRRR |
| | interaction | |
| | domain- | |
| | consensus | |
| 15 | mammalian high | Lys-Arg-Pro-Arg-Gly-Arg-Pro-Lys-Lys (KRPRGRPKK) |
| | mobility group I | |
| | protein (HMG-I, | |
| | or a-protein) | |
| | AT-rich | |
| | interaction domain | |
| 16 | Drosophila | Lys/ Arg-Lys-Arg-Gly-Arg-Pro-Lys-Lys (K/RKRGRPKK) |
| | melanogaster DI | |
| | AT-rich | |
| | interaction | |
| | domain- | |
| | consensus | |
| 17 | Drosophila | Arg-Lys-Arg-Gly-Arg-Pro-Lys-Lys (RKRGRPKK) |
| | melanogaster DI | |
| | AT-rich | |
| | interaction domain | |
| 18 | Drosophila | Lys-Lys-Arg-Gly-Arg-Pro-Lys-Lys (KKRGRPKK) |
| | melanogaster DI | |
| | AT-rich | |
| | interaction domain | |
| 19 | AT-rich | Arg-Lys-Arg-Gly-Arg* (RKRGR*) |
| | interaction domain | |
| 20 | AT-rich | Pro-Lys-Arg-Pro (PKRP) |
| | interaction domain | |
| 21 | phage 434 | Lys-Arg-Pro-Arg* (KRPR*) |
| | repressor | |
| | AT-rich | |
| | interaction domain | |
| 22 | Hin recombinase | Gly-Arg*-Arg/Pro-Pro-Lys (GR*R/PPK) |
| | of Salmonella | |
| | typhimurium | |
| | AT-rich | |
| | interaction domain | |
| 23 | Hin recombinase | Gly-Arg*-Arg-Pro-Lys (GR*RPK) |
| | of Salmonella | |
| | typhimurium | |
| | AT-rich | |
| | interaction domain | |
| 24 | Hin recombinase | Gly-Arg*-Pro-Pro-Lys (GR*PPK) |
| | of Salmonella | |
| | typhimurium | |
| | AT-rich | |
| | interaction domain | |
| 25 | Dat1 | atggcgaaaactttggcacaaggaaggaaacctggaagcggcagaaagcccggaaaa |
| | Nucleic acid | gggaagacgttgagagagggaagaaagcctggcagtggtaggaggaggaggcaaga |
| | sequence | tactgggggtaaagagaccgacgggtctcagcaagatcaggagtcgcgtcttattagttc |
| | NC 001145.3: | cagggacatggaagctgtggacgcactgagagagttgacgcacagcccgtcgtctcatc |
| | 44045-44791 | agctcataattcatcagcagcaccaccgccgcatgcagcagcggcgtcaacttcattgcc |
| | Saccharomyces | tccatctctggattatacccatcagtcattcatggatcaacaacaacaacagcagcagcag |
| | cerevisiae S288C | cagcagcaacaactactacagcaacagagagtggacgtcgttccgcccaagccgttcat |
| | chromosome XIII | cacccacaagatcctgttatcttcaacgggaaactctggcggccacgtgaattcaaattac |
| | | aacgccgaccacagcattaatcataattcaaaccacaaccttaacagcaacgtgaacgtg |
| | | aacatgaactttactataaatgggagcaatcaagacccgtcatcttctttcctgatggggcc |
| | | ctacaactacttacagcggcctttcattgtgaaaccatatcttgatctgtccacaagcacag |
| | | cagcatcaaatcagccacggacgcaaccatcgccggcagcacatatcactaagaattcc |
| | | gattctacagagaaaaacgcaaccatatga |
| 26 | Linker | GGGGS (one non-limiting embodiment) |
| 27 | MMLV variant | MTWLSDFPQAWAETGGMGLAVRQAPLIIPLKATSTPVSI |
| | | KQYPLSQEARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLREFLGTAGFCRLWIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIKAPHAVEALVK |
| | | QPPDRWLSNARMTHYQALLLDTDRVQFGPVVALNPAT |
| | | LLPLPEEGLQHNCLDILAEAVGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIYCPGH |
| | | QKGHSAEARGNRMADQAARKAAITETPDTSTLLIENSSP |
| | | NSRLIN |
| 28 | MMLV RT | MTWLSDFPQAWAETGGMGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPPDRWLSKARMTHYQALLLDTDRVQFGPVVALNPAT |
| | | LLPLPEEGLQHNCLDILAEAVGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIYCPGH |
| | | QKGHSAEARGNRMADQAARKAAITETPDTSTLL |
| 29 | MMLV RT | MTWLSDFPQAWAETGGVGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIKAPHAVEALVK |
| | | QPPDRWLSKARMTHYQALLLDTDRVQFGPVVALNPAT |
| | | LLPLPEEGLQHNCLDILAEAVGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIYCPGH |
| | | QKGHSAEARGNRMADQAARKAAITETPDTSTLL |
| 30 | MMLV RT | MTWLSDFPQAWAETGGVGLAVRQAPLIIPLKATSTPVSI |
| | Variant | KQYPMSQKARLGIKPHIQRLLDQGILVPCQSPWNTPLLP |
| | | VKKPGTNDYRPVQDLREVNKRVEDIHPTVPNPYNLLSG |
| | | PPPSHQWYTVLDLKDAFFCLRLHPTSQPLFAFEWRDPE |
| | | MGISGQLTWTRLPQGFKNSPTLFNEALHRDLADFRIQHP |
| | | DLILLQYVDDLLLAATSELDCQQGTRALLQTLGNLGYR |
| | | ASAKKAQICQKQVKYLGYLLKEGQRWLTEARKETVMG |
| | | QPTPKTPRQLRRFLGKAGFCRLFIPGFAEMAAPLYPLTKP |
| | | GTLFNWGPDQQKAYQEIKQALLTAPALGLPDLTKPFELF |
| | | VDEKQGYAKGVLTQKLGPWRRPVAYLSKKLDPVAAG |
| | | WPPCLRMVAAIAVLTKDAGKLTMGQPLVIKAPHAVEAL |
| | | VKQPPDRWLSKARMTHYQALLLDTDRVQFGPVVALNP |
| | | ATLLPLPEEGLQHNCLDILAEAVGTRPDLTDQPLPDADH |
| | | TWYTNGSSLLQEGQRKAGAAVTTETEVIWAKALPAGTS |
| | | AQRAELIALTQALKMAEGKKLNVYTDSRYAFATAHIHG |
| | | EIYRRRGWLTSKGKEIKNKDEILALLKALFLPKRLSIIYCP |
| | | GHQKGHSAEARGNRMADQAARKAAITETPDTSTLL |
| 31 | MMLV RT | MTWLSDFPQAWAETGGMGLAVRQAPLIIPLKATSTPVSI |
| | Variant | KQYPMSQKARLGIKPHIQRLLDQGILVPCQSPWNTPLLP |
| | | VKKPGTNDYRPVQDLREVNKRVEDIHPTVPNPYNLLSG |
| | | PPPSHQWYTVLDLKDAFFCLRLHPTSQPLFAFEWRDPE |
| | | MGISGQLTWTRLPQGFKNSPTLFNEALHRDLADFRIQHP |
| | | DLILLQYVDDLLLAATSELDCQQGTRALLQTLGNLGYR |
| | | ASAKKAQICQKQVKYLGYLLKEGQRWLTEARKETVMG |
| | | QPTPKTPRQLRRFLGKAGFCRLFIPGFAEMAAPLYPLTKP |
| | | GTLFNWGPDQQKAYQEIKQALLTAPALGLPDLTKPFELF |
| | | VDEKQGYAKGVLTQKLGPWRRPVAYLSKKLDPVAAG |
| | | WPPCLRMVAAIAVLTKDAGKLTMGQPLVIKAPHAVEAL |
| | | VKQPPDRWLSKARMTHYQALLLDTDRVQFGPVVALNP |
| | | ATLLPLPEEGLQHNCLDILAEAVGTRPDLTDQPLPDADH |
| | | TWYTNGSSLLQEGQRKAGAAVTTETEVIWAKALPAGTS |
| | | AQRAELIALTQALKMAEGKKLNVYTDSRYAFATAHIHG |
| | | EIYRRRGWLTSKGKEIKNKDEILALLKALFLPKRLSIIYCP |
| | | GHQKGHSAEARGNRMADQAARKAAITETPDTSTLL |
| 32 | MMLV RT | MTWLSDFPQAWAETGGMGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPAGRWLSKARMTHYQALLLDTDRVQFGPVVALNPAT |
| | | LLPLPEEGLQHNCLDILAEAVGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIYCPGH |
| | | QKGHSAEARGNRMADQAARKAAITETPDTSTLL |
| 33 | MMLV RT | MTWLSDFPQAWAETGGVGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPAGRWLSKARMTHYQALLLDTDRVQFGPVVALNPAT |
| | | LLPLPEEGLQHNCLDILAEAVGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIYCPGH |
| | | QKGHSAEARGNRMADQAARKAAITETPDTSTLL |
| 34 | MMLV RT | TLNIEDEHRLHETSKEPDVSLGSTWLSDFPQAWAETGGV |
| | Variant | GLAVRQAPLIIPLKATSTPVSIKQYPMSQKARLGIKPHIQ |
| | | RLLDQGILVPCQSPWNTPLLPVKKPGTNDYRPVQDLRE |
| | | VNKRVEDIHPTVPNPYNLLSGPPPSHQWYTVLDLKDAFF |
| | | CLRLHPTSQPLFAFEWRDPEMGISGQLTWTRLPQGFKNS |
| | | PTLFNEALHRDLADFRIQHPDLILLQYVDDLLLAATSEL |
| | | DCQQGTRALLQTLGNLGYRASAKKAQICQKQVKYLGY |
| | | LLKEGQRWLTEARKETVMGQPTPKTPRQLRRFLGKAGF |
| | | CRLFIPGFAEMAAPLYPLTKPGTLFNWGPDQQKAYQEIK |
| | | QALLTAPALGLPDLTKPFELFVDEKQGYAKGVLTQKLG |
| | | PWRRPVAYLSKKLDPVAAGWPPCLRMVAAIAVLTKDA |
| | | GKLTMSQPLVIKAPHAVEALVKQPAGRWLSKARMTHY |
| | | QALLLDTDRVQFGPVVALNPATLLPLPEEGLQHNCLDIL |
| | | AEAHGTRPDLTDQPLPDADHTWYTNGSSLLQEGQRKA |
| | | GAAVTDETEVIWAKALPAGTSAQRAELIALTQALKMAE |
| | | GKKLNVYTNSRYAFATAHIHGEIYRRRGWLTSGGKEIK |
| | | NKDEILALLKALFLSKRLSIIHCPGHQKGHSVEARGNRM |
| | | AHQAARRAAITETPDTSTLP |
| 35 | MMLV RT | TLNIEDEHRLHETSKEPDVSLGSTWLSDFPQAWAETGGV |
| | Variant | GLAVRQAPLIIPLKATSTPVSIKQYPMSQKARLGIKPHIQ |
| | | RLLDQGILVPCQSPWNTPLLPVKKPGTNDYRPVQDLRE |
| | | VNKRVEDIHPTVPNPYNLLSGPPPSHQWYTVLDLKDAFF |
| | | CLRLHPTSQPLFAFEWRDPEMGISGQLTWTRLPQGFKNS |
| | | PTLFNEALHRDLADFRIQHPDLILLQYVDDLLLAATSEL |
| | | DCQQGTRALLQTLGNLGYRASAKKAQICQKQVKYLGY |
| | | LLKEGQRWLTEARKETVMGQPTPKTPRQLRRFLGKAGF |
| | | CRLFIPGFAEMAAPLYPLTKPGTLFNWGPDQQKAYQEIK |
| | | QALLTAPALGLPDLTKPFELFVDEKQGYAKGVLTQKLG |
| | | PWRRPVAYLSKKLDPVAAGWPPCLRMVAAIAVLTKDA |
| | | GKLTMSQPLVIKAPHAVEALVKQPAERWLSKARMTHY |
| | | QALLLDTDRVQFGPVVALNPATLLPLPEEGLQHNCLDIL |
| | | AEAHGTRPDLTDQPLPDADHTWYTNGSSLLQEGQRKA |
| | | GAAVTDETEVIWAKALPAGTSAQRAELIALTQALKMAE |
| | | GKKLNVYTDSRYAFATAHIHGEIYRRRGWLTSGGKEIK |
| | | NKDEILALLKALFLPKRLSIIHCPGHQKGHSVEARGNHM |
| | | AHQAARRAAITETPDTSTLP |
| 36 | MMLV RT | TLNIEDEHRLHETSKEPDVSLGSTWLSDFPQAWAETGGV |
| | Variant | GLAVRQAPLIIPLKATSTPVSIKQYPMSQKARLGIKPHIQ |
| | | RLLDQGILVPCQSPWNTPLLPVKKPGTNDYRPVQDLRE |
| | | VNKRVEDIHPTVPNPYNLLSGPPPSHQWYTVLDLKDAFF |
| | | CLRLHPTSQPLFAFEWRDPEMGISGQLTWTRLPQGFKNS |
| | | PTLFNEALHRDLADFRIQHPDLILLQYVDDLLLAATSEL |
| | | DCQQGTRALLQTLGNLGYRASAKKAQICQKQVKYLGY |
| | | LLKEGQRWLTEARKETVMGQPTPKTPRQLRRFLGKAGF |
| | | CRLFIPGFAEMAAPLYPLTKPGTLFNWGPDQQKAYQEIK |
| | | QALLTAPALGLPDLTKPFELFVDEKQGYAKGVLTQKLG |
| | | PWRRPVAYLSKKLDPVAAGWPPCLRMVAAIAVLTKDA |
| | | GKLTMSQPLVIKAPHAVEALVKQPAGRWLSKARMTHY |
| | | QALLLDTDRVQFGPVVALNPATLLPLPEEGLQHNCLDIL |
| | | AEAHGTRPDLTDQPLPDADHTWYTDGSSLLQEGQRKA |
| | | GAAVTTETGVIWAKALPAGTSAQRAELIALTQALKMAE |
| | | GKKLNVYTNSRYAFATAHIQGEIYRRRGFLTSKGKEIKN |
| | | KDEILALLKALFLPKRLSIIHCPGHQKGHSAEARGNRMA |
| | | DQAARKAAITETPDTSTLP |
| 37 | MMLV RT | TLNIEDEHRLHETSKEPDVSLGSTWLSDFPQAWAETGGV |
| | Variant | GLAVRQAPLIIPLKATSTPVSIKQYPMSQKARLGIKPHIQ |
| | | RLLDQGILVPCQSPWNTPLLPVKKPGTNDYRPVQDLRE |
| | | VNKRVEDIHPTVPNPYNLLSGPPPSHQWYTVLDLKDAFF |
| | | CLRLHPTSQPLFAFEWRDPEMGISGQLTWTRLPQGFKNS |
| | | PTLFNEALHRDLADFRIQHPDLILLQYVDDLLLAATSEL |
| | | DCQQGTRALLQTLGNLGYRASAKKAQICQKQVKYLGY |
| | | LLKEGQRWLTEARKETVMGQPTPKTPRQLRRFLGKAGF |
| | | CRLFIPGFAEMAAPLYPLTKPGTLFNWGPDQQKAYQEIK |
| | | QALLTAPALGLPDLTKPFELFVDEKQGYAKGVLTQKLG |
| | | PWRRPVAYLSKKLDPVAAGWPPCLRMVAAIAVLTKDA |
| | | GKLTMSQPLVIKAPHAVEALVKQPAERWLSKARMTHY |
| | | QALLLDTDRVQFGPVVALNPATLLPLPEEGLQHNCLDIL |
| | | AEAHGTRPDLTDQPLPDADHTWYTNGSSLLQEGQRKA |
| | | GAAVTDETEVIWAKALPAGTSAQRAELIALTQALKMAE |
| | | GKKLNVYTDSRYAFATAHIHGEIYRRRGWLTSGGKEIK |
| | | NKDEILALLKALFLPKRLSIIHCPGHQKGHSVEARGNRM |
| | | AHQAARRAAITETPDTSTLP |
| 38 | MMLV RT | TLNIEDEHRLHETSKEPDVSLGSTWLSDFPQAWAETGGV |
| | Variant | GLAVRQAPLIIPLKATSTPVSIKQYPMSQKARLGIKPHIQ |
| | | RLLDQGILVPCQSPWNTPLLPVKKPGTNDYRPVQDLRE |
| | | VNKRVEDIHPTVPNPYNLLSGPPPSHQWYTVLDLKDAFF |
| | | CLRLHPTSQPLFAFEWRDPEMGISGQLTWTRLPQGFKNS |
| | | PTLFEEALRRDLADFRIQHPDLILLQYVDDLLLAATSELD |
| | | CQQGTRALLQTLGNLGYRASAKKAQICQKQVKYLGYL |
| | | LKEGQRWLTEARKETVMGQPTPKTPRQLRRFLGKAGFC |
| | | RLFIPGFAEMAAPLYPLTKPGTLFNWGPDQQKAYQEIKQ |
| | | ALLTAPALGLPDLTKPFELFVDEKQGYAKGVLTQKLGP |
| | | WRRPVAYLSKKLDPVAAGWPPCLRMVAAIAVLTKDAG |
| | | KLTMSQPLVIKAPHAVEALVKQPAGRWLSKARMTHYQ |
| | | ALLLDTDRVQFGPVVALNPATLLPLPEEGLQHNCLDILA |
| | | EAHGTRPDLTDQPLPDADHTWYTNGSSLLQEGQRKAG |
| | | AAVTDETEVIWAKALPAGTSAQRAELIALTQALKMAEG |
| | | KKLNVYTNSRYAFATAHIHGEIYRRRGWLTSGGKEIKN |
| | | KDEILALLKALFLSKRLSIIHCPGHQKGHSVEARGNRMA |
| | | HQAARRAAITETPDTSTLP |
| 39 | MMLV RT | TLNIEDEHRLHETSKEPDVSLGSTWLSDFPQAWAETGGV |
| | Variant | GLAVRQAPLIIPLKATSTPVSIKQYPMSQKARLGIKPHIQ |
| | | RLLDQGILVPCQSPWNTPLLPVKKPGTNDYRPVQDLRE |
| | | VNKRVEDIHPTVPNPYNLLSGPPPSHQWYTVLDLKDAFF |
| | | CLRLHPTSQPLFAFEWRDPEMGISGQLTWTRLPQGFKNS |
| | | PTLFEEALRRDLADFRIQHPDLILLQYVDDLLLAATSELD |
| | | CQQGTRALLQTLGNLGYRASAKKAQICQKQVKYLGYL |
| | | LKEGQRWLTEARKETVMGQPTPKTPRQLRRFLGKAGFC |
| | | RLFIPGFAEMAAPLYPLTKPGTLFNWGPDQQKAYQEIKQ |
| | | ALLTAPALGLPDLTKPFELFVDEKQGYAKGVLTQKLGP |
| | | WRRPVAYLSKKLDPVAAGWPPCLRMVAAIAVLTKDAG |
| | | KLTMSQPLVIKAPHAVEALVKQPAGRWLSKARMTHYQ |
| | | ALLLDTDRVQFGPVVALNPATLLPLPEEGLOHNCLDILA |
| | | EAHGTRPDLTDQPLPDADHTWYTDGSSLLQEGORKAG |
| | | AAVTTETGVIWAKALPAGTSAQRAELIALTQALKMAEG |
| | | KKLNVYTNSRYAFATAHIQGEIYRRRGFLTSKGKEIKNK |
| | | DEILALLKALFLPKRLSIIHCPGHQKGHSAEARGNRMAD |
| | | QAARKAAITETPDTSTLP |
| 40 | MMLV RT | TLNIEDEHRLHETSKEPDVSLGSTWLSDFPQAWAETGGV |
| | Variant | GLAVRQALLIIPLKATSTPVSIKQYPMSQKARLGIKPHIQ |
| | | RLLDQGILVPCQSPWNTPLLPVKKPGTNDYRPVQDLRE |
| | | VNKRVEDIHPTVPNPYNLLSGPPPSHQWYTVLDLKDAFF |
| | | CLRLHPTSQPLFAFEWRDPEMGISGQLTWTRLPQGFKNS |
| | | PTLFNEALHRDLADFRIQHPDLILLQYVDDLLLAATSEL |
| | | DCQQGTRALLQTLGNLGYRASAKKAQICQKQVKYLGY |
| | | LLKEGQRWLTEARKETVMGQPTPKTPRQLRRFLGKAGF |
| | | CRLFIPGFAEMAAPLYPLTKPGTLFNWGPDQQKAYQEIK |
| | | QALLTAPALGLPDLTKPFELFVDEKQGYAKGVLTQKLG |
| | | PWRRPVAYLSKKLDPVAAGWPPCLRMVAAIAVLTKDA |
| | | GKLTMSQPLVIKAPHAVEALVKQPAGRWLSKARMTHY |
| | | QALLLDTDRVQFGPVVALNPATLLPLPEEGLQHNCLDIL |
| | | AEAHGTRPDLTDQPLPDADHTWYTNGSSLLQEGQRKA |
| | | GAAVTDETEVIWAKALPAGTSAQRAELIALTQALKMAE |
| | | GKKLNVYTNSRYAFATAHIHGEIYRRRGWLTSGGKEIK |
| | | NKDEILALLKALFLSKRLSIIHCPGHQKGHSVEARGNRM |
| | | AHQAARRAAITETPDTSTLP |
| 41 | MMLV RT | MTWLSDFPQAWAETGGMGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPAGRWLSKARMTHYQALLLDTDRVQFGPVVALNPAT |
| | | LLPLPEEGLQHNCLDILAEAHGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIHCPGH |
| | | QKGHSAEARGNRMADQAARKAAITETPDTSTLL |
| 42 | MMLV RT | MTWLSDFPQAWAETGGVGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPAGRWLSKARMTHYQALLLDTDRVQFGPVVALNPAT |
| | | LLPLPEEGLQHNCLDILAEAHGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIHCPGH |
| | | QKGHSAEARGNRMADQAARKAAITETPDTSTLL |
| 43 | MMLV RT | MTWLSDFPQAWAETGGMGLAVRQAPLIIPLKATSTPVSI |
| | Variant | KQYPMSQKARLGIKPHIQRLLDQGILVPCQSPWNTPLLP |
| | | VKKPGTNDYRPVQDLREVNKRVEDIHPTVPNPYNLLSG |
| | | PPPSHQWYTVLDLKDAFFCLRLHPTSQPLFAFEWRDPE |
| | | MGISGQLTWTRLPQGFKNSPTLFNEALHRDLADFRIQHP |
| | | DLILLQYVDDLLLAATSELDCQQGTRALLQTLGNLGYR |
| | | ASAKKAQICQKQVKYLGYLLKEGQRWLTEARKETVMG |
| | | QPTPKTPRQLRRFLGKAGFCRLFIPGFAEMAAPLYPLTKP |
| | | GTLFNWGPDQQKAYQEIKQALLTAPALGLPDLTKPFELF |
| | | VDEKQGYAKGVLTQKLGPWRRPVAYLSKKLDPVAAG |
| | | WPPCLRMVAAIAVLTKDAGKLTMGQPLVIGAPHAVEAL |
| | | VKQPPDRWLSKARMTHYQALLLDTDRVQFGPVVALNP |
| | | ATLLPLPEEGLOHNCLDILAEAVGTRPDLTDQPLPDADH |
| | | TWYTNGSSLLQEGQRKAGAAVTTETEVIWAKALPAGTS |
| | | AQRAELIALTQALKMAEGKKLNVYTDSRYAFATAHIHG |
| | | EIYRRRGWLTSKGKEIKNKDEILALLKALFLPKRLSIIYCP |
| | | GHQKGHSAEARGNRMADQAARKAAITETPDTSTLL |
| 44 | MMLV RT | MTWLSDFPQAWAETGGMGLAVRQAPLIIPLKATSTPVSI |
| | Variant | KQYPMSQKARLGIKPHIQRLLDQGILVPCQSPWNTPLLP |
| | | VKKPGTNDYRPVQDLREVNKRVEDIHPTVPNPYNLLSG |
| | | PPPSHQWYTVLDLKDAYFCLRLHPTSQPLFAFEWRDPE |
| | | MGISGQLTWTRLPQGFKNSPTLFNEALHRDLADFRIQHP |
| | | DLILLQYVDDLLLAATSELDCQQGTRALLQTLGNLGYR |
| | | ASAKKAQICQKQVKYLGYLLKEGQRWLTEARKETVMG |
| | | QPTPKTPRQLRRFLGKAGFCRLFIPGFAEMAAPLYPLTKP |
| | | GTLFNWGPDQQKAYQEIKQALLTAPALGLPDLTKPFELF |
| | | VDEKQGYAKGVLTQKLGPWRRPVAYLSKKLDPVAAG |
| | | WPPCLRMVAAIAVLTKDAGKLTMGQPLVIGAPHAVEAL |
| | | VKQPAGRWLSKARMTHYQALLLDTDRVQFGPVVALNP |
| | | ATLLPLPEEGLOHNCLDILAEAHGTRPDLTDQPLPDADH |
| | | TWYTNGSSLLQEGQRKAGAAVTTETEVIWAKALPAGTS |
| | | AQRAELIALTQALKMAEGKKLNVYTDSRYAFATAHIHG |
| | | EIYRRRGWLTSKGKEIKNKDEILALLKALFLPKRLSIIHCP |
| | | GHQKGHSAEARGNRMADQAARKAAITETPDTSTLL |
| 45 | MMLV RT | MTWLSDFPQAWAETGGVGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPPDRWLSKARMTHYQALLLDTDRVQFGPVVALNPAT |
| | | LLPLPEEGLQHNCLDILAEAVGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIYCPGH |
| | | QKGHSAEARGNRMADQAARKAAITETPDTSTLL |
| 46 | MMLV RT | MTWLSDFPQAWAETGGMGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPADRWLSKARMTHYQALLLDTDRVQFGPVVALNPAT |
| | | LLPLPEEGLQHNCLDILAEAHGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIHCPGH |
| | | QKGHSAEARGNRMADQAARKAAITETPDTSTLL |
| 47 | MMLV RT | MTWLSDFPQAWAETGGMGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPPGRWLSKARMTHYQALLLDTDRVQFGPVVALNPAT |
| | | LLPLPEEGLQHNCLDILAEAHGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIHCPGH |
| | | QKGHSAEARGNRMADQAARKAAITETPDTSTLL |
| 48 | MMLV RT | MTWLSDFPQAWAETGGMGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPPDRWLSKARMTHYQALLLDTDRVQFGPVVALNPAT |
| | | LLPLPEEGLQHNCLDILAEAVGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIHCPGH |
| | | QKGHSAEARGNRMADQAARKAAITETPDTSTLL |
| 49 | MMLV RT | MTWLSDFPQAWAETGGMGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPPDRWLSKARMTHYQALLLDTDRVQFGPVVALNPAT |
| | | LLPLPEEGLQHNCLDILAEAHGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIYCPGH |
| | | QKGHSAEARGNRMADQAARKAAITETPDTSTLL |
| 50 | MMLV RT | MTWLSDFPQAWAETGGMGLAVRQAPLIIPLKATSTPVSI |
| | Variant | KQYPMSQKARLGIKPHIQRLLDQGILVPCQSPWNTPLLP |
| | | VKKPGTNDYRPVQDLREVNKRVEDIHPTVPNPYNLLSG |
| | | PPPSHQWYTVLDLKDAFFCLRLHPTSQPLFAFEWRDPE |
| | | MGISGQLTWTRLPQGFKNSPTLFNEALHRDLADFRIQHP |
| | | DLILLQYVDDLLLAATSELDCQQGTRALLQTLGNLGYR |
| | | ASAKKAQICQKQVKYLGYLLKEGQRWLTEARKETVMG |
| | | QPTPKTPRQLRRFLGKAGFCRLFIPGFAEMAAPLYPLTKP |
| | | GTLFNWGPDQQKAYQEIKQALLTAPALGLPDLTKPFELF |
| | | VDEKQGYAKGVLTQKLGPWRRPVAYLSKKLDPVAAG |
| | | WPPCLRMVAAIAVLTKDAGKLTMGQPLVIGAPHAVEAL |
| | | VKQPPDRWLSKARMTHYQALLLDTDRVQFGPVVALNP |
| | | ATLLPLPEEGLQHNCLDILAEAHGTRPDLTDQPLPDADH |
| | | TWYTNGSSLLQEGQRKAGAAVTTETEVIWAKALPAGTS |
| | | AQRAELIALTQALKMAEGKKLNVYTDSRYAFATAHIHG |
| | | EIYRRRGWLTSKGKEIKNKDEILALLKALFLPKRLSIIHCP |
| | | GHQKGHSAEARGNRMADQAARKAAITETPDTSTLL |
| 51 | MMLV RT | MTWLSDFPQAWAETGGMGLAVRQAPLIIPLKATSTPVSI |
| | Variant | KQYPMSQKARLGIKPHIQRLLDQGILVPCQSPWNTPLLP |
| | | VKKPGTNDYRPVQDLREVNKRVEDIHPTVPNPYNLLSG |
| | | PPPSHQWYTVLDLKDAYFCLRLHPTSQPLFAFEWRDPE |
| | | MGISGQLTWTRLPQGFKNSPTLFNEALHRDLADFRIQHP |
| | | DLILLQYVDDLLLAATSELDCQQGTRALLQTLGNLGYR |
| | | ASAKKAQICQKQVKYLGYLLKEGQRWLTEARKETVMG |
| | | QPTPKTPRQLRRFLGKAGFCRLFIPGFAEMAAPLYPLTKP |
| | | GTLFNWGPDQQKAYQEIKQALLTAPALGLPDLTKPFELF |
| | | VDEKQGYAKGVLTQKLGPWRRPVAYLSKKLDPVAAG |
| | | WPPCLRMVAAIAVLTKDAGKLTMGQPLVIGAPHAVEAL |
| | | VKQPPDRWLSKARMTHYQALLLDTDRVQFGPVVALNP |
| | | ATLLPLPEEGLQHNCLDILAEAHGTRPDLTDQPLPDADH |
| | | TWYTNGSSLLQEGQRKAGAAVTTETEVIWAKALPAGTS |
| | | AQRAELIALTQALKMAEGKKLNVYTDSRYAFATAHIHG |
| | | EIYRRRGWLTSKGKEIKNKDEILALLKALFLPKRLSIIHCP |
| | | GHQKGHSAEARGNRMADQAARKAAITETPDTSTLL |
| 52 | MMLV RT | MTWLSDFPQAWAETGGMGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPPDRWLSKARMTHYQALLLDTDRVQFGPVVALNPAT |
| | | LLPLPEEGLQHNCLDILAEAHGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIHCPGG |
| | | QKGHSAEARGNRMADQAARKAAITETPDTSTLL |
| 53 | MMLV RT | MTWLSDFPQAWAETGGMGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAYFCLRLHPTSQPLFAFEWRDPEMG |
| | | ISGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPPDRWLSKARMTHYQALLLDTDRVQFGPVVALNPAT |
| | | LLPLPEEGLQHNCLDILAEAHGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIHCPGG |
| | | QKGHSAEARGNRMADQAARKAAITETPDTSTLL |
| 54 | MMLV RT | MTWLSDFPQAWAETGGVGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPPDRWLSKARMTHYQALLLDTDRVQFGPVVALNPAT |
| | | LLPLPEEGLQHNCLDILAEAHGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIHCPGH |
| | | QKGHSAEARGNRMADQAARKAAITETPDTSTLL |
| 55 | MMLV RT | MTWLSDFPQAWAETGGMGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAYFCLRLHPTSQPLFAFEWRDPEMG |
| | | ISGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPPDRWLSKARMTHYQALLLDTDRVQFGPVVALNPAT |
| | | LLPLPEEGLQHNCLDILAEAHGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIHCPGH |
| | | QKGHSAEARGNRMADQAARKAAITETPDTSTLL |
| 56 | MMLV RT | MTWLSDFPQAWAETGGMGLAVRQAPLIIPLKATSTPVSI |
| | Variant | KQYPLSQKARLGIKPHIQRLLDQGILVPCQSPWNTPLLPV |
| | | KKPGTNDYRPVQDLREVNKRVEDIHPTVPNPYNLLSGPP |
| | | PSHQWYTVLDLKDAFFCLRLHPTSQPLFAFEWRDPEMGI |
| | | SGQLTWTRLPQGFKNSPTLFNEALHRDLADFRIQHPDLI |
| | | LLQYVDDLLLAATSELDCQQGTRALLQTLGNLGYRASA |
| | | KKAQICQKQVKYLGYLLKEGQRWLTEARKETVMGQPT |
| | | PKTPRQLRRFLGKAGFCRLFIPGFAEMAAPLYPLTKPGT |
| | | LFNWGPDQQKAYQEIKQALLTAPALGLPDLTKPFELFV |
| | | DEKQGYAKGVLTQKLGPWRRPVAYLSKKLDPVAAGWP |
| | | PCLRMVAAIAVLTKDAGKLTMGQPLVIGAPHAVEALVK |
| | | QPPDRWLSKARMTHYQALLLDTDRVQFGPVVALNPAT |
| | | LLPLPEEGLQHNCLDILAEAHGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIHCPGH |
| | | QKGHSAEARGNRMADQAARKAAITETPDTSTLL |
| 57 | MMLV RT | MGSSHHHHHHSSGLVPRGSTWLSDFPQAWAETGGMGL |
| | Variant | AVRQAPLIIPLKATSTPVSIKQYPMSQKARLGIKPHIQRLL |
| | | DQGILVPCQSPWNTPLLPVKKPGTNDYRPVQDLREVNK |
| | | RVEDIHPTVPNPYNLLSGPPPSHQWYTVLDLKDAFFCLR |
| | | LHPTSQPLFAFEWRDPEMGISGQLTWTRLPQGFKNSPTL |
| | | FNEALHRDLADFRIQHPDLILLQYVDDLLLAATSELDCQ |
| | | QGTRALLQTLGNLGYRASAKKAQICQKQVKYLGYLLKE |
| | | GQRWLTEARKETVMGQPTPKTPRQLRRFLGKAGFCRLF |
| | | IPGFAEMAAPLYPLTKPGTLFNWGPDQQKAYQEIKQAL |
| | | LTAPALGLPDLTKPFELFVDEKQGYAKGVLTQKLGPWR |
| | | RPVAYLSKKLDPVAAGWPPCLRMVAAIAVLTKDAGKL |
| | | TMGQPLVIGAPHAVEALVKQPAGRWLSKARMTHYQAL |
| | | LLDTDRVQFGPVVALNPATLLPLPEEGLQHNCLDILAEA |
| | | HGTRPDLTDQPLPDADHTWYTNGSSLLQEGQRKAGAA |
| | | VTTETEVIWAKALPAGTSAQRAELIALTQALKMAEGKK |
| | | LNVYTDSRYAFATAHIHGEIYRRRGWLTSKGKEIKNKD |
| | | EILALLKALFLPKRLSIIHCPGHQKGHSAEARGNRMADQ |
| | | AARKAAITETPDTSTLLIENSSPNSRLIN |
| 58 | MMLV RT | MGSSHHHHHHSSGLVPRGSTWLSDFPQAWAETGGMGL |
| | Variant | AVRQAPLIIPLKATSTPVSIKQYPMSQKARLGIKPHIQRLL |
| | | DQGILVPCQSPWNTPLLPVKKPGTNDYRPVQDLREVNK |
| | | RVEDIHPTVPNPYNLLSGPPPSHQWYTVLDLKDAFFCLR |
| | | LHPTSQPLFAFEWRDPEMGISGQLTWTRLPQGFKNSPTL |
| | | FNEALHRDLADFRIQHPDLILLQYVDDLLLAATSELDCQ |
| | | QGTRALLQTLGNLGYRASAKKAQICQKQVKYLGYLLKE |
| | | GQRWLTEARKETVMGQPTPKTPRQLRRFLGKAGFCRLF |
| | | IPGFAEMAAPLYPLTKPGTLFNWGPDQQKAYQEIKQAL |
| | | LTAPALGLPDLTKPFELFVDEKQGYAKGVLTQKLGPWR |
| | | RPVAYLSKKLDPVAAGWPPCLRMVAAIAVLTKDAGKL |
| | | TMGQPLVIGAPHAVEALVKQPAGRWLSKARMTHYQAL |
| | | LLDTDRVQFGPVVALNPATLLPLPEEGLQHNCLDILAEA |
| | | HGTRPDLTDQPLPDADHTWYTNGSSLLQEGQRKAGAA |
| | | VTTETEVIWAKALPAGTSAQRAELIALTQALKMAEGKK |
| | | LNVYTDSRYAFATAHIHGEIYRRRGWLTSKGKEIKNKD |
| | | EILALLKALFLPKRLSIIHCPGHQKGHSAEARGNRMADQ |
| | | AARKAAITETPDTSTLLIENSSPNSRLIN |
| 59 | MMLV RT | MTWLSDFPQAWAETGGMGLAVRQAPLIIPLKATSTPVSI |
| | Variant | KQYPMSQKARLGIKPHIQRLLDQGILVPCQSPWNTPLLP |
| | | VKKPGTNDYRPVQDLREVNKRVEDIHPTVPNPYNLLSG |
| | | PPPSHQWYTVLDLKDAFFCLRLHPTSQPLFAFEWRDPE |
| | | MGISGQLTWTRLPQGFKNSPTLFNEALHRDLADFRIQHP |
| | | DLILLQYVDDLLLAATSELDCQQGTRALLQTLGNLGYR |
| | | ASAKKAQICQKQVKYLGYLLKEGQRWLTEARKETVMG |
| | | QPTPKTPRQLRRFLGKAGFCRLFIPGFAEMAAPLYPLTKP |
| | | GTLFNWGPDQQKAYQEIKQALLTAPALGLPDLTKPFELF |
| | | VDEKQGYAKGVLTQKLGPWRRPVAYLSKKLDPVAAG |
| | | WPPCLRMVAAIAVLTKDAGKLTMGQPLVIGAPHAVEAL |
| | | VKQPAGRWLSKARMTHYQALLLDTDRVQFGPVVALNP |
| | | ATLLPLPEEGLOHNCLDILAEAHGTRPDLTDQPLPDADH |
| | | TWYTNGSSLLQEGQRKAGAAVTTETEVIWAKALPAGTS |
| | | AQRAELIALTQALKMAEGKKLNVYTDSRYAFATAHIHG |
| | | EIYRRRGWLTSKGKEIKNKDEILALLKALFLPKRLSIIH |
| | | CPGHQKGHSAEARGNRMADQAARKAAITETPDTSTLLIEN |
| | | SSPNSRLIN |
| 60 | MMLV RT | GSTWLSDFPQAWAETGGMGLAVRQAPLIIPLKATSTPVS |
| | Variant | IKQYPMSQKARLGIKPHIQRLLDQGILVPCQSPWNTPLLP |
| | | VKKPGTNDYRPVQDLREVNKRVEDIHPTVPNPYNLLSG |
| | | PPPSHQWYTVLDLKDAFFCLRLHPTSQPLFAFEWRDPE |
| | | MGISGQLTWTRLPQGFKNSPTLFNEALHRDLADFRIQHP |
| | | DLILLQYVDDLLLAATSELDCQQGTRALLQTLGNLGYR |
| | | ASAKKAQICQKQVKYLGYLLKEGQRWLTEARKETVMG |
| | | QPTPKTPRQLRRFLGKAGFCRLFIPGFAEMAAPLYPLTKP |
| | | GTLFNWGPDQQKAYQEIKQALLTAPALGLPDLTKPFELF |
| | | VDEKQGYAKGVLTQKLGPWRRPVAYLSKKLDPVAAG |
| | | WPPCLRMVAAIAVLTKDAGKLTMGQPLVIGAPHAVEAL |
| | | VKQPAGRWLSKARMTHYQALLLDTDRVQFGPVVALNP |
| | | ATLLPLPEEGLOHNCLDILAEAHGTRPDLTDQPLPDADH |
| | | TWYTNGSSLLQEGQRKAGAAVTTETEVIWAKALPAGTS |
| | | AQRAELIALTQALKMAEGKKLNVYTDSRYAFATAHIHG |
| | | EIYRRRGWLTSKGKEIKNKDEILALLKALFLPKRLSIIHCP |
| | | GHQKGHSAEARGNRMADQAARKAAITETPDTSTLLIEN |
| | | SSPNSRLIN |
| 61 | MMLV RT | TWLSDFPQAWAETGGMGLAVRQAPLIIPLKATSTPVSIK |
| | Variant | QYPLSQKARLGIKPHIQRLLDQGILVPCQSPWNTPLLPVK |
| | | KPGTNDYRPVQDLREVNKRVEDIHPTVPNPYNLLSGPPP |
| | | SHQWYTVLDLKDAFFCLRLHPTSQPLFAFEWRDPEMGIS |
| | | GQLTWTRLPQGFKNSPTLFNEALHRDLADFRIQHPDLIL |
| | | LQYVDDLLLAATSELDCQQGTRALLQTLGNLGYRASAK |
| | | KAQICQKQVKYLGYLLKEGQRWLTEARKETVMGQPTP |
| | | KTPRQLRRFLGKAGFCRLFIPGFAEMAAPLYPLTKPGTL |
| | | FNWGPDQQKAYQEIKQALLTAPALGLPDLTKPFELFVD |
| | | EKQGYAKGVLTQKLGPWRRPVAYLSKKLDPVAAGWPP |
| | | CLRMVAAIAVLTKDAGKLTMGQPLVIGAPHAVEALVK |
| | | QPAGRWLSKARMTHYQALLLDTDRVQFGPVVALNPAT |
| | | LLPLPEEGLQHNCLDILAEAHGTRPDLTDQPLPDADHTW |
| | | YTNGSSLLQEGQRKAGAAVTTETEVIWAKALPAGTSAQ |
| | | RAELIALTQALKMAEGKKLNVYTDSRYAFATAHIHGEIY |
| | | RRRGWLTSKGKEIKNKDEILALLKALFLPKRLSIIHCPGH |
| | | QKGHSAEARGNRMADQAARKAAITETPDTSTLLIENSSP |
| | | NSRLIN |
| 62 | Affinity Tag | His His His His His |
| 193 | solubility | SEEDEEKEEDG |
| | enhancer tag | |
| 194 | endoprotein | ENLYFQ/G |
| | cleavage site | |
| 195 | endoprotein | DDDDK/ |
| | cleavage site | |
| 196 | endoprotein | IEGR/ |
| | cleavage site | |
| 197 | endoprotein | LVPR/GS |
| | cleavage site | |
| 198 | endoprotein | LEVLFQ/GP |
| | cleavage site | |
| 199 | linker | GSGGSG |
| 200 | linker | GGTTCAGGGGGTTCCGGT |

TABLE 2
Listing of amino acid and nucleic acid sequences of non-limiting embodiments
of the engineered RTs of the present disclosure

| SEQ | | | SEQ | |
|---|---|---|---|---|
| ID | | | ID | |
| NO: | Description | Amino Acid Sequence | NO: | Nucleic Acid Sequence |
| 63 | RTx | TWLSDFPQAWAETGGVGLAVRQAPLIIPLK | 64 | acttggctgtctgatttccctcaggcgtgggccgaaacgg |
| | SOLD 016 | ATSTPVSIKQYPMSQKARLGIKPHIQRLLD | | gtggcgtgggtctggcagtgcgtcaggcaccgctgattat |
| | N-Tag- His | QGILVPCQSPWNTPLLPVKKPGTNDYRPVQ | | tccgctgaaagcgacgtcgaccccggtgagcatcaagcaa |
| | | DLREVNKRVEDIHPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFEEAL | | tcagagcccgtggaataccccgcttctcccggtgaagaaa |
| | | RRDLADFRIQHPDLILLQYVDDLLLAATSE | | ccgggcacgaacgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMSQPLVIKAPHAVEAL | | cgctgtttgaagaagcgctgcgccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTDETEVIWAKALPAGTSAQRAEL | | gggttatcgcgctagcgcgaagaaagcgcagatttgccaa |
| | | IALTQALKMAEGKKLNVYTNSRYAFATAHI | | aaacaagttaagtatctgggctacctgttaaaggaaggcc |
| | | HGEIYRRRGWLTSGGKEIKNKDEILALLKA | | aacgttggctgaccgaagcccgcaaagaaactgtcatggg |
| | | LFLSKRLSIIHCPGHQKGHSVEARGNRMAH | | tcagccgaccccgaaaacgccacgccaactgcgtaggttc |
| | | QAARRAAITETPDTSTLPIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgagccaaccgctggtgatcaaagcac |
| | | | | cgcatgcagtcgaggcccttgttaagcaaccggcaggcag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccgatgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacgaac |
| | | | | agccgttatgcctttgcgaccgcccacattcacggcgaga |
| | | | | tctatcgccgtcgcggctggctgacgtccggcggcaaaga |
| | | | | gatcaagaataaagacgaaattctggcgctgctgaaagcg |
| | | | | ctgttcctgagcaaacgtctgtcgatcatccattgcccgg |
| | | | | gtcaccagaaaggccacagcgtggaggcgcgtggtaatcg |
| | | | | catggctcatcaggctgcgcgtcgcgccgcaattaccgaa |
| | | | | accccggacaccagcacgctgccgatcgagaatagcagcc |
| | | | | cgaacagccgtctgatcaat |
| 65 | RTx | TWLSDFPQAWAETGGLGLAVRQAPLIIPLK | 66 | acttggctgtctgatttccctcaggcgtgggccgaaacgg |
| | SOLD 001 | ATSTPVSIKQYPLSQKARLGIKPHIQRLLD | | gtggcctgggtctggcagtgcgtcaggcaccgctgattat |
| | FIG. | QGILVPCQSPWNTPLLPVKKPGTNDYRPVQ | | tccgctgaaagcgacgtcgaccccggtgagcatcaagcaa |
| | N-tag: His | DLREVNKRVEDIHPTVPNPYNLLSGPPPSH | | tatccgctgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcagagcccgtggaataccccgcttctcccggtgaagaaa |
| | | HRDLADFRIQHPDLILLQYVDDLLLAATSE | | ccgggcacgaacgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMGQPLVIGAPHAVEAL | | cgctgtttaacgaagcgctgcaccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTTETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaggttc |
| | | QAARKAAITETPDTSTLLIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgggtcaaccgctggtgatcggcgcac |
| | | | | cgcatgcagtcgaggcccttgttaagcaaccggcaggcag |
| | | | | atggctgagcaaggcgcgtatgacgcattaccaggcactg |
| | | | | ctgliggacaccgatcgtgigcagliiggcccggtcgilg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccaccgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccaaaggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgctgatcgagaatagcagcc |
| | | | | cgaacagccgtctgatcaat |
| 67 | RTx | TWLSDFPQAWAETGGLGLAVRQAPLIIPLK | 68 | acttggctgtctgatttccctcaggcgtgggccgaaacgg |
| | SOLD 002 | ATSTPVSIKQYPLSQKARLGIKPHIQRLLD | | gtggcctgggtctggcagtgcgtcaggcaccgctgattat |
| | N-Tag: his | QGILVPCQSPWNTPLLPVKKPGTSDYRPVQ | | tccgctgaaagcgacgtcgaccccggtgagcatcaagcaa |
| | | DLREVNKRVEDIHPTVPNPYNLLSGLPPSH | | tatccgctgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFVEAL | | tcagagcccgtggaataccccgcttctcccggtgaagaaa |
| | | RRDLADFRIQHPDLILLQYVDDLLLAATSE | | ccgggcacgagcgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtctgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMGQPLVIGAPHAVEAL | | cgctgtttgtggaagcgctgcgccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTTETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaggttc |
| | | QAARKAAITETPDTSTLLIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgggtcaaccgctggtgatcggcgcac |
| | | | | cgcatgcagtcgaggcccttgttaagcaaccggcaggcag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccaccgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccaaaggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgctgatcgagaatagcagcc |
| | | | | cgaacagccgtctgatcaat |
| 69 | RTx | TWLSDFPQAWAETGGVGLAVRQAPLIIPLK | 70 | acttggctgtctgatttccctcaggcgtgggccgaaacgg |
| | SOLD 003 | ATSTPVSIKQYPMSQKARLGIKPHIQRLLD | | gtggcgtgggtctggcagtgcgtcaggcaccgctgattat |
| | N-tag: His | QGILVPCQSPWNTPLLPVKKPGTSDYRPVQ | | tccgctgaaagcgacgtcgaccccggtgagcatcaagcaa |
| | | DLREVNKRVEDIHPTVPNPYNLLSGLPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcagagcccgtggaataccccgcttctcccggtgaagaaa |
| | | RRDLADFRIQHPDLILLQYVDDLLLAATSE | | ccgggcacgagcgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtctgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMGQPLVIGAPHAVEAL | | cgctgtttaacgaagcgctgcgccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTTETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaggttc |
| | | QAARKAAITETPDTSTLLIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgggtcaaccgctggtgatcggcgcac |
| | | | | cgcatgcagtcgaggcccttgttaagcaaccggcaggcag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccaccgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccaaaggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgctgatcgagaatagcagcc |
| | | | | cgaacagccgtctgatcaat |
| 71 | RTx | TWLSDFPQAWAETGGVGLAVRQAPLIIPLK | 72 | acttggctgtctgatttccctcaggcgtgggccgaaacgg |
| | SOLD 004 | ATSTPVSIKQYPMSQKARLGIKPHIQRLLD | | gtggcgtgggtctggcagtgcgtcaggcaccgctgattat |
| | N-tag- his | QGILVPCQSPWNTPLLPVKKPGTSDYRPVQ | | tccgctgaaagcgacgtcgaccccggtgagcatcaagcaa |
| | | DLREVNKRVEDIHPTVPNPYNLLSGLPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFVEAL | | tcagagcccgtggaataccccgcttctcccggtgaagaaa |
| | | HRDLADFRIQHPDLILLQYVDDLLLAATSE | | ccgggcacgagcgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtctgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMGQPLVIGAPHAVEAL | | cgctgtttgtggaagcgctgcaccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTTETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaggttc |
| | | QAARKAAITETPDTSTLLIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgggtcaaccgctggtgatcggcgcac |
| | | | | cgcatgcagtcgaggcccttgttaagcaaccggcaggcag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccaccgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccaaaggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgctgatcgagaatagcagcc |
| | | | | cgaacagccgtctgatcaat |
| 73 | RTx | TWLSDFPQAWAETGGVGLAVRQAPLIIPLK | 74 | acttggctgtctgatttccctcaggcgtgggccgaaacgg |
| | SOLD 005 | ATSTPVSIKQYPMSQKARLGIKPHIQRLLD | | gtggcgtgggtctggcagtgcgtcaggcaccgctgattat |
| | N-Tag: His | QGILVPCQSPWNTPLLPVKKPGTSDYRPVQ | | tccgctgaaagcgacgtcgaccccggtgagcatcaagcaa |
| | | DLREVNKRVEDIHPTVPNPYNLLSGLPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFVEAL | | tcagagcccgtggaataccccgcttctcccggtgaagaaa |
| | | RRDLADFRIQHPDLILLQYVDDLLLAATSE | | ccgggcacgagcgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtctgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMGQPLVIGAPHAVEAL | | cgctgtttgtggaagcgctgcgccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTTETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaggttc |
| | | QAARKAAITETPDTSTLLIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgggtcaaccgctggtgatcggcgcac |
| | | | | cgcatgcagtcgaggcccttgttaagcaaccggcaggcag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccaccgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccaaaggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgctgatcgagaatagcagcc |
| | | | | cgaacagccgtctgatcaat |
| 75 | RTx | TWLSDFPQAWAETGGVGLAVRQAPLIIPLK | 76 | acttggctgtctgatttccctcaggcgtgggccgaaacgg |
| | SOLD 006 | ATSTPVSIKQYPMSQKARLGIKPHIQRLLD | | gtggcgtgggtctggcagtgcgtcaggcaccgctgattat |
| | N-tag: His | QGILVPCQSPWNTPLLPVKKPGTNDYRPVQ | | tccgctgaaagcgacgtcgaccccggtgagcatcaagcaa |
| | | DLREVNKRVEDIHPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcagagcccgtggaataccccgcttctcccggtgaagaaa |
| | | HRDLADFRIQHPDLILLQYVDDLLLAATSE | | ccgggcacgaacgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtccgccgccaagccac |
| | | PKTPRQLRKFLGRAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMGQPLVIGAPHAVEAL | | cgctgtttaacgaagcgctgcaccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTTETEVIWAKALPAGTSAQRAEL | | gggttatcgcgctagcgcgaagaaagcgcagatttgccaa |
| | | IALTQALKMAEGKKLNVYTDSRYAFATAHT | | aaacaagttaagtatctgggctacctgttaaaggaaggcc |
| | | HGEIYRRRGWLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaaattc |
| | | QAARKAAITETPDTSTLLIENSSPNSRLIN | | ttgggccgcgcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgggtcaaccgctggtgatcggcgcac |
| | | | | cgcatgcagtcgaggcccttgttaagcaaccggcaggcag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccaccgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccaaaggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgctgatcgagaatagcagcc |
| | | | | cgaacagccgtctgatcaat |
| 77 | RTx | TWLSDFPQAWAETGGVGLAVRQAPLIIPLK | 78 | acttggctgtctgatttccctcaggcgtgggccgaaacgg |
| | SOLD 007 | ATSTPVSIKQYPMSQKARLGIKPHIQRLLD | | gtggcgtgggtctggcagtgcgtcaggcaccgctgattat |
| | N tag-His | QGILVPCQSPWNTPLLPVKKPGTNDYRPVQ | | tccgctgaaagcgacgtcgaccccggtgagcatcaagcaa |
| | | DLREVNKRVEDIHPTVPNPYNLLSGLPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcagagcccgtggaataccccgcttctcccggtgaagaaa |
| | | HRDLADFRIQHPDLILLQYVDDLLLAATSE | | ccgggcacgaacgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtctgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMGQPLVIGAPHAVEAL | | cgctgtttaacgaagcgctgcaccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTTETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaggttc |
| | | QAARKAAITETPDTSTLLIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgggtcaaccgctggtgatcggcgcac |
| | | | | cgcatgcagtcgaggcccttgttaagcaaccggcaggcag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccaccgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccaaaggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgctgatcgagaatagcagcc |
| | | | | cgaacagccgtctgatcaat |
| 79 | RTx | TWLSDFPQAWAETGGVGLAVRQAPLIIPLK | 80 | acttggctgtctgatttccctcaggcgtgggccgaaacgg |
| | SOLD 008 | ATSTPVSIKQYPMSQKARLGIKPHIQRLLD | | gtggcgtgggtctggcagtgcgtcaggcaccgctgattat |
| | N-tag: His | QGILVPCQSPWNTPLLPVKKPGTSDYRPVQ | | tccgctgaaagcgacgtcgaccccggtgagcatcaagcaa |
| | | DLREVNKRVEDIHPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcagagcccgtggaataccccgcttctcccggtgaagaaa |
| | | HRDLADFRIQHPDLILLQYVDDLLLAATSE | | ccgggcacgagcgattaccgtccagtccaagacttgcgcg |
| | | LDCRQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtccgccgccaagccac |
| | | PKTPRQLRKFLGRAGSCRLHIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMGQPLVIGAPHAVEAL | | cgctgtttaacgaagcgctgcaccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | gccagggcacccgtgcgctgttgcagaccttgggtaacct |
| | | RKAGAAVTTETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaaattc |
| | | QAARKAAITETPDTSTLLIENSSPNSRLIN | | ttgggccgcgcgggtagctgccgcctgcatatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgggtcaaccgctggtgatcggcgcac |
| | | | | cgcatgcagtcgaggcccttgttaagcaaccggcaggcag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccaccgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccaaaggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgctgatcgagaatagcagcc |
| | | | | cgaacagccgtctgatcaat |
| 81 | RTx | TWLSDFPQAWAETGGVGLAVRQAPLIIPLK | 82 | acttggctgtctgatttccctcaggcgtgggccgaaacgg |
| | SOLD 009 | ATSTPVSIKQYPMSQKARLGIKPHIQRLLD | | gtggcgtgggtctggcagtgcgtcaggcaccgctgattat |
| | N-Tag: His | QGILVPCQSPWNTPLLPVKKPGTNDYRPVQ | | tccgctgaaagcgacgtcgaccccggtgagcatcaagcaa |
| | | DLREVNKRVEDIHPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcagagcccgtggaataccccgcttctcccggtgaagaaa |
| | | HRDLADFRIQHPDLILLQYVDDLLLAATSE | | ccgggcacgaacgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMSQPLVIKAPHAVEAL | | cgctgtttaacgaagcgctgcaccgtgacctggcggattt |
| | | VKQPAERWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTDETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSGGKEIKNKDEILALLKA | | aacgttggctgaccgaagcccgcaaagaaactgtcatggg |
| | | LFLPKRLSIIHCPGHQKGHSVEARGNRMAH | | tcagccgaccccgaaaacgccacgccaactgcgtaggttc |
| | | QAARRAAITETPDTSTLPIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgagccaaccgctggtgatcaaagcac |
| | | | | cgcatgcagtcgaggcccttgttaagcaaccggcagaaag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccgatgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccggcggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgtggaggcgcgtggtaatcg |
| | | | | catggctcatcaggctgcgcgtcgcgccgcaattaccgaa |
| | | | | accccggacaccagcacgctgccgatcgagaatagcagcc |
| | | | | cgaacagccgtctgatcaat |
| 83 | RTx | TWLSDFPQAWAETWGVGLAVRQALLIIPLK | 84 | acttggctgtctgatttccctcaggcgtgggccgaaacgt |
| | SOLD 010 | ATSTPVSIKQYPMSQKARLGIKPHIQRLLD | | ggggcgtgggtctggcagtgcgtcaggcactgctgattat |
| | N-tag: His | QGILVPCRSPWNTPLLPVKKPGTSDYRPVQ | | tccgctgaaagcgacgtcgaccccggtgagcatcaagcaa |
| | | DLREVNKRVEDIRPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcgcagcccgtggaataccccgcttctcccggtgaagaaa |
| | | HRDLADFRIQHPDLILLQYVDDLLLAATSE | | ccgggcacgagcgattaccgtccagtccaagacttgcgcg |
| | | LDCQQGTRALLQTLGNLGYRASAKKAQICQ | | aagttaacaagcgcgttgaagatattcgcccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMSQPLVIKAPHAVEAL | | cgctgtttaacgaagcgctgcaccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTDETEVIWAKALPAGTSAQRAEL | | gggttatcgcgctagcgcgaagaaagcgcagatttgccaa |
| | | IALTQALKMAEGKKLNVYTNSRYAFATAHI | | aaacaagttaagtatctgggctacctgttaaaggaaggcc |
| | | HGEIYRRRGWLTSGGKEIKNKDEILALLKA | | aacgttggctgaccgaagcccgcaaagaaactgtcatggg |
| | | LFLSKRLSIIHCPGHQKGHSVEARGNRMAH | | tcagccgaccccgaaaacgccacgccaactgcgtaggttc |
| | | QAARRAAITETPDTSTLPIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgagccaaccgctggtgatcaaagcac |
| | | | | cgcatgcagtcgaggcccttgttaagcaaccggcaggcag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccgatgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacgaac |
| | | | | agccgttatgcctttgcgaccgcccacattcacggcgaga |
| | | | | tctatcgccgtcgcggctggctgacgtccggcggcaaaga |
| | | | | gatcaagaataaagacgaaattctggcgctgctgaaagcg |
| | | | | ctgttcctgagcaaacgtctgtcgatcatccattgcccgg |
| | | | | gtcaccagaaaggccacagcgtggaggcgcgtggtaatcg |
| | | | | catggctcatcaggctgcgcgtcgcgccgcaattaccgaa |
| | | | | accccggacaccagcacgctgccgatcgagaatagcagcc |
| | | | | cgaacagccgtctgatcaat |
| 85 | RTx | TWLSDFPQAWAETGGVGLAVRQAPLIIPLK | 86 | acttggctgtctgatttccctcaggcgtgggccgaaacgg |
| | SOLD 011 | ATSTPVSIKQYPMSQKARLGIKPHIQRLLD | | gtggcgtgggtctggcagtgcgtcaggcaccgctgattat |
| | N-Tag: His | QGILVPCQSPWNTPLLPVKKPGTNDYRPVQ | | tccgctgaaagcgacgtcgaccccggtgagcatcaagcaa |
| | | DLREVNKRVEDIHPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFEEAL | | tcagagcccgtggaataccccgcttctcccggtgaagaaa |
| | | RRDLADFRIQHPDLILLQYVDDLLLAATSE | | ccgggcacgaacgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMSQPLVIKAPHAVEAL | | cgctgtttgaagaagcgctgcgccgtgacctggcggattt |
| | | VKQPAERWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTDGSSLLQEGQ | | aacagggcacccgtgcgctgttgcagaccttgggtaacct |
| | | RKAGAAVTTETGVIWAKALPAGTSAQRAEL | | gggttatcgcgctagcgcgaagaaagcgcagatttgccaa |
| | | IALTQALKMAEGKKLNVYTNSRYAFATAHI | | aaacaagttaagtatctgggctacctgttaaaggaaggcc |
| | | QGEIYRRRGFLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaggttc |
| | | QAARKAAITETPDTSTLPIENSSPNPRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgagccaaccgctggtgatcaaagcac |
| | | | | cgcatgcagtcgaggcccttgttaagcaaccggcagaaag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacggatggtagcagcctgttgca |
| | | | | agagggtcagcgtaaggccggtgccgcggtcaccaccgag |
| | | | | actggcgtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacgaac |
| | | | | agccgttatgcctttgcgaccgcccacattcaggggagat |
| | | | | ctatcgccgtcgcggctttctgacgtccaaaggcaaagag |
| | | | | atcaagaataaagacgaaattctggcgctgctgaaagcgc |
| | | | | tgttcctgccgaaacgtctgtcgatcatccattgcccggg |
| | | | | tcaccagaaaggccacagcgcagaggcgcgtggtaatcgc |
| | | | | atggctgaccaggctgcgcgtaaagccgcaattaccgaaa |
| | | | | ccccggacaccagcacgctgccgatcgagaatagcagccc |
| | | | | gaacccgcgtctgatcaat |
| 87 | RTx | TWLSDFPQAWAETGGVGLAVRQAPLIIPLK | 88 | acttggctgtctgatttccctcaggcgtgggccgaaacgg |
| | SOLD 012 | ATSTPVSIKQYPMSQKARLGIKPHIQRLLD | | gtggcgtgggtctggcagtgcgtcaggcaccgctgattat |
| | N-tag: His | QGILVPCQSPWNTPLLPVKKPGTNDYRPVQ | | tccgctgaaagcgacgtcgaccccggtgagcatcaagcaa |
| | | DLREVNKRVEDIHPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcagagcccgtggaataccccgcttctcccggtgaagaaa |
| | | HRDLADFRIQHPDLILLQYVDDLLLAATSE | | ccgggcacgaacgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMSQPLVIKAPHAVEAL | | cgctgtttaacgaagcgctgcaccgtgacctggggatttc |
| | | VKQPAGRWLSKARMTHYQALLLDTDRVQFG | | cgtatccagcacccggacctgatcttgctgcagtacgttg |
| | | PVVALNPATLLPLPEEGLQHNCLDILAEAH | | atgacctgttgctggcggcgacgagcgagctggattgcca |
| | | GTRPDLTDQPLPDADHTWYTNGSSLLQEGQ | | acagggcacccgtgcgctgttgcagaccttgggtaacctg |
| | | RKAGAAVTDETEVIWAKALPAGTSAQRAEL | | ggttatcgcgctagcgcgaagaaagcgcagatttgccaaa |
| | | IALTQALKMAEGKKLNVYTNSRYAFATAHI | | aacaagttaagtatctgggctacctgttaaaggaaggcca |
| | | HGEIYRRRGWLTSGGKEIKNKDEILALLKA | | acgttggctgaccgaagcccgcaaagaaactgtcatgggt |
| | | LFLSKRLSIIHCPGHQKGHSVEARGNRMAH | | cagccgaccccgaaaacgccacgccaactgcgtaggttct |
| | | QAARRAAITETPDTSTLPIENSSPNSRLIN | | tgggcaaagcgggtttctgccgcctgttcatcccgggctt |
| | | | | tgccgaaatggcagccccgctgtatccgttgaccaagccg |
| | | | | ggcaccctgttcaactggggtccggaccagcagaaagcgt |
| | | | | accaagaaattaaacaagcactgctgacggcaccggcgct |
| | | | | gggtctgccggacctgaccaagccgtttgagctgttcgtg |
| | | | | gatgagaagcaaggttacgcgaagggcgtgttgacccaga |
| | | | | aattgggtccgtggcgtcgtccggttgcatacctgtccaa |
| | | | | gaaactggacccggttgctgctggttggccgccttgcctg |
| | | | | cgcatggttgccgctatcgcggtgctgactaaagacgcgg |
| | | | | gtaagctgacgatgagccaaccgctggtgatcaaagcacc |
| | | | | gcatgcagtcgaggcccttgttaagcaaccggcaggcaga |
| | | | | tggctgagcaaggcgcgtatgacgcattaccaggcactgc |
| | | | | tgttggacaccgatcgtgtgcagtttggcccggtcgttgc |
| | | | | gctcaacccggcgaccctgctgccgctcccggaagaaggc |
| | | | | ttgcagcacaactgtttggacatcctggcagaggcgcacg |
| | | | | gcactcgcccggatctgacggaccagccgctgccggacgc |
| | | | | cgatcatacctggtatacgaatggtagcagcctgttgcaa |
| | | | | gagggtcagcgtaaggccggtgccgcggtcaccgatgaga |
| | | | | ctgaagtgatttgggctaaagcattgcctgcgggtaccag |
| | | | | cgcgcagcgtgccgagctgatcgcactgacccaagcgctg |
| | | | | aaaatggctgagggtaagaaactgaatgtgtacacgaaca |
| | | | | gccgttatgcctttgcgaccgcccacattcacggcgagat |
| | | | | ctatcgccgtcgcggctggctgacgtccggcggcaaagag |
| | | | | atcaagaataaagacgaaattctggcgctgctgaaagcgc |
| | | | | tgttcctgagcaaacgtctgtcgatcatccattgcccggg |
| | | | | tcaccagaaaggccacagcgtggaggcgcgtggtaatcgc |
| | | | | atggctcatcaggctgcgcgtcgcgccgcaattaccgaaa |
| | | | | ccccggacaccagcacgctgccgatcgagaatagcagccc |
| | | | | gaacagccgtctgatcaat |
| 89 | RTx | TWLSDFPQAWAETWGVGLAVRQALLIIPLK | 90 | acttggctgtctgatttccctcaggcgtgggccgaaacgt |
| | SOLD 013 | ATSTPVSIKQYPMSQKARLGIKPHIQRLLD | | ggggcgtgggtctggcagtgcgtcaggcactgctgattat |
| | N-tag: His | QGILVPCRSPWNTPLLPVKKPGTSDYRPVQ | | tccgctgaaagcgacgtcgaccccggtgagcatcaagcaa |
| | | DLREVNKRVEDIRPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcgcagcccgtggaataccccgcttctcccggtgaagaaa |
| | | HRDLADFRIQHPDLILLQYVDDLLLAATSE | | ccgggcacgagcgattaccgtccagtccaagacttgcgcg |
| | | LDCQQGTRALLQTLGNLGYRASAKKAQICQ | | aagttaacaagcgcgttgaagatattcgcccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMSQPLVIKAPHAVEAL | | cgctgtttaacgaagcgctgcaccgtgacctggcggattt |
| | | VKQPAERWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTDETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSGGKEIKNKDEILALLKA | | aacgttggctgaccgaagcccgcaaagaaactgtcatggg |
| | | LFLPKRLSIIHCPGHQKGHSVEARGNRMAH | | tcagccgaccccgaaaacgccacgccaactgcgtaggttc |
| | | QAARRAAITETPDTSTLPIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgagccaaccgctggtgatcaaagcac |
| | | | | cgcatgcagtcgaggcccttgttaagcaaccggcagaaag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccgatgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccggcggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgtggaggcgcgtggtaatcg |
| | | | | catggctcatcaggctgcgcgtcgcgccgcaattaccgaa |
| | | | | accccggacaccagcacgctgccgatcgagaatagcagcc |
| | | | | cgaacagccgtctgatcaat |
| 91 | RTx | TWLSDFPQAWAETGGVGLAVRQAPLIIPLK | 92 | acttggctgtctgatttccctcaggcgtgggccgaaacgg |
| | SOLD 014 | ATSTPVSIKQYPMSQKARLGIKPHIQRLLD | | gggcgtgggtctggcagtgcgtcaggcaccgctgattatt |
| | N-Tag: His | QGILVPCQSPWNTPLLPVKKPGTNDYRPVQ | | ccgctgaaagcgacgtcgaccccggtgagcatcaagcaat |
| | | DLREVNKRVEDIHPTVPNPYNLLSGPPPSH | | atccgatgtcccaaaaggcgcgcttaggtattaagccgca |
| | | QWYTVLDLKDAFFCLRLHPTSQPLFAFEWR | | cattcagcgtctgctggatcaaggtattctggttccgtgt |
| | | DPEMGISGQLTWTRLPQGFKNSPTLFEEAL | | cagagcccgtggaataccccgcttctcccggtgaagaaac |
| | | RRDLADFRIQHPDLILLQYVDDLLLAATSE | | cgggcacgaacgattaccgtccagtccaagacttgcgcga |
| | | LDCQQGTRALLQTLGNLGYRASAKKAQICQ | | agttaacaagcgcgttgaagatattcacccgaccgtcccg |
| | | KQVKYLGYLLKEGQRWLTEARKETVMGQPT | | aacccgtacaatctgctgagcggtccgccgccaagccacc |
| | | PKTPRQLRRFLGKAGFCRLFIPGFAEMAAP | | aatggtacaccgtgctggatctgaaagatgctttcttctg |
| | | LYPLTKPGTLFNWGPDQQKAYQEIKQALLT | | tctgcgtctgcacccaaccagccagcctctgtttgcattt |
| | | APALGLPDLTKPFELFVDEKQGYAKGVLTQ | | gagtggcgtgaccctgagatgggtattagcggccagctga |
| | | KLGPWRRPVAYLSKKLDPVAAGWPPCLRMV | | cgtggacccgcctgccgcaaggttttaagaattcccctac |
| | | AAIAVLTKDAGKLTMSQPLVIKAPHAVEAL | | gctgtttgaagaagcgctgcgccgtgacctggcggatttc |
| | | VKQPAERWLSKARMTHYQALLLDTDRVQFG | | cgtatccagcacccggacctgatcttgctgcagtacgttg |
| | | PVVALNPATLLPLPEEGLQHNCLDILAEAH | | atgacctgttgctggcggcgacgagcgagctggattgcca |
| | | GTRPDLTDQPLPDADHTWYTNGSSLLQEGQ | | acagggcacccgtgcgctgttgcagaccttgggtaacctg |
| | | RKAGAAVTDETEVIWAKALPAGTSAQRAEL | | ggttatcgcgctagcgcgaagaaagcgcagatttgccaaa |
| | | IALTQALKMAEGKKLNVYTDSRYAFATAHI | | aacaagttaagtatctgggctacctgttaaaggaaggcca |
| | | HGEIYRRRGWLTSGGKEIKNKDEILALLKA | | acgttggctgaccgaagcccgcaaagaaactgtcatgggt |
| | | LFLPKRLSIIHCPGHQKGHSVEARGNRMAH | | cagccgaccccgaaaacgccacgccaactgcgtaggttct |
| | | QAARRAAITETPDTSTLPIENSSPNSRLIN | | tgggcaaagcgggtttctgccgcctgttcatcccgggctt |
| | | | | tgccgaaatggcagccccgctgtatccgttgaccaagccg |
| | | | | ggcaccctgttcaactggggtccggaccagcagaaagcgt |
| | | | | accaagaaattaaacaagcactgctgacggcaccggcgct |
| | | | | gggtctgccggacctgaccaagccgtttgagctgttcgtg |
| | | | | gatgagaagcaaggttacgcgaagggcgtgttgacccaga |
| | | | | aattgggtccgtggcgtcgtccggttgcatacctgtccaa |
| | | | | gaaactggacccggttgctgctggttggccgccttgcctg |
| | | | | cgcatggttgccgctatcgcggtgctgactaaagacgcgg |
| | | | | gtaagctgacgatgagccaaccgctggtgatcaaagcacc |
| | | | | gcatgcagtcgaggcccttgttaagcaaccggcagaaaga |
| | | | | tggctgagcaaggcgcgtatgacgcattaccaggcactgc |
| | | | | tgttggacaccgatcgtgtgcagtttggcccggtcgttgc |
| | | | | gctcaacccggcgaccctgctgccgctcccggaagaaggc |
| | | | | ttgcagcacaactgtttggacatcctggcagaggcgcacg |
| | | | | gcactcgcccggatctgacggaccagccgctgccggacgc |
| | | | | cgatcatacctggtatacgaatggtagcagcctgttgcaa |
| | | | | gagggtcagcgtaaggccggtgccgcggtcaccgatgaga |
| | | | | ctgaagtgatttgggctaaagcattgcctgcgggtaccag |
| | | | | cgcgcagcgtgccgagctgatcgcactgacccaagcgctg |
| | | | | aaaatggctgagggtaagaaactgaatgtgtacacggata |
| | | | | gccgttatgcctttgcgaccgcccacattcacggcgagat |
| | | | | ctatcgccgtcgcggctggctgacgtccggggcaaagaga |
| | | | | tcaagaataaagacgaaattctggcgctgctgaaagcgct |
| | | | | gttcctgccgaaacgtctgtcgatcatccattgcccgggt |
| | | | | caccagaaaggccacagcgtggaggcgcgtggtaatcgca |
| | | | | tggctcatcaggctgcgcgtcgcgccgcaattaccgaaac |
| | | | | cccggacaccagcacgctgccgatcgagaatagcagcccg |
| | | | | aacagccgtctgatcaat |
| 93 | RTx | TWLSDFPQAWAETGGVGLAVRQAPLIIPLK | 94 | acttggctgtctgatttccctcaggcgtgggccgaaacgg |
| | SOLD 015 | ATSTPVSIKQYPMSQKARLGIKPHIQRLLD | | gtggcgtgggtctggcagtgcgtcaggcaccgctgattat |
| | N-Tag: His | QGILVPCQSPWNTPLLPVKKPGTNDYRPVQ | | tccgctgaaagcgacgtcgaccccggtgagcatcaagcaa |
| | | DLREVNKRVEDIHPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcagagcccgtggaataccccgcttctcccggtgaagaaa |
| | | HRDLADFRIQHPDLILLQYVDDLLLAATSE | | ccgggcacgaacgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMSQPLVIKAPHAVEAL | | cgctgtttaacgaagcgctgcaccgtgacctggcggattt |
| | | VKQPAERWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTDGSSLLQEGQ | | aacagggcacccgtgcgctgttgcagaccttgggtaacct |
| | | RKAGAAVTTETGVIWAKALPAGTSAQRAEL | | gggttatcgcgctagcgcgaagaaagcgcagatttgccaa |
| | | IALTQALKMAEGKKLNVYTNSRYAFATAHI | | aaacaagttaagtatctgggctacctgttaaaggaaggcc |
| | | QGEIYRRRGFLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaggttc |
| | | QAARKAAITETPDTSTLPIENSSPNPRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgagccaaccgctggtgatcaaagcac |
| | | | | cgcatgcagtcgaggcccttgttaagcaaccggcagaaag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacggatggtagcagcctgttgca |
| | | | | agagggtcagcgtaaggccggtgccgcggtcaccaccgag |
| | | | | actggcgtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacgaac |
| | | | | agccgttatgcctttgcgaccgcccacattcagggcgaga |
| | | | | tctatcgccgtcgcggctttctgacgtccaaaggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgccgatcgagaatagcagcc |
| | | | | cgaacccgcgtctgatcaat |
| 95 | RTx | TWLSDFPQAWAETWGVGLAVRQALLIIPLK | 96 | acttggctgtctgatttccctcaggcgtgggccgaaacgt |
| | SOLD 017 | ATSTPVSIKQYPMSQKARLGIKPHIQRLLD | | ggggcgtgggtctggcagtgcgtcaggcactgctgattat |
| | N-Tag: His | QGILVPCRSPWNTPLLPVKKPGTSDYRPVQ | | tccgctgaaagcgacgtcgaccccggtgagcatcaagcaa |
| | | DLREVNKRVEDIRPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcgcagcccgtggaataccccgcttctcccggtgaagaaa |
| | | HRDLADFRIQHPDLILLQYVDDLLLAATSE | | ccgggcacgagcgattaccgtccagtccaagacttgcgcg |
| | | LDCQQGTRALLQTLGNLGYRASAKKAQICQ | | aagttaacaagcgcgttgaagatattcgcccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMSQPLVIKAPHAVEAL | | cgctgtttaacgaagcgctgcaccgtgacctggcggattt |
| | | VKQPAERWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTDGSSLLQEGQ | | aacagggcacccgtgcgctgttgcagaccttgggtaacct |
| | | RKAGAAVTTETGVIWAKALPAGTSAQRAEL | | gggttatcgcgctagcgcgaagaaagcgcagatttgccaa |
| | | IALTQALKMAEGKKLNVYTNSRYAFATAHI | | aaacaagttaagtatctgggctacctgttaaaggaaggcc |
| | | QGEIYRRRGFLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaggttc |
| | | QAARKAAITETPDTSTLPIENSSPNPRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgagccaaccgctggtgatcaaagcac |
| | | | | cgcatgcagtcgaggcccttgttaagcaaccggcagaaag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacggatggtagcagcctgttgca |
| | | | | agagggtcagcgtaaggccggtgccgcggtcaccaccgag |
| | | | | actggtgtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacgaac |
| | | | | agccgttatgcctttgcgaccgcccacattcagggcgaga |
| | | | | tctatcgccgtcgcggctttctgacgtccaaaggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgccgatcgagaatagcagcc |
| | | | | cgaacccgcgtctgatcaat |
| 97 | RTx | TWLSDFPQAWAETGGVGLAVRQAPLIIPLK | 98 | acttggctgtctgatttccctcaggcgtgggccgaaacgg |
| | SOLD 018 | ATSTPVSIKQYPMSQKARLGIKPHIQRLLD | | gtggcgtgggtctggcagtgcgtcaggcaccgctgattat |
| | N-Tag: His | QGILVPCQSPWNTPLLPVKKPGTNDYRPVQ | | tccgctgaaagcgacgtcgaccccggtgagcatcaagcaa |
| | | DLREVNKRVEDIHPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcagagcccgtggaataccccgcttctcccggtgaagaaa |
| | | HRDLADFRIQHPDLILLQYVDDLLLAATSE | | ccgggcacgaacgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMSQPLVIKAPHAVEAL | | cgctgtttaacgaagcgctgcaccgtgacctggcggattt |
| | | VKQPAERWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTDETEVIWAKALPAGTSAQRAEL | | gggttatcgcgctagcgcgaagaaagcgcagatttgccaa |
| | | IALTQALKMAEGKKLNVYTNSRYAFATAHI | | aaacaagttaagtatctgggctacctgttaaaggaaggcc |
| | | HGEIYRRRGWLTSGGKEIKNKDEILALLKA | | aacgttggctgaccgaagcccgcaaagaaactgtcatggg |
| | | LFLSKRLSIIHCPGHQKGHSVEARGNRMAH | | tcagccgaccccgaaaacgccacgccaactgcgtaggttc |
| | | QAARRAAITETPDTSTLPIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgagccaaccgctggtgatcaaagcac |
| | | | | cgcatgcagtcgaggcccttgttaagcaaccggcagaaag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccgatgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacgaac |
| | | | | agccgttatgcctttgcgaccgcccacattcacggcgaga |
| | | | | tctatcgccgtcgcggctggctgacgtccggcggcaaaga |
| | | | | gatcaagaataaagacgaaattctggcgctgctgaaagcg |
| | | | | ctgttcctgagcaaacgtctgtcgatcatccattgcccgg |
| | | | | gtcaccagaaaggccacagcgtggaggcgcgtggtaatcg |
| | | | | catggctcatcaggctgcgcgtcgcgccgcaattaccgaa |
| | | | | accccggacaccagcacgctgccgatcgagaatagcagcc |
| | | | | cgaacagccgtctgatcaat |
| 99 | RTx | TWLSDFPQAWAETGGVGLAVRQAPLIIPLK | 100 | acttggctgtctgatttccctcaggcgtgggccgaaacgg |
| | SOLD 019 | ATSTPVSIKQYPMSQKARLGIKPHIQRLLD | | gtggcgtgggtctggcagtgcgtcaggcaccgctgattat |
| | N-tag: His | QGILVPCRSPWNTPLLPVKKPGTSDYRPVQ | | tccgctgaaagcgacgtcgaccccggtgagcatcaagcaa |
| | | DLREVNKRVEDIRPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcgcagcccgtggaataccccgcttctcccggtgaagaaa |
| | | HRDLADFRIQHPDLILLQYVDDLLLAATSE | | ccgggcacgagcgattaccgtccagtccaagacttgcgcg |
| | | LDCQQGTRALLQTLGNLGYRASAKKAQICQ | | aagttaacaagcgcgttgaagatattcgcccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMSQPLVIKAPHAVEAL | | cgctgtttaacgaagcgctgcaccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTDETEVIWAKALPAGTSAQRAEL | | gggttatcgcgctagcgcgaagaaagcgcagatttgccaa |
| | | IALTQALKMAEGKKLNVYTNSRYAFATAHI | | aaacaagttaagtatctgggctacctgttaaaggaaggcc |
| | | HGEIYRRRGWLTSGGKEIKNKDEILALLKA | | aacgttggctgaccgaagcccgcaaagaaactgtcatggg |
| | | LFLSKRLSIIHCPGHQKGHSVEARGNRMAH | | tcagccgaccccgaaaacgccacgccaactgcgtaggttc |
| | | QAARRAAITETPDTSTLPIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgagccaaccgctggtgatcaaagcac |
| | | | | cgcatgcagtcgaggcccttgttaagcaaccggcaggcag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccgatgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacgaac |
| | | | | agccgttatgcctttgcgaccgcccacattcacggcgaga |
| | | | | tctatcgccgtcgcggctggctgacgtccggcggcaaaga |
| | | | | gatcaagaataaagacgaaattctggcgctgctgaaagcg |
| | | | | ctgttcctgagcaaacgtctgtcgatcatccattgcccgg |
| | | | | gtcaccagaaaggccacagcgtggaggcgcgtggtaatcg |
| | | | | catggctcatcaggctgcgcgtcgcgccgcaattaccgaa |
| | | | | accccggacaccagcacgctgccgatcgagaatagcagcc |
| | | | | cgaacagccgtctgatcaat |
| 101 | RTx | TWLSDFPQAWAETWGVGLAVRQALLIIPLK | 102 | acttggctgtctgatttccctcaggcgtgggccgaaacgt |
| | SOLD 020 | ATSTPVSIKQYPMSQKARLGIKPHIQRLLD | | ggggcgtgggtctggcagtgcgtcaggcactgctgattat |
| | N-Tag: His | QGILVPCRSPWNTPLLPVKKPGTSDYRPVQ | | tccgctgaaagcgacgtcgaccccggtgagcatcaagcaa |
| | | DLREVNKRVEDIRPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcgcagcccgtggaataccccgcttctcccggtgaagaaa |
| | | HRDLADFRIQHPDLILLQYVDDLLLAATSE | | ccgggcacgagcgattaccgtccagtccaagacttgcgcg |
| | | LDCQQGTRALLQTLGNLGYRASAKKAQICQ | | aagttaacaagcgcgttgaagatattcgcccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMSQPLVIKAPHAVEAL | | cgctgtttaacgaagcgctgcaccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTDETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSGGKEIKNKDEILALLKA | | aacgttggctgaccgaagcccgcaaagaaactgtcatggg |
| | | LFLPKRLSIIHCPGHQKGHSVEARGNRMAH | | tcagccgaccccgaaaacgccacgccaactgcgtaggttc |
| | | QAARRAAITETPDTSTLPIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgagccaaccgctggtgatcaaagcac |
| | | | | cgcatgcagtcgaggcccttgttaagcaaccggcaggcag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccgatgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccggcggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgtggaggcgcgtggtaatcg |
| | | | | catggctcatcaggctgcgcgtcgcgccgcaattaccgaa |
| | | | | accccggacaccagcacgctgccgatcgagaatagcagcc |
| | | | | cgaacagccgtctgatcaat |
| 103 | RTx | TWLSDFPQAWAETGGVGLAVRQAPLIIPLK | 104 | acttggctgtctgatttccctcaggcgtgggccgaaacgg |
| | SOLD 021 | ATSTPVSIKQYPMSQKARLGIKPHIQRLLD | | gggcgtgggtctggcagtgcgtcaggcaccgctgattatt |
| | N-tag: His | QGILVPCQSPWNTPLLPVKKPGTNDYRPVQ | | ccgctgaaagcgacgtcgaccccggtgagcatcaagcaat |
| | | DLREVNKRVEDIHPTVPNPYNLLSGPPPSH | | atccgatgtcccaaaaggcgcgcttaggtattaagccgca |
| | | QWYTVLDLKDAFFCLRLHPTSQPLFAFEWR | | cattcagcgtctgctggatcaaggtattctggttccgtgt |
| | | DPEMGISGQLTWTRLPQGFKNSPTLFEEAL | | cagagcccgtggaataccccgcttctcccggtgaagaaac |
| | | RRDLADFRIQHPDLILLQYVDDLLLAATSE | | cgggcacgaacgattaccgtccagtccaagacttgcgcga |
| | | LDCQQGTRALLQTLGNLGYRASAKKAQICQ | | agttaacaagcgcgttgaagatattcacccgaccgtcccg |
| | | KQVKYLGYLLKEGQRWLTEARKETVMGQPT | | aacccgtacaatctgctgagcggtccgccgccaagccacc |
| | | PKTPRQLRRFLGKAGFCRLFIPGFAEMAAP | | aatggtacaccgtgctggatctgaaagatgctttcttctg |
| | | LYPLTKPGTLFNWGPDQQKAYQEIKQALLT | | tctgcgtctgcacccaaccagccagcctctgtttgcattt |
| | | APALGLPDLTKPFELFVDEKQGYAKGVLTQ | | gagtggcgtgaccctgagatgggtattagcggccagctga |
| | | KLGPWRRPVAYLSKKLDPVAAGWPPCLRMV | | cgtggacccgcctgccgcaaggttttaagaattcccctac |
| | | AAIAVLTKDAGKLTMSQPLVIKAPHAVEAL | | gctgtttgaagaagcgctgcgccgtgacctggcggatttc |
| | | VKQPAERWLSKARMTHYQALLLDTDRVQFG | | cgtatccagcacccggacctgatcttgctgcagtacgttg |
| | | PVVALNPATLLPLPEEGLQHNCLDILAEAH | | atgacctgttgctggcggcgacgagcgagctggattgcca |
| | | GTRPDLTDQPLPDADHTWYTNGSSLLQEGQ | | acagggcacccgtgcgctgttgcagaccttgggtaacctg |
| | | RKAGAAVTDETEVIWAKALPAGTSAQRAEL | | ggttatcgcgctagcgcgaagaaagcgcagatttgccaaa |
| | | IALTQALKMAEGKKLNVYTNSRYAFATAHI | | aacaagttaagtatctgggctacctgttaaaggaaggcca |
| | | HGEIYRRRGWLTSGGKEIKNKDEILALLKA | | acgttggctgaccgaagcccgcaaagaaactgtcatgggt |
| | | LFLSKRLSIIHCPGHQKGHSVEARGNRMAH | | cagccgaccccgaaaacgccacgccaactgcgtaggttct |
| | | QAARRAAITETPDTSTLPIENSSPNSRLIN | | tgggcaaagcgggtttctgccgcctgttcatcccgggctt |
| | | | | tgccgaaatggcagccccgctgtatccgttgaccaagccg |
| | | | | ggcaccctgttcaactggggtccggaccagcagaaagcgt |
| | | | | accaagaaattaaacaagcactgctgacggcaccggcgct |
| | | | | gggtctgccggacctgaccaagccgtttgagctgttcgtg |
| | | | | gatgagaagcaaggttacgcgaagggcgtgttgacccaga |
| | | | | aattgggtccgtggcgtcgtccggttgcatacctgtccaa |
| | | | | gaaactggacccggttgctgctggttggccgccttgcctg |
| | | | | cgcatggttgccgctatcgcggtgctgactaaagacgcgg |
| | | | | gtaagctgacgatgagccaaccgctggtgatcaaagcacc |
| | | | | gcatgcagtcgaggcccttgttaagcaaccggcagaaaga |
| | | | | tggctgagcaaggcgcgtatgacgcattaccaggcactgc |
| | | | | tgttggacaccgatcgtgtgcagtttggcccggtcgttgc |
| | | | | gctcaacccggcgaccctgctgccgctcccggaagaaggc |
| | | | | ttgcagcacaactgtttggacatcctggcagaggcgcacg |
| | | | | gcactcgcccggatctgacggaccagccgctgccggacgc |
| | | | | cgatcatacctggtatacgaatggtagcagcctgttgcaa |
| | | | | gagggtcagcgtaaggccggtgccgcggtcaccgatgaga |
| | | | | ctgaagtgatttgggctaaagcattgcctgcgggtaccag |
| | | | | cgcgcagcgtgccgagctgatcgcactgacccaagcgctg |
| | | | | aaaatggctgagggtaagaaactgaatgtgtacacgaaca |
| | | | | gccgttatgcctttgcgaccgcccacattcacggcgagat |
| | | | | ctatcgccgtcgcggctggctgacgtccggcggcaaagag |
| | | | | atcaagaataaagacgaaattctggcgctgctgaaagcgc |
| | | | | tgttcctgagcaaacgtctgtcgatcatccattgcccggg |
| | | | | tcaccagaaaggccacagcgtggaggcgcgtggtaatcgc |
| | | | | atggctcatcaggctgcgcgtcgcgccgcaattaccgaaa |
| | | | | ccccggacaccagcacgctgccgatcgagaatagcagccc |
| | | | | gaacagccgtctgatcaat |
| 105 | RTx | TWLSDFPQAWAETGGVGLAVRQAPLIIPLK | 106 | acctggctgagcgattttccgcaggcatgggcagaaaccg |
| | SOLD 022 | ATSTPVSIKQYPMSQKARLGIKPHIQRLLD | | gtggtgtgggtctggcagtgcgccaggcaccgctgattat |
| | N-tag: His | QGILVPCQSPWNTPLLPVKKPGTNDYRPVQ | | tccgctgaaagcaaccagcaccccggtgagcattaaacag |
| | | DLREVNKRVEDIHPTVPNPYNLLSGPPPSH | | tatccgatgagccagaaagcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccagagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRKFLGAAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttaacgaagcactgcatcgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTDGSSLLQEGQ | | agcagggtacccgcgcactgctgcagaccctgggtaacct |
| | | RKAGAAVTTETGVIWAKALPAGTSAQRAEL | | gggttatcgcgcaagcgcaaaaaaagcacagatttgccag |
| | | IALTQALKMAEGKKLNVYTNSRYAFATAHI | | aaacaggtgaaatatctgggttatctgctgaaagaaggtc |
| | | QGEIYRRRGFLTSKGKEIKNKDEILALLKA | | agcgctggctgaccgaagcacgcaaagaaaccgtgatggg |
| | | LFLPKRLSIIHCPGHQKGHSAEARGNRMAD | | tcagccgaccccgaaaaccccgcgccagctgcgcaaattt |
| | | QAARKAAITETPDTSTLPIENSSPNPRLIN | | ctgggtgcagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgggtcagccgctggtgattggtgcac |
| | | | | cgcatgcagtggaagcactggtgaaacagccggcaggtcg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccgatggtagcagcctgctgca |
| | | | | ggaaggtcagcgcaaagcaggtgcagcagtgaccaccgaa |
| | | | | accggtgtgatttgggcaaaagcactgccggcaggtacca |
| | | | | gcgcacagcgcgcagaactgattgcactgacccaggcact |
| | | | | gaaaatggcagaaggtaaaaaactgaacgtgtataccaac |
| | | | | agccgctatgcatttgcaaccgcacatattcagggtgaaa |
| | | | | tttatcgccgccgcggttttctgaccagcaaaggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgcagaagcacgcggtaaccg |
| | | | | catggcagatcaggcagcacgcaaagcagcaattaccgaa |
| | | | | accccggataccagcaccctgccgattgaaaacagcagcc |
| | | | | cgaacccgcgcctgattaac |
| 107 | RTx | TWLSDFPQAWAETGGVGLAVRQAPLTIPLK | 108 | acctggctgagcgattttccgcaggcatgggcagaaaccg |
| | SOLD 023 | ATSTPVSIKQYPMSQKARLGIKPHIQRLLD | | gtggtgtgggtctggcagtgcgccaggcaccgctgattat |
| | N-Tag: His | QGILVPCQSPWNTPLLPVKKPGTNDYRPVQ | | tccgctgaaagcaaccagcaccccggtgagcattaaacag |
| | | DLREVNKRVEDIHPTVPNPYNLLSGPPPSH | | tatccgatgagccagaaagcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccagagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttaacgaagcactgcatcgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTTETEVIWAKALPAGTSAQRAEL | | gggttatcgcgcaagcgcaaaaaaagcacagatttgccag |
| | | IALTQALKMAEGKKLNVYTDSRYAFATAHI | | aaacaggtgaaatatctgggttatctgctgaaagaaggtc |
| | | HGEIYRRRGWLTSKGKEIKNKDEILALLKA | | agcgctggctgaccgaagcacgcaaagaaaccgtgatggg |
| | | LFLPKRLSIIHCPGHQKGYSAEARGNRMAV | | tcagccgaccccgaaaaccccgcgccagctgcgccgcttt |
| | | QAARKAAITETPDTSTLLIENSSPNPRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgggtcagccgctggtgattggtgcac |
| | | | | cgcatgcagtggaagcactggtgaaacagccggcaggtcg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccaacggtagcagcctgctgca |
| | | | | ggaaggtcagcgcaaagcaggtgcagcagtgaccaccgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccgat |
| | | | | agccgctatgcatttgcaaccgcacatattcatggtgaaa |
| | | | | tttatcgccgccgcggttggctgaccagcaaaggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggttatagcgcagaagcacgcggtaaccg |
| | | | | catggcagtgcaggcagcacgcaaagcagcaattaccgaa |
| | | | | accccggataccagcaccctgctgattgaaaacagcagcc |
| | | | | cgaacccgcgcctgattaac |
| 109 | RTx | TWLSDFPQAWAETGGVGLAVRQAPLIIPLK | 110 | acctggctgagcgattttccgcaggcatgggcagaaaccg |
| | SOLD 024 | ATSTPVSIKQYPMSQRARLGIKPHIQRLLD | | gtggtgtgggtctggcagtgcgccaggcaccgctgattat |
| | N-tag: His | QGILVPCRSPWNTPLLPVKKPGTSDYRPVQ | | tccgctgaaagcaaccagcaccccggtgagcattaaacag |
| | | DLREVNKRVEDIRPTVPNPYNLLSGPPPSH | | tatccgatgagccagcgcgcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFEEAL | | ccgcagcccgtggaacaccccgctgctgccggtgaaaaaa |
| | | RRDLADFRIQHPDLILLQYVDDLLLAATSE | | ccgggtaccagcgattatcgcccggtgcaggatctgcgcg |
| | | LDCQQGTRALLQTLGNLGYRASAKKAQICQ | | aagtgaacaaacgcgtggaagatattcgcccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttgaagaagcactgcgccgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTTETEVIWAKALPAGTSAQRAEL | | gggttatcgcgcaagcgcaaaaaaagcacagatttgccag |
| | | IALTQALKMAEGKKLNVYTDSRYAFATAHI | | aaacaggtgaaatatctgggttatctgctgaaagaaggtc |
| | | HGEIYRRRGWLTSKGKEIKNKDEILALLKA | | agcgctggctgaccgaagcacgcaaagaaaccgtgatggg |
| | | LFLPKRLSIIHCPGHQKGHSAEARGNRMAD | | tcagccgaccccgaaaaccccgcgccagctgcgccgcttt |
| | | QAARKAAITETPDTSTLLIENSSPNSRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgggtcagccgctggtgattggtgcac |
| | | | | cgcatgcagtggaagcactggtgaaacagccggcaggtcg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccaacggtagcagcctgctgca |
| | | | | ggaaggtcagcgcaaagcaggtgcagcagtgaccaccgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccgat |
| | | | | agccgctatgcatttgcaaccgcacatattcatggtgaaa |
| | | | | tttatcgccgccgcggttggctgaccagcaaaggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgcagaagcacgcggtaaccg |
| | | | | catggcagatcaggcagcacgcaaagcagcaattaccgaa |
| | | | | accccggataccagcaccctgctgattgaaaacagcagcc |
| | | | | cgaacagccgcctgattaac |
| 111 | RTx | TWLSDFPQAWAETGGVGLAVRQAPLIIPLK | 112 | acctggctgagcgattttccgcaggcatgggcagaaaccg |
| | SOLD 025 | ATSTPVSIKQYPMSQKARLGIKPHIQRLLD | | gtggtgtgggtctggcagtgcgccaggcaccgctgattat |
| | N-tag: His | QGILVPCQSPWNTPLLPVKKPGTNDYRPVQ | | tccgctgaaagcaaccagcaccccggtgagcattaaacag |
| | | DLREVNKRVEDIHPTVPNPYNLLSGPPPSH | | tatccgatgagccagaaagcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccagagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttaacgaagcactgcatcgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTDETEVIWAKALPAGTSAQRAEL | | gggttatcgcgcaagcgcaaaaaaagcacagatttgccag |
| | | IALTQALKMAEGKKLNVYTNSRYAFATAHI | | aaacaggtgaaatatctgggttatctgctgaaagaaggtc |
| | | HGEIYRRRGWLTSGGKEIKNKDEILALLKA | | agcgctggctgaccgaagcacgcaaagaaaccgtgatggg |
| | | LFLPKRLSIIHCPGHQKGHSVEARGNRMAH | | tcagccgaccccgaaaaccccgcgccagctgcgccgcttt |
| | | QAARRAAITETPDTSTLPIENSSPNSRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgggtcagccgctggtgattggtgcac |
| | | | | cgcatgcagtggaagcactggtgaaacagccggcaggtcg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccaacggtagcagcctgctgca |
| | | | | ggaaggtcagcgcaaagcaggtgcagcagtgaccgatgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccaac |
| | | | | agccgctatgcatttgcaaccgcacatattcatggtgaaa |
| | | | | tttatcgccgccgcggttggctgaccagcggtggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgtggaagcacgcggtaaccg |
| | | | | catggcacatcaggcagcacgccgcgcagcaattaccgaa |
| | | | | accccggataccagcaccctgccgattgaaaacagcagcc |
| | | | | cgaacagccgcctgattaac |
| 113 | RTx | TWLSDFPQAWAETGGVGLAVRQAPLIIPLK | 114 | acctggctgagcgattttccgcaggcatgggcagaaaccg |
| | SOLD 026 | ATSTPVSIKQYPMSQKARLGIKPHIQRLLD | | gtggtgtgggtctggcagtgcgccaggcaccgctgattat |
| | N-Tag: His | QGILVPCQSPWNTPLLPVKKPGTNDYRPVQ | | tccgctgaaagcaaccagcaccccggtgagcattaaacag |
| | | DLREVNKRVEDIHPTVPNPYNLLSGPPPSH | | tatccgatgagccagaaagcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccagagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttaacgaagcactgcatcgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTDGSSLLQEGQ | | agcagggtacccgcgcactgctgcagaccctgggtaacct |
| | | RKAGAAVTTETGVIWAKALPAGTSAQRAEL | | gggttatcgcgcaagcgcaaaaaaagcacagatttgccag |
| | | IALTQALKMAEGKKLNVYTNSRYAFATAHI | | aaacaggtgaaatatctgggttatctgctgaaagaaggtc |
| | | QGEIYRRRGFLTSKGKEIKNKDEILALLKA | | agcgctggctgaccgaagcacgcaaagaaaccgtgatggg |
| | | LFLPKRLSIIHCPGHQKGHSAEARGNRMAD | | tcagccgaccccgaaaaccccgcgccagctgcgccgcttt |
| | | QAARKAAITETPDTSTLPIENSSPNPRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgggtcagccgctggtgattggtgcac |
| | | | | cgcatgcagtggaagcactggtgaaacagccggcaggtcg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccgatggtagcagcctgctgca |
| | | | | ggaaggtcagcgcaaagcaggtgcagcagtgaccaccgaa |
| | | | | accggtgtgatttgggcaaaagcactgccggcaggtacca |
| | | | | gcgcacagcgcgcagaactgattgcactgacccaggcact |
| | | | | gaaaatggcagaaggtaaaaaactgaacgtgtataccaac |
| | | | | agccgctatgcatttgcaaccgcacatattcagggtgaaa |
| | | | | tttatcgccgccgcggttttctgaccagcaaaggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgcagaagcacgcggtaaccg |
| | | | | catggcagatcaggcagcacgcaaagcagcaattaccgaa |
| | | | | accccggataccagcaccctgccgattgaaaacagcagcc |
| | | | | cgaacccgcgcctgattaac |
| 115 | RTx | TWLSDFPQAWAETGGVGLAVRQAPLIIPLK | 116 | acctggctgagcgattttccgcaggcatgggcagaaaccg |
| | SOLD 027 | ATSTPVSIKQYPMSQRARLGIKPHIQRLLD | | gtggtgtgggtctggcagtgcgccaggcaccgctgattat |
| | N-tag: His | QGILVPCRSPWNTPLLPVKKPGTSDYRPVQ | | tccgctgaaagcaaccagcaccccggtgagcattaaacag |
| | | DLREVNKRVEDIRPTVPNPYNLLSGPPPSH | | tatccgatgagccagcgcgcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccgcagcccgtggaacaccccgctgctgccggtgaaaaaa |
| | | HRDLADFRIQHPDLILLQYVDDLLLAATSE | | ccgggtaccagcgattatcgcccggtgcaggatctgcgcg |
| | | LDCQQGTRALLQTLGNLGYRASAKKAQICQ | | aagtgaacaaacgcgtggaagatattcgcccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttaacgaagcactgcatcgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTTETEVIWAKALPAGTSAQRAEL | | gggttatcgcgcaagcgcaaaaaaagcacagatttgccag |
| | | IALTQALKMAEGKKLNVYTDSRYAFATAHI | | aaacaggtgaaatatctgggttatctgctgaaagaaggtc |
| | | HGEIYRRRGWLTSKGKEIKNKDEILALLKA | | agcgctggctgaccgaagcacgcaaagaaaccgtgatggg |
| | | LFLPKRLSIIHCPGHQKGHSAEARGNRMAD | | tcagccgaccccgaaaaccccgcgccagctgcgccgcttt |
| | | QAARKAAITETPDTSTLLIENSSPNSRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgggtcagccgctggtgattggtgcac |
| | | | | cgcatgcagtggaagcactggtgaaacagccggcaggtcg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccaacggtagcagcctgctgca |
| | | | | ggaaggtcagcgcaaagcaggtgcagcagtgaccaccgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccgat |
| | | | | agccgctatgcatttgcaaccgcacatattcatggtgaaa |
| | | | | tttatcgccgccgcggttggctgaccagcaaaggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgcagaagcacgcggtaaccg |
| | | | | catggcagatcaggcagcacgcaaagcagcaattaccgaa |
| | | | | accccggataccagcaccctgctgattgaaaacagcagcc |
| | | | | cgaacagccgcctgattaac |
| 117 | RTx | TWLSDFPQAWAETGGVGLAVRQAPLIIPLK | 118 | acctggctgagcgattttccgcaggcatgggcagaaaccg |
| | SOLD 028 | ATSTPVSIKQYPMSQRARLGIKPHIQRLLD | | gtggtgtgggtctggcagtgcgccaggcaccgctgattat |
| | N-tag: His | QGILVPCQSPWNTPLLPVKKPGTNDYRPVQ | | tccgctgaaagcaaccagcaccccggtgagcattaaacag |
| | | DLREVNKRVEDIHPTVPNPYNLLSGPPPSH | | tatccgatgagccagcgcgcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccagagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttaacgaagcactgcatcgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTTETEVIWAKALPAGTSAQRAEL | | gggttatcgcgcaagcgcaaaaaaagcacagatttgccag |
| | | IALTQALKMAEGKKLNVYTDSRYAFATAHI | | aaacaggtgaaatatctgggttatctgctgaaagaaggtc |
| | | HGEIYRRRGWLTSKGKEIKNKDEILALLKA | | agcgctggctgaccgaagcacgcaaagaaaccgtgatggg |
| | | LFLPKRLSIIHCPGHQKGHSAEARGNRMAD | | tcagccgaccccgaaaaccccgcgccagctgcgccgcttt |
| | | QAARKAAITETPDTSTLLIENSSPNSRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgggtcagccgctggtgattggtgcac |
| | | | | cgcatgcagtggaagcactggtgaaacagccggcaggtcg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccaacggtagcagcctgctgca |
| | | | | ggaaggtcagcgcaaagcaggtgcagcagtgaccaccgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccgat |
| | | | | agccgctatgcatttgcaaccgcacatattcatggtgaaa |
| | | | | tttatcgccgccgcggttggctgaccagcaaaggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgcagaagcacgcggtaaccg |
| | | | | catggcagatcaggcagcacgcaaagcagcaattaccgaa |
| | | | | accccggataccagcaccctgctgattgaaaacagcagcc |
| | | | | cgaacagccgcctgattaac |
| 119 | RTx | TWLSDFPQAWAETGGVGLAVRQAPLIIPLK | 120 | acctggctgagcgattttccgcaggcatgggcagaaaccg |
| | SOLD 029 | ATSTPVSIKQYPMSQRARLGIKPHIQRLLD | | gtggtgtgggtctggcagtgcgccaggcaccgctgattat |
| | N-Tag: His | QGILVPCRSPWNTPLLPVKKPGTSDYRPVQ | | tccgctgaaagcaaccagcaccccggtgagcattaaacag |
| | | DLREVNKRVEDIRPTVPNPYNLLSGPPPSH | | tatccgatgagccagcgcgcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFEEAL | | ccgcagcccgtggaacaccccgctgctgccggtgaaaaaa |
| | | RRDLADFRIQHPDLILLQYVDDLLLAATSE | | ccgggtaccagcgattatcgcccggtgcaggatctgcgcg |
| | | LDCQQGTRALLQTLGNLGYRASAKKAQICQ | | aagtgaacaaacgcgtggaagatattcgcccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMSQPLVIKAPHAVEAL | | ccctgtttgaagaagcactgcgccgcgatctggcagattt |
| | | VKQPAERWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTDETEVIWAKALPAGTSAQRAEL | | gggttatcgcgcaagcgcaaaaaaagcacagatttgccag |
| | | IALTQALKMAEGKKLNVYTNSRYAFATAHI | | aaacaggtgaaatatctgggttatctgctgaaagaaggtc |
| | | HGEIYRRRGWLTSGGKEIKNKDEILALLKA | | agcgctggctgaccgaagcacgcaaagaaaccgtgatggg |
| | | LFLPKRLSIIHCPGHQKGHSVEARGNRMAH | | tcagccgaccccgaaaaccccgcgccagctgcgccgcttt |
| | | QAARRAAITETPDTSTLPIENSSPNSRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgagccagccgctggtgattaaagcac |
| | | | | cgcatgcagtggaagcactggtgaaacagccggcagaacg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccaacggtagcagcctgctgca |
| | | | | ggaaggtcagcgcaaagcaggtgcagcagtgaccgatgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccaac |
| | | | | agccgctatgcatttgcaaccgcacatattcatggtgaaa |
| | | | | tttatcgccgccgcggttggctgaccagcggtggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgtggaagcacgcggtaaccg |
| | | | | catggcacatcaggcagcacgccgcgcagcaattaccgaa |
| | | | | accccggataccagcaccctgccgattgaaaacagcagcc |
| | | | | cgaacagccgcctgattaac |
| 121 | RTx | TWLSDFPQAWAETGGVGLAVRQAPLIIPLK | 122 | acctggctgagcgattttccgcaggcatgggcagaaaccg |
| | SOLD 030 | ATSTPVSIKQYPMSQRARLGIKPHIQRLLD | | gtggtgtgggtctggcagtgcgccaggcaccgctgattat |
| | N-tag: His | QGILVPCRSPWNTPLLPVKKPGTNDYRPVQ | | tccgctgaaagcaaccagcaccccggtgagcattaaacag |
| | | DLREVNKRVEDIHPTVPNPYNLLSGPPPSH | | tatccgatgagccagcgcgcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccgcagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttaacgaagcactgcatcgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTTETEVIWAKALPAGTSAQRAEL | | gggttatcgcgcaagcgcaaaaaaagcacagatttgccag |
| | | IALTQALKMAEGKKLNVYTDSRYAFATAHI | | aaacaggtgaaatatctgggttatctgctgaaagaaggtc |
| | | HGEIYRRRGWLTSKGKEIKNKDEILALLKA | | agcgctggctgaccgaagcacgcaaagaaaccgtgatggg |
| | | LFLPKRLSIIHCPGHQKGHSAEARGNRMAD | | tcagccgaccccgaaaaccccgcgccagctgcgccgcttt |
| | | QAARKAAITETPDTSTLLIENSSPNSRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgggtcagccgctggtgattggtgcac |
| | | | | cgcatgcagtggaagcactggtgaaacagccggcaggtcg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccaacggtagcagcctgctgca |
| | | | | ggaaggtcagcgcaaagcaggtgcagcagtgaccaccgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccgat |
| | | | | agccgctatgcatttgcaaccgcacatattcatggtgaaa |
| | | | | tttatcgccgccgcggttggctgaccagcaaaggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgcagaagcacgcggtaaccg |
| | | | | catggcagatcaggcagcacgcaaagcagcaattaccgaa |
| | | | | accccggataccagcaccctgctgattgaaaacagcagcc |
| | | | | cgaacagccgcctgattaac |
| 123 | RTx | TWLSDFPQAWAETGGVGLAVRQAPLIIPLK | 124 | acctggctgagcgattttccgcaggcatgggcagaaaccg |
| | SOLD 031 | ATSTPVSIKQYPMSQKARLGIKPHIQRLLD | | gtggtgtgggtctggcagtgcgccaggcaccgctgattat |
| | N-tag: His | QGILVPCQSPWNTPLLPVKKPGTNDYRPVQ | | tccgctgaaagcaaccagcaccccggtgagcattaaacag |
| | | DLREVNKRVEDIHPTVPNPYNLLSGPPPSH | | tatccgatgagccagaaagcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccagagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttaacgaagcactgcatcgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTTETEVIWAKALPAGTSAQRAEL | | gggttatcgcgcaagcgcaaaaaaagcacagatttgccag |
| | | IALTQALKMAEGKKLNVYTDSRYAFATAHI | | aaacaggtgaaatatctgggttatctgctgaaagaaggtc |
| | | HGEIYRRRGWLTSKGKEIKNKDEILALLKA | | agcgctggctgaccgaagcacgcaaagaaaccgtgatggg |
| | | LFLPKRLSIIHCPGHQKGHSVEARGNRMAH | | tcagccgaccccgaaaaccccgcgccagctgcgccgcttt |
| | | QAARRAAITETPDTSTLPIENSSPNSRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgggtcagccgctggtgattggtgcac |
| | | | | cgcatgcagtggaagcactggtgaaacagccggcaggtcg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccaacggtagcagcctgctgca |
| | | | | ggaaggtcagcgcaaagcaggtgcagcagtgaccaccgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccgat |
| | | | | agccgctatgcatttgcaaccgcacatattcatggtgaaa |
| | | | | tttatcgccgccgcggttggctgaccagcaaaggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgtggaagcacgcggtaaccg |
| | | | | catggcacatcaggcagcacgccgcgcagcaattaccgaa |
| | | | | accccggataccagcaccctgccgattgaaaacagcagcc |
| | | | | cgaacagccgcctgattaac |
| 125 | RTx | TWLSDFPQAWAETGGVGLAVRQAPLTIPLK | 126 | acctggctgagcgattttccgcaagcatgggcagaaaccg |
| | SOLD 032 | ATSTPVSIKQYPMSQKARLGIKPHIQRLLD | | gtggtgtgggtctggcagtgcgccaggcaccgctgattat |
| | N-tag: His | QGILVPCQSPWNTPLLPVKKPGTNDYRPVQ | | tccgctgaaagcaaccagcaccccggtgagcattaaacag |
| | | DLREVNKRVEDIHPTVPNPYNLLSGPPPSH | | tatccgatgagccagaaagcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFEEAL | | ccagagcccgtggaacaccccgctgctgccggtgaaaaaa |
| | | RRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttgaagaagcactgcgccgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTTETEVIWAKALPAGTSAQRAEL | | gggttatcgcgcaagcgcaaaaaaagcacagatttgccag |
| | | IALTQALKMAEGKKLNVYTNSRYAFATAHI | | aaacaggtgaaatatctgggttatctgttaaaagaaggtc |
| | | QGEIYRRRGFLTSKGKEIKNKDEILALLKA | | agcgctggctgaccgaagcacgcaaagaaaccgtgatggg |
| | | LFLPKRLSIIHCPGHQKGYSAEARVNRMAD | | tcagccgaccccgaaaaccccgcgccagctgcgccgcttt |
| | | QAARKAAITETPDTSTLPIENSSPNSRLIN | | ctgggtaaagcaggtttttgccgcctgtttattccgggtt |
| | | | | ttgcagaaatggcagcaccgctgtatccgctgaccaaacc |
| | | | | gggtactctgtttaactggggtccggatcagcagaaagca |
| | | | | tatcaggaaattaaacaggcactgctgaccgcaccggcac |
| | | | | tgggtctgccggatctgaccaaaccttttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggttgcagcaattgcagtgctgaccaaagatgca |
| | | | | ggtaaactgaccatgggtcagcctctggtgattggtgcac |
| | | | | cgcatgcagtggaagcactggtgaaacagccggcaggtcg |
| | | | | ctggctgagcaaagcacgcatgacccattatcaggcactg |
| | | | | ttactggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgactgatcagccgctgcctgatg |
| | | | | cagatcatacctggtataccaacggtagcagcctgttaca |
| | | | | ggaaggtcagcgtaaagcaggtgcagcagtgaccaccgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccaac |
| | | | | agccgctatgcatttgcaaccgcacatattcagggtgaaa |
| | | | | tctatcgccgccgcggtttcctgaccagcaaaggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggttatagcgcagaagcacgcgtgaaccg |
| | | | | catggcagatcaggcagcacgcaaagcagcaattaccgaa |
| | | | | accccggataccagcaccctgccgattgaaaacagcagcc |
| | | | | cgaacagccgcctgattaac |
| 127 | RTx | TWLSDFPQAWAETGGFGLAVRQAPLIIPLK | 128 | acctggctgagcgattttccgcaggcatgggcagaaaccg |
| | SOLD 033 | ATSTPVSIKQYPISQKARLGIKPHIQRLLD | | gtggttttggtctggcagtgcgccaggcaccgctgattat |
| | N-tag: His | QGILVPCRSPWNTPLLPVKKPGTNDYRPVQ | | tccgctgaaagcaaccagcaccccggtgagcattaaacag |
| | | DLREVNKRVEDIHPTVPNPYNLLSGPPPSH | | tatccgattagccagaaagcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccgcagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttaacgaagcactgcatcgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTTETEVIWAKALPAGTSAQRAEL | | gggttatcgcgcaagcgcaaaaaaagcacagatttgccag |
| | | IALTQALKMAEGKKLNVYTNSRYAFATAHI | | aaacaggtgaaatatctgggttatctgctgaaagaaggtc |
| | | HGEIYRRRGWLTSGGKEIKNKDEILALLKA | | agcgctggctgaccgaagcacgcaaagaaaccgtgatggg |
| | | LFLPKRLSIIHCPGHQKGHSVEARDNRMAH | | tcagccgaccccgaaaaccccgcgccagctgcgccgcttt |
| | | QAARRAAITETPDTSTLPIENSSPNSRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgggtcagccgctggtgattggtgcac |
| | | | | cgcatgcagtggaagcactggtgaaacagccggcaggtcg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccaacggtagcagcctgctgca |
| | | | | ggaaggtcagcgcaaagcaggtgcagcagtgaccaccgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccaac |
| | | | | agccgctatgcatttgcaaccgcacatattcatggtgaaa |
| | | | | tttatcgccgccgcggttggctgaccagcggtggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgtggaagcacgcgataaccg |
| | | | | catggcacatcaggcagcacgccgcgcagcaattaccgaa |
| | | | | accccggataccagcaccctgccgattgaaaacagcagcc |
| | | | | cgaacagccgcctgattaac |
| 129 | RTx | TWLSDFPQAWAETGGVGLAVRQAPLIIPLK | 130 | acctggctgagcgattttccgcaggcatgggcagaaaccg |
| | SOLD 034 | ATSTPVSIKQYPISQKARLGIKPHIQRLLD | | gtggtgtgggtctggcagtgcgccaggcaccgctgattat |
| | N-Tag: his | QGILVPCRSPWNTPLLPVKKPGTNDYRPVQ | | tccgctgaaagcaaccagcaccccggtgagcattaaacag |
| | | DLREVNKRVEDIHPTVPNPYNLLSGPPPSH | | tatccgattagccagaaagcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccgcagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEVKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttaacgaagcactgcatcgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTTETEVIWAKALPAGTSAQRAEL | | gggttatcgcgcaagcgcaaaaaaagcacagatttgccag |
| | | IALTQALKMAEGKKLNVYTDSRYAFATAHI | | aaacaggtgaaatatctgggttatctgctgaaagaaggtc |
| | | QGEIYRRRGWLTSKGKEIKNKDEILALLKA | | agcgctggctgaccgaagcacgcaaagaaaccgtgatggg |
| | | LFLPKRLSIIHCPGHQKGHSAEARGNRMAD | | tcagccgaccccgaaaaccccgcgccagctgcgccgcttt |
| | | QAARKAAITETPDTSTLLIENSSPNSRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaagtgaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgggtcagccgctggtgattggtgcac |
| | | | | cgcatgcagtggaagcactggtgaaacagccggcaggtcg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccaacggtagcagcctgctgca |
| | | | | ggaaggtcagcgcaaagcaggtgcagcagtgaccaccgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccgat |
| | | | | agccgctatgcatttgcaaccgcacatattcagggtgaaa |
| | | | | tttatcgccgccgcggttggctgaccagcaaaggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgcagaagcacgcggtaaccg |
| | | | | catggcagatcaggcagcacgcaaagcagcaattaccgaa |
| | | | | accccggataccagcaccctgctgattgaaaacagcagcc |
| | | | | cgaacagccgcctgattaac |
| 131 | RTx | TWLSDFPQAWAETGGVGLAVRQAPLIIPLK | 132 | acctggctgagcgattttccgcaggcatgggcagaaaccg |
| | SOLD 035 | ATSTPVSIKQYPMSQKARLGIKPHIQRLLD | | gtggtgtgggtctggcagtgcgccaggcaccgctgattat |
| | N-tag: His | QGILVPCQSPWNTPLLPVKKPGTNDYRPVQ | | tccgctgaaagcaaccagcaccccggtgagcattaaacag |
| | | DLREVNKRVEDIHPTVPNPYNLLSGPPPSH | | tatccgatgagccagaaagcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccagagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMSQPLVIKAPHAVEAL | | ccctgtttaacgaagcactgcatcgcgatctggcagattt |
| | | VKQPAERWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTDETEVIWAKALPAGTSAQRAEL | | gggttatcgcgcaagcgcaaaaaaagcacagatttgccag |
| | | IALTQALKMAEGKKLNVYTNSRYAFATAHI | | aaacaggtgaaatatctgggttatctgctgaaagaaggtc |
| | | HGEIYRRRGWLTSGGKEIKNKDEILALLKA | | agcgctggctgaccgaagcacgcaaagaaaccgtgatggg |
| | | LFLPKRLSIIHCPGHQKGHSVEARGNRMAH | | tcagccgaccccgaaaaccccgcgccagctgcgccgcttt |
| | | QAARRAAITETPDTSTLPIENSSPNSRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgagccagccgctggtgattaaagcac |
| | | | | cgcatgcagtggaagcactggtgaaacagccggcagaacg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccaacggtagcagcctgctgca |
| | | | | ggaaggtcagcgcaaagcaggtgcagcagtgaccgatgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccaac |
| | | | | agccgctatgcatttgcaaccgcacatattcatggtgaaa |
| | | | | tttatcgccgccgcggttggctgaccagcggtggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgtggaagcacgcggtaaccg |
| | | | | catggcacatcaggcagcacgccgcgcagcaattaccgaa |
| | | | | accccggataccagcaccctgccgattgaaaacagcagcc |
| | | | | cgaacagccgcctgattaac |
| 133 | RTx | TWLSDFPQAWAETGGVGLAVRQAPLIIPLK | 134 | acctggctgagcgattttccgcaggcatgggcagaaaccg |
| | SOLD 036 | ATSTPVSIKQYPMSQKARLGIKPHIQRLLD | | gtggtgtgggtctggcagtgcgccaggcaccgctgattat |
| | N-tag: His | QGILVPCQSPWNTPLLPVKKPGTNDYRPVQ | | tccgctgaaagcaaccagcaccccggtgagcattaaacag |
| | | DLREVNKRVEDIHPTVPNPYNLLSGPPPSH | | tatccgatgagccagaaagcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccagagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMSQPLVIKAPHAVEAL | | ccctgtttaacgaagcactgcatcgcgatctggcagattt |
| | | VKQPAERWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTTETEVIWAKALPAGTSAQRAEL | | gggttatcgcgcaagcgcaaaaaaagcacagatttgccag |
| | | IALTQALKMAEGKKLNVYTDSRYAFATAHI | | aaacaggtgaaatatctgggttatctgctgaaagaaggtc |
| | | HGEIYRRRGWLTSKGKEIKNKDEILALLKA | | agcgctggctgaccgaagcacgcaaagaaaccgtgatggg |
| | | LFLPKRLSIIHCPGHQKGHSAEARGNRMAD | | tcagccgaccccgaaaaccccgcgccagctgcgccgcttt |
| | | QAARKAAITETPDTSTLLIENSSPNSRLIN | | ctgggtaaagcaggtttttgccgcctgtttattccgggtt |
| | | | | ttgcagaaatggcagcaccgctgtatccgctgaccaaacc |
| | | | | gggtaccctgtttaactggggtccggatcagcagaaagca |
| | | | | tatcaggaaattaaacaggcactgctgaccgcaccggcac |
| | | | | tgggtctgccggatctgaccaaaccgtttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggtggcagcaattgcagtgctgaccaaagatgca |
| | | | | ggtaaactgaccatgagccagccgctggtgattaaagcac |
| | | | | cgcatgcagtggaagcactggtgaaacagccggcagaacg |
| | | | | ctggctgagcaaagcacgcatgacccattatcaggcactg |
| | | | | ctgctggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgaccgatcagccgctgccggatg |
| | | | | cagatcatacctggtataccaacggtagcagcctgctgca |
| | | | | ggaaggtcagcgcaaagcaggtgcagcagtgaccaccgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccgat |
| | | | | agccgctatgcatttgcaaccgcacatattcatggtgaaa |
| | | | | tttatcgccgccgcggttggctgaccagcaaaggtaaaga |
| | | | | aattaaaaacaaagatgaaattctggcactgctgaaagca |
| | | | | ctgtttctgccgaaacgcctgagcattattcattgcccgg |
| | | | | gtcatcagaaaggtcatagcgcagaagcacgcggtaaccg |
| | | | | catggcagatcaggcagcacgcaaagcagcaattaccgaa |
| | | | | accccggataccagcaccctgctgattgaaaacagcagcc |
| | | | | cgaacagccgcctgattaac |
| 135 | RTx | TWLSDFPQAWAETGGMGLAVRQAPLIIPLK | 136 | acctggctgtctgactttccgcaggcttgggcagagaccg |
| | 42B L - Sto7 | ATSTPVSIKQYPLSQKARLGIKPHIQRLLD | | gggggatgggtttagcggtacggcaggcaccactgattat |
| | Trunc 586 | QGILVPCQSPWNTPLLPVKKPGTNDYRPVQ | | accgctgaaagcaacatcgactcctgtctcgataaagcaa |
| | N-Tag: His | DLREVNKRVEDIHPTVPNPYNLLSGPPPSH | | taccctctgtcccaaaaggctcgcttaggcattaagccac |
| | C-Tag: Sto7 | QWYTVLDLKDAFFCLRLHPTSQPLFAFEWR | | atatccagcgtctgctagatcaaggtattctcgttccttg |
| | | DPEMGISGQLTWTRLPQGFKNSPTLFNEAL | | tcagtctccgtggaataccccgttgctgccagttaagaaa |
| | | HRDLADFRIQHPDLILLQYVDDLLLAATSE | | ccgggcacaaacgactatcgcccggttcaggacctgcgag |
| | | LDCQQGTRALLQTLGNLGYRASAKKAQICQ | | aggtaaacaaacgtgtggaagatatccatccgacagtgcc |
| | | KQVKYLGYLLKEGQRWLTEARKETVMGQPT | | caacccctacaatctgttgtctggtcctcctccaagccac |
| | | PKTPRQLRRFLGKAGFCRLFIPGFAEMAAP | | cagtggtacacagtcctggacttaaaagatgcattcttct |
| | | LYPLTKPGTLFNWGPDQQKAYQEIKQALLT | | gcctccgtctgcatccaactagccaacctctgttcgcgtt |
| | | APALGLPDLTKPFELFVDEKQGYAKGVLTQ | | tgaatggcgggacccggaaatgggaattagcggtcagctc |
| | | KLGPWRRPVAYLSKKLDPVAAGWPPCLRMV | | acctggacaagactcccgcaaggattcaagaactccccga |
| | | AAIAVLTKDAGKLTMGQPLVIGAPHAVEAL | | ctctgtttaatgaagcgctgcaccgcgacctggccgactt |
| | | VKQPAGRWLSKARMTHYQALLLDTDRVQFG | | tagaatacagcacccagatcttatccttctgcagtacgtc |
| | | PVVALNPATLLPLPEEGLQHNCLDILAEAH | | gatgatcttttgctggccgcaacaagcgagctggattgtc |
| | | GTRPDLTDQPLPDADHTWYTNGSSLLQEGQ | | aacaggggacgcgtgctcttttacagaccctgggtaatct |
| | | RKAGAAVTTETEVIWAKALPAGTSAQRAEL | | cggttaccgcgcgagcgcaaaaaaagcccaaatatgtcaa |
| | | IALTQALKMAEGKKLNVYTDSRYGGGGSVT | | aaacaggtaaagtatctgggatacctgcttaaagagggtc |
| | | VKFKYKGEELEVDISKIKKVWRVGKMISFT | | agcgatggctcactgaggcacgtaaagagaccgttatggg |
| | | YDDNGKTGRGAVSEKDAPKELLQMLEKSGK | | acagccgacaccaaaaacaccgcgacaactgcggcggttt |
| | | K | | ctgggaaaagcgggtttttgtcgattatttatccctgggt |
| | | | | ttgccgaaatggcggcacctttatatccactcactaagcc |
| | | | | aggcacactgtttaattggggaccggatcaacagaaggcg |
| | | | | taccaggagataaaacaagcgctgttaaccgcccccgctt |
| | | | | taggtctgcctgacctgactaagccgttcgaactgtttgt |
| | | | | agatgaaaaacaaggttacgcgaaaggtgttttaactcag |
| | | | | aaacttgggccatggcgtcgtcctgtcgcgtatctctcta |
| | | | | aaaaacttgacccggttgcggctggctggcctccatgcct |
| | | | | gagaatggtggcagctatagcagtcctgactaaagatgcc |
| | | | | ggtaaattgaccatgggtcagccacttgtgatcggtgctc |
| | | | | cccacgcggtggaggcgttggttaaacagcctgccgggcg |
| | | | | ttggttgtcgaaagctcgcatgacacattatcaggcctta |
| | | | | ttacttgatacggacagagttcagtttggaccggtagtcg |
| | | | | ccctgaacccggccacgttacttccgctgccagaagaagg |
| | | | | tttacagcataattgtctcgatatccttgccgaggctcat |
| | | | | ggaacccgcccagaccttactgaccaaccacttcccgatg |
| | | | | ctgaccacacctggtataccaatggttcatctctgctaca |
| | | | | ggagggtcaacgcaaagcgggtgcagcagttaccactgaa |
| | | | | actgaggttatttgggcgaaagcgctgccagcaggaacga |
| | | | | gcgctcagagagccgagttaattgcgttaacacaggcgct |
| | | | | gaagatggctgaagggaaaaaactgaacgtctataccgac |
| | | | | agccgctatggtggaggtgggtcagtaacagtaaaattta |
| | | | | aatataaaggtgaagagttggaggttgatatttcaaagat |
| | | | | aaaaaaagtgtggagagtcggtaaaatgataagttttacg |
| | | | | tatgatgacaacgggaaaaccggtcggggcgccgtgtccg |
| | | | | aaaaagacgctccgaaggaactactccaaatgcttgagaa |
| | | | | aagtggaaaaaaa |
| 137 | RTx | TWLSDFPQAWAETGGMGLAVRQAPLIIPLK | 138 | acctggctgtctgactttccgcaggcttgggcagagaccg |
| | 42B L - Sto7 | ATSTPVSIKQYPLSQKARLGIKPHIQRLLD | | gggggatgggtttagcggtacggcaggcaccactgattat |
| | Trunc 497 | QGILVPCQSPWNTPLLPVKKPGTNDYRPVQ | | accgctgaaagcaacatcgactcctgtctcgataaagcaa |
| | N-Tag: His | DLREVNKRVEDIHPTVPNPYNLLSGPPPSH | | taccctctgtcccaaaaggctcgcttaggcattaagccac |
| | C-Tag: Sto7 | QWYTVLDLKDAFFCLRLHPTSQPLFAFEWR | | atatccagcgtctgctagatcaaggtattctcgttccttg |
| | | DPEMGISGQLTWTRLPQGFKNSPTLFNEAL | | tcagtctccgtggaataccccgttgctgccagttaagaaa |
| | | HRDLADFRIQHPDLILLQYVDDLLLAATSE | | ccgggcacaaacgactatcgcccggttcaggacctgcgag |
| | | LDCQQGTRALLQTLGNLGYRASAKKAQICQ | | aggtaaacaaacgtgtggaagatatccatccgacagtgcc |
| | | KQVKYLGYLLKEGQRWLTEARKETVMGQPT | | caacccctacaatctgttgtctggtcctcctccaagccac |
| | | PKTPRQLRRFLGKAGFCRLFIPGFAEMAAP | | cagtggtacacagtcctggacttaaaagatgcattcttct |
| | | LYPLTKPGTLFNWGPDQQKAYQEIKQALLT | | gcctccgtctgcatccaactagccaacctctgttcgcgtt |
| | | APALGLPDLTKPFELFVDEKQGYAKGVLTQ | | tgaatggcgggacccggaaatgggaattagcggtcagctc |
| | | KLGPWRRPVAYLSKKLDPVAAGWPPCLRMV | | acctggacaagactcccgcaaggattcaagaactccccga |
| | | AAIAVLTKDAGKLTMGQPLVIGAPHAVEAL | | ctctgtttaatgaagcgctgcaccgcgacctggccgactt |
| | | VKQPAGRWLSKARMTHYQALLLDTDRVQFG | | tagaatacagcacccagatcttatccttctgcagtacgtc |
| | | PVVALNPATLLPLPEEGLQHNCLDGTGGGG | | gatgatcttttgctggccgcaacaagcgagctggattgtc |
| | | VTVKFKYKGEELEVDISKIKKVWRVGKMIS | | aacaggggacgcgtgctcttttacagaccctgggtaatct |
| | | FTYDDNGKTGRGAVSEKDAPKELLQMLEKS | | cggttaccgcgcgagcgcaaaaaaagcccaaatatgtcaa |
| | | GKK | | aaacaggtaaagtatctgggatacctgcttaaagagggtc |
| | | | | agcgatggctcactgaggcacgtaaagagaccgttatggg |
| | | | | acagccgacaccaaaaacaccgcgacaactgcggcggttt |
| | | | | ctgggaaaagcgggtttttgtcgattatttatccctgggt |
| | | | | ttgccgaaatggcggcacctttatatccactcactaagcc |
| | | | | aggcacactgtttaattggggaccggatcaacagaaggcg |
| | | | | taccaggagataaaacaagcgctgttaaccgcccccgctt |
| | | | | taggtctgcctgacctgactaagccgttcgaactgtttgt |
| | | | | agatgaaaaacaaggttacgcgaaaggtgttttaactcag |
| | | | | aaacttgggccatggcgtcgtcctgtcgcgtatctctcta |
| | | | | aaaaacttgacccggttgcggctggctggcctccatgcct |
| | | | | gagaatggtggcagctatagcagtcctgactaaagatgcc |
| | | | | ggtaaattgaccatgggtcagccacttgtgatcggtgctc |
| | | | | cccacgcggtggaggcgttggttaaacagcctgccgggcg |
| | | | | ttggttgtcgaaagctcgcatgacacattatcaggcctta |
| | | | | ttacttgatacggacagagttcagtttggaccggtagtcg |
| | | | | ccctgaacccggccacgttacttccgctgccagaagaagg |
| | | | | tttacagcataattgtctcgatgggacgggaggaggcgga |
| | | | | gtaacagtaaaatttaaatataaaggtgaagagttggagg |
| | | | | ttgatatttcaaagataaaaaaagtgtggagagtcggtaa |
| | | | | aatgataagttttacgtatgatgacaacgggaaaaccggt |
| | | | | cggggcgccgtgtccgaaaaagacgctccgaaggaactac |
| | | | | tccaaatgcttgagaaaagtggaaaaaaa |
| 139 | RTx | TWLSDFPQAWAETGGMGLAVRQAPLIIPLK | 140 | acctggctgtctgactttccgcaggcttgggcagagaccg |
| | 42B L - Sto7 | ATSTPVSIKQYPLSQKARLGIKPHIQRLLD | | gggggatgggtttagcggtacggcaggcaccactgattat |
| | Trunc 515 | QGILVPCQSPWNTPLLPVKKPGTNDYRPVQ | | accgctgaaagcaacatcgactcctgtctcgataaagcaa |
| | N-tag: His | DLREVNKRVEDIHPTVPNPYNLLSGPPPSH | | taccctctgtcccaaaaggctcgcttaggcattaagccac |
| | C-tag: Sto7 | QWYTVLDLKDAFFCLRLHPTSQPLFAFEWR | | atatccagcgtctgctagatcaaggtattctgttccttgt |
| | | DPEMGISGQLTWTRLPQGFKNSPTLFNEAL | | cagtctccgtggaataccccgttgctgccagttaagaaac |
| | | HRDLADFRIQHPDLILLQYVDDLLLAATSE | | cgggcacaaacgactatcgcccggttcaggacctgcgaga |
| | | LDCQQGTRALLQTLGNLGYRASAKKAQICQ | | ggtaaacaaacgtgtggaagatatccatccgacagtgccc |
| | | KQVKYLGYLLKEGQRWLTEARKETVMGQPT | | aacccctacaatctgttgtctggtcctcctccaagccacc |
| | | PKTPRQLRRFLGKAGFCRLFIPGFAEMAAP | | agtggtacacagtcctggacttaaaagatgcattcttctg |
| | | LYPLTKPGTLFNWGPDQQKAYQEIKQALLT | | cctccgtctgcatccaactagccaacctctgttcgcgttt |
| | | APALGLPDLTKPFELFVDEKQGYAKGVLTQ | | gaatggcgggacccggaaatgggaattagcggtcagctca |
| | | KLGPWRRPVAYLSKKLDPVAAGWPPCLRMV | | cctggacaagactcccgcaaggattcaagaactccccgac |
| | | AAIAVLTKDAGKLTMGQPLVIGAPHAVEAL | | tctgtttaatgaagcgctgcaccgcgacctggccgacttt |
| | | VKQPAGRWLSKARMTHYQALLLDTDRVQFG | | agaatacagcacccagatcttatccttctgcagtacgtcg |
| | | PVVALNPATLLPLPEEGLQHNCLDILAEAH | | atgatcttttgctggccgcaacaagcgagctggattgtca |
| | | GTRPDLTDQPLPGGGGSVTVKFKYKGEELE | | acaggggacgcgtgctcttttacagaccctgggtaatctc |
| | | VDISKIKKVWRVGKMISFTYDDNGKTGRGA | | ggttaccgcgcgagcgcaaaaaaagcccaaatatgtcaaa |
| | | VSEKDAPKELLQMLEKSGKK | | aacaggtaaagtatctgggatacctgcttaaagagggtca |
| | | | | gcgatggctcactgaggcacgtaaagagaccgttatggga |
| | | | | cagccgacaccaaaaacaccgcgacaactgcggcggtttc |
| | | | | tgggaaaagcgggtttttgtcgattatttatccctgggtt |
| | | | | tgccgaaatggcggcacctttatatccactcactaagcca |
| | | | | ggcacactgtttaattggggaccggatcaacagaaggcgt |
| | | | | accaggagataaaacaagcgctgttaaccgcccccgcttt |
| | | | | aggtctgcctgacctgactaagccgttcgaactgtttgta |
| | | | | gatgaaaaacaaggttacgcgaaaggtgttttaactcaga |
| | | | | aacttgggccatggcgtcgtcctgtcgcgtatctctctaa |
| | | | | aaaacttgacccggttgcggctggctggcctccatgcctg |
| | | | | agaatggtggcagctatagcagtcctgactaaagatgccg |
| | | | | gtaaattgaccatgggtcagccacttgtgatcggtgctcc |
| | | | | ccacgcggtggaggcgttggttaaacagcctgccgggcgt |
| | | | | tggttgtcgaaagctcgcatgacacattatcaggccttat |
| | | | | tacttgatacggacagagttcagtttggaccggtagtcgc |
| | | | | cctgaacccggccacgttacttccgctgccagaagaaggt |
| | | | | ttacagcataattgtctcgatatccttgccgaggctcatg |
| | | | | gaacccgcccagaccttactgaccaaccacttcccggtgg |
| | | | | aggtgggtcagtaacagtaaaatttaaatataaaggtgaa |
| | | | | gagttggaggttgatatttcaaagataaaaaaagtgtgga |
| | | | | gagtcggtaaaatgataagttttacgtatgatgacaacgg |
| | | | | gaaaaccggtcggggcgccgtgtccgaaaaagacgctccg |
| | | | | aaggaactactccaaatgcttgagaaaagtggaaaaaaa |
| 141 | RTx | TWLSDFPQAWAETGGVGLAVRQAPLIIPLK | 142 | acctggctgagcgattttccgcaagcatgggcagaaaccg |
| | SOLD 037 | ATSTPVSIKQYPMSQKARLGIKPHIQRLLD | | gtggtgtgggtctggcagtgcgccaggcaccgctgattat |
| | N-tag: His | QGILVPCQSPWNTPLLPVKKPGTSDYRPVQ | | tccgctgaaagcaaccagcaccccggtgagcattaaacag |
| | | DLREVNKRVEDIHPTVPNPYNLLSGPPPSH | | tatccgatgagccagaaagcacgcctgggtattaaaccgc |
| | | QWYTVLDLKDAFFCLRLHPTSQPLFAFEWR | | atattcagcgcctgctggatcagggtattctggtgccgtg |
| | | DPEMGISGQLTWTRLPQGFKNSPTLFVEAL | | ccagagcccgtggaacaccccgctgctgccggtgaaaaaa |
| | | RRDLADFRIQHPDLILLQYVDDLLLAATSE | | ccgggtaccagcgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttgtggaagcactgcgccgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTTETEVIWAKALPAGTSAQRAEL | | gggttatcgcgcaagcgcaaaaaaagcacagatttgccag |
| | | IALTQALKMAEGKKLNVYTDSRYAFATAHI | | aaacaggtgaaatatctgggttatctgttaaaagaaggtc |
| | | HGEIYRRRGWLTSKGKEIKNKDEILALLKA | | agcgctggctgaccgaagcacgcaaagaaaccgtgatggg |
| | | LFLPKRLSIIHCPGHQKGHSAEARGNRMAD | | tcagccgaccccgaaaaccccgcgccagctgcgccgcttt |
| | | QAARKAAITETPDTSTLLIENSSPNSRLIN | | ctgggtaaagcaggtttttgccgcctgtttattccgggtt |
| | | | | ttgcagaaatggcagcaccgctgtatccgctgaccaaacc |
| | | | | gggtactctgtttaactggggtccggatcagcagaaagca |
| | | | | tatcaggaaattaaacaggcactgctgaccgcaccggcac |
| | | | | tgggtctgccggatctgaccaaaccttttgaactgtttgt |
| | | | | ggatgaaaaacagggttatgcaaaaggtgtgctgacccag |
| | | | | aaactgggtccgtggcgccgcccggtggcatatctgagca |
| | | | | aaaaactggatccggtggcagcaggttggccgccgtgcct |
| | | | | gcgcatggttgcagcaattgcagtgctgaccaaagatgca |
| | | | | ggtaaactgaccatgggtcagcctctggtgattggtgcac |
| | | | | cgcatgcagtggaagcattagtgaaacagccggcaggtcg |
| | | | | ctggctgagcaaagcacgcatgacccattatcaggcactg |
| | | | | ttactggataccgatcgcgtgcagtttggtccggtggtgg |
| | | | | cactgaacccggcaaccctgctgccgctgccggaagaagg |
| | | | | tctgcagcataactgcctggatattctggcagaagcacat |
| | | | | ggtacccgcccggatctgactgatcagccgctgcctgatg |
| | | | | cagatcatacctggtataccaacggtagcagcctgttaca |
| | | | | ggaaggtcagcgtaaagcaggtgcagcagtgaccaccgaa |
| | | | | accgaagtgatttgggcaaaagcactgccggcaggtacca |
| | | | | gcgcacagcgcgcagaactgattgcactgacccaggcact |
| | | | | gaaaatggcagaaggtaaaaaactgaacgtgtataccgat |
| | | | | agccgctatgcatttgcaaccgcacatattcatggtgaaa |
| | | | | tctatcgccgccgcggttggctgaccagcaaaggtaaaga |
| | | | | aattaaaaacaaagatgagatcctggcactgctgaaagca |
| | | | | ctgtttctgccgaaacgcctgagcattattcattgcccgg |
| | | | | gtcatcagaaaggtcatagcgcagaagcacgcggtaaccg |
| | | | | catggcagatcaggcagcacgcaaagcagcaattaccgaa |
| | | | | accccggataccagcaccctgctgattgaaaacagcagcc |
| | | | | cgaacagccgcctgattaac |
| 143 | RTx | TWLSDFPQAWAETGGMGLAVRQAPLIIPLK | 144 | acttggctgtctgatttccctcaggcgtgggccgaaacgg |
| | 42B | ATSTPVSIKQYPMSQKARLGIKPHIQRLLD | | gtggcatgggtctggcagtgcgtcaggcaccgctgattat |
| | N-tag: His | QGILVPCQSPWNTPLLPVKKPGTNDYRPVQ | | tccgctgaaagcgacgtcgaccccggtgagcatcaagcaa |
| | | DLREVNKRVEDIHPTVPNPYNLLSGPPPSH | | tatccgatgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcagagcccgtggaataccccgcttctcccggtgaagaaa |
| | | HRDLADFRIQHPDLILLQYVDDLLLAATSE | | ccgggcacgaacgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMGQPLVIGAPHAVEAL | | cgctgtttaacgaagcgctgcaccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTTETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaggttc |
| | | QAARKAAITETPDTSTLLIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgggtcaaccgctggtgatcggcgcac |
| | | | | cgcatgcagtcgaggcccttgttaagcaaccggcaggaag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccaccgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccaaaggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgctgatcgagaatagcagcc |
| | | | | cgaacagccgtctgatcaat |
| 145 | RTx | TWLSDFPQAWAETGGMGLAVRQAPLIIPLK | 146 | acttggctgtctgatttccctcaggcgtgggccgaaacgg |
| | 42B L | ATSTPVSIKQYPLSQKARLGIKPHIQRLLD | | gtggcatgggtctggcagtgcgtcaggcaccgctgattat |
| | N-tag: His | QGILVPCQSPWNTPLLPVKKPGTNDYRPVQ | | tccgctgaaagcgacgtcgaccccggtgagcatcaagcaa |
| | | DLREVNKRVEDIHPTVPNPYNLLSGPPPSH | | tatccgctgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcagagcccgtggaataccccgcttctcccggtgaagaaa |
| | | HRDLADFRIQHPDLILLQYVDDLLLAATSE | | ccgggcacgaacgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMGQPLVIGAPHAVEAL | | cgctgtttaacgaagcgctgcaccgtgacctggcggattt |
| | | VKQPAGRWLSKARMTHYQALLLDTDRVQFG | | ccgtatccagcacccggacctgatcttgctgcagtacgtt |
| | | PVVALNPATLLPLPEEGLQHNCLDILAEAH | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTTETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSKGKEIKNKDEILALLKA | | aacgttggctgaccgaagcccgcaaagaaactgtcatggg |
| | | LFLPKRLSIIHCPGHQKGHSAEARGNRMAD | | tcagccgaccccgaaaacgccacgccaactgcgtaggttc |
| | | QAARKAAITETPDTSTLLIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgggtcaaccgctggtgatcggcgcac |
| | | | | cgcatgcagtcgaggcccttgttaagcaaccggcaggaag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcgcac |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccaccgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccaaaggcaaaga |
| | | | | gatcaagaataaagacgaaattctggcgctgctgaaagcg |
| | | | | ctgttcctgccgaaacgtctgtcgatcatccattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgctgatcgagaatagcagcc |
| | | | | cgaacagccgtctgatcaat |
| 147 | RTx | TWLSDFPQAWAETGGMGLAVRQAPLIIPLK | 148 | acttggctgtctgatttccctcaggcgtgggccgaaacgg |
| | 50A+G | ATSTPVSIKQYPLSQKARLGIKPHIQRLLD | | gtggcatgggtctggcagtgcgtcaggcaccgctgattat |
| | N-tag: His | QGILVPCQSPWNTPLLPVKKPGTNDYRPVQ | | tccgctgaaagcgacgtcgaccccggtgagcatcaagcaa |
| | | DLREVNKRVEDIHPTVPNPYNLLSGPPPSH | | tatccgctgtcccaaaaggcgcgcttaggtattaagccgc |
| | | QWYTVLDLKDAFFCLRLHPTSQPLFAFEWR | | acattcagcgtctgctggatcaaggtattctggttccgtg |
| | | DPEMGISGQLTWTRLPQGFKNSPTLFNEAL | | tcagagcccgtggaataccccgcttctcccggtgaagaaa |
| | | HRDLADFRIQHPDLILLQYVDDLLLAATSE | | ccgggcacgaacgattaccgtccagtccaagacttgcgcg |
| | | LDCQQGTRALLQTLGNLGYRASAKKAQICQ | | aagttaacaagcgcgttgaagatattcacccgaccgtccc |
| | | KQVKYLGYLLKEGQRWLTEARKETVMGQPT | | gaacccgtacaatctgctgagcggtccgccgccaagccac |
| | | PKTPRQLRRFLGKAGFCRLFIPGFAEMAAP | | caatggtacaccgtgctggatctgaaagatgctttcttct |
| | | LYPLTKPGTLFNWGPDQQKAYQEIKQALLT | | gtctgcgtctgcacccaaccagccagcctctgtttgcatt |
| | | APALGLPDLTKPFELFVDEKQGYAKGVLTQ | | tgagtggcgtgaccctgagatgggtattagcggccagctg |
| | | KLGPWRRPVAYLSKKLDPVAAGWPPCLRMV | | acgtggacccgcctgccgcaaggttttaagaattccccta |
| | | AAIAVLTKDAGKLTMGQPLVIGAPHAVEAL | | cgctgtttaacgaagcgctgcaccgtgacctggcggattt |
| | | VKQPPDRWLSKARMTHYQALLLDTDRVQFG | | ccgtatccagcacccggacctgatcttgctgcagtacgtt |
| | | PVVALNPATLLPLPEEGLQHNCLDILAEAV | | gatgacctgttgctggcggcgacgagcgagctggattgcc |
| | | GTRPDLTDQPLPDADHTWYTNGSSLLQEGQ | | aacagggcacccgtgcgctgttgcagaccttgggtaacct |
| | | RKAGAAVTTETEVIWAKALPAGTSAQRAEL | | gggttatcgcgctagcgcgaagaaagcgcagatttgccaa |
| | | IALTQALKMAEGKKLNVYTDSRYAFATAHI | | aaacaagttaagtatctgggctacctgttaaaggaaggcc |
| | | HGEIYRRRGWLTSKGKEIKNKDEILALLKA | | aacgttggctgaccgaagcccgcaaagaaactgtcatggg |
| | | LFLPKRLSIIYCPGHQKGHSAEARGNRMAD | | tcagccgaccccgaaaacgccacgccaactgcgtaggttc |
| | | QAARKAAITETPDTSTLLIENSSPNSRLIN | | ttgggcaaagcgggtttctgccgcctgttcatcccgggct |
| | | | | ttgccgaaatggcagccccgctgtatccgttgaccaagcc |
| | | | | gggcaccctgttcaactggggtccggaccagcagaaagcg |
| | | | | taccaagaaattaaacaagcactgctgacggcaccggcgc |
| | | | | tgggtctgccggacctgaccaagccgtttgagctgttcgt |
| | | | | ggatgagaagcaaggttacgcgaagggcgtgttgacccag |
| | | | | aaattgggtccgtggcgtcgtccggttgcatacctgtcca |
| | | | | agaaactggacccggttgctgctggttggccgccttgcct |
| | | | | gcgcatggttgccgctatcgcggtgctgactaaagacgcg |
| | | | | ggtaagctgacgatgggtcaaccgctggtgatcggtgcac |
| | | | | cgcatgcagtcgaggcccttgttaagcaaccgccggatag |
| | | | | atggctgagcaaggcgcgtatgacgcattaccaggcactg |
| | | | | ctgttggacaccgatcgtgtgcagtttggcccggtcgttg |
| | | | | cgctcaacccggcgaccctgctgccgctcccggaagaagg |
| | | | | cttgcagcacaactgtttggacatcctggcagaggcggtg |
| | | | | ggcactcgcccggatctgacggaccagccgctgccggacg |
| | | | | ccgatcatacctggtatacgaatggtagcagcctgttgca |
| | | | | agagggtcagcgtaaggccggtgccgcggtcaccaccgag |
| | | | | actgaagtgatttgggctaaagcattgcctgcgggtacca |
| | | | | gcgcgcagcgtgccgagctgatcgcactgacccaagcgct |
| | | | | gaaaatggctgagggtaagaaactgaatgtgtacacggat |
| | | | | agccgttatgcctttgcgaccgcccacattcacggcgaga |
| | | | | tctatcgccgtcgcggctggctgacgtccaaaggcaaaga |
| | | | | gatcaagaataaagacgaaattctggcgctgctgaaagcg |
| | | | | ctgttcctgccgaaacgtctgtcgatcatctattgcccgg |
| | | | | gtcaccagaaaggccacagcgcagaggcgcgtggtaatcg |
| | | | | catggctgaccaggctgcgcgtaaagccgcaattaccgaa |
| | | | | accccggacaccagcacgctgctgatcgagaatagcagcc |
| | | | | cgaacagccgtctgatcaat |
| 149 | RTx | TWLSDFPQAWAETGGVGLAVRQAPLIIPLK | 150 | acctggctgagcgattttccgcaggcatgggcagaaaccg |
| | SOLD 34 - | ATSTPVSIKQYPISQKARLGIKPHIQRLLD | | gtggtgtgggtctggcagtgcgccaggcaccgctgattat |
| | Sto7 (K13L) | QGILVPCRSPWNTPLLPVKKPGTNDYRPVQ | | tccgctgaaagcaaccagcaccccggtgagcattaaacag |
| | N-tag: His | DLREVNKRVEDIHPTVPNPYNLLSGPPPSH | | tatccgattagccagaaagcacgcctgggtattaaaccgc |
| | C-Tag: Sto7 | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccgcagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEVKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttaacgaagcactgcatcgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTTETEVIWAKALPAGTSAQRAEL | | gggttatcgcgcaagcgcaaaaaaagcacagatttgccag |
| | | IALTQALKMAEGKKLNVYTDSRYAFATAHI | | aaacaggtgaaatatctgggttatctgctgaaagaaggtc |
| | | QGEIYRRRGWLTSKGKEIKNKDEILALLKA | | agcgctggctgaccgaagcacgcaaagaaaccgtgatggg |
| | | LFLPKRLSIIHCPGHQKGHSAEARGNRMAD | | tcagccgaccccgaaaaccccgcgccagctgcgccgcttt |
| | | QAARKAAITETPDTSTLLIENSSPNSRLIN | | ctgggtaaagcaggtttttgccgcctgtttattccgggtt |
| | | GGGGSVTVKFKYKGEELEVDISKIKKVWRV | | ttgcagaaatggcagcaccgctgtatccgctgaccaaacc |
| | | GKMISFTYDDNGKTGRGAVSEKDAPKELLQ | | gggtaccctgtttaactggggtccggatcagcagaaagca |
| | | MLEKSGKK | | tatcaggaagtgaaacaggcactgclgaccgcaccggcac |
| | | | | igggicigccggatctgaccaaaccgittgaactgittgg |
| | | | | gatgaaaaacagggttatgcaaaaggtgtgctgacccaga |
| | | | | aactgggtccgtggcgccgcccggtggcatatctgagcaa |
| | | | | aaaactggatccggtggcagcaggttggccgccgtgcctg |
| | | | | cgcatggtggcagcaattgcagtgctgaccaaagatgcag |
| | | | | gtaaactgaccatgggtcagccgctggtgattggtgcacc |
| | | | | gcatgcagtggaagcactggtgaaacagccggcaggtcgc |
| | | | | tggctgagcaaagcacgcatgacccattatcaggcactgc |
| | | | | tgctggataccgatcgcgtgcagtttggtccggtggtggc |
| | | | | actgaacccggcaaccctgctgccgctgccggaagaaggt |
| | | | | ctgcagcataactgcctggatattctggcagaagcacatg |
| | | | | gtacccgcccggatctgaccgatcagccgctgccggatgc |
| | | | | agatcatacctggtataccaacggtagcagcctgctgcag |
| | | | | gaaggtcagcgcaaagcaggtgcagcagtgaccaccgaaa |
| | | | | ccgaagtgatttgggcaaaagcactgccggcaggtaccag |
| | | | | cgcacagcgcgcagaactgattgcactgacccaggcactg |
| | | | | aaaatggcagaaggtaaaaaactgaacgtgtataccgata |
| | | | | gccgctatgcatttgcaaccgcacatattcagggtgaaat |
| | | | | ttatcgccgccgcggttggctgaccagcaaaggtaaagaa |
| | | | | attaaaaacaaagatgaaattctggcactgctgaaagcac |
| | | | | tgtttctgccgaaacgcctgagcattattcattgcccggg |
| | | | | tcatcagaaaggtcatagcgcagaagcacgcggtaaccgc |
| | | | | atggcagatcaggcagcacgcaaagcagcaattaccgaaa |
| | | | | ccccggataccagcaccctgctgattgaaaacagcagccc |
| | | | | gaacagccgcctgattaacggtggaggtgggtcagtaaca |
| | | | | gtaaaatttaaatataaaggtgaagagttggaggttgata |
| | | | | tttcaaagataaaaaaagtgtggagagtcggtaaaatgat |
| | | | | aagttttacgtatgatgacaacgggaaaaccggtcggggc |
| | | | | gccgtgtccgaaaaagacgctccgaaggaactactccaaa |
| | | | | tgcttgagaaaagtggaaaaaaa |
| 151 | RTx | TWLSDFPQAWAETGGVGLAVRQAPLIIPLK | 152 | acctggctgagcgattttccgcaggcatgggcagaaaccg |
| | SOLD 025 - | ATSTPVSIKQYPMSQKARLGIKPHIQRLLD | | gtggtgtgggtctggcagtgcgccaggcaccgctgattat |
| | Sto7 (K13L) | QGILVPCQSPWNTPLLPVKKPGTNDYRPVQ | | tccgctgaaagcaaccagcaccccggtgagcattaaacag |
| | N-tag: his | DLREVNKRVEDIHPTVPNPYNLLSGPPPSH | | tatccgatgagccagaaagcacgcctgggtattaaaccgc |
| | C-tag: Sto7 | QWYTVLDLKDAFFCLRLHPTSQPLFAFEWR | | atattcagcgcctgctggatcagggtattctggtgccgtg |
| | | DPEMGISGQLTWTRLPQGFKNSPTLFNEAL | | ccagagcccgtggaacaccccgctgctgccggtgaaaaaa |
| | | HRDLADFRIQHPDLILLQYVDDLLLAATSE | | ccgggtaccaacgattatcgcccggtgcaggatctgcgcg |
| | | LDCQQGTRALLQTLGNLGYRASAKKAQICQ | | aagtgaacaaacgcgtggaagatattcatccgaccgtgcc |
| | | KQVKYLGYLLKEGQRWLTEARKETVMGQPT | | gaacccgtataacctgctgagcggtccgccgccgagccat |
| | | PKTPRQLRRFLGKAGFCRLFIPGFAEMAAP | | cagtggtataccgtgctggatctgaaagatgcattttttt |
| | | LYPLTKPGTLFNWGPDQQKAYQEIKQALLT | | gcctgcgcctgcatccgaccagccagccgctgtttgcatt |
| | | APALGLPDLTKPFELFVDEKQGYAKGVLTQ | | tgaatggcgcgatccggaaatgggtattagcggtcagctg |
| | | KLGPWRRPVAYLSKKLDPVAAGWPPCLRMV | | acctggacccgcctgccgcagggttttaaaaacagcccga |
| | | AAIAVLTKDAGKLTMGQPLVIGAPHAVEAL | | ccctgtttaacgaagcactgcatcgcgatctggcagattt |
| | | VKQPAGRWLSKARMTHYQALLLDTDRVQFG | | tcgcattcagcatccggatctgattctgctgcagtatgtg |
| | | PVVALNPATLLPLPEEGLQHNCLDILAEAH | | gatgatctgctgctggcagcaaccagcgaactggattgcc |
| | | GTRPDLTDQPLPDADHTWYTNGSSLLQEGQ | | agcagggtacccgcgcactgctgcagaccctgggtaacct |
| | | RKAGAAVTDETEVIWAKALPAGTSAQRAEL | | gggttatcgcgcaagcgcaaaaaaagcacagatttgccag |
| | | IALTQALKMAEGKKLNVYTNSRYAFATAHI | | aaacaggtgaaatatctgggttatctgctgaaagaaggtc |
| | | HGEIYRRRGWLTSGGKEIKNKDEILALLKA | | agcgctggctgaccgaagcacgcaaagaaaccgtgatggg |
| | | LFLPKRLSIIHCPGHQKGHSVEARGNRMAH | | tcagccgaccccgaaaaccccgcgccagctgcgccgcttt |
| | | QAARRAAITETPDTSTLPIENSSPNSRLIN | | ctgggtaaagcaggtttttgccgcctgtttattccgggtt |
| | | GGGGSVTVKFKYKGEELEVDISKIKKVWRV | | ttgcagaaatggcagcaccgctgtatccgctgaccaaacc |
| | | GKMISFTYDDNGKTGRGAVSEKDAPKELLQ | | gggtaccctgtttaactggggtccggatcagcagaaagca |
| | | MLEKSGKK | | tatcaggaaattaaacaggcactgctgaccgcaccggcac |
| | | | | tgggtctgccggatctgaccaaaccgtttgaactgtttgt |
| | | | | ggatgaaaaacagggitatgcaaaaggigigctgacccag |
| | | | | aaactgggtccglggcgccgcccggggcatatclgagcaa |
| | | | | aaaactggatccggtggcagcaggttggccgccgtgcctg |
| | | | | cgcatggtggcagcaattgcagtgctgaccaaagatgcag |
| | | | | gtaaactgaccatgggtcagccgctggtgattggtgcacc |
| | | | | gcatgcagtggaagcactggtgaaacagccggcaggtcgc |
| | | | | tggctgagcaaagcacgcatgacccattatcaggcactgc |
| | | | | tgctggataccgatcgcgtgcagtttggtccggtggtggc |
| | | | | actgaacccggcaaccctgctgccgctgccggaagaaggt |
| | | | | ctgcagcataactgcctggatattctggcagaagcacatg |
| | | | | gtacccgcccggatctgaccgatcagccgctgccggatgc |
| | | | | agatcatacctggtataccaacggtagcagcctgctgcag |
| | | | | gaaggtcagcgcaaagcaggtgcagcagtgaccgatgaaa |
| | | | | ccgaagtgatttgggcaaaagcactgccggcaggtaccag |
| | | | | cgcacagcgcgcagaactgattgcactgacccaggcactg |
| | | | | aaaatggcagaaggtaaaaaactgaacgtgtataccaaca |
| | | | | gccgctatgcatttgcaaccgcacatattcatggtgaaat |
| | | | | ttatcgccgccgcggttggctgaccagcggtggtaaagaa |
| | | | | attaaaaacaaagatgaaattctggcactgctgaaagcac |
| | | | | tgtttctgccgaaacgcctgagcattattcattgcccggg |
| | | | | tcatcagaaaggtcatagcgtggaagcacgcggtaaccgc |
| | | | | atggcacatcaggcagcacgccgcgcagcaattaccgaaa |
| | | | | ccccggataccagcaccctgccgattgaaaacagcagccc |
| | | | | gaacagccgcctgattaacggtggaggtgggtcagtaaca |
| | | | | gtaaaatttaaatataaaggtgaagagttggaggttgata |
| | | | | tttcaaagataaaaaaagtgtggagagtcggtaaaatgat |
| | | | | aagttttacgtatgatgacaacgggaaaaccggtcggggc |
| | | | | gccgtgtccgaaaaagacgctccgaaggaactactccaaa |
| | | | | tgcttgagaaaagtggaaaaaaa |
| 153 | RTx | TWLSDFPQAWAETGGMGLAVRQAPLIIPLK | 154 | acctggctgtctgactttccgcaggcttgggcagagaccg |
| | 42B - Sto7 | ATSTPVSIKQYPMSQKARLGIKPHIQRLLD | | gggggatgggtttagcggtacggcaggcaccactgattat |
| | K13L | QGILVPCQSPWNTPLLPVKKPGTNDYRPVQ | | accgctgaaagcaacatcgactcctgtctcgataaagcaa |
| | N-tag: His | DLREVNKRVEDIHPTVPNPYNLLSGPPPSH | | taccctatgtcccaaaaggctcgcttaggcattaagccac |
| | C-Tag: Sto7 | QWYTVLDLKDAFFCLRLHPTSQPLFAFEWR | | atatccagcgtctgctagatcaaggtattctcgttccttg |
| | | DPEMGISGQLTWTRLPQGFKNSPTLFNEAL | | tcagtctccgtggaataccccgttgctgccagttaagaaa |
| | | HRDLADFRIQHPDLILLQYVDDLLLAATSE | | ccgggcacaaacgactatcgcccggttcaggacctgcgag |
| | | LDCQQGTRALLQTLGNLGYRASAKKAQICQ | | aggtaaacaaacgtgtggaagatatccatccgacagtgcc |
| | | KQVKYLGYLLKEGQRWLTEARKETVMGQPT | | caacccctacaatctgttgtctggtcctcctccaagccac |
| | | PKTPRQLRRFLGKAGFCRLFIPGFAEMAAP | | cagtggtacacagtcctggacttaaaagatgcattcttct |
| | | LYPLTKPGTLFNWGPDQQKAYQEIKQALLT | | gcctccgtctgcatccaactagccaacctctgttcgcgtt |
| | | APALGLPDLTKPFELFVDEKQGYAKGVLTQ | | tgaatgggggacccggaaatgggaattagcggtcagctca |
| | | KLGPWRRPVAYLSKKLDPVAAGWPPCLRMV | | cctggacaagactcccgcaaggattcaagaactccccgac |
| | | AAIAVLTKDAGKLTMGQPLVIGAPHAVEAL | | tctgtttaatgaagcgctgcaccgcgacctggccgacttt |
| | | VKQPAGRWLSKARMTHYQALLLDTDRVQFG | | agaatacagcacccagatcttatccttctgcagtacgtcg |
| | | PVVALNPATLLPLPEEGLQHNCLDILAEAH | | atgatcttttgctggccgcaacaagcgagctggattgtca |
| | | GTRPDLTDQPLPDADHTWYTNGSSLLQEGQ | | acaggggacgcgtgctcttttacagaccctgggtaatctc |
| | | RKAGAAVTTETEVIWAKALPAGTSAQRAEL | | ggttaccgcgcgagcgcaaaaaaagcccaaatatgtcaaa |
| | | IALTQALKMAEGKKLNVYTDSRYAFATAHI | | aacaggtaaagtatctgggatacctgcttaaagagggtca |
| | | HGEIYRRRGWLTSKGKEIKNKDEILALLKA | | gcgatggctcactgaggcacgtaaagagaccgttatggga |
| | | LFLPKRLSIIHCPGHQKGHSAEARGNRMAD | | cagccgacaccaaaaacaccgcgacaactgcggcggtttc |
| | | QAARKAAITETPDTSTLLIENSSPNSRLIN | | tgggaaaagcgggtttttgtcgattatttatccctgggtt |
| | | GGGGSVTVKFKYKGEELEVDISKIKKVWRV | | tgccgaaatggcggcacctttatatccactcactaagcca |
| | | GKMISFTYDDNGKTGRGAVSEKDAPKELLQ | | ggcacactgtttaattggggaccggatcaacagaaggcgt |
| | | MLEKSGKK | | accaggagataaaacaagcgctgttaaccgcccccgcttt |
| | | | | aggtctgcctgacctgactaagccgttcgaactgtttgta |
| | | | | gatgaaaaacaaggttacgcgaaaggtgttttaactcaga |
| | | | | aacttgggccatggcgtcgtcctgtcgcgtatctctctaa |
| | | | | aaaacttgacccggttgcggctggctggcctccatgcctg |
| | | | | agaatggtggcagctatagcagtcctgactaaagatgccg |
| | | | | gtaaattgaccatgggtcagccacttgtgatcggtgctcc |
| | | | | ccacgcggtggaggcgttggttaaacagcctgccgggcgt |
| | | | | tggttgtcgaaagctcgcatgacacattatcaggccttat |
| | | | | tacttgatacggacagagttcagtttggaccggtagtcgc |
| | | | | cctgaacccggccacgttacttccgctgccagaagaaggt |
| | | | | ttacagcataattgtctcgatatccttgccgaggctcatg |
| | | | | gaacccgcccagaccttactgaccaaccacttcccgatgc |
| | | | | tgaccacacctggtataccaatggttcatctctgctacag |
| | | | | gagggtcaacgcaaagcgggtgcagcagttaccactgaaa |
| | | | | ctgaggttatttgggcgaaagcgctgccagcaggaacgag |
| | | | | cgctcagagagccgagttaattgcgttaacacaggcgctg |
| | | | | aagatggctgaagggaaaaaactgaacgtctataccgaca |
| | | | | gccgctatgcatttgctacagcccatattcatggagaaat |
| | | | | ttatcgccggcgaggttggctcacgtcgaaaggtaaagaa |
| | | | | ataaaaaataaggatgaaattctggcacttctgaaagcgt |
| | | | | tatttctgcctaaacgcttaagtatcatacactgtcctgg |
| | | | | tcaccaaaaaggtcatagtgctgaagcgcgcgggaaccgc |
| | | | | atggccgatcaggccgcccgtaaagctgcaattaccgaaa |
| | | | | cccccgatacatctacgctgctgattgaaaactctagccc |
| | | | | aaatagcagactgattaatggtggaggtgggtcagtaaca |
| | | | | gtaaaatttaaatataaaggtgaagagttggaggttgata |
| | | | | tttcaaagataaaaaaagtgtggagagtcggtaaaatgat |
| | | | | aagttttacgtatgatgacaacgggaaaaccggtcggggc |
| | | | | gccgtgtccgaaaaagacgctccgaaggaactactccaaa |
| | | | | tgcttgagaaaagtggaaaaaaa |
| 155 | RTx | TWLSDFPQAWAETGGMGLAVRQAPLIIPLK | 156 | acctggctgtctgactttccgcaggcttgggcagagaccg |
| | 42B L - | ATSTPVSIKQYPLSQKARLGIKPHIQRLLD | | gggggatgggtttagcggtacggcaggcaccactgattat |
| | Sto7 | QGILVPCQSPWNTPLLPVKKPGTNDYRPVQ | | accgctgaaagcaacatcgactcctgtctcgataaagcaa |
| | K13L | DLREVNKRVEDIHPTVPNPYNLLSGPPPSH | | taccctctgtcccaaaaggctcgcttaggcattaagccac |
| | N-tag: His | QWYTVLDLKDAFFCLRLHPTSQPLFAFEWR | | atatccagcgtctgctagatcaaggtattctcgttccttg |
| | C-tag: Sto7 | DPEMGISGQLTWTRLPQGFKNSPTLFNEAL | | tcagtctccgtggaataccccgttgctgccagttaagaaa |
| | | HRDLADFRIQHPDLILLQYVDDLLLAATSE | | ccgggcacaaacgactatcgcccggttcaggacctgcgag |
| | | LDCQQGTRALLQTLGNLGYRASAKKAQICQ | | aggtaaacaaacgtgtggaagatatccatccgacagtgcc |
| | | KQVKYLGYLLKEGQRWLTEARKETVMGQPT | | caacccctacaatctgttgtctggtcctcctccaagccac |
| | | PKTPRQLRRFLGKAGFCRLFIPGFAEMAAP | | cagtggtacacagtcctggacttaaaagatgcattcttct |
| | | LYPLTKPGTLFNWGPDQQKAYQEIKQALLT | | gcctccgtctgcatccaactagccaacctctgttcgcgtt |
| | | APALGLPDLTKPFELFVDEKQGYAKGVLTQ | | tgaatgggggacccggaaatgggaattagcggtcagctca |
| | | KLGPWRRPVAYLSKKLDPVAAGWPPCLRMV | | cctggacaagactcccgcaaggattcaagaactccccgac |
| | | AAIAVLTKDAGKLTMGQPLVIGAPHAVEAL | | tctgtttaatgaagcgctgcaccgcgacctggccgacttt |
| | | VKQPAGRWLSKARMTHYQALLLDTDRVQFG | | agaatacagcacccagatcttatccttctgcagtacgtcg |
| | | PVVALNPATLLPLPEEGLQHNCLDILAEAH | | atgatcttttgctggccgcaacaagcgagctggattgtca |
| | | GTRPDLTDQPLPDADHTWYTNGSSLLQEGQ | | acaggggacgcgtgctcttttacagaccctgggtaatctc |
| | | RKAGAAVTTETEVIWAKALPAGTSAQRAEL | | ggttaccgcgcgagcgcaaaaaaagcccaaatatgtcaaa |
| | | IALTQALKMAEGKKLNVYTDSRYAFATAHI | | aacaggtaaagtatctgggatacctgcttaaagagggtca |
| | | HGEIYRRRGWLTSKGKEIKNKDEILALLKA | | gcgatggctcactgaggcacgtaaagagaccgttatggga |
| | | LFLPKRLSIIHCPGHQKGHSAEARGNRMAD | | cagccgacaccaaaaacaccgcgacaactgcggcggtttc |
| | | QAARKAAITETPDTSTLLIENSSPNSRLIN | | tgggaaaagcgggtttttgtcgattatttatccctgggtt |
| | | GGGGSVTVKFKYKGEELEVDISKIKKVWRV | | tgccgaaatggcggcacctttatatccactcactaagcca |
| | | GKMISFTYDDNGKTGRGAVSEKDAPKELLQ | | ggcacactgtttaattggggaccggatcaacagaaggcgt |
| | | MLEKSGKK | | accaggagataaaacaagcgctgttaaccgcccccgcttt |
| | | | | aggtctgcctgacctgactaagccgttcgaactgtttgta |
| | | | | gatgaaaaacaaggttacgcgaaaggtgttttaactcaga |
| | | | | aacttgggccatggcgtcgtcctgtcgcgtatctctctaa |
| | | | | aaaacttgacccggttgcggctggctggcctccatgcctg |
| | | | | agaatggtggcagctatagcagtcctgactaaagatgccg |
| | | | | gtaaattgaccatgggtcagccacttgtgatcggtgctcc |
| | | | | ccacgcggtggaggcgttggttaaacagcctgccgggcgt |
| | | | | tggttgtcgaaagctcgcatgacacattatcaggccttat |
| | | | | tacttgatacggacagagttcagtttggaccggtagtcgc |
| | | | | cctgaacccggccacgttacttccgctgccagaagaaggt |
| | | | | ttacagcataattgtctcgatatccttgccgaggctcatg |
| | | | | gaacccgcccagaccttactgaccaaccacttcccgatgc |
| | | | | tgaccacacctggtataccaatggttcatctctgctacag |
| | | | | gagggtcaacgcaaagcgggtgcagcagttaccactgaaa |
| | | | | ctgaggttatttgggcgaaagcgctgccagcaggaacgag |
| | | | | cgctcagagagccgagttaattgcgttaacacaggcgctg |
| | | | | aagatggctgaagggaaaaaactgaacgtctataccgaca |
| | | | | gccgctatgcatttgctacagcccatattcatggagaaat |
| | | | | ttatcgccggcgaggttggctcacgtcgaaaggtaaagaa |
| | | | | ataaaaaataaggatgaaattctggcacttctgaaagcgt |
| | | | | tatttctgcctaaacgcttaagtatcatacactgtcctgg |
| | | | | tcaccaaaaaggtcatagtgctgaagcgcgcgggaaccgc |
| | | | | atggccgatcaggccgcccgtaaagctgcaattaccgaaa |
| | | | | cccccgatacatctacgctgctgattgaaaactctagccc |
| | | | | aaatagcagactgattaatggtggaggtgggtcagtaaca |
| | | | | gtaaaatttaaatataaaggtgaagagttggaggttgata |
| | | | | tttcaaagataaaaaaagtgtggagagtcggtaaaatgat |
| | | | | aagttttacgtatgatgacaacgggaaaaccggtcggggc |
| | | | | gccgtgtccgaaaaagacgctccgaaggaactactccaaa |
| | | | | tgcttgagaaaagtggaaaaaaa |
| 157 | RTx | TWLSDFPQAWAETGGVGLAVRQAPLIIPLK | 158 | acctggctgtctgactttccgcaggcttgggcagagaccg |
| | 42B V - | ATSTPVSIKQYPMSQKARLGIKPHIQRLLD | | gggggggggtttagcggtacggcaggcaccactgattata |
| | Sto7 K13L | QGILVPCQSPWNTPLLPVKKPGTNDYRPVQ | | ccgctgaaagcaacatcgactcctgtctcgataaagcaat |
| | N-Tag: His | DLREVNKRVEDIHPTVPNPYNLLSGPPPSH | | accctatgtcccaaaaggctcgcttaggcattaagccaca |
| | C-tag: Sto7 | QWYTVLDLKDAFFCLRLHPTSQPLFAFEWR | | tatccagcgtctgctagatcaaggtattctcgttccttgt |
| | | DPEMGISGQLTWTRLPQGFKNSPTLFNEAL | | cagtctccgtggaataccccgttgctgccagttaagaaac |
| | | HRDLADFRIQHPDLILLQYVDDLLLAATSE | | cgggcacaaacgactatcgcccggttcaggacctgcgaga |
| | | LDCQQGTRALLQTLGNLGYRASAKKAQICQ | | ggtaaacaaacgtgtggaagatatccatccgacagtgccc |
| | | KQVKYLGYLLKEGQRWLTEARKETVMGQPT | | aacccctacaatctgttgtctggtcctcctccaagccacc |
| | | PKTPRQLRRFLGKAGFCRLFIPGFAEMAAP | | agtggtacacagtcctggacttaaaagatgcattcttctg |
| | | LYPLTKPGTLFNWGPDQQKAYQEIKQALLT | | cctccgtctgcatccaactagccaacctctgttcgcgttt |
| | | APALGLPDLTKPFELFVDEKQGYAKGVLTQ | | gaatggcgggacccggaaatgggaattagcggtcagctca |
| | | KLGPWRRPVAYLSKKLDPVAAGWPPCLRMV | | cctggacaagactcccgcaaggattcaagaactccccgac |
| | | AAIAVLTKDAGKLTMGQPLVIGAPHAVEAL | | tctgtttaatgaagcgctgcaccgcgacctggccgacttt |
| | | VKQPAGRWLSKARMTHYQALLLDTDRVQFG | | agaatacagcacccagatcttatccttctgcagtacgtcg |
| | | PVVALNPATLLPLPEEGLQHNCLDILAEAH | | atgatcttttgctggccgcaacaagcgagctggattgtca |
| | | GTRPDLTDQPLPDADHTWYTNGSSLLQEGQ | | acaggggacgcgtgctcttttacagaccctgggtaatctc |
| | | RKAGAAVTTETEVIWAKALPAGTSAQRAEL | | ggttaccgcgcgagcgcaaaaaaagcccaaatatgtcaaa |
| | | IALTQALKMAEGKKLNVYTDSRYAFATAHI | | aacaggtaaagtatctgggatacctgcttaaagagggtca |
| | | HGEIYRRRGWLTSKGKEIKNKDEILALLKA | | gcgatggctcactgaggcacgtaaagagaccgttatggga |
| | | LFLPKRLSIIHCPGHQKGHSAEARGNRMAD | | cagccgacaccaaaaacaccgcgacaactgcggcggtttc |
| | | QAARKAAITETPDTSTLLIENSSPNSRLIN | | tgggaaaagcgggtttttgtcgattatttatccctgggtt |
| | | GGGGSVTVKFKYKGEELEVDISKIKKVWRV | | tgccgaaatggcggcacctttatatccactcactaagcca |
| | | GKMISFTYDDNGKTGRGAVSEKDAPKELLQ | | ggcacactgtttaattggggaccggatcaacagaaggcgt |
| | | MLEKSGKK | | accaggagataaaacaagcgctgttaaccgcccccgcttt |
| | | | | aggtctgcctgacctgactaagccgttcgaactgtttgta |
| | | | | gatgaaaaacaaggttacgcgaaaggtgttttaactcaga |
| | | | | aacttgggccatggcgtcgtcctgtcgcgtatctctctaa |
| | | | | aaaacttgacccggttgcggctggctggcctccatgcctg |
| | | | | agaatggtggcagctatagcagtcctgactaaagatgccg |
| | | | | gtaaattgaccatgggtcagccacttgtgatcggtgctcc |
| | | | | ccacgcggtggaggcgttggttaaacagcctgccgggcgt |
| | | | | tggttgtcgaaagctcgcatgacacattatcaggccttat |
| | | | | tacttgatacggacagagttcagtttggaccggtagtcgc |
| | | | | cctgaacccggccacgttacttccgctgccagaagaaggt |
| | | | | ttacagcataattgtctcgatatccttgccgaggctcatg |
| | | | | gaacccgcccagaccttactgaccaaccacttcccgatgc |
| | | | | tgaccacacctggtataccaatggttcatctctgctacag |
| | | | | gagggtcaacgcaaagcgggtgcagcagttaccactgaaa |
| | | | | ctgaggttatttgggcgaaagcgctgccagcaggaacgag |
| | | | | cgctcagagagccgagttaattgcgttaacacaggcgctg |
| | | | | aagatggctgaagggaaaaaactgaacgtctataccgaca |
| | | | | gccgctatgcatttgctacagcccatattcatggagaaat |
| | | | | ttatcgccggcgaggttggctcacgtcgaaaggtaaagaa |
| | | | | ataaaaaataaggatgaaattctggcacttctgaaagcgt |
| | | | | tatttctgcctaaacgcttaagtatcatacactgtcctgg |
| | | | | tcaccaaaaaggtcatagtgctgaagcgcgcgggaaccgc |
| | | | | atggccgatcaggccgcccgtaaagctgcaattaccgaaa |
| | | | | cccccgatacatctacgctgctgattgaaaactctagccc |
| | | | | aaatagcagactgattaatggtggaggtgggtcagtaaca |
| | | | | gtaaaatttaaatataaaggtgaagagttggaggttgata |
| | | | | tttcaaagataaaaaaagtgtggagagtcggtaaaatgat |
| | | | | aagttttacgtatgatgacaacgggaaaaccggtcggggc |
| | | | | gccgtgtccgaaaaagacgctccgaaggaactactccaaa |
| | | | | tgcttgagaaaagtggaaaaaaa |
| 159 | RTx | TWLSDFPQAWAETGGMGLAVRQAPLIIPLK | 160 | acctggctgtctgactttccgcaggcttgggcagagaccg |
| | N-tag: His | ATSTPVSIKQYPLSQKARLGIKPHIQRLLD | | gggggatgggtttagcggtacggcaggcaccactgattat |
| | C-tag: Sto 7 | QGILVPCQSPWNTPLLPVKKPGTNDYRPVQ | | accgctgaaagcaacatcgactcctgtctcgataaagcaa |
| | 42B L - Sto7 | DLREVNKRVEDIHPTVPNPYNLLSGPPPSH | | taccctctgtcccaaaaggctcgcttaggcattaagccac |
| | Trunc 529 | QWYTVLDLKDAFFCLRLHPTSQPLFAFEWR | | atatccagcgtctgctagatcaaggtattctcgttccttg |
| | | DPEMGISGQLTWTRLPQGFKNSPTLFNEAL | | tcagtctccgtggaataccccgttgctgccagttaagaaa |
| | | HRDLADFRIQHPDLILLQYVDDLLLAATSE | | ccgggcacaaacgactatcgcccggttcaggacctgcgag |
| | | LDCQQGTRALLQTLGNLGYRASAKKAQICQ | | aggtaaacaaacgtgtggaagatatccatccgacagtgcc |
| | | KQVKYLGYLLKEGQRWLTEARKETVMGQPT | | caacccctacaatctgttgtctggtcctcctccaagccac |
| | | PKTPRQLRRFLGKAGFCRLFIPGFAEMAAP | | cagtggtacacagtcctggacttaaaagatgcattcttct |
| | | LYPLTKPGTLFNWGPDQQKAYQEIKQALLT | | gcctccgtctgcatccaactagccaacctctgttcgcgtt |
| | | APALGLPDLTKPFELFVDEKQGYAKGVLTQ | | tgaatggcgggacccggaaatgggaattagcggtcagctc |
| | | KLGPWRRPVAYLSKKLDPVAAGWPPCLRMV | | acctggacaagactcccgcaaggattcaagaactccccga |
| | | AAIAVLTKDAGKLTMGQPLVIGAPHAVEAL | | ctctgtttaatgaagcgctgcaccgcgacctggccgactt |
| | | VKQPAGRWLSKARMTHYQALLLDTDRVQFG | | tagaatacagcacccagatcttatccttctgcagtacgtc |
| | | PVVALNPATLLPLPEEGLQHNCLDILAEAH | | gatgatcttttgctggccgcaacaagcgagctggattgtc |
| | | GTRPDLTDQPLPDADHTWYTNGSSLLGGGG | | aacaggggacgcgtgctcttttacagaccctgggtaatct |
| | | SVTVKFKYKGEELEVDISKIKKVWRVGKMI | | cggttaccgcgcgagcgcaaaaaaagcccaaatatgtcaa |
| | | SFTYDDNGKTGRGAVSEKDAPKELLQMLEK | | aaacaggtaaagtatctgggatacctgcttaaagagggtc |
| | | SGKK | | agcgatggctcactgaggcacgtaaagagaccgttatggg |
| | | | | acagccgacaccaaaaacaccgcgacaactgcggcggttt |
| | | | | ctgggaaaagcgggtttttgtcgattatttatccctgggt |
| | | | | ttgccgaaatggcggcacctttatatccactcactaagcc |
| | | | | aggcacactgtttaattggggaccggatcaacagaagggt |
| | | | | accaggagataaaacaagcgctgttaaccgcccccgcttt |
| | | | | aggtctgcctgacctgactaagccgttcgaactgtttgta |
| | | | | gatgaaaaacaaggttacgcgaaaggtgttttaactcaga |
| | | | | aacttgggccatggcgtcgtcctgtcgcgtatctctctaa |
| | | | | aaaacttgacccggttgcggctggctggcctccatgcctg |
| | | | | agaatggtggcagctatagcagtcctgactaaagatgccg |
| | | | | gtaaattgaccatgggtcagccacttgtgatcggtgctcc |
| | | | | ccacgcggtggaggcgttggttaaacagcctgccgggcgt |
| | | | | tggttgtcgaaagctcgcatgacacattatcaggccttat |
| | | | | tacttgatacggacagagttcagtttggaccggtagtcgc |
| | | | | cctgaacccggccacgttacttccgctgccagaagaaggt |
| | | | | ttacagcataattgtctcgatatccttgccgaggctcatg |
| | | | | gaacccgcccagaccttactgaccaaccacttcccgatgc |
| | | | | tgaccacacctggtataccaatggttcatctctgctaggt |
| | | | | ggaggtgggtcagtaacagtaaaatttaaatataaaggtg |
| | | | | aagagttggaggttgatatttcaaagataaaaaaagtgtg |
| | | | | gagagtcggtaaaatgataagttttacgtatgatgacaac |
| | | | | gggaaaaccggtcggggcgccgtgtccgaaaaagacgctc |
| | | | | cgaaggaactactccaaatgcttgagaaaagtggaaaaaa |
| | | | | a |
| 161 | RTx | TWLSDFPQAWAETGGMGLAVRQAPLIIPLK | 162 | acctggctgtctgactttccgcaggcttgggcagagaccg |
| | 42B L - Sto7 | ATSTPVSIKQYPLSQKARLGIKPHIQRLLD | | gggggatgggtttagcggtacggcaggcaccactgattat |
| | Trunc 534 | QGILVPCQSPWNTPLLPVKKPGTNDYRPVQ | | accgctgaaagcaacatcgactcctgtctcgataaagcaa |
| | N-tag: His | DLREVNKRVEDIHPTVPNPYNLLSGPPPSH | | taccctctgtcccaaaaggctcgcttaggcattaagccac |
| | C-tag: Sto7 | QWYTVLDLKDAFFCLRLHPTSQPLFAFEWR | | atatccagcgtctgctagatcaaggtattctcgttccttg |
| | | DPEMGISGQLTWTRLPQGFKNSPTLFNEAL | | tcagtctccgtggaataccccgttgctgccagttaagaaa |
| | | HRDLADFRIQHPDLILLQYVDDLLLAATSE | | ccgggcacaaacgactatcgcccggttcaggacctgcgag |
| | | LDCQQGTRALLQTLGNLGYRASAKKAQICQ | | aggtaaacaaacgtgtggaagatatccatccgacagtgcc |
| | | KQVKYLGYLLKEGQRWLTEARKETVMGQPT | | caacccctacaatctgttgtctggtcctcctccaagccac |
| | | PKTPRQLRRFLGKAGFCRLFIPGFAEMAAP | | cagtggtacacagtcctggacttaaaagatgcattcttct |
| | | LYPLTKPGTLFNWGPDQQKAYQEIKQALLT | | gcctccgtctgcatccaactagccaacctctgttcgcgtt |
| | | APALGLPDLTKPFELFVDEKQGYAKGVLTQ | | tgaatgggggacccggaaatgggaattagcggtcagctca |
| | | KLGPWRRPVAYLSKKLDPVAAGWPPCLRMV | | cctggacaagactcccgcaaggattcaagaactccccgac |
| | | AAIAVLTKDAGKLTMGQPLVIGAPHAVEAL | | tctgtttaatgaagcgctgcaccgcgacctggccgacttt |
| | | VKQPAGRWLSKARMTHYQALLLDTDRVQFG | | agaatacagcacccagatcttatccttctgcagtacgtcg |
| | | PVVALNPATLLPLPEEGLQHNCLDILAEAH | | atgatcttttgctggccgcaacaagcgagctggattgtca |
| | | GTRPDLTDQPLPDADHTWYTNGSSLLQEGQ | | acaggggacgcgtgctcttttacagaccctgggtaatctc |
| | | RGGGGSVTVKFKYKGEELEVDISKIKKVWR | | ggttaccgcgcgagcgcaaaaaaagcccaaatatgtcaaa |
| | | VGKMISFTYDDNGKTGRGAVSEKDAPKELL | | aacaggtaaagtatctgggatacctgcttaaagagggtca |
| | | QMLEKSGKK | | gcgatggctcactgaggcacgtaaagagaccgttatggga |
| | | | | cagccgacaccaaaaacaccgcgacaactgcggcggtttc |
| | | | | tgggaaaagcgggtttttgtcgattatttatccctgggtt |
| | | | | tgccgaaatggcggcacctttatatccactcactaagcca |
| | | | | ggcacactgtttaattggggaccggatcaacagaaggcgt |
| | | | | accaggagataaaacaagcgctgttaaccgcccccgcttt |
| | | | | aggtctgcctgacctgactaagccgttcgaactgtttgta |
| | | | | gatgaaaaacaaggttacgcgaaaggtgttttaactcaga |
| | | | | aacttgggccatggcgtcgtcctgtcgcgtatctctctaa |
| | | | | aaaacttgacccggttgcggctggctggcctccatgcctg |
| | | | | agaatggtggcagctatagcagtcctgactaaagatgccg |
| | | | | gtaaattgaccatgggtcagccacttgtgatcggtgctcc |
| | | | | ccacgcggtggaggcgttggttaaacagcctgccgggcgt |
| | | | | tggttgtcgaaagctcgcatgacacattatcaggccttat |
| | | | | tacttgatacggacagagttcagtttggaccggtagtcgc |
| | | | | cctgaacccggccacgttacttccgctgccagaagaaggt |
| | | | | ttacagcataattgtctcgatatccttgccgaggctcatg |
| | | | | gaacccgcccagaccttactgaccaaccacttcccgatgc |
| | | | | tgaccacacctggtataccaatggttcatctctgctacag |
| | | | | gagggtcaacgcggtggaggtgggtcagtaacagtaaaat |
| | | | | ttaaatataaaggtgaagagttggaggttgatatttcaaa |
| | | | | gataaaaaaagtgtggagagtcggtaaaatgataagtttt |
| | | | | acgtatgatgacaacgggaaaaccggtcggggcgccgtgt |
| | | | | ccgaaaaagacgctccgaaggaactactccaaatgcttga |
| | | | | gaaaagtggaaaaaaa |
| 163 | RTx | TWLSDFPQAWAETGGMGLAVRQAPLIIPLK | 164 | acctggctgtctgactttccgcaggcttgggcagagaccg |
| | 42B L - Sto7 | ATSTPVSIKQYPLSQKARLGIKPHIQRLLD | | gggggatgggtttagcggtacggcaggcaccactgattat |
| | Trunc 560 | QGILVPCQSPWNTPLLPVKKPGTNDYRPVQ | | accgctgaaagcaacatcgactcctgtctcgataaagcaa |
| | N-tag: his | DLREVNKRVEDIHPTVPNPYNLLSGPPPSH | | taccctctgtcccaaaaggctcgcttaggcattaagccac |
| | C-tag: Sto7 | QWYTVLDLKDAFFCLRLHPTSQPLFAFEWR | | atatccagcgtctgctagatcaaggtattctcgttccttg |
| | | DPEMGISGQLTWTRLPQGFKNSPTLFNEAL | | tcagtctccgtggaataccccgttgctgccagttaagaaa |
| | | HRDLADFRIQHPDLILLQYVDDLLLAATSE | | ccgggcacaaacgactatcgcccggttcaggacctgcgag |
| | | LDCQQGTRALLQTLGNLGYRASAKKAQICQ | | aggtaaacaaacgtgtggaagatatccatccgacagtgcc |
| | | KQVKYLGYLLKEGQRWLTEARKETVMGQPT | | caacccctacaatctgttgtctggtcctcctccaagccac |
| | | PKTPRQLRRFLGKAGFCRLFIPGFAEMAAP | | cagtggtacacagtcctggacttaaaagatgcattcttct |
| | | LYPLTKPGTLFNWGPDQQKAYQEIKQALLT | | gcctccgtctgcatccaactagccaacctctgttcgcgtt |
| | | APALGLPDLTKPFELFVDEKQGYAKGVLTQ | | tgaatgggggacccggaaatgggaattagcggtcagctca |
| | | KLGPWRRPVAYLSKKLDPVAAGWPPCLRMV | | cctggacaagactcccgcaaggattcaagaactccccgac |
| | | AAIAVLTKDAGKLTMGQPLVIGAPHAVEAL | | tctgtttaatgaagcgctgcaccgcgacctggccgacttt |
| | | VKQPAGRWLSKARMTHYQALLLDTDRVQFG | | agaatacagcacccagatcttatccttctgcagtacgtcg |
| | | PVVALNPATLLPLPEEGLQHNCLDILAEAH | | atgatcttttgctggccgcaacaagcgagctggattgtca |
| | | GTRPDLTDQPLPDADHTWYTNGSSLLQEGQ | | acaggggacgcgtgctcttttacagaccctgggtaatctc |
| | | RKAGAAVTTETEVIWAKALPAGTSAQRGGG | | ggttaccgcgcgagcgcaaaaaaagcccaaatatgtcaaa |
| | | GSVTVKFKYKGEELEVDISKIKKVWRVGKM | | aacaggtaaagtatctgggatacctgcttaaagagggtca |
| | | ISFTYDDNGKTGRGAVSEKDAPKELLQMLE | | gcgatggctcactgaggcacgtaaagagaccgttatggga |
| | | KSGKK | | cagccgacaccaaaaacaccgcgacaactgcggcggtttc |
| | | | | tgggaaaagcgggtttttgtcgattatttatccctgggtt |
| | | | | tgccgaaatggcggcacctttatatccactcactaagcca |
| | | | | ggcacactgtttaattggggaccggatcaacagaaggcgt |
| | | | | accaggagataaaacaagcgctgttaaccgcccccgcttt |
| | | | | aggtctgcctgacctgactaagccgttcgaactgtttgta |
| | | | | gatgaaaaacaaggttacgcgaaaggtgttttaactcaga |
| | | | | aacttgggccatggcgtcgtcctgtcgcgtatctctctaa |
| | | | | aaaacttgacccggttgcggctggctggcctccatgcctg |
| | | | | agaatggtggcagctatagcagtcctgactaaagatgccg |
| | | | | gtaaattgaccatgggtcagccacttgtgatcggtgctcc |
| | | | | ccacgcggtggaggcgttggttaaacagcctgccgggcgt |
| | | | | tggttgtcgaaagctcgcatgacacattatcaggccttat |
| | | | | tacttgatacggacagagttcagtttggaccggtagtcgc |
| | | | | cctgaacccggccacgttacttccgctgccagaagaaggt |
| | | | | ttacagcataattgtctcgatatccttgccgaggctcatg |
| | | | | gaacccgcccagaccttactgaccaaccacttcccgatgc |
| | | | | tgaccacacctggtataccaatggttcatctctgctacag |
| | | | | gagggtcaacgcaaagcgggtgcagcagttaccactgaaa |
| | | | | ctgaggttatttgggcgaaagcgctgccagcaggaacgag |
| | | | | cgctcagagaggtggaggtgggtcagtaacagtaaaattt |
| | | | | aaatataaaggtgaagagttggaggttgatatttcaaaga |
| | | | | taaaaaaagtgtggagagtcggtaaaatgataagttttac |
| | | | | gtatgatgacaacgggaaaaccggtcgggggccgtgtccg |
| | | | | aaaaagacgctccgaaggaactactccaaatgcttgagaa |
| | | | | aagtggaaaaaaa |
| 166 | RTx | TWLSDFPQAWAETGGMGLAVRQAPLIIPLK | 167 | acctggctgtctgactttccgcaggcttgggcagagaccg |
| | 42B L - Sto7 | ATSTPVSIKQYPLSQKARLGIKPHIQRLLD | | gggggatgggtttagcggtacggcaggcaccactgattat |
| | Trunc 613 | QGILVPCQSPWNTPLLPVKKPGTNDYRPVQ | | accgctgaaagcaacatcgactcctgtctcgataaagcaa |
| | N-tag: his | DLREVNKRVEDIHPTVPNPYNLLSGPPPSH | | taccctctgtcccaaaaggctcgcttaggcattaagccac |
| | C-tag: Sto7 | QWYTVLDLKDAFFCLRLHPTSQPLFAFEWR | | atatccagcgtctgctagatcaaggtattctcgttccttg |
| | | DPEMGISGQLTWTRLPQGFKNSPTLFNEAL | | tcagtctccgtggaataccccgttgctgccagttaagaaa |
| | | HRDLADFRIQHPDLILLQYVDDLLLAATSE | | ccgggcacaaacgactatcgcccggttcaggacctgcgag |
| | | LDCQQGTRALLQTLGNLGYRASAKKAQICQ | | aggtaaacaaacgtgtggaagatatccatccgacagtgcc |
| | | KQVKYLGYLLKEGQRWLTEARKETVMGQPT | | caacccctacaatctgttgtctggtcctcctccaagccac |
| | | PKTPRQLRRFLGKAGFCRLFIPGFAEMAAP | | cagtggtacacagtcctggacttaaaagatgcattcttct |
| | | LYPLTKPGTLFNWGPDQQKAYQEIKQALLT | | gcctccgtctgcatccaactagccaacctctgttcgcgtt |
| | | APALGLPDLTKPFELFVDEKQGYAKGVLTQ | | tgaatgggggacccggaaatgggaattagcggtcagctca |
| | | KLGPWRRPVAYLSKKLDPVAAGWPPCLRMV | | cctggacaagactcccgcaaggattcaagaactccccgac |
| | | AAIAVLTKDAGKLTMGQPLVIGAPHAVEAL | | tctgtttaatgaagcgctgcaccgcgacctggccgacttt |
| | | VKQPAGRWLSKARMTHYQALLLDTDRVQFG | | agaatacagcacccagatcttatccttctgcagtacgtcg |
| | | PVVALNPATLLPLPEEGLQHNCLDILAEAH | | atgatcttttgctggccgcaacaagcgagctggattgtca |
| | | GTRPDLTDQPLPDADHTWYTNGSSLLQEGQ | | acaggggacgcgtgctcttttacagaccctgggtaatctc |
| | | RKAGAAVTTETEVIWAKALPAGTSAQRAEL | | ggttaccgcgcgagcgcaaaaaaagcccaaatatgtcaaa |
| | | IALTQALKMAEGKKLNVYTDSRYAFATAHI | | aacaggtaaagtatctgggatacctgcttaaagagggtca |
| | | HGEIYRRRGWLTSKGKEIKNGGGGSVTVKF | | gcgatggctcactgaggcacgtaaagagaccgttatggga |
| | | KYKGEELEVDISKIKKVWRVGKMISFTYDD | | cagccgacaccaaaaacaccgcgacaactgcggcggtttc |
| | | NGKTGRGAVSEKDAPKELLQMLEKSGKK | | tgggaaaagcgggtttttgtcgattatttatccctgggtt |
| | | | | tgccgaaatggcggcacctttatatccactcactaagcca |
| | | | | ggcacactgtttaattggggaccggatcaacagaaggcgt |
| | | | | accaggagataaaacaagcgctgttaaccgcccccgcttt |
| | | | | aggtctgcctgacctgactaagccgttcgaactgtttgta |
| | | | | gatgaaaaacaaggttacgcgaaaggtgttttaactcaga |
| | | | | aacttgggccatggcgtcgtcctgtcgcgtatctctctaa |
| | | | | aaaacttgacccggttgcggctggctggcctccatgcctg |
| | | | | agaatggtggcagctatagcagtcctgactaaagatgccg |
| | | | | gtaaattgaccatgggtcagccacttgtgatcggtgctcc |
| | | | | ccacgcggtggaggcgttggttaaacagcctgccgggcgt |
| | | | | tggttgtcgaaagctcgcatgacacattatcaggccttat |
| | | | | tacttgatacggacagagttcagtttggaccggtagtcgc |
| | | | | cctgaacccggccacgttacttccgctgccagaagaaggt |
| | | | | ttacagcataattgtctcgatatccttgccgaggctcatg |
| | | | | gaacccgcccagaccttactgaccaaccacttcccgatgc |
| | | | | tgaccacacctggtataccaatggttcatctctgctacag |
| | | | | gagggtcaacgcaaagcgggtgcagcagttaccactgaaa |
| | | | | ctgaggttatttgggcgaaagcgctgccagcaggaacgag |
| | | | | cgctcagagagccgagttaattgcgttaacacaggcgctg |
| | | | | aagatggctgaagggaaaaaactgaacgtctataccgaca |
| | | | | gccgctatgcatttgctacagcccatattcatggagaaat |
| | | | | ttatcgccggcgaggttggctcacgtcgaaaggtaaagaa |
| | | | | ataaaaaatggtggaggtgggtcagtaacagtaaaattta |
| | | | | aatataaaggtgaagagttggaggttgatatttcaaagat |
| | | | | aaaaaaagtgtggagagtcggtaaaatgataagttttacg |
| | | | | tatgatgacaacgggaaaaccggtcggggcgccgtgtccg |
| | | | | aaaaagacgctccgaaggaactactccaaatgcttgagaa |
| | | | | aagtggaaaaaaa |
| 168 | RTx | TWLSDFPQAWAETGGMGLAVRQAPLIIPLK | 169 | acctggctgtctgactttccgcaggcttgggcagagaccg |
| | 42B L - Sto7 | ATSTPVSIKQYPLSQKARLGIKPHIQRLLD | | gggggatgggtttagcggtacggcaggcaccactgattat |
| | Trunc 609 | QGILVPCQSPWNTPLLPVKKPGTNDYRPVQ | | accgctgaaagcaacatcgactcctgtctcgataaagcaa |
| | N-tag: His | DLREVNKRVEDIHPTVPNPYNLLSGPPPSH | | taccctctgtcccaaaaggctcgcttaggcattaagccac |
| | C-tag: Sto7 | QWYTVLDLKDAFFCLRLHPTSQPLFAFEWR | | atatccagcgtctgctagatcaaggtattctcgttccttg |
| | | DPEMGISGQLTWTRLPQGFKNSPTLFNEAL | | tcagtctccgtggaataccccgttgctgccagttaagaaa |
| | | HRDLADFRIQHPDLILLQYVDDLLLAATSE | | ccgggcacaaacgactatcgcccggttcaggacctgcgag |
| | | LDCQQGTRALLQTLGNLGYRASAKKAQICQ | | aggtaaacaaacgtgtggaagatatccatccgacagtgcc |
| | | KQVKYLGYLLKEGQRWLTEARKETVMGQPT | | caacccctacaatctgttgtctggtcctcctccaagccac |
| | | PKTPRQLRRFLGKAGFCRLFIPGFAEMAAP | | cagtggtacacagtcctggacttaaaagatgcattcttct |
| | | LYPLTKPGTLFNWGPDQQKAYQEIKQALLT | | gcctccgtctgcatccaactagccaacctctgttcgcgtt |
| | | APALGLPDLTKPFELFVDEKQGYAKGVLTQ | | tgaatggcgggacccggaaatgggaattagcggtcagctc |
| | | KLGPWRRPVAYLSKKLDPVAAGWPPCLRMV | | acctggacaagactcccgcaaggattcaagaactccccga |
| | | AAIAVLTKDAGKLTMGQPLVIGAPHAVEAL | | ctctgtttaatgaagcgctgcaccgcgacctggccgactt |
| | | VKQPAGRWLSKARMTHYQALLLDTDRVQFG | | tagaatacagcacccagatcttatccttctgcagtacgtc |
| | | PVVALNPATLLPLPEEGLQHNCLDILAEAH | | gatgatcttttgctggccgcaacaagcgagctggattgtc |
| | | GTRPDLTDQPLPDADHTWYTNGSSLLQEGQ | | aacaggggacgcgtgctcttttacagaccctgggtaatct |
| | | RKAGAAVTTETEVIWAKALPAGTSAQRAEL | | cggttaccgcgcgagcgcaaaaaaagcccaaatatgtcaa |
| | | IALTQALKMAEGKKLNVYTDSRYAFATAHI | | aaacaggtaaagtatctgggatacctgcttaaagagggtc |
| | | HGEIYRRRGWLTSKGKGGGGSVTVKFKYKG | | agcgatggctcactgaggcacgtaaagagaccgttatggg |
| | | EELEVDISKIKKVWRVGKMISFTYDDNGKT | | acagccgacaccaaaaacaccgcgacaactgcggcggttt |
| | | GRGAVSEKDAPKELLQMLEKSGKK | | ctgggaaaagcgggtttttgtcgattatttatccctgggt |
| | | | | ttgccgaaatggcggcacctttatatccactcactaagcc |
| | | | | aggcacactgtttaattggggaccggatcaacagaaggcg |
| | | | | taccaggagataaaacaagcgctgttaaccgcccccgctt |
| | | | | taggtctgcctgacctgactaagccgttcgaactgtttgt |
| | | | | agatgaaaaacaaggttacgcgaaagglgtittaactcag |
| | | | | aaactigggccalggcgtcgtcclgtcgcgtatctctcta |
| | | | | aaaaacttgacccggttgcggctggctggcctccatgcct |
| | | | | gagaatggtggcagctatagcagtcctgactaaagatgcc |
| | | | | ggtaaattgaccatgggtcagccacttgtgatcggtgctc |
| | | | | cccacgcggtggaggcgttggttaaacagcctgccgggcg |
| | | | | ttggttgtcgaaagctcgcatgacacattatcaggcctta |
| | | | | ttacttgatacggacagagttcagtttggaccggtagtcg |
| | | | | ccctgaacccggccacgttacttccgctgccagaagaagg |
| | | | | tttacagcataattgtctcgatatccttgccgaggctcat |
| | | | | ggaacccgcccagaccttactgaccaaccacttcccgatg |
| | | | | ctgaccacacctggtataccaatggttcatctctgctaca |
| | | | | ggagggtcaacgcaaagcgggtgcagcagttaccactgaa |
| | | | | actgaggttatttgggcgaaagcgctgccagcaggaacga |
| | | | | gcgctcagagagccgagttaattgcgttaacacaggcgct |
| | | | | gaagatggctgaagggaaaaaactgaacgtctataccgac |
| | | | | agccgctatgcatttgctacagcccatattcatggagaaa |
| | | | | tttatcgccggcgaggttggctcacgtcgaaaggtaaagg |
| | | | | tggaggtgggtcagtaacagtaaaatttaaatataaaggt |
| | | | | gaagagttggaggttgatatttcaaagataaaaaaagtgt |
| | | | | ggagagtcggtaaaatgataagttttacgtatgatgacaa |
| | | | | cgggaaaaccggtcggggcgccgtgtccgaaaaagacgct |
| | | | | ccgaaggaactactccaaatgcttgagaaaagtggaaaaa |
| | | | | aa |
| 170 | RTx | TWLSDFPQAWAETGGMGLAVRQAPLIIPLK | 171 | acttggctgtctgatttccctcaggcgtgggccgaaacgg |
| | 50A+G - | ATSTPVSIKQYPLSQKARLGIKPHIQRLLD | | gggcatgggtctggcagtgcgtcaggcaccgctgattatt |
| | Sto7 K13L | QGILVPCQSPWNTPLLPVKKPGTNDYRPVQ | | ccgctgaaagcgacgtcgaccccggtgagcatcaagcaat |
| | N-tag: his | DLREVNKRVEDIHPTVPNPYNLLSGPPPSH | | atccgctgtcccaaaaggcgcgcttaggtattaagccgca |
| | C-Tag: Sto7 | QWYTVLDLKDAFFCLRLHPTSQPLFAFEWR | | cattcagcgtctgctggatcaaggtattctggttccgtgt |
| | | DPEMGISGQLTWTRLPQGFKNSPTLFNEAL | | cagagcccgtggaataccccgcttctcccggtgaagaaac |
| | | HRDLADFRIQHPDLILLQYVDDLLLAATSE | | cgggcacgaacgattaccgtccagtccaagacttgcgcga |
| | | LDCQQGTRALLQTLGNLGYRASAKKAQICQ | | agttaacaagcgcgttgaagatattcacccgaccgtcccg |
| | | KQVKYLGYLLKEGQRWLTEARKETVMGQPT | | aacccgtacaatctgctgagcggtccgccgccaagccacc |
| | | PKTPRQLRRFLGKAGFCRLFIPGFAEMAAP | | aatggtacaccgtgctggatctgaaagatgctttcttctg |
| | | LYPLTKPGTLFNWGPDQQKAYQEIKQALLT | | tctgcgtctgcacccaaccagccagcctctgtttgcattt |
| | | APALGLPDLTKPFELFVDEKQGYAKGVLTQ | | gagtggcgtgaccctgagatgggtattagcggccagctga |
| | | KLGPWRRPVAYLSKKLDPVAAGWPPCLRMV | | cgtggacccgcctgccgcaaggttttaagaattcccctac |
| | | AAIAVLTKDAGKLTMGQPLVIGAPHAVEAL | | gctgtttaacgaagcgctgcaccgtgacctggcggatttc |
| | | VKQPPDRWLSKARMTHYQALLLDTDRVQFG | | cgtatccagcacccggacctgatcttgctgcagtacgttg |
| | | PVVALNPATLLPLPEEGLQHNCLDILAEAV | | atgacctgttgctggcggcgacgagcgagctggattgcca |
| | | GTRPDLTDQPLPDADHTWYTNGSSLLQEGQ | | acagggcacccgtgcgctgttgcagaccttgggtaacctg |
| | | RKAGAAVTTETEVIWAKALPAGTSAQRAEL | | ggttatcgcgctagcgcgaagaaagcgcagatttgccaaa |
| | | IALTQALKMAEGKKLNVYTDSRYAFATAHI | | aacaagttaagtatctgggctacctgttaaaggaaggcca |
| | | HGEIYRRRGWLTSKGKEIKNKDEILALLKA | | acgttggctgaccgaagcccgcaaagaaactgtcatgggt |
| | | LFLPKRLSIIYCPGHQKGHSAEARGNRMAD | | cagccgaccccgaaaacgccacgccaactgcgtaggttct |
| | | QAARKAAITETPDTSTLLIENSSPNSRLIN | | tgggcaaagcgggtttctgccgcctgttcatcccgggctt |
| | | GGGGSVTVKFKYKGEELEVDISKIKKVWRV | | tgccgaaatggcagccccgctgtatccgttgaccaagccg |
| | | GKMISFTYDDNGKTGRGAVSEKDAPKELLQ | | ggcaccctgttcaactggggtccggaccagcagaaagcgt |
| | | MLEKSGKK | | accaagaaattaaacaagcactgctgacggcaccggcgct |
| | | | | gggtctgccggacctgaccaagccgtttgagctgttcgtg |
| | | | | gatgagaagcaaggttacgcgaagggcgtgttgacccaga |
| | | | | aattgggtccgtggcgtcgtccggttgcatacctgtccaa |
| | | | | gaaactggacccggttgctgctggttggccgccttgcctg |
| | | | | cgcatggttgccgctatcgcggtgctgactaaagacgcgg |
| | | | | gtaagctgacgatgggtcaaccgctggtgatcggtgcacc |
| | | | | gcatgcagtcgaggcccttgttaagcaaccgccggataga |
| | | | | tggctgagcaaggcgcgtatgacgcattaccaggcactgc |
| | | | | tgttggacaccgatcgtgtgcagtttggcccggtcgttgc |
| | | | | gctcaacccggcgaccctgctgccgctcccggaagaaggc |
| | | | | ttgcagcacaactgtttggacatcctggcagaggctgtgg |
| | | | | gaacccgcccagaccttactgaccaaccacttcccgatgc |
| | | | | tgatcatacctggtatacgaatggtagcagcctgttgcaa |
| | | | | gagggtcagcgtaaggccggtgccgcggtcaccaccgaga |
| | | | | ctgaagtgatttgggctaaagcattgcctgcgggtaccag |
| | | | | cgcgcagcgtgccgagctgatcgcactgacccaagcgctg |
| | | | | aaaatggctgagggtaagaaactgaatgtgtacacggata |
| | | | | gccgttatgcctttgcgaccgcccacattcacggcgagat |
| | | | | ctatcgccgtcgcggctggctgacgtccaaaggcaaagag |
| | | | | atcaagaataaagacgaaattctggcgctgctgaaagcgc |
| | | | | tgttcctgccgaaacgtctgtcgatcatctattgcccggg |
| | | | | tcaccagaaaggccacagcgcagaggcgcgtggtaatcgc |
| | | | | atggctgaccaggctgcgcgtaaagccgcaattaccgaaa |
| | | | | ccccggacaccagcacgctgctgatcgagaatagcagccc |
| | | | | gaacagccgtctgatcaatggtggaggtgggtcagtaaca |
| | | | | gtaaaatttaaatataaaggtgaagagttggaggttgata |
| | | | | tttcaaagataaaaaaagtgtggagagtcggtaaaatgat |
| | | | | aagttttacgtatgatgacaacgggaaaaccggtcggggc |
| | | | | gccgtgtccgaaaaagacgctccgaaggaactactccaaa |
| | | | | tgcttgagaaaagtggaaaaaaa |
| 172 | RTx | TLNIEDEHRLHETSKEPDVSLGSTWLSDFP | | |
| | MMLV-WT | QAWAETGGMGLAVRQAPLIIPLKATSTPVS | | |
| | | IKQYPMSQEARLGIKPHIQRLLDQGILVPC | | |
| | | QSPWNTPLLPVKKPGTNDYRPVQDLREVNK | | |
| | | RVEDIHPTVPNPYNLLSGLPPSHQWYTVLD | | |
| | | LKDAFFCLRLHPTSQPLFAFEWRDPEMGIS | | |
| | | GQLTWTRLPQGFKNSPTLFDEALHRDLADF | | |
| | | RIQHPDLILLQYVDDLLLAATSELDCQQGT | | |
| | | RALLQTLGNLGYRASAKKAQICQKQVKYLG | | |
| | | YLLKEGQRWLTEARKETVMGQPTPKTPRQL | | |
| | | REFLGTAGFCRLWIPGFAEMAAPLYPLTKT | | |
| | | GTLFNWGPDQQKAYQEIKQALLTAPALGLP | | |
| | | DLTKPFELFVDEKQGYAKGVLTQKLGPWRR | | |
| | | PVAYLSKKLDPVAAGWPPCLRMVAAIAVLT | | |
| | | KDAGKLTMGQPLVILAPHAVEALVKQPPDR | | |
| | | WLSNARMTHYQALLLDTDRVQFGPVVALNP | | |
| | | ATLLPLPEEGLQHNCLDILAEAHGTRPDLT | | |
| | | DQPLPDADHTWYTDGSSLLQEGQRKAGAAV | | |
| | | TTETEVIWAKALPAGTSAQRAELIALTQAL | | |
| | | KMAEGKKLNVYTDSRYAFATAHIHGEIYRR | | |
| | | RGLLTSEGKEIKNKDEILALLKALFLPKRL | | |
| | | SIIHCPGHQKGHSAEARGNRMADQAARKAA | | |
| | | ITETPDTSTLL | | |
| 173 | RTx | TWLSDFPQAWAETGGVGLAVRQAPLIIPLK | | |
| | N-tag: His | ATSTPVSIKQYPISQKARLGIKPHIQRLLD | | |
| | SOLD 33 | QGILVPCRSPWNTPLLPVKKPGTNDYRPVQ | | |
| | VDG | DLREVNKRVEDIHPTVPNPYNLLSGPPPSH | | |
| | SOLD 33 | QWYTVLDLKDAFFCLRLHPTSQPLFAFEWR | | |
| | VDG | DPEMGISGQLTWTRLPQGFKNSPTLFNEAL | | |
| | | HRDLADFRIQHPDLILLQYVDDLLLAATSE | | |
| | | LDCQQGTRALLQTLGNLGYRASAKKAQICQ | | |
| | | KQVKYLGYLLKEGQRWLTEARKETVMGQPT | | |
| | | PKTPRQLRRFLGKAGFCRLFIPGFAEMAAP | | |
| | | LYPLTKPGTLFNWGPDQQKAYQEIKQALLT | | |
| | | APALGLPDLTKPFELFVDEKQGYAKGVLTQ | | |
| | | KLGPWRRPVAYLSKKLDPVAAGWPPCLRMV | | |
| | | AAIAVLTKDAGKLTMGQPLVIGAPHAVEAL | | |
| | | VKQPAGRWLSKARMTHYQALLLDTDRVQFG | | |
| | | PVVALNPATLLPLPEEGLQHNCLDILAEAH | | |
| | | GTRPDLTDQPLPDADHTWYTNGSSLLQEGQ | | |
| | | RKAGAAVTDETEVIWAKALPAGTSAQRAEL | | |
| | | IALTQALKMAEGKKLNVYTNSRYAFATAHI | | |
| | | HGEIYRRRGWLTSGGKEIKNKDEILALLKA | | |
| | | LFLPKRLSIIHCPGHQKGHSVEARGNRMAH | | |
| | | QAARRAAITETPDTSTLPIENSSPNSRLIN | | |
| 174 | RTx | TWLSDFPQAWAETGGMGLAVRQAPLIIPLK | 189 | ACTTGGCTGTCTGATTTCCCTCAGGCGTGGGCCGAAACGG |
| | N-tag: His | ATSTPVSIKQYPLSQKARLGIKPHIQRLLD | | GTGGCATGGGTCTGGCAGTGCGTCAGGCACCGCTGATTAT |
| | C-Tag: | QGILVPCQSPWNTPLLPVKKPGTNDYRPVQ | | TCCGCTGAAAGCGACGTCGACCCCGGTGAGCATCAAGCAA |
| | Dat90 | DLREVNKRVEDIHPTVPNPYNLLSGPPPSH | | TATCCGCTGTCCCAAAAGGCGCGCTTAGGTATTAAGCCGC |
| | C-DAT 42B | QWYTVLDLKDAFFCLRLHPTSQPLFAFEWR | | ACATTCAGCGTCTGCTGGATCAAGGTATTCTGGTTCCGTG |
| | L | DPEMGISGQLTWTRLPQGFKNSPTLFNEAL | | TCAGAGCCCGTGGAATACCCCGCTTCTCCCGGTGAAGAAA |
| | 42BL Dat1- | HRDLADFRIQHPDLILLQYVDDLLLAATSE | | CCGGGCACGAACGATTACCGTCCAGTCCAAGACTTGCGCG |
| | 90 | LDCQQGTRALLQTLGNLGYRASAKKAQICQ | | AAGTTAACAAGCGCGTTGAAGATATTCACCCGACCGTCCC |
| | | KQVKYLGYLLKEGQRWLTEARKETVMGQPT | | GAACCCGTACAATCTGCTGAGCGGTCCGCCGCCAAGCCAC |
| | | PKTPRQLRRFLGKAGFCRLFIPGFAEMAAP | | CAATGGTACACCGTGCTGGATCTGAAAGATGCTTTCTTCT |
| | | LYPLTKPGTLFNWGPDQQKAYQEIKQALLT | | GTCTGCGTCTGCACCCAACCAGCCAGCCTCTGTTTGCATT |
| | | APALGLPDLTKPFELFVDEKQGYAKGVLTQ | | TGAGTGGCGTGACCCTGAGATGGGTATTAGCGGCCAGCTG |
| | | KLGPWRRPVAYLSKKLDPVAAGWPPCLRMV | | ACGTGGACCCGCCTGCCGCAAGGTTTTAAGAATTCCCCTA |
| | | AAIAVLTKDAGKLTMGQPLVIGAPHAVEAL | | CGCTGTTTAACGAAGCGCTGCACCGTGACCTGGCGGATTT |
| | | VKQPAGRWLSKARMTHYQALLLDTDRVQFG | | CCGTATCCAGCACCCGGACCTGATCTTGCTGCAGTACGTT |
| | | PVVALNPATLLPLPEEGLQHNCLDILAEAH | | GATGACCTGTTGCTGGCGGCGACGAGCGAGCTGGATTGCC |
| | | GTRPDLTDQPLPDADHTWYTNGSSLLQEGQ | | AACAGGGCACCCGTGCGCTGTTGCAGACCTTGGGTAACCT |
| | | RKAGAAVTTETEVIWAKALPAGTSAQRAEL | | GGGTTATCGCGCTAGCGCGAAGAAAGCGCAGATTTGCCAA |
| | | IALTQALKMAEGKKLNVYTDSRYAFATAHI | | AAACAAGTTAAGTATCTGGGCTACCTGTTAAAGGAAGGCC |
| | | HGEIYRRRGWLTSKGKEIKNKDEILALLKA | | AACGTTGGCTGACCGAAGCCCGCAAAGAAACTGTCATGGG |
| | | LFLPKRLSIIHCPGHQKGHSAEARGNRMAD | | TCAGCCGACCCCGAAAACGCCACGCCAACTGCGTAGGTTC |
| | | QAARKAAITETPDTSTLLIENSSPNSRLIN | | TTGGGCAAAGCGGGTTTCTGCCGCCTGTTCATCCCGGGCT |
| | | GSGGSGAKTLAQGRKPGSGRKPGKGKTLRE | | TTGCCGAAATGGCAGCCCCGCTGTATCCGTTGACCAAGCC |
| | | GRKPGSGRRRRQDTGGKETDGSQQDQESRL | | GGGCACCCTGTTCAACTGG |
| | | ISSRDMEAVDALRELTHSPSSHSAHNSSAA | | GGTCCGGACCAGCAGAAAGCGTACCAAGAAATTAAACAAG |
| | | PPPHA | | CACTGCTGACGGCACCGGCGCTGGGTCTGCCGGACCTGAC |
| | | | | CAAGCCGTTTGAGCTGTTCGTGGATGAGAAGCAAGGTTAC |
| | | | | GCGAAGGGCGTGTTGACCCAGAAATTGGGTCCGTGGCGTC |
| | | | | GTCCGGTTGCATACCTGTCCAAGAAACTGGACCCGGTTGC |
| | | | | TGCTGGTTGGCCGCCTTGCCTGCGCATGGTTGCCGCTATC |
| | | | | GCGGTGCTGACTAAAGACGCGGGTAAGCTGACGATGGGTC |
| | | | | AACCGCTGGTGATCGGCGCACCGCATGCAGTCGAGGCCCT |
| | | | | TGTTAAGCAACCGGCAGGAAGATGGCTGAGCAAGGCGCGT |
| | | | | ATGACGCATTACCAGGCACTGCTGTTGGACACCGATCGTG |
| | | | | TGCAGTTTGGCCCGGTCGTTGCGCTCAACCCGGCGACCCT |
| | | | | GCTGCCGCTCCCGGAAGAAGGCTTGCAGCACAACTGTTTG |
| | | | | GACATCCTGGCAGAGGCGCACGGCACTCGCCCGGATCTGA |
| | | | | CGGACCAGCCGCTGCCGGACGCCGATCATACCTGGTATAC |
| | | | | GAATGGTAGCAGCCTGTTGCAAGAGGGTCAGCGTAAGGCC |
| | | | | GGTGCCGCGGTCACCACCGAGACTGAAGTGATTTGGGCTA |
| | | | | AAGCATTGCCTGCGGGTACCAGCGCGCAGCGTGCCGAGCT |
| | | | | GATCGCACTGACCCAAGCGCTGAAAATGGCTGAGGGTAAG |
| | | | | AAACTGAATGTGTACACGGATAGCCGTTATGCCTTTGCGA |
| | | | | CCGCCCACATTCACGGCGAGATCTATCGCCGTCGCGGCTG |
| | | | | GCTGACGTCCAAAGGCAAAGAGATCAAGAATAAAGACGAA |
| | | | | ATTCTGGCGCTGCTGAAAGCGCTGTTCCTGCCGAAACGTC |
| | | | | TGTCGATCATCCATTGCCCGGGTCACCAGAAAGGCCACAG |
| | | | | CGCAGAGGCGCGTGGTAATCGCATGGCTGACCAGGCTGCG |
| | | | | CGTAAAGCCGCAATTACCGAAACCCCGGACACCAGCACGC |
| | | | | TGCTGATCGAGAATAGCAGCCCGAACAGCCGTCTGATCAA |
| | | | | TGGTTCAGGGGGTTCCGGTGCAAAGACTTTGGCCCAGGGG |
| | | | | CGCAAACCGGGTAGCGGTCGCAAACCCGGAAAGGGCAAAA |
| | | | | CTTTACGTGAAGGCCGCAAGCCTGGATCTGGTCGTCGTCG |
| | | | | CCGCCAAGACACAGGGGGTAAGGAGACTGATGGATCACAA |
| | | | | CAAGACCAGGAGTCCCGTCTGATCTCATCGCGCGACATGG |
| | | | | AGGCAGTAGACGCTTTGCGCGAATTAACCCATAGTCCGAG |
| | | | | TTCACACTCTGCCCACAATTCCTCTGCTGCACCACCACCG |
| | | | | CACGCCTGA |
| 175 | RTx | AKTLAQGRKPGSGRKPGKGKTLREGRKPGS | 190 | GCAAAGACTTTGGCCCAGGGGCGCAAACCGGGTAGCGGTC |
| | N-tag: His- | GRRRRQDTGGKETDGSQQDQESRLISSRDM | | GCAAACCCGGAAAGGGCAAAACTTTACGTGAAGGCCGCAA |
| | Dat90 | EAVDALRELTHSPSSHSAHNSSAAPPPHAG | | GCCTGGATCTGGTCGTCGTCGCCGCCAAGACACAGGGGGT |
| | N-DAT 42B | SGGSGTWLSDFPQAWAETGGMGLAVRQAPL | | AAGGAGACTGATGGATCACAACAAGACCAGGAGTCCCGTC |
| | L | IIPLKATSTPVSIKQYPLSQKARLGIKPHI | | TGATCTCATCGCGCGACATGGAGGCAGTAGACGCTTTGCG |
| | Dat1- | QRLLDQGILVPCQSPWNTPLLPVKKPGTND | | CGAATTAACCCATAGTCCGAGTTCACACTCTGCCCACAAT |
| | 90_42BL | YRPVQDLREVNKRVEDIHPTVPNPYNLLSG | | TCCTCTGCTGCACCACCACCGCACGCCGGTTCAGGGGGTT |
| | | PPPSHQWYTVLDLKDAFFCLRLHPTSQPLF | | CCGGTACTTGGCTGTCTGATTTCCCTCAGGCGTGGGCCGA |
| | | AFEWRDPEMGISGQLTWTRLPQGFKNSPTL | | AACGGGTGGCATGGGTCTGGCAGTGCGTCAGGCACCGCTG |
| | | FNEALHRDLADFRIQHPDLILLQYVDDLLL | | ATTATTCCGCTGAAAGCGACGTCGACCCCGGTGAGCATCA |
| | | AATSELDCQQGTRALLQTLGNLGYRASAKK | | AGCAATATCCGCTGTCCCAAAAGGCGCGCTTAGGTATTAA |
| | | AQICQKQVKYLGYLLKEGQRWLTEARKETV | | GCCGCACATTCAGCGTCTGCTGGATCAAGGTATTCTGGTT |
| | | MGQPTPKTPRQLRRFLGKAGFCRLFIPGFA | | CCGTGTCAGAGCCCGTGGAATACCCCGCTTCTCCCGGTGA |
| | | EMAAPLYPLTKPGTLFNWGPDQQKAYQEIK | | AGAAACCGGGCACGAACGATTACCGTCCAGTCCAAGACTT |
| | | QALLTAPALGLPDLTKPFELFVDEKQGYAK | | GCGCGAAGTTAACAAGCGCGTTGAAGATATTCACCCGACC |
| | | GVLTQKLGPWRRPVAYLSKKLDPVAAGWPP | | GTCCCGAACCCGTACAATCTGCTGAGCGGTCCGCCGCCAA |
| | | CLRMVAAIAVLTKDAGKLTMGQPLVIGAPH | | GCCACCAATGGTACACCGTGCTGGATCTGAAAGATGCTTT |
| | | AVEALVKQPAGRWLSKARMTHYQALLLDTD | | CTTCTGTCTGCGTCTGCACCCAACCAGCCAGCCTCTGTTT |
| | | RVQFGPVVALNPATLLPLPEEGLQHNCLDI | | GCATTTGAGTGGCGTGACCCTGAGATGGGTATTAGCGGCC |
| | | LAEAHGTRPDLTDQPLPDADHTWYTNGSSL | | AGCTGACGTGGACCCGCCTGCCGCAAGGTTTTAAGAATTC |
| | | LQEGQRKAGAAVTTETEVIWAKALPAGTSA | | CCCTACGCTGTTTAACGAAGCGCTGCACCGTGACCTGGCG |
| | | QRAELIALTQALKMAEGKKLNVYTDSRYAF | | GATTTCCGTATCCAGCACCCGGACCTGATCTTGCTGCAGT |
| | | ATAHIHGEIYRRRGWLTSKGKEIKNKDEIL | | ACGTTGATGACCTGTTGCTGGCGGCGACGAGCGAGCTGGA |
| | | ALLKALFLPKRLSIIHCPGHQKGHSAEARG | | TTGCCAACAGGGCACCCGTGCGCTGTTGCAGACCTTGGGT |
| | | NRMADQAARKAAITETPDTSTLLIENSSPN | | AACCTGGGTTATCGCGCTAGCGCGAAGAAAGCGCAGATTT |
| | | SRLIN | | GCCAAAAACAAGTTAAGTATCTGGGCTACCTGTTAAAGGA |
| | | | | AGGCCAACGTTGGCTGACCGAAGCCCGCAAAGAAACTGTC |
| | | | | ATGGGTCAGCCGACCCCGAAAACGCCACGCCAACTGCGTA |
| | | | | GGTTCTTGGGCAAAGCGGGTTTCTGCCGCCTGTTCATCCC |
| | | | | GGGCTTTGCCGAAATGGCAGCCCCGCTGTATCCGTTGACC |
| | | | | AAGCCGGGCACCCTGTTCAACTGGGGTCCGGACCAGCAGA |
| | | | | AAGCGTACCAAGAAATTAAACAAGCACTGCTGACGGCACC |
| | | | | GGCGCTGGGTCTGCCGGACCTGACCAAGCCGTTTGAGCTG |
| | | | | TTCGTGGATGAGAAGCAAGGTTACGCGAAGGGCGTGTTGA |
| | | | | CCCAGAAATTGGGTCCGTGGCGTCGTCCGGTTGCATACCT |
| | | | | GTCCAAGAAACTGGACCCGGTTGCTGCTGGTTGGCCGCCT |
| | | | | TGCCTGCGCATGGTTGCCGCTATCGCGGTGCTGACTAAAG |
| | | | | ACGCGGGTAAGCTGACGATGGGTCAACCGCTGGTGATCGG |
| | | | | CGCACCGCATGCAGTCGAGGCCCTTGTTAAGCAACCGGCA |
| | | | | GGAAGATGGCTGAGCAAGGCGCGTATGACGCATTACCAGG |
| | | | | CACTGCTGTTGGACACCGATCGTGTGCAGTTTGGCCCGGT |
| | | | | CGTTGCGCTCAACCCGGCGACCCTGCTGCCGCTCCCGGAA |
| | | | | GAAGGCTTGCAGCACAACTGTTTGGACATCCTGGCAGAGG |
| | | | | CGCACGGCACTCGCCCGGATCTGACGGACCAGCCGCTGCC |
| | | | | GGACGCCGATCATACCTGGTATACGAATGGTAGCAGCCTG |
| | | | | TTGCAAGAGGGTCAGCGTAAGGCCGGTGCCGCGGTCACCA |
| | | | | CCGAGACTGAAGTGATTTGGGCTAAAGCATTGCCTGCGGG |
| | | | | TACCAGCGCGCAGCGTGCCGAGCTGATCGCACTGACCCAA |
| | | | | GCGCTGAAAATGGCTGAGGGTAAGAAACTGAATGTGTACA |
| | | | | CGGATAGCCGTTATGCCTTTGCGACCGCCCACATTCACGG |
| | | | | CGAGATCTATCGCCGTCGCGGCTGGCTGACGTCCAAAGGC |
| | | | | AAAGAGATCAAGAATAAAGACGAAATTCTGGCGCTGCTGA |
| | | | | AAGCGCTGTTCCTGCCGAAACGTCTGTCGATCATCCATTG |
| | | | | CCCGGGTCACCAGAAAGGCCACAGCGCAGAGGCGCGTGGT |
| | | | | AATCGCATGGCTGACCAGGCTGCGCGTAAAGCCGCAATTA |
| | | | | CCGAAACCCCGGACACCAGCACGCTGCTGATCGAGAATAG |
| | | | | CAGCCCGAACAGCCGTCTGATCAATTGA |
| 176 | RTx | AKTLAQGRKPGSGRKPGKGKTLREGRKPGS | | |
| | N-tag: His- | GRRRRQDTGGKETDGSQQDQESRLISSRDM | | |
| | Dat90 | EAVDALRELTHSPSSHSAHNSSAAPPPHAG | | |
| | N-DAT-90 | SGGSGTWLSDFPQAWAETGGLGLAVRQAPL | | |
| | SOLD 01 | IIPLKATSTPVSIKQYPLSQKARLGIKPHI | | |
| | N-DAT-90 | QRLLDQGILVPCQSPWNTPLLPVKKPGTND | | |
| | SOLD 01 | YRPVQDLREVNKRVEDIHPTVPNPYNLLSG | | |
| | | PPPSHQWYTVLDLKDAFFCLRLHPTSQPLF | | |
| | | AFEWRDPEMGISGQLTWTRLPQGFKNSPTL | | |
| | | FNEALHRDLADFRIQHPDLILLQYVDDLLL | | |
| | | AATSELDCQQGTRALLQTLGNLGYRASAKK | | |
| | | AQICQKQVKYLGYLLKEGQRWLTEARKETV | | |
| | | MGQPTPKTPRQLRRFLGKAGFCRLFIPGFA | | |
| | | EMAAPLYPLTKPGTLFNWGPDQQKAYQEIK | | |
| | | QALLTAPALGLPDLTKPFELFVDEKQGYAK | | |
| | | GVLTQKLGPWRRPVAYLSKKLDPVAAGWPP | | |
| | | CLRMVAAIAVLTKDAGKLTMGQPLVIGAPH | | |
| | | AVEALVKQPAGRWLSKARMTHYQALLLDTD | | |
| | | RVQFGPVVALNPATLLPLPEEGLQHNCLDI | | |
| | | LAEAHGTRPDLTDQPLPDADHTWYTNGSSL | | |
| | | LQEGQRKAGAAVTTETEVIWAKALPAGTSA | | |
| | | QRAELIALTQALKMAEGKKLNVYTDSRYAF | | |
| | | ATAHIHGEIYRRRGWLTSKGKEIKNKDEIL | | |
| | | ALLKALFLPKRLSIIHCPGHQKGHSAEARG | | |
| | | NRMADQAARKAAITETPDTSTLLIENSSPN | | |
| | | SRLIN | | |
| 177 | RTx | TWLSDFPQAWAETGGLGLAVRQAPLIIPLK | | |
| | N-tag: His | ATSTPVSIKQYPLSQKARLGIKPHIQRLLD | | |
| | C-Tag: | QGILVPCQSPWNTPLLPVKKPGTNDYRPVQ | | |
| | Dat90 | DLREVNKRVEDIHPTVPNPYNLLSGPPPSH | | |
| | C-DAT-90 | QWYTVLDLKDAFFCLRLHPTSQPLFAFEWR | | |
| | SOLD 01 | DPEMGISGQLTWTRLPQGFKNSPTLFNEAL | | |
| | C-DAT-90 | HRDLADFRIQHPDLILLQYVDDLLLAATSE | | |
| | SOLD 01 | LDCQQGTRALLQTLGNLGYRASAKKAQICQ | | |
| | | KQVKYLGYLLKEGQRWLTEARKETVMGQPT | | |
| | | PKTPRQLRRFLGKAGFCRLFIPGFAEMAAP | | |
| | | LYPLTKPGTLFNWGPDQQKAYQEIKQALLT | | |
| | | APALGLPDLTKPFELFVDEKQGYAKGVLTQ | | |
| | | KLGPWRRPVAYLSKKLDPVAAGWPPCLRMV | | |
| | | AAIAVLTKDAGKLTMGQPLVIGAPHAVEAL | | |
| | | VKQPAGRWLSKARMTHYQALLLDTDRVQFG | | |
| | | PVVALNPATLLPLPEEGLQHNCLDILAEAH | | |
| | | GTRPDLTDQPLPDADHTWYTNGSSLLQEGQ | | |
| | | RKAGAAVTTETEVIWAKALPAGTSAQRAEL | | |
| | | IALTQALKMAEGKKLNVYTDSRYAFATAHI | | |
| | | HGEIYRRRGWLTSKGKEIKNKDEILALLKA | | |
| | | LFLPKRLSIIHCPGHQKGHSAEARGNRMAD | | |
| | | QAARKAAITETPDTSTLLIENSSPNSRLIN | | |
| | | GSGGSGAKTLAQGRKPGSGRKPGKGKTLRE | | |
| | | GRKPGSGRRRRQDTGGKETDGSQQDQESRL | | |
| | | ISSRDMEAVDALRELTHSPSSHSAHNSSAA | | |
| | | PPPHA | | |
| 178 | RTx | AKTLAQGRKPGSGRKPGKGKTLREGRKPGS | | |
| | N-tag: His- | GRRRRQDTGGKETDGSQQDQESRLISSRDM | | |
| | Dat90 | EAVDALRELTHSPSSHSAHNSSAAPPPHAG | | |
| | N-DAT 42B | SGGSGTWLSDFPQAWAETGGMGLAVRQAPL | | |
| | N-DAT-90 | IIPLKATSTPVSIKQYPMSQKARLGIKPHI | | |
| | 42B | QRLLDQGILVPCQSPWNTPLLPVKKPGTND | | |
| | | YRPVQDLREVNKRVEDIHPTVPNPYNLLSG | | |
| | | PPPSHQWYTVLDLKDAFFCLRLHPTSQPLF | | |
| | | AFEWRDPEMGISGQLTWTRLPQGFKNSPTL | | |
| | | FNEALHRDLADFRIQHPDLILLQYVDDLLL | | |
| | | AATSELDCQQGTRALLQTLGNLGYRASAKK | | |
| | | AQICQKQVKYLGYLLKEGQRWLTEARKETVM | | |
| | | GQPTPKTPRQLRRFLGKAGFCRLFIPGFAEM | | |
| | | AAPLYPLTKPGTLFNWGPDQQKAYQEIKQAL | | |
| | | LTAPALGLPDLTKPFELFVDEKQGYAKGVL | | |
| | | TQKLGPWRRPVAYLSKKLDPVAAGWPPCLR | | |
| | | MVAAIAVLTKDAGKLTMGQPLVIGAPHAVE | | |
| | | ALVKQPAGRWLSKARMTHYQALLLDTDRVQ | | |
| | | FGPVVALNPATLLPLPEEGLQHNCLDILAE | | |
| | | AHGTRPDLTDQPLPDADHTWYTNGSSLLQE | | |
| | | GQRKAGAAVTTETEVIWAKALPAGTSAQRA | | |
| | | ELIALTQALKMAEGKKLNVYTDSRYAFATA | | |
| | | HIHGEIYRRRGWLTSKGKEIKNKDEILALL | | |
| | | KALFLPKRLSIIHCPGHQKGHSAEARGNRM | | |
| | | ADQAARKAAITETPDTSTLLIENSSPNSRL | | |
| | | IN | | |
| 179 | RTx | AKTLAQGRKPGSGRKPGKGKTLREGRKPGS | | |
| | N-tag: His- | GRRRRQDTGGKETDGSQQDQESRLISSRDM | | |
| | Dat90 | EAVDALRELTHSPSSHSAHNSSAAPPPHAG | | |
| | N-DAT | SGGSGTWLSDFPQAWAETGGMGLAVRQAPL | | |
| | 50A+ G | IIPLKATSTPVSIKQYPLSQKARLGIKPHI | | |
| | N-DAT-90 | QRLLDQGILVPCQSPWNTPLLPVKKPGTND | | |
| | 50A+ G | YRPVQDLREVNKRVEDIHPTVPNPYNLLSG | | |
| | | PPPSHQWYTVLDLKDAFFCLRLHPTSQPLF | | |
| | | AFEWRDPEMGISGQLTWTRLPQGFKNSPTL | | |
| | | FNEALHRDLADFRIQHPDLILLQYVDDLLL | | |
| | | AATSELDCQQGTRALLQTLGNLGYRASAKK | | |
| | | AQICQKQVKYLGYLLKEGQRWLTEARKETV | | |
| | | MGQPTPKTPRQLRRFLGKAGFCRLFIPGFA | | |
| | | EMAAPLYPLTKPGTLFNWGPDQQKAYQEIK | | |
| | | QALLTAPALGLPDLTKPFELFVDEKQGYAK | | |
| | | GVLTQKLGPWRRPVAYLSKKLDPVAAGWPP | | |
| | | CLRMVAAIAVLTKDAGKLTMGQPLVIGAPH | | |
| | | AVEALVKQPPDRWLSKARMTHYQALLLDTD | | |
| | | RVQFGPVVALNPATLLPLPEEGLQHNCLDI | | |
| | | LAEAVGTRPDLTDQPLPDADHTWYTNGSSL | | |
| | | LQEGQRKAGAAVTTETEVIWAKALPAGTSA | | |
| | | QRAELIALTQALKMAEGKKLNVYTDSRYAF | | |
| | | ATAHIHGEIYRRRGWLTSKGKEIKNKDEIL | | |
| | | ALLKALFLPKRLSIIYCPGHQKGHSAEARG | | |
| | | NRMADQAARKAAITETPDTSTLLIENSSPN | | |
| | | SRLIN | | |
| 180 | N-DAT-90 | AKTLAQGRKPGSGRKPGKGKTLREGRKPGS | | |
| | SOLD 33 | GRRRRQDTGGKETDGSQQDQESRLISSRDM | | |
| | VDG | EAVDALRELTHSPSSHSAHNSSAAPPPHAG | | |
| | | SGGSGTWLSDFPQAWAETGGVGLAVRQAPL | | |
| | | IIPLKATSTPVSIKQYPISQKARLGIKPHI | | |
| | | QRLLDQGILVPCRSPWNTPLLPVKKPGTND | | |
| | | YRPVQDLREVNKRVEDIHPTVPNPYNLLSG | | |
| | | PPPSHQWYTVLDLKDAFFCLRLHPTSQPLF | | |
| | | AFEWRDPEMGISGQLTWTRLPQGFKNSPTL | | |
| | | FNEALHRDLADFRIQHPDLILLQYVDDLLL | | |
| | | AATSELDCQQGTRALLQTLGNLGYRASAKK | | |
| | | AQICQKQVKYLGYLLKEGQRWLTEARKETV | | |
| | | MGQPTPKTPRQLRRFLGKAGFCRLFIPGFA | | |
| | | EMAAPLYPLTKPGTLFNWGPDQQKAYQEIK | | |
| | | QALLTAPALGLPDLTKPFELFVDEKQGYAK | | |
| | | GVLTQKLGPWRRPVAYLSKKLDPVAAGWPP | | |
| | | CLRMVAAIAVLTKDAGKLTMGQPLVIGAPH | | |
| | | AVEALVKQPAGRWLSKARMTHYQALLLDTD | | |
| | | RVQFGPVVALNPATLLPLPEEGLQHNCLDI | | |
| | | LAEAHGTRPDLTDQPLPDADHTWYTNGSSL | | |
| | | LQEGQRKAGAAVTDETEVIWAKALPAGTSA | | |
| | | QRAELIALTQALKMAEGKKLNVYTNSRYAF | | |
| | | ATAHIHGEIYRRRGWLTSGGKEIKNKDEIL | | |
| | | ALLKALFLPKRLSIIHCPGHQKGHSVEARG | | |
| | | NRMAHQAARRAAITETPDTSTLPIENSSPN | | |
| 181 | N-Dat36 | AKTLAQGRKPGSGRKPGKGKTLREGRKPGS | | |
| | 42BL | GRRRRGSGGSGTWLSDFPQAWAETGGMGLA | | |
| | | VRQAPLIIPLKATSTPVSIKQYPLSQKARL | | |
| | | GIKPHIQRLLDQGILVPCQSPWNTPLLPVK | | |
| | | KPGTNDYRPVQDLREVNKRVEDIHPTVPNP | | |
| | | YNLLSGPPPSHQWYTVLDLKDAFFCLRLHP | | |
| | | TSQPLFAFEWRDPEMGISGQLTWTRLPQGF | | |
| | | KNSPTLFNEALHRDLADFRIQHPDLILLQY | | |
| | | VDDLLLAATSELDCQQGTRALLQTLGNLGY | | |
| | | RASAKKAQICQKQVKYLGYLLKEGQRWLTE | | |
| | | ARKETVMGQPTPKTPRQLRRFLGKAGFCRL | | |
| | | FIPGFAEMAAPLYPLTKPGTLFNWGPDQQK | | |
| | | AYQEIKQALLTAPALGLPDLTKPFELFVDE | | |
| | | KQGYAKGVLTQKLGPWRRPVAYLSKKLDPV | | |
| | | AAGWPPCLRMVAAIAVLTKDAGKLTMGQPL | | |
| | | VIGAPHAVEALVKQPAGRWLSKARMTHYQA | | |
| | | LLLDTDRVQFGPVVALNPATLLPLPEEGLQ | | |
| | | HNCLDILAEAHGTRPDLTDQPLPDADHTWY | | |
| | | TNGSSLLQEGQRKAGAAVTTETEVIWAKAL | | |
| | | PAGTSAQRAELIALTQALKMAEGKKLNVYT | | |
| | | DSRYAFATAHIHGEIYRRRGWLTSKGKEIK | | |
| | | NKDEILALLKALFLPKRLSIIHCPGHQKGH | | |
| | | SAEARGNRMADQAARKAAITETPDTSTLLI | | |
| | | ENSSPNSRLIN | | |
| 182 | C-Dat36 | TWLSDFPQAWAETGGMGLAVRQAPLIIPLK | | |
| | 42BL | ATSTPVSIKQYPLSQKARLGIKPHIQRLLD | | |
| | | QGILVPCQSPWNTPLLPVKKPGTNDYRPVQ | | |
| | | DLREVNKRVEDIHPTVPNPYNLLSGPPPSH | | |
| | | QWYTVLDLKDAFFCLRLHPTSQPLFAFEWR | | |
| | | DPEMGISGQLTWTRLPQGFKNSPTLFNEAL | | |
| | | HRDLADFRIQHPDLILLQYVDDLLLAATSE | | |
| | | LDCQQGTRALLQTLGNLGYRASAKKAQICQ | | |
| | | KQVKYLGYLLKEGQRWLTEARKETVMGQPT | | |
| | | PKTPRQLRRFLGKAGFCRLFIPGFAEMAAP | | |
| | | LYPLTKPGTLFNWGPDQQKAYQEIKQALLT | | |
| | | APALGLPDLTKPFELFVDEKQGYAKGVLTQ | | |
| | | KLGPWRRPVAYLSKKLDPVAAGWPPCLRMV | | |
| | | AAIAVLTKDAGKLTMGQPLVIGAPHAVEAL | | |
| | | VKQPAGRWLSKARMTHYQALLLDTDRVQFG | | |
| | | PVVALNPATLLPLPEEGLQHNCLDILAEAH | | |
| | | GTRPDLTDQPLPDADHTWYTNGSSLLQEGQ | | |
| | | RKAGAAVTTETEVIWAKALPAGTSAQRAEL | | |
| | | IALTQALKMAEGKKLNVYTDSRYAFATAHI | | |
| | | HGEIYRRRGWLTSKGKEIKNKDEILALLKA | | |
| | | LFLPKRLSIIHCPGHQKGHSAEARGNRMAD | | |
| | | QAARKAAITETPDTSTLLIENSSPNSRLIN | | |
| | | GSGGSGAKTLAQGRKPGSGRKPGKGKTLRE | | |
| | | GRKPGSGRRRR | | |
| 183 | N-Dat1full | AKTLAQGRKPGSGRKPGKGKTLREGRKPGS | 191 | GCAAAAACTTTGGCTCAAGGGCGCAAACCTGGATCAGGCC |
| | 42BL | GRRRRQDTGGKETDGSQQDQESRLISSRDM | | GCAAGCCGGGGAAGGGTAAAACTTTGCGCGAGGGGCGTAA |
| | | EAVDALRELTHSPSSHSAHNSSAAPPPHAA | | GCCTGGATCGGGGCGTCGTCGTCGTCAGGATACTGGAGGG |
| | | AASTSLPPSLDYTHQSFMDQQQQQQQQQQL | | AAAGAAACTGACGGCTCCCAACAGGATCAAGAAAGCCGCT |
| | | LQQQRVDVVPPKPFITHKILLSSTGNSGGH | | TGATTTCCTCCCGCGACATGGAAGCCGTCGATGCACTTCG |
| | | VNSNYNADHSINHNSNHNLNSNVNVNMNFT | | TGAGCTTACACATAGCCCGTCAAGCCATAGCGCCCATAAT |
| | | INGSNQDPSSSFLMGPYNYLQRPFIVKPYL | | AGCTCAGCCGCACCCCCACCACATGCCGCAGCGGCTAGCA |
| | | DLSTSTAASNQPRTQPSPAAHITKNSDSTE | | CTTCCCTTCCACCATCGTTAGATTACACGCATCAATCGTT |
| | | KNATIGSGGSGTWLSDFPQAWAETGGMGLA | | CATGGATCAGCAACAACAACAACAGCAACAGCAACAGCAG |
| | | VRQAPLIIPLKATSTPVSIKQYPLSQKARL | | CAGCTTTTACAGCAACAGCGTGTCGACGTTGTCCCTCCGA |
| | | GIKPHIQRLLDQGILVPCQSPWNTPLLPVK | | AGCCCTTTATCACCCATAAGATCCTGTTGAGCTCTACTGG |
| | | KPGTNDYRPVQDLREVNKRVEDIHPTVPNP | | CAACTCAGGCGGGCACGTTAACAGCAACTACAACGCAGAT |
| | | YNLLSGPPPSHQWYTVLDLKDAFFCLRLHP | | CATTCGATCAACCATAATTCAAATCACAACCTTAATTCCA |
| | | TSQPLFAFEWRDPEMGISGQLTWTRLPQGF | | ACGTTAACGTGAACATGAACTTTACGATTAACGGTAGCAA |
| | | KNSPTLFNEALHRDLADFRIQHPDLILLQY | | TCAAGACCCATCATCCAGTTTCTTGATGGGCCCATACAAC |
| | | VDDLLLAATSELDCQQGTRALLQTLGNLGY | | TATTTACAACGCCCTTTCATCGTGAAGCCCTATTTGGATT |
| | | RASAKKAQICQKQVKYLGYLLKEGQRWLTE | | TATCGACTTCTACGGCAGCCAGTAACCAACCGCGTACACA |
| | | ARKETVMGQPTPKTPRQLRRFLGKAGFCRL | | GCCTTCCCCAGCGGCACATATCACAAAGAATAGCGATAGT |
| | | FIPGFAEMAAPLYPLTKPGTLFNWGPDQQK | | ACGGAGAAGAACGCCACAATTGGTTCAGGGGGTTCCGGTA |
| | | AYQEIKQALLTAPALGLPDLTKPFELFVDE | | CTTGGCTGTCTGATTTCCCTCAGGCGTGGGCCGAAACGGG |
| | | KQGYAKGVLTQKLGPWRRPVAYLSKKLDPV | | TGGCATGGGTCTGGCAGTGCGTCAGGCACCGCTGATTATT |
| | | AAGWPPCLRMVAAIAVLTKDAGKLTMGQPL | | CCGCTGAAAGCGACGTCGACCCCGGTGAGCATCAAGCAAT |
| | | VIGAPHAVEALVKQPAGRWLSKARMTHYQA | | ATCCGCTGTCCCAAAAGGCGCGCTTAGGTATTAAGCCGCA |
| | | LLLDTDRVQFGPVVALNPATLLPLPEEGLQ | | CATTCAGCGTCTGCTGGATCAAGGTATTCTGGTTCCGTGT |
| | | HNCLDILAEAHGTRPDLTDQPLPDADHTWY | | CAGAGCCCGTGGAATACCCCGCTTCTCCCGGTGAAGAAAC |
| | | TNGSSLLQEGQRKAGAAVTTETEVIWAKAL | | CGGGCACGAACGATTACCGTCCAGTCCAAGACTTGCGCGA |
| | | PAGTSAQRAELIALTQALKMAEGKKLNVYT | | AGTTAACAAGCGCGTTGAAGATATTCACCCGACCGTCCCG |
| | | DSRYAFATAHIHGEIYRRRGWLTSKGKEIK | | AACCCGTACAATCTGCTGAGCGGTCCGCCGCCAAGCCACC |
| | | NKDEILALLKALFLPKRLSIIHCPGHQKGH | | AATGGTACACCGTGCTGGATCTGAAAGATGCTTTCTTCTG |
| | | SAEARGNRMADQAARKAAITETPDTSTLLI | | TCTGCGTCTGCACCCAACCAGCCAGCCTCTGTTTGCATTT |
| | | ENSSPNSRLIN | | GAGTGGCGTGACCCTGAGATGGGTATTAGCGGCCAGCTGA |
| | | | | CGTGGACCCGCCTGCCGCAAGGTTTTAAGAATTCCCCTAC |
| | | | | GCTGTTTAACGAAGCGCTGCACCGTGACCTGGCGGATTTC |
| | | | | CGTATCCAGCACCCGGACCTGATCTTGCTGCAGTACGTTG |
| | | | | ATGACCTGTTGCTGGCGGCGACGAGCGAGCTGGATTGCCA |
| | | | | ACAGGGCACCCGTGCGCTGTTGCAGACCTTGGGTAACCTG |
| | | | | GGTTATCGCGCTAGCGCGAAGAAAGCGCAGATTTGCCAAA |
| | | | | AACAAGTTAAGTATCTGGGCTACCTGTTAAAGGAAGGCCA |
| | | | | ACGTTGGCTGACCGAAGCCCGCAAAGAAACTGTCATGGGT |
| | | | | CAGCCGACCCCGAAAACGCCACGCCAACTGCGTAGGTTCT |
| | | | | TGGGCAAAGCGGGTTTCTGCCGCCTGTTCATCCCGGGCTT |
| | | | | TGCCGAAATGGCAGCCCCGCTGTATCCGTTGACCAAGCCG |
| | | | | GGCACCCTGTTCAACTGGGGTCCGGACCAGCAGAAAGCGT |
| | | | | ACCAAGAAATTAAACAAGCACTGCTGACGGCACCGGCGCT |
| | | | | GGGTCTGCCGGACCTGACCAAGCCGTTTGAGCTGTTCGTG |
| | | | | GATGAGAAGCAAGGTTACGCGAAGGGCGTGTTGACCCAGA |
| | | | | AATTGGGTCCGTGGCGTCGTCCGGTTGCATACCTGTCCAA |
| | | | | GAAACTGGACCCGGTTGCTGCTGGTTGGCCGCCTTGCCTG |
| | | | | CGCATGGTTGCCGCTATCGCGGTGCTGACTAAAGACGCGG |
| | | | | GTAAGCTGACGATGGGTCAACCGCTGGTGATCGGCGCACC |
| | | | | GCATGCAGTCGAGGCCCTTGTTAAGCAACCGGCAGGAAGA |
| | | | | TGGCTGAGCAAGGCGCGTATGACGCATTACCAGGCACTGC |
| | | | | TGTTGGACACCGATCGTGTGCAGTTTGGCCCGGTCGTTGC |
| | | | | GCTCAACCCGGCGACCCTGCTGCCGCTCCCGGAAGAAGGC |
| | | | | TTGCAGCACAACTGTTTGGACATCCTGGCAGAGGCGCACG |
| | | | | GCACTCGCCCGGATCTGACGGACCAGCCGCTGCCGGACGC |
| | | | | CGATCATACCTGGTATACGAATGGTAGCAGCCTGTTGCAA |
| | | | | GAGGGTCAGCGTAAGGCCGGTGCCGCGGTCACCACCGAGA |
| | | | | CTGAAGTGATTTGGGCTAAAGCATTGCCTGCGGGTACCAG |
| | | | | CGCGCAGCGTGCCGAGCTGATCGCACTGACCCAAGCGCTG |
| | | | | AAAATGGCTGAGGGTAAGAAACTGAATGTGTACACGGATA |
| | | | | GCCGTTATGCCTTTGCGACCGCCCACATTCACGGCGAGAT |
| | | | | CTATCGCCGTCGCGGCTGGCTGACGTCCAAAGGCAAAGAG |
| | | | | ATCAAGAATAAAGACGAAATTCTGGCGCTGCTGAAAGCGC |
| | | | | TGTTCCTGCCGAAACGTCTGTCGATCATCCATTGCCCGGG |
| | | | | TCACCAGAAAGGCCACAGCGCAGAGGCGCGTGGTAATCGC |
| | | | | ATGGCTGACCAGGCTGCGCGTAAAGCCGCAATTACCGAAA |
| | | | | CCCCGGACACCAGCACGCTGCTGATCGAGAATAGCAGCCC |
| | | | | GAACAGCCGTCTGATCAATTGA |
| 184 | C-Dat 1full | TWLSDFPQAWAETGGMGLAVRQAPLIIPLK | 192 | ACTTGGCTGTCTGATTTCCCTCAGGCGTGGGCCGAAACGG |
| | 42BL | ATSTPVSIKQYPLSQKARLGIKPHIQRLLD | | GTGGCATGGGTCTGGCAGTGCGTCAGGCACCGCTGATTAT |
| | | QGILVPCQSPWNTPLLPVKKPGTNDYRPVQ | | TCCGCTGAAAGCGACGTCGACCCCGGTGAGCATCAAGCAA |
| | | DLREVNKRVEDIHPTVPNPYNLLSGPPPSH | | TATCCGCTGTCCCAAAAGGCGCGCTTAGGTATTAAGCCGC |
| | | QWYTVLDLKDAFFCLRLHPTSQPLFAFEWR | | ACATTCAGCGTCTGCTGGATCAAGGTATTCTGGTTCCGTG |
| | | DPEMGISGQLTWTRLPQGFKNSPTLFNEAL | | TCAGAGCCCGTGGAATACCCCGCTTCTCCCGGTGAAGAAA |
| | | HRDLADFRIQHPDLILLQYVDDLLLAATSE | | CCGGGCACGAACGATTACCGTCCAGTCCAAGACTTGCGCG |
| | | LDCQQGTRALLQTLGNLGYRASAKKAQICQ | | AAGTTAACAAGCGCGTTGAAGATATTCACCCGACCGTCCC |
| | | KQVKYLGYLLKEGQRWLTEARKETVMGQPT | | GAACCCGTACAATCTGCTGAGCGGTCCGCCGCCAAGCCAC |
| | | PKTPRQLRRFLGKAGFCRLFIPGFAEMAAP | | CAATGGTACACCGTGCTGGATCTGAAAGATGCTTTCTTCT |
| | | LYPLTKPGTLFNWGPDQQKAYQEIKQALLT | | GTCTGCGTCTGCACCCAACCAGCCAGCCTCTGTTTGCATT |
| | | APALGLPDLTKPFELFVDEKQGYAKGVLTQ | | TGAGTGGCGTGACCCTGAGATGGGTATTAGCGGCCAGCTG |
| | | KLGPWRRPVAYLSKKLDPVAAGWPPCLRMV | | ACGTGGACCCGCCTGCCGCAAGGTTTTAAGAATTCCCCTA |
| | | AAIAVLTKDAGKLTMGQPLVIGAPHAVEAL | | CGCTGTTTAACGAAGCGCTGCACCGTGACCTGGCGGATTT |
| | | VKQPAGRWLSKARMTHYQALLLDTDRVQFG | | CCGTATCCAGCACCCGGACCTGATCTTGCTGCAGTACGTT |
| | | PVVALNPATLLPLPEEGLQHNCLDILAEAH | | GATGACCTGTTGCTGGCGGCGACGAGCGAGCTGGATTGCC |
| | | GTRPDLTDQPLPDADHTWYTNGSSLLQEGQ | | AACAGGGCACCCGTGCGCTGTTGCAGACCTTGGGTAACCT |
| | | RKAGAAVTTETEVIWAKALPAGTSAQRAEL | | GGGTTATCGCGCTAGCGCGAAGAAAGCGCAGATTTGCCAA |
| | | IALTQALKMAEGKKLNVYTDSRYAFATAHI | | AAACAAGTTAAGTATCTGGGCTACCTGTTAAAGGAAGGCC |
| | | HGEIYRRRGWLTSKGKEIKNKDEILALLKA | | AACGTTGGCTGACCGAAGCCCGCAAAGAAACTGTCATGGG |
| | | LFLPKRLSIIHCPGHQKGHSAEARGNRMAD | | TCAGCCGACCCCGAAAACGCCACGCCAACTGCGTAGGTTC |
| | | QAARKAAITETPDTSTLLIENSSPNSRLIN | | TTGGGCAAAGCGGGTTTCTGCCGCCTGTTCATCCCGGGCT |
| | | GSGGSGAKTLAQGRKPGSGRKPGKGKTLRE | | TTGCCGAAATGGCAGCCCCGCTGTATCCGTTGACCAAGCC |
| | | GRKPGSGRRRRQDTGGKETDGSQQDQESRL | | GGGCACCCTGTTCAACTGGGGTCCGGACCAGCAGAAAGCG |
| | | ISSRDMEAVDALRELTHSPSSHSAHNSSAA | | TACCAAGAAATTAAACAAGCACTGCTGACGGCACCGGCGC |
| | | PPPHAAAASTSLPPSLDYTHQSFMDQQQQQ | | TGGGTCTGCCGGACCTGACCAAGCCGTTTGAGCTGTTCGT |
| | | QQQQQQQLLQQQRVDVVPPKPFITHKILLS | | GGATGAGAAGCAAGGTTACGCGAAGGGCGTGTTGACCCAG |
| | | STGNSGGHVNSNYNADHSINHNSNHNLNSN | | AAATTGGGTCCGTGGCGTCGTCCGGTTGCATACCTGTCCA |
| | | VNVNMNFTINGSNQDPSSSFLMGPYNYLQR | | AGAAACTGGACCCGGTTGCTGCTGGTTGGCCGCCTTGCCT |
| | | PFIVKPYLDLSTSTAASNQPRTQPSPAAHI | | GCGCATGGTTGCCGCTATCGCGGTGCTGACTAAAGACGCG |
| | | TKNSDSTEKNATI | | GGTAAGCTGACGATGGGTCAACCGCTGGTGATCGGCGCAC |
| | | | | CGCATGCAGTCGAGGCCCTTGTTAAGCAACCGGCAGGAAG |
| | | | | ATGGCTGAGCAAGGCGCGTATGACGCATTACCAGGCACTG |
| | | | | CTGTTGGACACCGATCGTGTGCAGTTTGGCCCGGTCGTTG |
| | | | | CGCTCAACCCGGCGACCCTGCTGCCGCTCCCGGAAGAAGG |
| | | | | CTTGCAGCACAACTGTTTGGACATCCTGGCAGAGGCGCAC |
| | | | | GGCACTCGCCCGGATCTGACGGACCAGCCGCTGCCGGACG |
| | | | | CCGATCATACCTGGTATACGAATGGTAGCAGCCTGTTGCA |
| | | | | AGAGGGTCAGCGTAAGGCCGGTGCCGCGGTCACCACCGAG |
| | | | | ACTGAAGTGATTTGGGCTAAAGCATTGCCTGCGGGTACCA |
| | | | | GCGCGCAGCGTGCCGAGCTGATCGCACTGACCCAAGCGCT |
| | | | | GAAAATGGCTGAGGGTAAGAAACTGAATGTGTACACGGAT |
| | | | | AGCCGTTATGCCTTTGCGACCGCCCACATTCACGGCGAGA |
| | | | | TCTATCGCCGTCGCGGCTGGCTGACGTCCAAAGGCAAAGA |
| | | | | GATCAAGAATAAAGACGAAATTCTGGCGCTGCTGAAAGCG |
| | | | | CTGTTCCTGCCGAAACGTCTGTCGATCATCCATTGCCCGG |
| | | | | GTCACCAGAAAGGCCACAGCGCAGAGGCGCGTGGTAATCG |
| | | | | CATGGCTGACCAGGCTGCGCGTAAAGCCGCAATTACCGAA |
| | | | | ACCCCGGACACCAGCACGCTGCTGATCGAGAATAGCAGCC |
| | | | | CGAACAGCCGTCTGATCAATGGTTCAGGGGGTTCCGGTGC |
| | | | | AAAAACTTTGGCTCAAGGGCGCAAACCTGGATCAGGCCGC |
| | | | | AAGCCGGGGAAGGGTAAAACTTTGCGCGAGGGGCGTAAGC |
| | | | | CTGGATCGGGGCGTCGTCGTCGTCAGGATACTGGAGGGAA |
| | | | | AGAAACTGACGGCTCCCAACAGGATCAAGAAAGCCGCTTG |
| | | | | ATTTCCTCCCGCGACATGGAAGCCGTCGATGCACTTCGTG |
| | | | | AGCTTACACATAGCCCGTCAAGCCATAGCGCCCATAATAG |
| | | | | CTCAGCCGCACCCCCACCACATGCCGCAGCGGCTAGCACT |
| | | | | TCCCTTCCACCATCGTTAGATTACACGCATCAATCGTTCA |
| | | | | TGGATCAGCAACAACAACAACAGCAACAGCAACAGCAGCA |
| | | | | GCTTTTACAGCAACAGCGTGTCGACGTTGTCCCTCCGAAG |
| | | | | CCCTTTATCACCCATAAGATCCTGTTGAGCTCTACTGGCA |
| | | | | ACTCAGGCGGGCACGTTAACAGCAACTACAACGCAGATCA |
| | | | | TTCGATCAACCATAATTCAAATCACAACCTTAATTCCAAC |
| | | | | GTTAACGTGAACATGAACTTTACGATTAACGGTAGCAATC |
| | | | | AAGACCCATCATCCAGTTTCTTGATGGGCCCATACAACTA |
| | | | | TTTACAACGCCCTTTCATCGTGAAGCCCTATTTGGATTTA |
| | | | | TCGACTTCTACGGCAGCCAGTAACCAACCGCGTACACAGC |
| | | | | CTTCCCCAGCGGCACATATCACAAAGAATAGCGATAGTAC |
| | | | | GGAGAAGAACGCCACAATTTGA |
| 185 | N-Dat1-TL- | AKTLAQGRKPGSGRKPGKGKTLREGRKPGS | | |
| | QID040 | GRRRRQDIGGRDADASQQDQESRPISSRDM | | |
| | 42BL | EAVDALRELTHSPSSHPAHNPTTAPPHVAG | | |
| | | SGGSGTWLSDFPQAWAETGGMGLAVRQAPL | | |
| | | IIPLKATSTPVSIKQYPLSQKARLGIKPHI | | |
| | | QRLLDQGILVPCQSPWNTPLLPVKKPGTND | | |
| | | YRPVQDLREVNKRVEDIHPTVPNPYNLLSG | | |
| | | PPPSHQWYTVLDLKDAFFCLRLHPTSQPLF | | |
| | | AFEWRDPEMGISGQLTWTRLPQGFKNSPTL | | |
| | | FNEALHRDLADFRIQHPDLILLQYVDDLLL | | |
| | | AATSELDCQQGTRALLQTLGNLGYRASAKK | | |
| | | AQICQKQVKYLGYLLKEGQRWLTEARKETV | | |
| | | MGQPTPKTPRQLRRFLGKAGFCRLFIPGFA | | |
| | | EMAAPLYPLTKPGTLFNWGPDQQKAYQEIK | | |
| | | QALLTAPALGLPDLTKPFELFVDEKQGYAK | | |
| | | GVLTQKLGPWRRPVAYLSKKLDPVAAGWPP | | |
| | | CLRMVAAIAVLTKDAGKLTMGQPLVIGAPH | | |
| | | AVEALVKQPAGRWLSKARMTHYQALLLDTD | | |
| | | RVQFGPVVALNPATLLPLPEEGLQHNCLDI | | |
| | | LAEAHGTRPDLTDQPLPDADHTWYTNGSSL | | |
| | | LQEGQRKAGAAVTTETEVIWAKALPAGTSA | | |
| | | QRAELIALTQALKMAEGKKLNVYTDSRYAF | | |
| | | ATAHIHGEIYRRRGWLTSKGKEIKNKDEIL | | |
| | | ALLKALFLPKRLSIIHCPGHQKGHSAEARG | | |
| | | NRMADQAARKAAITETPDTSTLLIENSSPN | | |
| | | SRLIN | | |
| 186 | N-Dat1-TL- | AKTLAQGRKPGSGRKPGKGKTLREGRKPGS | | |
| | XP361 | GRRRRLDEPDRAVRSKSQDAISSRDLEAVD | | |
| | 42BL | ALRELNNSPSGSGGSGTWLSDFPQAWAETG | | |
| | | GMGLAVRQAPLIIPLKATSTPVSIKQYPLS | | |
| | | QKARLGIKPHIQRLLDQGILVPCQSPWNTP | | |
| | | LLPVKKPGTNDYRPVQDLREVNKRVEDIHP | | |
| | | TVPNPYNLLSGPPPSHQWYTVLDLKDAFFC | | |
| | | LRLHPTSQPLFAFEWRDPEMGISGQLTWTR | | |
| | | LPQGFKNSPTLFNEALHRDLADFRIQHPDL | | |
| | | ILLQYVDDLLLAATSELDCQQGTRALLQTL | | |
| | | GNLGYRASAKKAQICQKQVKYLGYLLKEGQ | | |
| | | RWLTEARKETVMGQPTPKTPRQLRRFLGKA | | |
| | | GFCRLFIPGFAEMAAPLYPLTKPGTLFNWG | | |
| | | PDQQKAYQEIKQALLTAPALGLPDLTKPFE | | |
| | | LFVDEKQGYAKGVLTQKLGPWRRPVAYLSK | | |
| | | KLDPVAAGWPPCLRMVAAIAVLTKDAGKLT | | |
| | | MGQPLVIGAPHAVEALVKQPAGRWLSKARM | | |
| | | THYQALLLDTDRVQFGPVVALNPATLLPLP | | |
| | | EEGLQHNCLDILAEAHGTRPDLTDQPLPDA | | |
| | | DHTWYTNGSSLLQEGQRKAGAAVTTETEVI | | |
| | | WAKALPAGTSAQRAELIALTQALKMAEGKK | | |
| | | LNVYTDSRYAFATAHIHGEIYRRRGWLTSK | | |
| | | GKEIKNKDEILALLKALFLPKRLSIIHCPG | | |
| | | HQKGHSAEARGNRMADQAARKAAITETPDT | | |
| | | STLLIENSSPNSRLIN | | |
| 187 | RTx | AKTLAQGRKPGSGRKPGKGKTLREGRKPGS | | |
| | N-Dat1-TL- | GRRRRVDEEATKIQSSELHGIEISNECILG | | |
| | XP558 | SLQRNINNELSTRDMEAVDALRGLNTSPSG | | |
| | 42BL | SGGSGTWLSDFPQAWAETGGMGLAVRQAPL | | |
| | | IIPLKATSTPVSIKQYPLSQKARLGIKPHI | | |
| | | QRLLDQGILVPCQSPWNTPLLPVKKPGTND | | |
| | | YRPVQDLREVNKRVEDIHPTVPNPYNLLSG | | |
| | | PPPSHQWYTVLDLKDAFFCLRLHPTSQPLF | | |
| | | AFEWRDPEMGISGQLTWTRLPQGFKNSPTL | | |
| | | FNEALHRDLADFRIQHPDLILLQYVDDLLL | | |
| | | AATSELDCQQGTRALLQTLGNLGYRASAKK | | |
| | | AQICQKQVKYLGYLLKEGQRWLTEARKETV | | |
| | | MGQPTPKTPRQLRRFLGKAGFCRLFIPGFA | | |
| | | EMAAPLYPLTKPGTLFNWGPDQQKAYQEIK | | |
| | | QALLTAPALGLPDLTKPFELFVDEKQGYAK | | |
| | | GVLTQKLGPWRRPVAYLSKKLDPVAAGWPP | | |
| | | CLRMVAAIAVLTKDAGKLTMGQPLVIGAPH | | |
| | | AVEALVKQPAGRWLSKARMTHYQALLLDTD | | |
| | | RVQFGPVVALNPATLLPLPEEGLQHNCLDI | | |
| | | LAEAHGTRPDLTDQPLPDADHTWYTNGSSL | | |
| | | LQEGQRKAGAAVTTETEVIWAKALPAGTSA | | |
| | | QRAELIALTQALKMAEGKKLNVYTDSRYAF | | |
| | | ATAHIHGEIYRRRGWLTSKGKEIKNKDEIL | | |
| | | ALLKALFLPKRLSIIHCPGHQKGHSAEARG | | |
| | | NRMADQAARKAAITETPDTSTLLIENSSPN | | |
| | | SRLIN | | |
| 188 | RTx | AKTLAQGRKPGSGRKPGKGKTLREGRKPGS | | |
| | N-Dat1-TL- | GRKRRDGNISNENHLIHSNDTVTSTVNSSR | | |
| | XP683 | NGSVSNAFTEQDLEALQALNSLNKSSTSPR | | |
| | 42BL | STSANTSATNILPSGSGGSGTWLSDFPQAW | | |
| | N-Dat1-TL- | AETGGMGLAVRQAPLIIPLKATSTPVSIKQ | | |
| | XP683 | YPLSQKARLGIKPHIQRLLDQGILVPCQSP | | |
| | 42BL | WNTPLLPVKKPGTNDYRPVQDLREVNKRVE | | |
| | | DIHPTVPNPYNLLSGPPPSHQWYTVLDLKD | | |
| | | AFFCLRLHPTSQPLFAFEWRDPEMGISGQL | | |
| | | TWTRLPQGFKNSPTLFNEALHRDLADFRIQ | | |
| | | HPDLILLQYVDDLLLAATSELDCQQGTRAL | | |
| | | LQTLGNLGYRASAKKAQICQKQVKYLGYLL | | |
| | | KEGQRWLTEARKETVMGQPTPKTPRQLRRF | | |
| | | LGKAGFCRLFIPGFAEMAAPLYPLTKPGTL | | |
| | | FNWGPDQQKAYQEIKQALLTAPALGLPDLT | | |
| | | KPFELFVDEKQGYAKGVLTQKLGPWRRPVA | | |
| | | YLSKKLDPVAAGWPPCLRMVAAIAVLTKDA | | |
| | | GKLTMGQPLVIGAPHAVEALVKQPAGRWLS | | |
| | | KARMTHYQALLLDTDRVQFGPVVALNPATL | | |
| | | LPLPEEGLQHNCLDILAEAHGTRPDLTDQP | | |
| | | LPDADHTWYTNGSSLLQEGQRKAGAAVTTE | | |
| | | TEVIWAKALPAGTSAQRAELIALTQALKMA | | |
| | | EGKKLNVYTDSRYAFATAHIHGEIYRRRGW | | |
| | | LTSKGKEIKNKDEILALLKALFLPKRLSII | | |
| | | HCPGHQKGHSAEARGNRMADQAARKAAITE | | |
| | | TPDTSTLLIENSSPNSRLIN | | |

TABLE 5
Non-limiting embodiments of RT variants of the invention

| SEQ ID | | |
|---|---|---|
| NO: | Description | Sequences |
| 201 | GAPDH | aga ggg aaa gca gcc cgc agc cuc ccg cuu cgc ucu cug cuc cuc |
| | (RNA) | cug uuc gac agu cag ccg cau cuu cuu uug cgu |
| | Template | cgc cag ccg agc cac auc gcu cag aca cca ugg gga agg uga agg |
| | | ucg gag uca acg gau uug guc gua uug ggc gcc |
| | | ugg uca cca ggg cug cuu uua acu cug gua aag ugg aua uug uug |
| | | cca uca aug acc ccu uca uug acc uca acu aca |
| | | ugg uuu aca ugu ucc aau aug auu cca ccc aug gca aau ucc aug |
| | | gca ccg uca agg cug aga acg gga agc uug uca |
| | | uca aug gaa auc cca uca cca ucu ucc agg agc gag auc ccu cca |
| | | aaa uca agu ggg gcg aug cug gcg cug agu acg |
| | | ucg ugg agu cca cug gcg ucu uca cca cca ugg aga agg cug ggg |
| | | cuc auu ugc agg ggg gag cca aaa ggg uca uca |
| | | ucu cug ccc ccu cug cug aug ccc cca ugu ucg uca ugg gug uga |
| | | acc aug aga agu aug aca aca gcc uca aga uca |
| | | uca gca aug ccu ccu gca cca cca acu gcu uag cac ccc ugg cca |
| | | agg uca ucc aug aca acu uug gua ucg ugg aag |
| | | gac uca uga cca cag ucc aug cca uca cug cca ccc aga aga cug |
| | | ugg aug gcc ccu ccg gga aac ugu ggc gug aug |
| | | gcc gcg ggg cuc ucc aga aca uca ucc cug ccu cua cug gcg cug |
| | | cca agg cug ugg gca agg uca ucc cug agc uga |
| | | acg gga agc uca cug gca ugg ccu ucc gug ucc cca cug cca acg |
| | | ugu cag ugg ugg acc uga ccu gcc guc uag aaa |
| | | aac cug cca aau aug aug aca uca aga agg ugg uga agc agg cgu |
| | | cgg agg gcc ccc uca agg gca ucc ugg gcu aca |
| | | cug agc acc agg ugg ucu ccu cug acu uca aca gcg aca ccc acu |
| | | ccu cca ccu uug acg cug ggg cug gca uug ccc |
| | | uca acg acc acu uug uca agc uca uuu ccu ggu aug aca acg aau |
| | | uug gcu aca gca aca ggg ugg ugg acc uca ugg |
| | | ccc aca ugg ccu cca agg agu aag acc ccu gga cca cca gcc cca |
| | | gca aga gca caa gag gaa gag aga gac ccu cac ugc |
| | | ugg gga guc ccu gcc aca cuc agu ccc cca cca cac uga auc ucc |
| | | ccu ccu cac agu ugc cau gua gac ccc uug aag |
| | | agg gga ggg gcc uag gga gcc gca ccu ugu cau gua cca uca aua |
| | | aag uac ccu gug cuc aac c |
| 202 | GAPDH | /56-FAM/tgg ttg agc aca ggg tac ttt att gat |
| | Primer-56 | gg |
| | FAM | |
| 203 | TSO1 | aag cag tgg tat caa cgc aga gta cat rgrgrg |
| | Oligo | |
| 204 | GAPDH | TGG TTG AGC ACA GGG TAC TTT ATT GAT GG |
| | Primer | |
| 205 | 5x His tag | His His His His His |
| | amino | |
| | acid | |
| 206 | Wild Type | TLNIEDEHRLHETSKEPDVSLGSTWLSDFPQAWAETGGMGLAVRQAPLIIPLKATSTPVSIKQYPMSQEARLGI |
| | MMLV | KPHIQRLLDQGILVPCQSPWNTPLLPVKKPGTNDYRPVQDLREVNKRVEDIHPTVPNPYNLLSGLPPSHQWYT |
| | Identical | VLDLKDAFFCLRLHPTSQPLFAFEWRDPEMGISGQLTWTRLPQGFKNSPTLFDEALHRDLADFRIQHPDLILLQ |
| | to SEQ ID | YVDDLLLAATSELDCQQGTRALLQTLGNLGYRASAKKAQICQKQVKYLGYLLKEGQRWLTEARKETVMGQP |
| | NO: 7 | TPKTPRQLREFLGTAGFCRLWIPGFAEMAAPLYPLTKTGTLFNWGPDQQKAYQEIKQALLTAPALGLPDLTKP |
| | | FELFVDEKQGYAKGVLTQKLGPWRRPVAYLSKKLDPVAAGWPPCLRMVAAIAVLTKDAGKLTMGQPLVILA |
| | | PHAVEALVKQPPDRWLSNARMTHYQALLLDTDRVQFGPVVALNPATLLPLPEEGLQHNCLDILAEAHGTRPD |
| | | LTDQPLPDADHTWYTDGSSLLQEGQRKAGAAVTTETEVIWAKALPAGTSAQRAELIALTQALKMAEGKKLN |
| | | VYTDSRYAFATAHIHGEIYRRRGLLTSEGKEIKNKDEILALLKALFLPKRLSIIHCPGHQKGHSAEARGNRMAD |
| | | QAARKAAITETPDTSTLL |
| 207 | MMLV | MTWLSDFPQAWAETGGMGLAVRQAPLIIPLKATSTPVSIKQYPMSQKARLGIKPHIQRLLDQGILVPCQSPWN |
| | RT | TPLLPVKKPGTNDYRPVQDLREVNKRVEDIHPTVPNPYNLLSGPPPSHQWYTVLDLKDAFFCLRLHPTSQPLF |
| | Variant | AFEWRDPEMGISGQLTWTRLPQGFKNSPTLFNEALHRDLADFRIQHPDLILLQYVDDLLLAATSELDCQQGTR |
| | (42B) | ALLQTLGNLGYRASAKKAQICQKQVKYLGYLLKEGQRWLTEARKETVMGQPTPKTPRQLRRFLGKAGFCRL |
| | Identical | FIPGFAEMAAPLYPLTKPGTLFNWGPDQQKAYQEIKQALLTAPALGLPDLTKPFELFVDEKQGYAKGVLTQKL |
| | to SEQ ID | GPWRRPVAYLSKKLDPVAAGWPPCLRMVAAIAVLTKDAGKLTMGQPLVIGAPHAVEALVKQPAGRWLSKA |
| | NO: 1 | RMTHYQALLLDTDRVQFGPVVALNPATLLPLPEEGLQHNCLDILAEAHGTRPDLTDQPLPDADHTWYTNGSS |
| | | LLQEGQRKAGAAVTTETEVIWAKALPAGTSAQRAELIALTQALKMAEGKKLNVYTDSRYAFATAHIHGEIYR |
| | | RRGWLTSKGKEIKNKDEILALLKALFLPKRLSIIHCPGHQKGHSAEARGNRMADQAARKAAITETPDTSTLLIE |
| | | NSSPNSRLIN |
| 208 | MMLV | MTWLSDFPQAWAETGGMGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPPDRWLSKARM |
| | | THYQALLLDTDRVQFGPVVALNPATLLPLPEEGLQHNCLDILAEAVGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIYCPGHQKGHSAEARGNRMADQAARKAAITETPDTSTLLIENSSP |
| | | NSRLIN |
| 209 | MMLV | MTWLSDFPQAWAETGGVGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIKAPHAVEALVKQPPDRWLSKARM |
| | | THYQALLLDTDRVQFGPVVALNPATLLPLPEEGLQHNCLDILAEAVGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIYCPGHQKGHSAEARGNRMADQAARKAAITETPDTSTLLIENSSP |
| | | NSRLIN |
| 210 | MMLV | MTWLSDFPQAWAETGGVGLAVRQAPLIIPLKATSTPVSIKQYPMSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIKAPHAVEALVKQPPDRWLSKARM |
| | | THYQALLLDTDRVQFGPVVALNPATLLPLPEEGLQHNCLDILAEAVGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIYCPGHQKGHSAEARGNRMADQAARKAAITETPDTSTLLIENSSP |
| | | NSRLIN* |
| 211 | MMLV | MTWLSDFPQAWAETGGMGLAVRQAPLIIPLKATSTPVSIKQYPMSQKARLGIKPHIQRLLDQGILVPCQSPWN |
| | RT | TPLLPVKKPGTNDYRPVQDLREVNKRVEDIHPTVPNPYNLLSGPPPSHQWYTVLDLKDAFFCLRLHPTSQPLF |
| | Variant | AFEWRDPEMGISGQLTWTRLPQGFKNSPTLFNEALHRDLADFRIQHPDLILLQYVDDLLLAATSELDCQQGTR |
| | | ALLQTLGNLGYRASAKKAQICQKQVKYLGYLLKEGQRWLTEARKETVMGQPTPKTPRQLRRFLGKAGFCRL |
| | | FIPGFAEMAAPLYPLTKPGTLFNWGPDQQKAYQEIKQALLTAPALGLPDLTKPFELFVDEKQGYAKGVLTQKL |
| | | GPWRRPVAYLSKKLDPVAAGWPPCLRMVAAIAVLTKDAGKLTMGQPLVIKAPHAVEALVKQPPDRWLSKA |
| | | RMTHYQALLLDTDRVQFGPVVALNPATLLPLPEEGLQHNCLDILAEAVGTRPDLTDQPLPDADHTWYTNGSS |
| | | LLQEGQRKAGAAVTTETEVIWAKALPAGTSAQRAELIALTQALKMAEGKKLNVYTDSRYAFATAHIHGEIYR |
| | | RRGWLTSKGKEIKNKDEILALLKALFLPKRLSIIYCPGHQKGHSAEARGNRMADQAARKAAITETPDTSTLLIE |
| | | NSSPNSRLIN* |
| 212 | MMLV | MTWLSDFPQAWAETGGMGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPAGRWLSKARM |
| | | THYQALLLDTDRVQFGPVVALNPATLLPLPEEGLQHNCLDILAEAVGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIYCPGHQKGHSAEARGNRMADQAARKAAITETPDTSTLLIENSSP |
| | | NSRLIN* |
| 213 | MMLV | MTWLSDFPQAWAETGGVGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPAGRWLSKARM |
| | | THYQALLLDTDRVQFGPVVALNPATLLPLPEEGLQHNCLDILAEAVGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIYCPGHQKGHSAEARGNRMADQAARKAAITETPDTSTLLIENSSP |
| | | NSRLIN* |
| 214 | MMLV | TLNIEDEHRLHETSKEPDVSLGSTWLSDFPQAWAETGGVGLAVRQAPLIIPLKATSTPVSIKQYPMSQKARLGI |
| | RT | KPHIQRLLDQGILVPCQSPWNTPLLPVKKPGTNDYRPVQDLREVNKRVEDIHPTVPNPYNLLSGPPPSHQWYT |
| | Variant | VLDLKDAFFCLRLHPTSQPLFAFEWRDPEMGISGQLTWTRLPQGFKNSPTLFNEALHRDLADFRIQHPDLILLQ |
| | | YVDDLLLAATSELDCQQGTRALLQTLGNLGYRASAKKAQICQKQVKYLGYLLKEGQRWLTEARKETVMGQP |
| | | TPKTPRQLRRFLGKAGFCRLFIPGFAEMAAPLYPLTKPGTLFNWGPDQQKAYQEIKQALLTAPALGLPDLTKPF |
| | | ELFVDEKQGYAKGVLTQKLGPWRRPVAYLSKKLDPVAAGWPPCLRMVAAIAVLTKDAGKLTMSQPLVIKAP |
| | | HAVEALVKQPAGRWLSKARMTHYQALLLDTDRVQFGPVVALNPATLLPLPEEGLQHNCLDILAEAHGTRPDL |
| | | TDQPLPDADHTWYTNGSSLLQEGQRKAGAAVTDETEVIWAKALPAGTSAQRAELIALTQALKMAEGKKLNV |
| | | YTNSRYAFATAHIHGEIYRRRGWLTSGGKEIKNKDEILALLKALFLSKRLSIIHCPGHQKGHSVEARGNRMAH |
| | | QAARRAAITETPDTSTLP |
| 215 | MMLV | TLNIEDEHRLHETSKEPDVSLGSTWLSDFPQAWAETGGVGLAVRQAPLIIPLKATSTPVSIKQYPMSQKARLGI |
| | RT | KPHIQRLLDQGILVPCQSPWNTPLLPVKKPGTNDYRPVQDLREVNKRVEDIHPTVPNPYNLLSGPPPSHQWYT |
| | Variant | VLDLKDAFFCLRLHPTSQPLFAFEWRDPEMGISGQLTWTRLPQGFKNSPTLFNEALHRDLADFRIQHPDLILLQ |
| | | YVDDLLLAATSELDCQQGTRALLQTLGNLGYRASAKKAQICQKQVKYLGYLLKEGQRWLTEARKETVMGQP |
| | | TPKTPRQLRRFLGKAGFCRLFIPGFAEMAAPLYPLTKPGTLFNWGPDQQKAYQEIKQALLTAPALGLPDLTKPF |
| | | ELFVDEKQGYAKGVLTQKLGPWRRPVAYLSKKLDPVAAGWPPCLRMVAAIAVLTKDAGKLTMSQPLVIKAP |
| | | HAVEALVKQPAERWLSKARMTHYQALLLDTDRVQFGPVVALNPATLLPLPEEGLQHNCLDILAEAHGTRPDL |
| | | TDQPLPDADHTWYTNGSSLLQEGQRKAGAAVTDETEVIWAKALPAGTSAQRAELIALTQALKMAEGKKLNV |
| | | YTDSRYAFATAHIHGEIYRRRGWLTSGGKEIKNKDEILALLKALFLPKRLSIIHCPGHQKGHSVEARGNHMAH |
| | | QAARRAAITETPDTSTLP |
| 216 | MMLV | TLNIEDEHRLHETSKEPDVSLGSTWLSDFPQAWAETGGVGLAVRQAPLIIPLKATSTPVSIKQYPMSQKARLGI |
| | RT | KPHIQRLLDQGILVPCQSPWNTPLLPVKKPGTNDYRPVQDLREVNKRVEDIHPTVPNPYNLLSGPPPSHQWYT |
| | Variant | VLDLKDAFFCLRLHPTSQPLFAFEWRDPEMGISGQLTWTRLPQGFKNSPTLFNEALHRDLADFRIQHPDLILLQ |
| | | YVDDLLLAATSELDCQQGTRALLQTLGNLGYRASAKKAQICQKQVKYLGYLLKEGQRWLTEARKETVMGQP |
| | | TPKTPRQLRRFLGKAGFCRLFIPGFAEMAAPLYPLTKPGTLFNWGPDQQKAYQEIKQALLTAPALGLPDLTKPF |
| | | ELFVDEKQGYAKGVLTQKLGPWRRPVAYLSKKLDPVAAGWPPCLRMVAAIAVLTKDAGKLTMSQPLVIKAP |
| | | HAVEALVKQPAGRWLSKARMTHYQALLLDTDRVQFGPVVALNPATLLPLPEEGLQHNCLDILAEAHGTRPDL |
| | | TDQPLPDADHTWYTDGSSLLQEGQRKAGAAVTTETGVIWAKALPAGTSAQRAELIALTQALKMAEGKKLNV |
| | | YTNSRYAFATAHIQGEIYRRRGFLTSKGKEIKNKDEILALLKALFLPKRLSIIHCPGHQKGHSAEARGNRMADQ |
| | | AARKAAITETPDTSTLP |
| 217 | MMLV | TLNIEDEHRLHETSKEPDVSLGSTWLSDFPQAWAETGGVGLAVRQAPLIIPLKATSTPVSIKQYPMSQKARLGI |
| | RT | KPHIQRLLDQGILVPCQSPWNTPLLPVKKPGTNDYRPVQDLREVNKRVEDIHPTVPNPYNLLSGPPPSHQWYT |
| | Variant | VLDLKDAFFCLRLHPTSQPLFAFEWRDPEMGISGQLTWTRLPQGFKNSPTLFNEALHRDLADFRIQHPDLILLQ |
| | | YVDDLLLAATSELDCQQGTRALLQTLGNLGYRASAKKAQICQKQVKYLGYLLKEGQRWLTEARKETVMGQP |
| | | TPKTPRQLRRFLGKAGFCRLFIPGFAEMAAPLYPLTKPGTLFNWGPDQQKAYQEIKQALLTAPALGLPDLTKPF |
| | | ELFVDEKQGYAKGVLTQKLGPWRRPVAYLSKKLDPVAAGWPPCLRMVAAIAVLTKDAGKLTMSQPLVIKAP |
| | | HAVEALVKQPAERWLSKARMTHYQALLLDTDRVQFGPVVALNPATLLPLPEEGLQHNCLDILAEAHGTRPDL |
| | | TDQPLPDADHTWYTNGSSLLQEGQRKAGAAVTDETEVIWAKALPAGTSAQRAELIALTQALKMAEGKKLNV |
| | | YTDSRYAFATAHIHGEIYRRRGWLTSGGKEIKNKDEILALLKALFLPKRLSIIHCPGHQKGHSVEARGNRMAH |
| | | QAARRAAITETPDTSTLP |
| 218 | MMLV | TLNIEDEHRLHETSKEPDVSLGSTWLSDFPQAWAETGGVGLAVRQAPLIIPLKATSTPVSIKQYPMSQKARLGI |
| | RT | KPHIQRLLDQGILVPCQSPWNTPLLPVKKPGTNDYRPVQDLREVNKRVEDIHPTVPNPYNLLSGPPPSHQWYT |
| | Variant | VLDLKDAFFCLRLHPTSQPLFAFEWRDPEMGISGQLTWTRLPQGFKNSPTLFEEALRRDLADFRIQHPDLILLQ |
| | | YVDDLLLAATSELDCQQGTRALLQTLGNLGYRASAKKAQICQKQVKYLGYLLKEGQRWLTEARKETVMGQP |
| | | TPKTPRQLRRFLGKAGFCRLFIPGFAEMAAPLYPLTKPGTLFNWGPDQQKAYQEIKQALLTAPALGLPDLTKPF |
| | | ELFVDEKQGYAKGVLTQKLGPWRRPVAYLSKKLDPVAAGWPPCLRMVAAIAVLTKDAGKLTMSQPLVIKAP |
| | | HAVEALVKQPAGRWLSKARMTHYQALLLDTDRVQFGPVVALNPATLLPLPEEGLQHNCLDILAEAHGTRPDL |
| | | TDQPLPDADHTWYTNGSSLLQEGQRKAGAAVTDETEVIWAKALPAGTSAQRAELIALTQALKMAEGKKLNV |
| | | YTNSRYAFATAHIHGEIYRRRGWLTSGGKEIKNKDEILALLKALFLSKRLSIIHCPGHQKGHSVEARGNRMAH |
| | | QAARRAAITETPDTSTLP |
| 219 | MMLV | TLNIEDEHRLHETSKEPDVSLGSTWLSDFPQAWAETGGVGLAVRQAPLIIPLKATSTPVSIKQYPMSQKARLGI |
| | RT | KPHIQRLLDQGILVPCQSPWNTPLLPVKKPGTNDYRPVQDLREVNKRVEDIHPTVPNPYNLLSGPPPSHQWYT |
| | Variant | VLDLKDAFFCLRLHPTSQPLFAFEWRDPEMGISGQLTWTRLPQGFKNSPTLFEEALRRDLADFRIQHPDLILLQ |
| | | YVDDLLLAATSELDCQQGTRALLQTLGNLGYRASAKKAQICQKQVKYLGYLLKEGQRWLTEARKETVMGQP |
| | | TPKTPRQLRRFLGKAGFCRLFIPGFAEMAAPLYPLTKPGTLFNWGPDQQKAYQEIKQALLTAPALGLPDLTKPF |
| | | ELFVDEKQGYAKGVLTQKLGPWRRPVAYLSKKLDPVAAGWPPCLRMVAAIAVLTKDAGKLTMSQPLVIKAP |
| | | HAVEALVKQPAGRWLSKARMTHYQALLLDTDRVQFGPVVALNPATLLPLPEEGLQHNCLDILAEAHGTRPDL |
| | | TDQPLPDADHTWYTDGSSLLQEGQRKAGAAVTTETGVIWAKALPAGTSAQRAELIALTQALKMAEGKKLNV |
| | | YTNSRYAFATAHIQGEIYRRRGFLTSKGKEIKNKDEILALLKALFLPKRLSIIHCPGHQKGHSAEARGNRMADQ |
| | | AARKAAITETPDTSTLP |
| 220 | MMLV | TLNIEDEHRLHETSKEPDVSLGSTWLSDFPQAWAETGGVGLAVRQALLIIPLKATSTPVSIKQYPMSQKARLGI |
| | RT | KPHIQRLLDQGILVPCQSPWNTPLLPVKKPGTNDYRPVQDLREVNKRVEDIHPTVPNPYNLLSGPPPSHQWYT |
| | Variant | VLDLKDAFFCLRLHPTSQPLFAFEWRDPEMGISGQLTWTRLPQGFKNSPTLFNEALHRDLADFRIQHPDLILLQ |
| | | YVDDLLLAATSELDCQQGTRALLQTLGNLGYRASAKKAQICQKQVKYLGYLLKEGQRWLTEARKETVMGQP |
| | | TPKTPRQLRRFLGKAGFCRLFIPGFAEMAAPLYPLTKPGTLFNWGPDQQKAYQEIKQALLTAPALGLPDLTKPF |
| | | ELFVDEKQGYAKGVLTQKLGPWRRPVAYLSKKLDPVAAGWPPCLRMVAAIAVLTKDAGKLTMSQPLVIKAP |
| | | HAVEALVKQPAGRWLSKARMTHYQALLLDTDRVQFGPVVALNPATLLPLPEEGLQHNCLDILAEAHGTRPDL |
| | | TDQPLPDADHTWYTNGSSLLQEGQRKAGAAVTDETEVIWAKALPAGTSAQRAELIALTQALKMAEGKKLNV |
| | | YTNSRYAFATAHIHGEIYRRRGWLTSGGKEIKNKDEILALLKALFLSKRLSIIHCPGHQKGHSVEARGNRMAHQ |
| | | AARRAAITETPDTSTLP |
| 221 | MMLV | MTWLSDFPQAWAETGGMGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPAGRWLSKARM |
| | | THYQALLLDTDRVQFGPVVALNPATLLPLPEEGLQHNCLDILAEAHGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIHCPGHQKGHSAEARGNRMADQAARKAAITETPDTSTLLIENSSP |
| | | NSRLIN |
| 222 | MMLV | MTWLSDFPQAWAETGGVGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPAGRWLSKARM |
| | | THYQALLLDTDRVQFGPVVALNPATLLPLPEEGLQHNCLDILAEAHGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIHCPGHQKGHSAEARGNRMADQAARKAAITETPDTSTLLIENSSP |
| | | NSRLIN |
| 223 | MMLV | MTWLSDFPQAWAETGGMGLAVRQAPLIIPLKATSTPVSIKQYPMSQKARLGIKPHIQRLLDQGILVPCQSPWN |
| | RT | TPLLPVKKPGTNDYRPVQDLREVNKRVEDIHPTVPNPYNLLSGPPPSHQWYTVLDLKDAFFCLRLHPTSQPLF |
| | Variant | AFEWRDPEMGISGQLTWTRLPQGFKNSPTLFNEALHRDLADFRIQHPDLILLQYVDDLLLAATSELDCQQGTR |
| | | ALLQTLGNLGYRASAKKAQICQKQVKYLGYLLKEGQRWLTEARKETVMGQPTPKTPRQLRRFLGKAGFCRL |
| | | FIPGFAEMAAPLYPLTKPGTLFNWGPDQQKAYQEIKQALLTAPALGLPDLTKPFELFVDEKQGYAKGVLTQKL |
| | | GPWRRPVAYLSKKLDPVAAGWPPCLRMVAAIAVLTKDAGKLTMGQPLVIGAPHAVEALVKQPPDRWLSKA |
| | | RMTHYQALLLDTDRVQFGPVVALNPATLLPLPEEGLQHNCLDILAEAVGTRPDLTDQPLPDADHTWYTNGSS |
| | | LLQEGQRKAGAAVTTETEVIWAKALPAGTSAQRAELIALTQALKMAEGKKLNVYTDSRYAFATAHIHGEIYR |
| | | RRGWLTSKGKEIKNKDEILALLKALFLPKRLSIIYCPGHQKGHSAEARGNRMADQAARKAAITETPDTSTLLIE |
| | | NSSPNSRLIN |
| 224 | MMLV | MTWLSDFPQAWAETGGMGLAVRQAPLIIPLKATSTPVSIKQYPMSQKARLGIKPHIQRLLDQGILVPCQSPWN |
| | RT | TPLLPVKKPGTNDYRPVQDLREVNKRVEDIHPTVPNPYNLLSGPPPSHQWYTVLDLKDAYFCLRLHPTSQPLF |
| | Variant | AFEWRDPEMGISGQLTWTRLPQGFKNSPTLFNEALHRDLADFRIQHPDLILLQYVDDLLLAATSELDCQQGTR |
| | | ALLQTLGNLGYRASAKKAQICQKQVKYLGYLLKEGQRWLTEARKETVMGQPTPKTPRQLRRFLGKAGFCRL |
| | | FIPGFAEMAAPLYPLTKPGTLFNWGPDQQKAYQEIKQALLTAPALGLPDLTKPFELFVDEKQGYAKGVLTQKL |
| | | GPWRRPVAYLSKKLDPVAAGWPPCLRMVAAIAVLTKDAGKLTMGQPLVIGAPHAVEALVKQPAGRWLSKA |
| | | RMTHYQALLLDTDRVQFGPVVALNPATLLPLPEEGLQHNCLDILAEAHGTRPDLTDQPLPDADHTWYTNGSS |
| | | LLQEGQRKAGAAVTTETEVIWAKALPAGTSAQRAELIALTQALKMAEGKKLNVYTDSRYAFATAHIHGEIYR |
| | | RRGWLTSKGKEIKNKDEILALLKALFLPKRLSIIHCPGHQKGHSAEARGNRMADQAARKAAITETPDTSTLLIE |
| | | NSSPNSRLIN |
| 225 | MMLV | MTWLSDFPQAWAETGGVGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPPDRWLSKARM |
| | | THYQALLLDTDRVQFGPVVALNPATLLPLPEEGLQHNCLDILAEAVGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIYCPGHQKGHSAEARGNRMADQAARKAAITETPDTSTLLIENSSP |
| | | NSRLIN |
| 226 | MMLV | MTWLSDFPQAWAETGGMGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPADRWLSKARM |
| | | THYQALLLDTDRVQFGPVVALNPATLLPLPEEGLQHNCLDILAEAHGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIHCPGHQKGHSAEARGNRMADQAARKAAITETPDTSTLLIENSSP |
| | | NSRLIN |
| 227 | MMLV | MTWLSDFPQAWAETGGMGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPPGRWLSKARM |
| | | THYQALLLDTDRVQFGPVVALNPATLLPLPEEGLQHNCLDILAEAHGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIHCPGHQKGHSAEARGNRMADQAARKAAITETPDTSTLLIENSSP |
| | | NSRLIN |
| 228 | MMLV | MTWLSDFPQAWAETGGMGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPPDRWLSKARM |
| | | THYQALLLDTDRVQFGPVVALNPATLLPLPEEGLQHNCLDILAEAVGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIHCPGHQKGHSAEARGNRMADQAARKAAITETPDTSTLLIENSSP |
| | | NSRLIN |
| 229 | MMLV | MTWLSDFPQAWAETGGMGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPPDRWLSKARM |
| | | THYQALLLDTDRVQFGPVVALNPATLLPLPEEGLQHNCLDILAEAHGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIYCPGHQKGHSAEARGNRMADQAARKAAITETPDTSTLLIENSSP |
| | | NSRLIN |
| 230 | MMLV | MTWLSDFPQAWAETGGMGLAVRQAPLIIPLKATSTPVSIKQYPMSQKARLGIKPHIQRLLDQGILVPCQSPWN |
| | RT | TPLLPVKKPGTNDYRPVQDLREVNKRVEDIHPTVPNPYNLLSGPPPSHQWYTVLDLKDAFFCLRLHPTSQPLF |
| | Variant | AFEWRDPEMGISGQLTWTRLPQGFKNSPTLFNEALHRDLADFRIQHPDLILLQYVDDLLLAATSELDCQQGTR |
| | | ALLQTLGNLGYRASAKKAQICQKQVKYLGYLLKEGQRWLTEARKETVMGQPTPKTPRQLRRFLGKAGFCRL |
| | | FIPGFAEMAAPLYPLTKPGTLFNWGPDQQKAYQEIKQALLTAPALGLPDLTKPFELFVDEKQGYAKGVLTQKL |
| | | GPWRRPVAYLSKKLDPVAAGWPPCLRMVAAIAVLTKDAGKLTMGQPLVIGAPHAVEALVKQPPDRWLSKA |
| | | RMTHYQALLLDTDRVQFGPVVALNPATLLPLPEEGLQHNCLDILAEAHGTRPDLTDQPLPDADHTWYTNGSS |
| | | LLQEGQRKAGAAVTTETEVIWAKALPAGTSAQRAELIALTQALKMAEGKKLNVYTDSRYAFATAHIHGEIYR |
| | | RRGWLTSKGKEIKNKDEILALLKALFLPKRLSIIHCPGHQKGHSAEARGNRMADQAARKAAITETPDTSTLLIE |
| | | NSSPNSRLIN |
| 231 | MMLV | MTWLSDFPQAWAETGGMGLAVRQAPLIIPLKATSTPVSIKQYPMSQKARLGIKPHIQRLLDQGILVPCQSPWN |
| | RT | TPLLPVKKPGTNDYRPVQDLREVNKRVEDIHPTVPNPYNLLSGPPPSHQWYTVLDLKDAYFCLRLHPTSQPLF |
| | Variant | AFEWRDPEMGISGQLTWTRLPQGFKNSPTLFNEALHRDLADFRIQHPDLILLQYVDDLLLAATSELDCQQGTR |
| | | ALLQTLGNLGYRASAKKAQICQKQVKYLGYLLKEGQRWLTEARKETVMGQPTPKTPRQLRRFLGKAGFCRL |
| | | FIPGFAEMAAPLYPLTKPGTLFNWGPDQQKAYQEIKQALLTAPALGLPDLTKPFELFVDEKQGYAKGVLTQKL |
| | | GPWRRPVAYLSKKLDPVAAGWPPCLRMVAAIAVLTKDAGKLTMGQPLVIGAPHAVEALVKQPPDRWLSKA |
| | | RMTHYQALLLDTDRVQFGPVVALNPATLLPLPEEGLQHNCLDILAEAHGTRPDLTDQPLPDADHTWYTNGSS |
| | | LLQEGQRKAGAAVTTETEVIWAKALPAGTSAQRAELIALTQALKMAEGKKLNVYTDSRYAFATAHIHGEIYR |
| | | RRGWLTSKGKEIKNKDEILALLKALFLPKRLSIIHCPGHQKGHSAEARGNRMADQAARKAAITETPDTSTLLIE |
| | | NSSPNSRLIN |
| 232 | MMLV | MTWLSDFPQAWAETGGMGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPPDRWLSKARM |
| | | THYQALLLDTDRVQFGPVVALNPATLLPLPEEGLQHNCLDILAEAHGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIHCPGGQKGHSAEARGNRMADQAARKAAITETPDTSTLLIENSSP |
| | | NSRLIN |
| 233 | MMLV | MTWLSDFPQAWAETGGMGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAYFCLRLHPTSQPLFA |
| | Variant | FEWRDPEMGISGQLTWTRLPQGFKNSPTLFNEALHRDLADFRIQHPDLILLQYVDDLLLAATSELDCQQGTRA |
| | | LLQTLGNLGYRASAKKAQICQKQVKYLGYLLKEGQRWLTEARKETVMGQPTPKTPRQLRRFLGKAGFCRLFI |
| | | PGFAEMAAPLYPLTKPGTLFNWGPDQQKAYQEIKQALLTAPALGLPDLTKPFELFVDEKQGYAKGVLTQKLG |
| | | PWRRPVAYLSKKLDPVAAGWPPCLRMVAAIAVLTKDAGKLTMGQPLVIGAPHAVEALVKQPPDRWLSKAR |
| | | MTHYQALLLDTDRVQFGPVVALNPATLLPLPEEGLQHNCLDILAEAHGTRPDLTDQPLPDADHTWYTNGSSL |
| | | LQEGQRKAGAAVTTETEVIWAKALPAGTSAQRAELIALTQALKMAEGKKLNVYTDSRYAFATAHIHGEIYRR |
| | | RGWLTSKGKEIKNKDEILALLKALFLPKRLSIIHCPGGQKGHSAEARGNRMADQAARKAAITETPDTSTLLIEN |
| | | SSPNSRLIN |
| 234 | MMLV | MTWLSDFPQAWAETGGVGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPPDRWLSKARM |
| | | THYQALLLDTDRVQFGPVVALNPATLLPLPEEGLQHNCLDILAEAHGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIHCPGHQKGHSAEARGNRMADQAARKAAITETPDTSTLLIENSSP |
| | | NSRLIN |
| 235 | MMLV | MTWLSDFPQAWAETGGMGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAYFCLRLHPTSQPLFA |
| | Variant | FEWRDPEMGISGQLTWTRLPQGFKNSPTLFNEALHRDLADFRIQHPDLILLQYVDDLLLAATSELDCQQGTRA |
| | | LLQTLGNLGYRASAKKAQICQKQVKYLGYLLKEGQRWLTEARKETVMGQPTPKTPRQLRRFLGKAGFCRLFI |
| | | PGFAEMAAPLYPLTKPGTLFNWGPDQQKAYQEIKQALLTAPALGLPDLTKPFELFVDEKQGYAKGVLTQKLG |
| | | PWRRPVAYLSKKLDPVAAGWPPCLRMVAAIAVLTKDAGKLTMGQPLVIGAPHAVEALVKQPPDRWLSKAR |
| | | MTHYQALLLDTDRVQFGPVVALNPATLLPLPEEGLQHNCLDILAEAHGTRPDLTDQPLPDADHTWYTNGSSL |
| | | LQEGQRKAGAAVTTETEVIWAKALPAGTSAQRAELIALTQALKMAEGKKLNVYTDSRYAFATAHIHGEIYRR |
| | | RGWLTSKGKEIKNKDEILALLKALFLPKRLSIIHCPGHQKGHSAEARGNRMADQAARKAAITETPDTSTLLIEN |
| | | SSPNSRLIN |
| 236 | MMLV | MTWLSDFPQAWAETGGMGLAVRQAPLIIPLKATSTPVSIKQYPLSQKARLGIKPHIQRLLDQGILVPCQSPWNT |
| | RT | PLLPVKKPGTNDYRPVQDLREVNKRVEDIHPTVPNPYNLLSGPPPSHQWYTVLDLKDAFFCLRLHPTSQPLFAF |
| | Variant | EWRDPEMGISGQLTWTRLPQGFKNSPTLFNEALHRDLADFRIQHPDLILLQYVDDLLLAATSELDCQQGTRAL |
| | | LQTLGNLGYRASAKKAQICQKQVKYLGYLLKEGQRWLTEARKETVMGQPTPKTPRQLRRFLGKAGFCRLFIP |
| | | GFAEMAAPLYPLTKPGTLFNWGPDQQKAYQEIKQALLTAPALGLPDLTKPFELFVDEKQGYAKGVLTQKLGP |
| | | WRRPVAYLSKKLDPVAAGWPPCLRMVAAIAVLTKDAGKLTMGQPLVIGAPHAVEALVKQPPDRWLSKARM |
| | | THYQALLLDTDRVQFGPVVALNPATLLPLPEEGLQHNCLDILAEAHGTRPDLTDQPLPDADHTWYTNGSSLLQ |
| | | EGQRKAGAAVTTETEVIWAKALPAGTSAQRAELIALTQALKMAEGKKLNVYTDSRYAFATAHIHGEIYRRRG |
| | | WLTSKGKEIKNKDEILALLKALFLPKRLSIIHCPGHQKGHSAEARGNRMADQAARKAAITETPDTSTLLIENSSP |
| | | NSRLIN |

What is claimed is:

1. An engineered reverse transcriptase (RT) polypeptide comprising:
(a) an RT polypeptide;
(b) a DNA binding domain,
wherein the DNA binding domain is from a molecule capable of binding a minor groove of a nucleic acid; and
(c) a linker connecting the RT polypeptide and the DNA binding domain.

2. The engineered RT polypeptide of claim 1, wherein the DNA binding domain is located at the N-terminus of the RT polypeptide.

3. The engineered RT polypeptide of claim 1, wherein the DNA binding domain is located at the C-terminus of the RT polypeptide sequence.

4. The engineered RT polypeptide of any one of claims 1-3, wherein the linker is:
(a) a glycine-serine linker selected from the group consisting of (GS)ₙ, (GSGGS)ₙ, (SGGSG)ₙ, (GGGS)ₙ, (GGSG)ₙ, (GGSGG)ₙ, (GSGSG)ₙ, (GSGGG)ₙ, GGGSG)ₙ, and (GSSSG)ₙ, and wherein n represents an integer of at least 1; or
(b)
GGGS;
or
(c)
SGGSG.

5. The engineered RT polypeptide of any one of claims 1-4, wherein the DNA binding domain specifically recognizes adenine-thymine-rich region on a nucleic acid molecule.

6. The engineered RT polypeptide of claim 5, wherein the DNA binding domain specifically recognizes oligo(dA) or oligo(dT) tracts on a nucleic acid molecule.

7. The engineered RT polypeptide of any one of claims 1-6, wherein the DNA binding domain comprises at least one AT-rich interaction domain.

8. The engineered RT polypeptide of claim 7, wherein the DNA binding domain comprises at least 2, at least 3, at least 4, at least 5, at least 6, at least 7, at least 8, at least 9, or at least 10 AT-rich interaction domains.

9. The engineered RT polypeptide of 7 or 8, wherein the AT-rich interaction domain comprises a core sequence, wherein the core sequence is a two base core sequence, a three base core sequence, a four base core sequence, or a five base core sequence.

10. The engineered RT polypeptide of claim 9, wherein at least one of the bases of the core sequence comprises an arginine; a glycine and an arginine; a proline and an arginine; a lysine and an arginine; or any combination thereof.

11. The engineered RT polypeptide of any one of claims 7-10, wherein the AT-rich interaction domain comprises a GRKPG (Gly-Arg-Lys-Pro-Gly) repeat, a RKRGRPKK repeat, a KKRGRPKK repeat, a RKRGR repeat, a GR*R/PPK repeat, a GR*RPK repeat, a GR*PPK repeat, a KRPR* repeat, or a K/RKRGRPKK repeat.

12. The engineered RT polypeptide of any one of claims 7-11, wherein the AT-rich interaction domain comprises a core sequence comprising an amino acid selected from the group consisting of SEQ ID NO: 11-24.

13. The engineered RT polypeptide of any one of claims 1-12, wherein the DNA binding domain is a DNA binding domain of any one of Saccharomyces cerevisiae datin (DAT1), high mobility group AT hook 1 (HMGA1), lysine-specific methyltransferase 2a (KMT2A), Myocyte Enhancer Factor 2C (MEF2C), Heterogeneous Nuclear Ribonucleoprotein D (HNRNPD), Structural Maintenance of Chromosomes 1A (SMC1), Structural Maintenance Of Chromosomes 2 (SMC2), Caenorhabditis elegans tbp-1, Drosophila melanogaster D1 protein, Salmonella typhimurium Hin recombinase, S. typhimurium Gin recombinase, S. typhimurium Pin recombinase, or S. typhimurium Cin recombinase, or a combination thereof.

14. The engineered RT polypeptide of any one of claims 1-13, wherein the DNA binding domain is from a S. cerevisiae DAT1.

15. The engineered RT polypeptide of any one of claims 1-14, wherein the amino acid sequence of the DNA binding domain comprises a DNA binding domain consensus motif set forth in SEQ ID NO: 13, 14, 16, or 22.

16. The engineered RT polypeptide of any one of claims 3-15, wherein the DNA binding domain comprises:
(a) a full-length DAT1 sequence or SEQ ID NO: 2;
(b) an N-terminal truncated variant of DAT1 (D90) comprising the first 90 amino acids of the full length DAT1, or SEQ ID NO: 3;
(c) a truncated variant of DAT1 (D60) comprising the first 60 amino acids of the full length DAT1 or SEQ ID NO: 5;
(d) a truncated variant of DAT1 (D48) comprising the first 48 amino acids of the full length DAT1 or SEQ ID NO: 6;
(e) a truncated variant of DAT1 (D36) comprising the first 36 amino acids of the full length DAT1 or SEQ ID NO: 8;
(f) a truncated variant of DAT1 (D35) comprising the first 35 amino acids of full length DAT1 or SEQ ID NO: 9;
(g) an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 2, 3, 5, 6, 8, or 9; or
(h) an amino acid sequence having 90%, 91%, 92%, 93%, 94%, 95%, 96%, 97%, 98%, or 99% sequence identity to any one of SEQ ID NO: 2, 3, 5, 6, 8, or 9.

17. The engineered RT polypeptide of claim 16, wherein the DNA binding domain comprises the amino acid sequence of SEQ ID NO: 11 (GRKPG); optionally wherein the DNA binding domain comprise at least 2 domains or at least 3 domains comprising SEQ ID NO: 11.

18. The engineered RT of claim 16, wherein the DNA binding domain comprises a mutation in any of one of SEQ ID NO: 2, 3, 5, 6, 8, 9, or 11, and wherein the mutation is selected from a substitution, an insertion, a deletion, or any combination thereof.

19. The engineered RT polypeptide of claim 16, wherein the DNA binding domain comprises SEQ ID NO: 2.

20. The engineered RT polypeptide of claim 16, wherein the DNA binding domain comprises the amino acid sequence of SEQ ID NO: 3, 8, or 9.

21. The engineered RT polypeptide of any one of claims 1-20, wherein the RT polypeptide sequence comprises the amino acid sequence of SEQ ID NO: 7, and
further comprises a combination of mutations selected from the group consisting of:
(i) E69K, L139P, E302R, T306K, W313F, T330P, and N454K; and additionally one or more of M39V, P47L, Q91R, M66L, F155Y, D200N, D200E, H204R, G429S, L435G, L435K, P448A, D449G, H503V, D524N, T542D, E545G, D583N, H594Q, L603W, L603F, E607K, E607G, P627S, H634Y, H638G, A644V, D653H, K658R and L671P; and
(ii) E69K, L139P, D200N, E302R, T306K, W313F, T330P, L435G, P448A, D449G, N454K, D524N, L603W, and E607K; and additionally one or more of M39V, P47L, M66L, Q91R, F155Y, H204R, G429S, H503V, T542D, E545G, D583N, H594Q, P627S, H634Y, H638G, A644V, D653H, K658R and L671P.

22. The engineered RT polypeptide of any one of claims 1-21, wherein the amino acid sequence of the RT polypeptide sequence is:
(a) at least 90% identical to SEQ ID NO: 1 or 143;
(b) about 90% to about 99.99% identical to SEQ ID NO: 1 or 143, about 92% to about 99.99% identical to SEQ ID NO: 1 or 143, about 93% to about 99.99% identical to SEQ ID NO: 1 or 143, about 94% to about 99.99% identical to SEQ ID NO: 1 or 143, about 95% to about 99.99% identical to SEQ ID NO: 1 or 143, about 96% to about 99.99% identical to SEQ ID NO: 1 or 143, about 97% to about 99.99% identical to SEQ ID NO: 1 or 143, or about 98% to about 99.99% identical to SEQ ID NO: 1 or 143; or
(c) about 90%, about 91%, about 92%, about 93%, about 94%, about 95%, about 96%, about 97%, about 98%, about 99% or about 99.5% identical to SEQ ID NO: 1 or 143.

23. The engineered RT polypeptide of claim of any one of claims 1-22, wherein the RT polypeptide sequence comprises:
(a) an amino acid sequence that is at least 95% identical to SEQ ID NO:1, 7, or 179, and;
(b) a combination of mutations indexed to SEQ ID NO:7 or 178 selected from the group consisting of:
(i) a combination of variants consisting of a T542D mutation, a D583N mutation, an E607G mutation, an A644V mutation, a D653H mutation, and a K658R mutation; and
(ii) a combination of variants consisting of an E545G mutation, a D583N mutation, an H594Q mutation, an L603F mutation, and a S679P mutation.

24. The engineered RT polypeptide of any one of claims 1-23, wherein the amino acid sequence of the RT polypeptide sequence comprises E69K, L139P, D200N, E302R, T306K, W313F, T330P, N454K, H503V, D524N, L603W, E607K, and H634Y.

25. The engineered RT polypeptide of any one of claims 1-24, wherein the amino acid variation(s) are at any one position or combination thereof as identified in an alignment of SEQ ID NO: 1 or 143 to any one of the RT polypeptide sequences in Table 1 or Table 2.

26. The engineered RT of any one of claims 1-25, wherein the RT polypeptide sequence comprises:
(a) M39V, M66I, Q91R, I347V, and H594Q substitution in SEQ ID NO: 143; or
(b) SEQ ID NO: 129 (SOLD 034).

27. The engineered RT polypeptide of any one of claims 1-26, wherein the RT polypeptide sequence comprises M39V, T542D, D583N, E607G, A644V, D653H, K658R, and L671P in SEQ ID NO: 143.

28. The engineered RT polypeptide of any one of claims 1-27, wherein the RT polypeptide sequence comprises:
(a) M39V, T542D, D583N, E607G, A644V, D653H, K658R, L671P in SEQ ID NO: 143; or
(b) SEQ ID NO: 111 (SOLD 025).

29. The engineered RT polypeptide of any one of claims 1-28, wherein the RT polypeptide sequence comprises T542D, D583N, E607G, A644V, D653H, K658R, E545G, D583N, H594Q, and a L603F in SEQ ID NO: 143.

30. An engineered RT polypeptide comprising:
(a) an amino acid sequence that is at least 90%, at least 92%, at least 95%, at least 97%, at least 98%, or at least 99% identical to:
(i) an amino acid sequence of an RT disclosed in Table 1, or Table 2; or
(ii) SEQ ID NOs: 27-61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, 172, or 173; and
(b) a DNA binding domain comprising an amino acid selected from the group consisting of SEQ ID NO: 2, 3, 5, 6, 8, and 9.

31. An engineered RT polypeptide comprising:
(a) an amino acid sequence of an RT disclosed in Table 1 or Table 2; and
(b) an amino acid sequence of DNA binding domain disclosed in Table 1.

32. The engineered RT polypeptide of any one of claims 1-31, wherein the engineered RT polypeptide comprises:
(a) the amino acid sequence of any one of SEQ ID NO: 174-188;
(b) an amino acid sequence having at least 90%, at least 91%, at least 92%, at least 93%, at least 94%, at least 95%, at least 96%, at least 97%, at least 98%, or at least 99% sequence identity to any one of SEQ ID NO: SEQ ID NO: 174-188; or
(c) an amino acid sequence having 90%, 91%, 92%, 93%, 94%, 95%, 96%, 97%, 98%, or 99% sequence identity to any one of SEQ ID NO: SEQ ID NO: 174-188.

33. The engineered RT polypeptide of any one of claims 1-32, wherein the engineered RT comprises an amino acid sequence that is at least about 90% identical to an amino acid sequence selected from the group consisting of SEQ ID NO: 27-61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, 172, and 173.

34. The engineered RT polypeptide of any one of claims 1-33, wherein the RT polypeptide is 42B L (SEQ ID NO: 145), 50A+G (SEQ ID NO: 147), SOLD 022 (SEQ ID NO: 105), SOLD 023 (SEQ ID NO: 107), SOLD 025 (SEQ ID NO: 111), SOLD 031 (SEQ ID NO: 123, SOLD 033 (SEQ ID NO: 127), SOLD 034 (SEQ ID NO: 129), SOLD 035 (SEQ ID NO: 131), SOLD 001 (SEQ ID NO: 65), and SOLD 33 VDG (SEQ ID NO: 173), or an RT polypeptide set forth in SEQ ID NO: 143, or SEQ ID NO: 172.

35. The engineered RT polypeptide of any one of claims 1-34, wherein the engineered RT comprises at least two DNA binding domains.

36. The engineered RT polypeptide of claim 35, wherein at least one DNA binding domain is located at the N-terminus of the engineered RT and at least one DNA binding domain is located at the C-terminus of the engineered RT.

37. The engineered RT polypeptide of claim 36, wherein the at least two DNA binding domains are both located at the C-terminus or N-terminus of the engineered RT.

38. A recombinant reverse transcriptase (RT) protein comprising a RT polypeptide, fused to a DNA binding domain, wherein:
(a) the RT polypeptide and the DNA binding domain are separated by an amino acid linker, and
(b) the DNA binding domain is fused to the C-terminus of the RT polypeptide.

39. A recombinant reverse transcriptase (RT) protein comprising a RT polypeptide, fused to a DNA binding domain, wherein:
(a) the RT polypeptide and the DNA binding domain are separated by an amino acid linker, and
(b) the DNA binding domain is fused to the N-terminus of the RT polypeptide.

40. The recombinant RT protein of claim 38 or 39, wherein the RT polypeptide is any one of the RT polypeptides listed in Table 1 or Table 2.

41. The recombinant RT protein of any one of claims 38-40, wherein the DNA binding domain is a DNA binding protein selected from the group consisting of S. cerevisiae datin (DAT1); high mobility group AT hook 1 (HMGA1), lysine-specific methyltransferase 2a (KMT2A), Myocyte Enhancer Factor 2C (MEF2C), Heterogeneous Nuclear Ribonucleoprotein D (HNRNPD), Structural Maintenance of Chromosomes 1A (SMC1), Structural Maintenance Of Chromosomes 2 (SMC2), C. elegans tbp-1, D. melanogaster D1 protein, Salmonella typhimurium Hin recombinase, S. typhimurium Gin recombinase, S. typhimurium Pin recombinase, or S. typhimurium Cin recombinase.

42. The recombinant RT protein of any one of claims 38-41, wherein the linker:
(a) a glycine-serine linker selected from the group consisting of (GS)ₙ, (GSGGS)ₙ, (SGGSG)ₙ, (GGGS)ₙ, (GGSG)ₙ, (GGSGG)ₙ, (GSGSG)ₙ, (GSGGG)ₙ, GGGSG)ₙ, and (GSSSG)ₙ, and wherein n represents an integer of at least 1; or
(b) GGGS; or
(c) SGGSG.

43. The recombinant RT protein of any one of claims 38-42, wherein the DNA binding domain is a S. cerevisiae datin (DAT1) DNA binding domain or fragment thereof.

44. The recombinant RT protein of any one of claims 38-43, wherein the DNA binding domain comprises:
(a) an amino acid sequence selected from the group consisting of SEQ ID NO: 2, 3, 5, 6, 8, 9, and 11-24; or
(b) a nucleic acid sequence of SEQ ID NO: 25.

45. The recombinant RT protein of any one of claims 38-44, wherein the RT polypeptide is selected from the group consisting of 42B L (SEQ ID NO: 145), 50A+G (SEQ ID NO: 147), SOLD 022 (SEQ ID NO: 105), SOLD 023 (SEQ ID NO: 107), SOLD 025 (SEQ ID NO: 111), SOLD 031 (SEQ ID NO: 123), SOLD 033 (SEQ ID NO: 127), SOLD 034 (SEQ ID NO: 129), SOLD 035 (SEQ ID NO: 131), SOLD 001 (SEQ ID NO: 65), SOLD 33 VDG (SEQ ID NO: 173), and an RT polypeptide set forth in SEQ ID NO: 143, SEQ ID NO: 172.

46. The recombinant RT protein of any one of claims 38-45 comprising, consisting essentially of, or consisting of SEQ ID NO: 174-188.

47. The engineered RT polypeptide of any one of claims 1-37, or the recombinant RT protein of any one of claims 38-46, wherein the engineered RT polypeptide or the recombinant RT protein further comprises a tag protein selected from the group consisting of an affinity tag, a fluorescent tag, or an expression and/or solubility enhancement tag.

48. The engineered RT polypeptide or the recombinant RT protein of claim 47, wherein the tag is selected from hexahistidine tag (his-tag), small ubiquitin-like modifier tag (SUMO), a VariFlex C-Terminal solubility enhancement tag, a short peptide C-terminal tag, Thioredoxin (Trx) tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

49. The engineered RT polypeptide or the recombinant RT protein of claim 47, wherein the tag is an affinity tag selected from hexahistidine tag (his-tag), Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

50. The engineered RT polypeptide or the recombinant RT protein of claim 47, wherein the engineered RT polypeptide or the recombinant RT protein comprises:
(a) an hexahistidine tag (his-tag); or
(b) an amino acid sequence of SEQ ID NO: 62; or an amino acid sequence having at least 90% sequence identity to the amino acid sequence of SEQ ID NO: 62.

51. The engineered RT polypeptide or the recombinant RT protein of claim 47, wherein the engineered RT polypeptide or the recombinant RT protein comprises a solubility enhancer tag selected from the group consisting of a SUMO tag, a GST tag, a Trx tag, a VariFlex C-Terminal solubility enhancement tag, a short peptide C-terminal tag, an Fh8 tag, MBP tag, SET tag, GB1 tag, ZZ tag, HaloTag, SNUT tag, Skp tag, T7PK tag, EspA tag, Mocr tag, Ecotin tag, CaBO tag, ArsC tag, IF2-domain I tag, Expressivity tag, RpoA, tag, SlyD, tag, Tsf tag, RpoS tag, PotD tag, Crr tag, msyB tag, yigD tag, and rpoD tag.

52. The engineered RT polypeptide or the recombinant RT protein of claim 47, wherein the engineered RT polypeptide or the recombinant RT protein comprises:
(a) a short peptide C-terminal tag;
(b) an amino acid sequence of SEQ ID NO: 193; or
(c) an amino acid sequence having at least 90% sequence identity to the amino acid sequence of SEQ ID NO: 193.

53. The engineered RT polypeptide or the recombinant RT protein of claim 47, wherein the tag further comprises:
(a) an endoprotein cleavage sequence;
(b) a cleavage sequence recognized by an endoprotein selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase (EnTK), gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin (Thr), tissue kallikrein, tobacco etch virus (TEV), togavirin, tryptophanyl aminopeptidase, U-plasminogen activator, V8, venombin A, venombin AB, factor Xa (Xa), and Xaa-pro aminopeptidase; or
(c) an endoprotein cleavage sequence comprising the amino acid sequence of SEQ ID NO: 194, SEQ ID NO: 195, SEQ ID NO: 196, SEQ ID NO: 197, or SEQ ID NO: 198.

54. The engineered RT polypeptide of any one of claim 1-37 or 47-53, or the recombinant RT protein of any one of claims 38-52, wherein the engineered RT polypeptide or the recombinant RT protein exhibits increased template switching (TS) efficiency, increased processivity efficiency, increased binding affinity, increased transcription efficiency, increased chemical tolerance, improved ability to yield mitochondrial unique molecular identity (UMI) counts, improved ability to yield ribosomal unique molecular identity (UMI) counts, longer shelf life, higher strand displacement, higher end-to-end template jumping, or any combination thereof, when compared to an RT polypeptide or a recombinant RT protein lacking a conjugated DNA binding domain.

55. The engineered RT polypeptide of any one of claim 1-37 or 47-53, or the recombinant RT protein of any one of claims 38-53, wherein the engineered RT polypeptide or the recombinant RT protein comprises at least two or more of increased template switching (TS) efficiency, increased processivity efficiency, increased binding affinity, increased transcription efficiency, increased chemical tolerance, improved ability to yield mitochondrial unique molecular identity (UMI) counts, longer shelf life, higher strand displacement, higher end-to-end template jumping, or improved ability to yield ribosomal unique molecular identity (UMI) counts, when compared to an RT polypeptide or a recombinant RT protein lacking a conjugated DNA binding domain.

56. The engineered RT polypeptide of any one of claims 1-37 and 47-53 or the recombinant RT protein of any one of claims 38-53, wherein the recombinant RT protein or the engineered RT exhibits increased transcript capture during amplification, when compared to an RT polypeptide or a recombinant RT protein lacking a conjugated DNA binding domain.

57. The engineered RT polypeptide or the recombinant RT protein of claim 56, wherein the DNA binding domain enhances the hybridization of a transcript and a primer during a nucleic acid amplification process, when compared to an RT polypeptide or a recombinant RT protein lacking a conjugated DNA binding domain.

58. The engineered RT polypeptide or the recombinant RT protein of claim 56 or 57, wherein the primer comprises a poly-dT or a poly(dT)VN sequence and a non-poly(dT) sequence; and the transcript comprises a poly-dA sequence.

59. The engineered RT polypeptide or the recombinant RT protein of any one of claims 56-58, wherein the DNA binding domain stabilizes the oligo(A)-oligo(T) based transcript-primer complex during a nucleic acid amplification process.

60. The engineered RT polypeptide or the recombinant RT protein of any one of claims 56-59, wherein the primer is a barcoded molecule.

61. The engineered RT polypeptide or the recombinant RT protein of any one of claims 56-60, wherein the transcript is a nucleic acid molecule selected from a RNA, a mRNA, or a DNA.

62. An isolated nucleic acid molecule encoding:
(a) the engineered RT polypeptide of any one of claim 1-37, or 47-61; or
(b) the recombinant RT protein of any one of claims 38-61.

63. The isolated nucleic acid molecule of claim 62, wherein the nucleic acid molecule comprises a sequence selected from SEQ ID NO: 25, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO:150, SEQ ID NO:152, SEQ ID NO:154, SEQ ID NO:156, SEQ ID NO:158, SEQ ID NO: 160, SEQ ID NO:162, SEQ ID NO: 164, SEQ ID NO: 167, SEQ ID NO: 169, or SEQ ID NO: 171; or a nucleic acid sequence of Table 2.

64. An expression vector comprising the isolated nucleic acid of claim 62 or 63.

65. A host cell transfected with the expression vector of claim 64 or the isolated nucleic acid of claim 62 or 63.

66. A composition comprising:
(a) the recombinant RT protein of any one of claims 38-61; or
(b) the engineered RT polypeptide of any one of claim 1-37 or 47-61; or
(d) an expression vector of claim 64; or
(e) a host cell of claim 65; and
(f) a buffer.

67. A method for performing a reverse transcription reaction for generating a nucleic acid product from an RNA template comprising contacting under suitable conditions a biological sample or extract thereof with an engineered RT polypeptide of any of claim 1-37 or 47-61, or the recombinant RT protein of any one of claims 38-61.

68. The method of claim 67, wherein the biological sample comprises a cell, optionally wherein the cell is permeabilized and/or optionally wherein the cell is fixed.

69. The method of claim 67, wherein the biological sample comprises a cell bead, optionally wherein the cell bead is fixed.

70. The method of claim 67, wherein the biological sample comprises a nucleus, optionally wherein the nucleus is permeabilized and optionally wherein the nucleus is fixed.

71. The method of claim 67, wherein the biological sample comprises (a) a suitable cellular preparation selected from cell populations and/or single cells, or
(b) a tissue.

72. The method of claim 71, wherein the sample comprises cells in suspension, fresh cells, fixed cells, or cells and tissues immobilized on various solid surfaces.

73. The method of claim 67, wherein the biological sample is a cell, a cell bead, or a nucleus, and the reverse transcription reaction is part of a single cell RNA sequencing assay.

74. The method of claim 73, wherein the single cell RNA sequencing assay further comprises, prior to the reverse transcription, partitioning the cell, cell bead, or nucleus into a partition.

75. The method of claim 74, wherein the single cell RNA sequencing assay further comprises, after the reverse transcription reaction, hybridizing the nucleic acid product to an oligonucleotide molecule comprising a partition-specific barcode.

76. The method of claim 67, wherein the biological sample is a cell or tissue sample immobilized on a surface, and the reverse transcription reaction is part of a spatial RNA sequencing assay.

77. The method of any one of claims 67-76, wherein the engineered RT polypeptide or the recombinant RT protein enhances template switching (TS) efficiency, processivity efficiency, binding affinity, transcription efficiency, chemical tolerance, ability to yield mitochondrial unique molecular identity (UMI) counts, ability to yield ribosomal unique molecular identity (UMI) counts, strand displacement, end-to-end template jumping, or any combination thereof, when compared to an RT polypeptide or a recombinant RT protein lacking a conjugated DNA binding domain.

78. The method of any one of claims 67-76, wherein the engineered RT polypeptide or the recombinant RT protein enhances at least two or more of template switching (TS) efficiency, processivity efficiency, binding affinity, transcription efficiency, chemical tolerance, ability to yield mitochondrial unique molecular identity (UMI) counts, strand displacement, end-to-end template jumping, or ability to yield ribosomal unique molecular identity (UMI) counts, when compared to an RT polypeptide or a recombinant RT protein lacking a conjugated DNA binding domain.

79. The method of any one of claims 67-76, wherein the recombinant RT protein or the engineered RT enhances transcript capture during amplification, when compared to an RT polypeptide or a recombinant RT protein lacking a conjugated DNA binding domain.

80. The method of any one of claims 67-79, wherein the DNA binding domain of the recombinant RT protein or the engineered RT enhances the hybridization of a transcript and a primer during the amplification process, when compared to an RT polypeptide or a recombinant RT protein lacking a conjugated DNA binding domain.

81. The method of any one of claims 67-80, wherein: the engineered RT polypeptide or the recombinant RT protein comprises:
(a) a DNA binding domain comprising an amino acid sequence selected from SEQ ID NO: 2, 3, 5, 6, 8, 9, or 11-24; and
(b) an amino acid sequence selected from SEQ ID NOs: 27-61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, 172, or 173.

82. The method of any one of claims 67-81, wherein the amino acid sequence of the engineered RT polypeptide or the recombinant RT protein comprises an amino acid sequence having at least about 90%, at least about 92%, at least about 95%, at least about 97%, at least about 98%, or at least about 99% sequence identity to SEQ ID NO: 174-188.

83. The method of any one of claims 67-82, wherein the engineered RT polypeptide or the recombinant RT protein comprises:
(a) M39V, M66I, Q91R, I347V, and H594Q substitution in SEQ ID NO: 143;
(b) SEQ ID NO: 129 (SOLD 034);
(c) SOLD 001 (SEQ ID NO: 65); or
(d) SOLD 33 VDG (SEQ ID NO: 173).

84. The method of any one of claims 67-83, wherein the engineered RT polypeptide or the recombinant RT protein comprises M39V, T542D, D583N, E607G, A644V, D653H, K658R, and L671P in SEQ ID NO: 1 or 143.

85. The method of any one of claims 67-84, wherein the engineered RT polypeptide or the recombinant RT protein comprises:
(a) M39V, T542D, D583N, E607G, A644V, D653H, K658R, L671P in SEQ ID NO: 143; or
(b) SEQ ID NO: 111 (SOLD 025).

86. The method of any one of claims 67-85, wherein the engineered RT or the recombinant RT protein comprises a M39V, M66I, Q91R, I347V, H594Q in SEQ ID NO: 143.

87. The method of any one of claims 67-86, wherein the engineered RT polypeptide or the recombinant RT protein comprises an amino acid sequence that is at least about 90%, at least about 92%, at least about 95%, at least about 97%, at least about 98%, or at least about 99% identical to an amino acid sequence disclosed in Table 1 or Table 2.

88. A method of using the engineered RT polypeptide of any one of claim 1-37 or 47-61, or the recombinant RT protein of any one of claims 38-61, the method comprising contacting the engineered RT polypeptide or the recombinant RT protein with a nucleic acid template under suitable conditions to produce a polymerized nucleic acid product, wherein the nucleic acid template comprises an RNA

89. A nucleic acid extension method comprising:
(a) contacting a target nucleic acid molecule with an engineered reverse transcriptase polypeptide or a recombinant RT protein and a plurality of nucleic acid barcoded molecules comprising a barcode sequence, and
(b) incubating the target nucleic acid, the engineered RT polypeptide or the recombinant RT protein and barcoded molecules under suitable conditions in which the barcoded molecules are extended by the engineered RT polypeptide or the recombinant RT protein, wherein the engineered RT polypeptide comprises the amino acid sequence of an engineered RT polypeptide of any one of claim 1-37 or 47-61, or a recombinant RT protein of any one of claims 38-61.

90. The method of any one of claims 67-89, wherein the recombinant RT protein or the engineered RT polypeptide exhibits increased transcript capture during amplification.

91. The method of any one of claims 67-90, wherein the DNA binding domain enhances the hybridization of a transcript and a primer during a nucleic acid amplification process.

92. The method of any one of claims 67-91, wherein the primer comprises a poly-dT or a poly(dT)VN sequence and a non-poly(dT) sequence; and the transcript comprises a poly-dA sequence.

93. The method of any one of claims 67-92, wherein the DNA binding domain stabilizes the oligo(A)-oligo(T) based transcript-primer complex during a nucleic acid amplification process.

94. The method of any one of claims 67-93, wherein the primer is a barcoded molecule.

95. The method of any one of claims 67-94, wherein the recombinant RT protein or the engineered RT polypeptide performs the first strand complementary DNA (cDNA) reaction.

96. The method of claim 95, wherein the first strand cDNA is amplified using a DNA polymerase to generate a second strand cDNA.

97. A kit comprising:
(a) a recombinant RT protein of any one of claims 38-61; or
(b) an engineered reverse transcriptase polypeptide of any one of claim 1-37 or 47-61; or
(c) the isolated nucleic acid of claim 62 or 63; or
(d) an expression vector of claim 64; or
(e) a host cell of claim 65; or
(f) the composition of claim 66; and
(g) instructions.
